(19) United States
(12) Patent Application Publication
(10) Pub. No.: US 2023/0398181 A1
(43) Pub. Date: Dec. 14, 2023

(54) TREATMENTS FOR COMPLICATIONS ASSOCIATED WITH CHRONIC LIVER DISEASE

(71) Applicant: Acceleron Pharma Inc., Cambridge, MA (US)

(72) Inventors: Rajasekhar Naga Venkata Sai Suragani, Wrentham, MA (US); Patrick Andre, Short Hills, NJ (US)

(73) Assignee: Acceleron Pharma Inc., Cambridge, MA (US)

(21) Appl. No.: 18/328,302

(22) Filed: Jun. 2, 2023

Related U.S. Application Data
(60) Provisional application No. 63/350,197, filed on Jun. 8, 2022.

(51) Int. Cl. A61K 38/17; A61P 11/00

(52) U.S. Cl. CPC A61K 38/179; A61P 11/00

(57) ABSTRACT

In certain aspects, the present disclosure relates to methods of treating chronic liver disease, portal hypertension, primary sclerosing cholangitis, and hepatopulmonary syndrome, particularly associated clinical complications such as, for example, pulmonary fibrosis and/or interstitial lung disease (ILD), methods comprising administering TβRII antagonists comprising a heterologous domain and a truncated, ligand-binding portion of the extracellular domain of TβRII polypeptide useful to selectively antagonize a TβRII ligand. The disclosure further provides methods for treating one or more complications associated with chronic liver disease, including interstitial lung disease (ILD) and pulmonary fibrosis, with TβRII antagonists of the present disclosure.

### CROSS-REFERENCE TO RELATED APPLICATIONS

This application claims the benefit of priority from U.S. Provisional Application No. 63/350,197, filed Jun. 8, 2022. The foregoing application is incorporated herein by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on May 8, 2023, is named 25519-US-NP.XML and is 139,264 bytes in size on disk.

### BACKGROUND OF THE INVENTION

Members of the transforming growth factor-beta (TGFβ) superfamily are pleiotropic cytokines involved in essential cellular functions such as proliferation, differentiation, apoptosis, motility, extracellular matrix production, tissue remodeling, angiogenesis, immune response, cell adhesion, and also play a key role in pathophysiology of disease states as different as chronic inflammatory conditions and cancer. Members of the TGFβ superfamily have been classified into major family groupings, which include TGFβs, bone morphogenetic proteins (BMP), osteogenic proteins (OP), growth and differentiation factors (GDF), inhibins/activins, mullerian inhibitory substances (MIS) and glial derived neurotrophic factors (GDNF).

TGFβ superfamily members transduce their signals across the plasma membrane by inducing the formation of heteromeric complexes of specific type I and type II serine/threonine kinase receptors, which in turn activate a particular subset of SMAD proteins (some inhibitory and some excitatory). The SMAD molecule compounds relay the signals into the nucleus where they direct transcriptional responses in concert with other proteins.

Dysfunctional TGFβ superfamily signaling has been linked to several clinical disorders including cancer, fibrosis, bone diseases, diabetic nephropathy, as well as chronic vascular diseases such as atherosclerosis. Recent studies have identified a high population (approximately 20%) of patients with liver disease (particularly hepatopulmonary syndrome) who have concurrent interstitial lung disease (ILD). The presence of liver disease limits the diagnostic process of ILD, and available treatments for ILD would be contraindicated due to the presence of liver disease. Treatment options for liver disease, particularly those concurrent with lung complications such as pulmonary fibrosis or ILD, are limited to liver transplant. Therefore, a need exists to develop new treatments for liver disease, particularly chronic liver disease with concurrent pulmonary fibrosis or ILD.

### SUMMARY OF THE INVENTION

In part, the disclosure relates to TβRII antagonists that can be used to treat chronic liver disease. In particular, the disclosure relates to TβRII antagonists that can be used to treat disorders associated with chronic liver disease, such as portal hypertension, primary sclerosing cholangitis, and hepatopulmonary syndrome. The disclosure also relates to TβRII antagonists that can be used to treat clinical complications associated with chronic liver disease, portal hypertension, primary sclerosing cholangitis, and hepatopulmonary syndrome, particularly clinical complications such as, for example, pulmonary fibrosis or interstitial lung disease (ILD). Accordingly, the disclosure provides methods for treating chronic liver disease, portal hypertension, primary sclerosing cholangitis, and hepatopulmonary syndrome, or a complication thereof (e.g., pulmonary fibrosis, ILD) comprising administering to a subject in need thereof one or more TβRII antagonists as described herein.

In some embodiments, the disclosure provides a method for treating hepatopulmonary syndrome (HPS), comprising administering to a subject in need thereof a transforming growth factor β receptor II (TβRII) antagonist. In some embodiments, the subject has one or more complications of the lungs associated with HPS. In some embodiments, the one or more complications of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, the one or more complications of the lungs is interstitial lung disease (ILD), pulmonary fibrosis, or idiopathic pulmonary fibrosis. In some embodiments, the subject has HPS and pulmonary fibrosis. In some embodiments, the subject has HPS and interstitial lung disease (ILD). In some embodiments, the subject has HPS and idiopathic pulmonary fibrosis. In some embodiments, the subject has HPS and a short telomere syndrome. In some embodiments, the subject has dyspnea. In some embodiments, the subject has platypnea-orthodeoxia syndrome. In some embodiments, the subject has type I or type II hepatopulmonary syndrome. In some embodiments, the subject has type I hepatopulmonary syndrome. In some embodiments, the subject has type II hepatopulmonary syndrome.

In some embodiments, the disclosure provides a method for treating chronic liver disease, comprising administering to a subject in need thereof a transforming growth factor β receptor II (TβRII) antagonist. In some embodiments, the chronic liver disease is selected from the group consisting of: primary sclerosing cholangitis, portal hypertension, cryptogenic cirrhosis, postnecrotic cirrhosis, alcoholic cirrhosis, autoimmune cirrhosis, primary biliary cirrhosis, primary biliary cholangitis, chronic active hepatitis, alpha-1 antitrypsin deficiency, cystic fibrosis, nonalcoholic fatty liver disease, galactosemia, alagille syndrome, autoimmune hepatitis, Wilson's disease, sarcoidosis, hemochromatosis, biliary atresia, portal hypertension, tyrosinemia, Gaucher disease, schistosomiasis, nodular regenerative hyperplasia of the liver, hepatic allograft rejection, Langerhans cell histiocytosis, hepatic graft-versus host disease following hematopoietic stem cell transplantation, and short telomere syndrome. In some embodiments, the chronic liver disease is primary sclerosing cholangitis. In some embodiments, the chronic liver disease is portal hypertension. In some embodiments, the subject has chronic liver disease and HPS. In some embodiments, the disclosure provides a method for treating one or more complications of chronic liver disease, comprising administering to a subject in need thereof a transforming growth factor β receptor II (TβRII) antagonist, wherein the one or more complications of chronic liver disease is selected from the group consisting of spider nevi (spider angiomata, spider angiomas, or nevi araneus), hyperdynamic circulation, clubbing of the fingers or toes, hypertrophic osteoarthropathy, orthodeoxia, cyanosis weakness, fatigue, anorexia, gastrointestinal bleeding, inability to concentrate, menstrual irregularities, decreased libido, skin fragility, bruising, pruritus, ascites, anasarca, nail changes, splenomegaly, a large or small liver, caput medusae, gynecomastia, palmar erythema, testicular atrophy, and/or jaundice. In some embodiments, the subject has one or more complications of the lungs associated with chronic liver disease. In some embodiments, the one or more complications of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, the one or more complications of the lungs is interstitial lung disease (ILD), pulmonary fibrosis, or idiopathic pulmonary fibrosis. In some embodiments, the subject has chronic liver disease and pulmonary fibrosis. In some embodiments, the subject has chronic liver disease and interstitial lung disease (ILD). In some embodiments, the subject has chronic liver disease and idiopathic pulmonary fibrosis. In some embodiments, the subject has chronic liver disease and a short telomere syndrome. In some embodiments, the subject has compensated chronic liver disease. In some embodiments, the subject has decompensated chronic liver disease.

In some embodiments, the disclosure provides a method for treating portal hypertension, comprising administering to a subject in need thereof a transforming growth factor β receptor II (TβRII) antagonist. In some embodiments, the subject has one or more complications of the lungs associated with portal hypertension. In some embodiments, the one or more complications of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, the one or more complications of the lungs is interstitial lung disease (ILD), pulmonary fibrosis, or idiopathic pulmonary fibrosis. In some embodiments, the subject has portal hypertension and pulmonary fibrosis. In some embodiments, the subject has portal hypertension and interstitial lung disease. In some embodiments, the subject has portal hypertension and idiopathic pulmonary fibrosis. In some embodiments, the subject has portal hypertension and a short telomere syndrome. In some embodiments, the subject has one or more complications associated with portal hypertension selected from the group consisting of gastrointestinal bleeding, esophageal varices, caput medusae, ascites, decreased albumin, and splanchnic vasodilation. In some embodiments, the subject has a portal venous pressure more than 5 mm Hg greater than the inferior vena cava pressure. In some embodiments, the subject has a portal venous pressure above 7 mm Hg. In some embodiments, administration of a TβRII antagonist reduces the portal venous pressure. In some embodiments, administration of a TβRII antagonist reduces the portal venous pressure below 7 mm Hg. In some embodiments, administration of a TβRII antagonist reduces the portal venous pressure to less than 5 mm Hg greater than the inferior vena cava pressure.

In some embodiments, the disclosure provides a method for treating primary sclerosing cholangitis (PCS), comprising administering to a subject in need thereof a transforming growth factor β receptor II (TβRII) antagonist. In some embodiments, the subject has one or more complications of the lungs associated with PCS. In some embodiments, the one or more complications of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, the one or more complications of the lungs is interstitial lung disease (ILD), pulmonary fibrosis, or idiopathic pulmonary fibrosis. In some embodiments, the subject has PCS and pulmonary fibrosis. In some embodiments, the subject has PCS and interstitial lung disease.

In some embodiments, the subject has PCS and idiopathic pulmonary fibrosis. In some embodiments, the subject has PCS and a short telomere syndrome. In some embodiments, the subject has obstructed bile flow from the liver. In some embodiments, the subject has cholestasis. In some embodiments, administration of a TβRII antagonist alleviates the cholestasis in the subject. In some embodiments, administration of a TβRII antagonist treats one or more diseases associated with primary sclerosing cholangitis. In some embodiments, the one or more diseases associated with primary sclerosing cholangitis is selected from the group consisting of sarcoidosis, thyroid disease, Peyronie's disease, retroperitoneal fibrosis, psoriasis, rheumatoid arthritis, Celiac disease, Sjogren's syndrome, chronic pancreatitis, lupus, diabetes mellitus, Wegener's granulomatosis, pyoderma gangrenosum, Grave's disease, and Langerhans cell histiocytosis.

In some embodiments of the present disclosure, the subject has diffuse intrapulmonary vascular dilatations at the capillary and pre-capillary levels. In some embodiments, the subject has dilated blood vessels that reach a diameter of at least 15-100 μm. In some embodiments, the subject has dilated blood vessels that reach a diameter greater than 100 μm. In some embodiments, the subject has intrapulmonary arteriovenous shunts. In some embodiments, the subject suffers from arterial hypoxemia. In some embodiments of the present disclosure, the subject is evaluated using contrast-enhanced transthoracic echocardiography (CTTE). In some embodiments of the present disclosure, the subject is evaluated using technetium 99m-labeled macroaggregated albumin (Tc-99m MAA) scanning. In some embodiments, the subject is evaluated using contrast transesophageal echocardiography. In some embodiments, the subject is evaluated using pulmonary angiography. In some embodiments, the subject exhibits type I or type II hepatopulmonary syndrome based on pulmonary angiography. In some embodiments, the subject exhibits type I HPS based on pulmonary angiography. In some embodiments, the subject exhibits type II HPS based on pulmonary angiography. In some embodiments, the subject has a pattern of finely diffuse infiltrates as visualized using pulmonary angiography. In some embodiments, the subject has type I HPS, and has a pattern of finely diffuse infiltrates as visualized using pulmonary angiography. In some embodiments, the subject has localized arteriovenous communications as visualized using pulmonary angiography. In some embodiments, the subject has type II HPS, and has localized arteriovenous communications as visualized using pulmonary angiography. In some embodiments, the subject has one or more intrapulmonary vascular dilatations and/or shunts. In some embodiments, the subject has a shunt fraction of at least greater than 5%. In some embodiments, administration of the TβRII antagonist decreases the shunt fraction to at least less than 20%.

In some embodiments of the present disclosure, the subject is evaluated using a chest radiograph. In some embodiments, the subject has a chest radiograph with one or more radiographic abnormalities selected from the group consisting of a nodular pattern, reticular pattern, a linear pattern, a combined reticular and nodular pattern, a destructive pattern, an alveolar pattern, a bronchial pattern, a vascular pattern, or a honeycombing pattern. In some embodiments, the subject has a pattern of nonspecific interstitial pneumonia (NSIP) in the lungs. In some embodiments, the subject has a pattern of usual interstitial pneumonia (UIP) in the lungs. In some embodiments, the subject is evaluated using high resolution computer topography (HRCT). In some embodiments, an amount of fibrosis or pattern of interstitial pneumonia is determined by HRCT. In some embodiments, the HRCT scan reveals lung parenchymal changes in the subject including any round glass opacity and fibrotic peripheral reticulations and honey combing without abnormalities. In some embodiments, the subject has at least 10% fibrosis of the lungs as determined by HRCT.

In some embodiments of the present disclosure, the subject is evaluated using FRI. In some embodiments, an amount of fibrosis or pattern of interstitial pneumonia is determined by FRI analysis of the lungs of the subject. In some embodiments, the subject has at least between about 10% and about 20%, about 20% and about 30%, about 30% and about 40%, or about 40% and about 50% fibrosis of the lungs.

In some embodiments of the present disclosure, the subject is evaluated using arterial blood gas measurements. In some embodiments, the severity of the subject's HPS is evaluated using arterial blood gas measurements. In some embodiments, the subject has mild HPS comprising an alveolar-arterial oxygen gradient (AaO2) above, or equal to, 15 mmHg, and a partial pressure of arterial oxygen above, or equal to, 80 mmHg. In some embodiments, the subject has moderate HPS comprising an AaO2 gradient above, or equal to, mmHg, and a partial pressure of oxygen above, or equal to, 60 mm Hg to 80 mm Hg. In some embodiments, the subject has severe HPS comprising an AaO2 gradient above, or equal to, 15 mmHg, and a partial pressure of oxygen above, or equal to, 50 to 60 mm Hg. In some embodiments, the subject has very severe HPS comprising an AaO2 gradient above, or equal to, 15 mmHg, and a partial pressure of oxygen below 50 mmHg (<300 mmHg while the subject is breathing 100% oxygen). In some embodiments, administration of a TβRII antagonist reduces the severity of HPS from very severe to severe. In some embodiments, administration of a TβRII antagonist reduces the severity of HPS from severe to moderate. In some embodiments, administration of a TβRII antagonist reduces the severity of HPS from moderate to mild. In some embodiments, administration of a TβRII antagonist reduces the severity of HPS in a subject with very severe HPS and an AaO2 gradient above, or equal to, mmHg, with a partial pressure of oxygen below 50 mmHg (<300 mmHg while the subject is breathing 100% oxygen), to severe HPS with an AaO2 gradient above, or equal to, 15 mmHg, with a partial pressure of oxygen above, or equal to, 50 to 60 mm Hg. In some embodiments, administration of a TβRII antagonist reduces the severity of HPS in a subject with severe HPS and an AaO2 gradient above, or equal to, 15 mmHg, with a partial pressure of oxygen above, or equal to, 50 to 60 mm Hg, to moderate HPS with an AaO2 gradient above, or equal to, 15 mmHg, with a partial pressure of oxygen above, or equal to, 60 mm Hg to 80 mm Hg. In some embodiments, administration of a TβRII antagonist reduces the severity of HPS in a subject with moderate HPS and an AaO2 gradient above, or equal to, 15 mmHg, with a partial pressure of oxygen above, or equal to, 60 mm Hg to 80 mm Hg, to mild HPS with an AaO2 gradient above, or equal to, 15 mmHg, with a partial pressure of arterial oxygen above, or equal to, 80 mmHg. In some embodiments, administration decreases the subject's AaO2 gradient to below 15 mm Hg, and increases the subject's partial pressure of arterial oxygen to above 80 mm Hg.

In some embodiments of the present disclosure, the subject is evaluated using a measurement of diffusing capacity of the lungs for carbon monoxide (DLCO). In some embodiments, the subject has a diffusing capacity for carbon monoxide (DLCO) of greater than or equal to 40% of predicted. In some embodiments, the subject has a DLCO of between about 90% and about 80% of predicted, about 80% and about 70% of predicted, about 70% and about 60% of predicted, about 60% and about 50% of predicted, about 50% and about 40% of predicted, about 40% and about 30% of predicted, or about 30% and about 20% of predicted. In some embodiments, administration of a TβRII antagonist restores the subject's lung diffusing capacity for carbon monoxide (DLCO) to normal values relative to a subject without hepatopulmonary syndrome.

In some embodiments of the present disclosure, administration of a TβRII antagonist decreases the subject's enhanced liver fibrosis (ELF) score below 9.8. In some embodiments, administration of a TβRII antagonist decreases the subject's ELF score below 11.3. In some embodiments, administration of a TβRII antagonist improves the subject's total lung capacity (TLC) to between about 90% and about 80% of predicted, about 80% and about 70% of predicted, about 70% and about 60% of predicted, about 60% and about 50% of predicted, about 50% and about 40% of predicted, or about 40% and about 30% of predicted. In some embodiments, administration of a TβRII antagonist decreases the Bonacini score to less than 7, less than 6, less than 5, less than 4, or less than 3. In some embodiments, the subject has an APRI score of less than or equal to 0.5. In some embodiments, the subject has an APRI score greater than or equal to 1.5. In some embodiments, administration of a TβRII antagonist reduces the subjects APRI score to less than 1.5 or less than 0.5. In some embodiments, the subject has a Forn's index score less than or equal to 6.9. In some embodiments, the subject has a Forn's index score greater than or equal to 6.9. In some embodiments, the subject has a Forn's index score less than 6.5, less than 5.0, or less than 3.0. In some embodiments, administration of a TβRII antagonist decreases the subject's Forn's index score to less than 6.9, less than 6.5, less than 5.5, less than 4.5, less than 3.0, less than 2.0, or less than 1.0.

In some embodiments of the present disclosure, the subject is evaluated using St. George's Respiratory Questionnaire (SGRQ). In some embodiments, the subject is evaluated for respiratory function using a St. George's Respiratory Questionnaire (SGRQ). In some embodiments, administration of a TβRII antagonist improves a St. George's Respiratory Questionnaire (SGRQ) score of the subject by between about 1% and about 3%, about 1% and about 5%, about 1% and about 8%, about 1% and about 10%, about 10% and about 15%, about 15% and about 20%, or about 20% and about 25%. In some embodiments, the SGRQ score is improved after administration of a TβRII antagonist, compared to a baseline measurement. In some embodiments, the SGRQ is decreased after administration of a TβRII antagonist, compared to a baseline measurement. In some embodiments, the administration of a TβRII antagonist improves a King's Brief Interstitial Lung Disease (KBILD) score of the subject. In some embodiments, the KBILD score is improved after administration of a TβRII antagonist, compared to a baseline measurement. In some embodiments, the KBILD score is increased after administration of a TβRII antagonist, compared to a baseline measurement. In some embodiments, the administration of a TβRII antagonist improves a physical/physician global assessment score of the subject. In some embodiments, the physical/physician global assessment score is improved after administration of a TβRII antagonist, compared to a baseline measurement. In some embodiments, administration of a TβRII antagonist improves a patient global assessment score of the subject, compared to a baseline measurement. In some embodiments, the patient global assessment score is improved after the administering, compared to a baseline measurement. In some embodiments, administration of a TβRII antagonist increases the subject's length of life.

In some embodiments of the present disclosure, the expression of one or more biomarkers is upregulated in the subject. In some embodiments, the one or more biomarkers is selected from the group consisting of albumin, bilirubin, AST (aspartate aminotransferase), ALT (alanine aminotransferase), γGT (gamma glutamyl transferase), ALP (alkaline phosphatase), prothrombin time, PICP (procollagen I carboxy peptide), PIIINP (procollagen III amino peptide), collagen type-IV, hyaluronic acid, laminin, YKL-40, MMP7, TIMP, TGFβ1, PDGF (platelet derived growth factor), microfibril associated protein-4, ADAM12, Anti-HSP70 IgG, BAFF, BLyS, C3M, C4M, C6M, Ca15.3, CC16, CCL2, CCL18, Colla1, Col3a1, CTGF, CXCL4, E-selectin, ET-1, fibronectin (FN1), Heat shock protein 70 (HSP70), ICAM, IL-6, IL-8, KL-6, MCP-1, Muc5B, Osteopontin, PAI-1, periostin, pro-C3, pro-C4, pro-C6, SerpinE1, SP-A, SP-D, Tnfa, VCAM, VEGF, WFDC2, and aSMA. In some embodiments, the one or more biomarkers is selected from the group consisting of SerpinE1, Colla1, and FN1. In some embodiments, administration of a TβRII antagonist reduces expression of one or more biomarkers selected from the group consisting of SerpinE1, Colla1, and FN1. In some embodiments, administration does not reduce expression of MMP12.

In some embodiments of the present disclosure, a dose of the TβRII antagonist comprises between about 0.75 mg/to about 6.0 mg/kg of the antagonist. In some embodiments, the TβRII antagonist is administered subcutaneously. In some embodiments, the TβRII antagonist is administered at a dose that achieves a serum concentration of antagonist of between about 10 and about 20 ug/mL in the subject. In some embodiments, the TβRII antagonist is administered at a dose that achieves a concentration of antagonist in the lung of between about 30 and about 50 ug/mL in the subject. In some embodiments, the subject has been treated with one or more of abatacept, abituzumab, ajulemic acid, ambrisentan, AVID200, AVID300, azathioprine, BCD-089, belimumab, BG00011, BMS-986020, bortezomib, bosentan, brentuximab, carlumab, CC-90001, cephalosporin, clazakizumab, COR-001, cyclophosphamide (CYC), cyclosporine A, dectrekumab, EHP-101, elzonris/SL-401, etanercept, FCX-013, fresolimumab, GLPG1690, GASK2126458, GSK2330811, GSK3008348, IBIO-CFB03, ifetroban, IFNγ, imatinib, immune globulin, IW001, lanifibranor, lebrikizumab, levilimab, losartan, macitentan, MEDI-5117, methotrexate, MSCs, mycophenolate mofetil (MMF), NAC, nandrolone decanoate, nintedanib (Ofev), olokizumab, pamrevlumab, pirfenidone, pirfenidone and vismodegib, pomalidomide, PRM-151. riociguat, rituximab, SAR156597, sildenafil, siltuximab, simtuzumab, sirolimus, sirukumab, tacrolimus, tadalafil, tanzisertib, TD139, tetrathiomolybdate, tocilizumab, tralokinumab, treprostinil, vobarilizumab, warfarin, zileuton, and ziltivekimab.

In some embodiments of the present disclosure, the TβRII antagonist comprises a TβRII extracellular domain, wherein the TβRII extracellular domain comprises an amino acid sequence at least 80% identical to: i) a sequence beginning at any of positions 23 to 35 of SEQ ID NO: 1 and ending at any of positions 153 to 159 of SEQ ID NO: 1 or; ii) a sequence beginning at any of positions 23 to 60 of SEQ ID NO: 2 and ending at any of positions 178 to 184 of SEQ ID NO: 2. In some embodiments, the TβRII extracellular domain comprises an amino acid sequence at least 90% identical to a sequence beginning at any of positions 23 to 35 of SEQ ID NO: 1 and ending at any of positions 153 to 159 of SEQ ID NO: 1. In some embodiments, the TβRII extracellular domain comprises an amino acid sequence at least 95% identical to a sequence beginning at any of positions 23 to 35 of SEQ ID NO: 1 and ending at any of positions 153 to 159 of SEQ ID NO: 1. In some embodiments, the TβRII extracellular domain comprises an amino acid sequence beginning at any of positions 23 to 35 of SEQ ID NO: 1 and ending at any of positions 153 to 159 of SEQ ID NO: 1. In some embodiments, the TβRII extracellular domain comprises an amino acid sequence at least 90% identical to a sequence beginning at any of positions 23 to 60 of SEQ ID NO: 2 and ending at any of positions 178 to 184 of SEQ ID NO: 2. In some embodiments, the TβRII extracellular domain comprises an amino acid sequence at least 95% identical to a sequence beginning at any of positions 23 to 60 of SEQ ID NO: 2 and ending at any of positions 178 to 184 of SEQ ID NO: 2. In some embodiments, the TβRII extracellular domain comprises an amino acid sequence beginning at any of positions 23 to 60 of SEQ ID NO: 2 and ending at any of positions 178 to 184 of SEQ ID NO: 2. In some embodiments, the TβRII extracellular domain comprises an amino acid sequence at least 90% identical to SEQ ID NO: 18. In some embodiments, the TβRII extracellular domain comprises an amino acid sequence at least 95% identical to SEQ ID NO: 18. In some embodiments, the TβRII extracellular domain comprises the amino acid sequence of SEQ ID NO: 18.

In some embodiments of the present disclosure, the TβRII antagonist is a fusion protein further comprising a heterologous domain. In some embodiments, the heterologous domain comprises an immunoglobulin Fc domain. In some embodiments, the immunoglobulin Fc domain is a human immunoglobulin Fc domain. In some embodiments, the heterologous domain comprises an amino acid sequence that is at least 90% identical to SEQ ID NO: 20. In some embodiments, the heterologous domain comprises an amino acid sequence that is at least 95% identical to SEQ ID NO: 20. In some embodiments, the heterologous domain comprises the amino acid sequence of SEQ ID NO: 20. In some embodiments, the heterologous domain comprises an amino acid sequence that is at least 90% identical to SEQ ID NO: 72. In some embodiments, the heterologous domain comprises an amino acid sequence that is at least 95% identical to SEQ ID NO: 72. In some embodiments, the heterologous domain comprises the amino acid sequence of SEQ ID NO: 72. In some embodiments, the TβRII antagonist further comprises a linker. In some embodiments, the linker comprises (GGGGS)ₙ, wherein n=≥4. In some embodiments, the linker comprises the amino acid sequence of SEQ ID NO: 6.

In some embodiments, the TβRII antagonist does not include amino acids 185-592 of SEQ ID NO: 2. In some embodiments, the TβRII antagonist does not include amino acids 1-22 of SEQ ID NO: 2. In some embodiments, the fusion protein consists of or consists essentially of: a) a TβRII polypeptide portion comprising an amino acid sequence that is at least 85%, 90%, 95%, 97%, or 99% identical to the amino acid sequence of SEQ ID NO: 18 and no more than 10, 9, 8, 7, 6, 5, 4, 3, 2 or 1 additional amino acids; b) a linker portion comprising an amino acid sequence that is at least 85%, 90%, 95%, 97%, or 99% identical to the amino acid sequence of SEQ ID NO: 6 and no more than 5, 4, 3, 2 or 1 additional amino acids; c) a heterologous portion comprising an amino acid sequence that is at least 85%, 90%, 95%, 97%, or 99% identical to the amino acid sequence of SEQ ID NO: 20 or SEQ ID NO: 72 and no more than 25, 20, 15, 10, 5, 4, 3, 2, or 1 additional amino acids; and d) optionally a leader sequence (e.g., SEQ ID NO: 23). In some embodiments, the fusion protein consists of or consists essentially of: a) a TβRII polypeptide portion comprising the amino acid sequence of SEQ ID NO: 18 and no more than 10, 9, 8, 7, 6, 5, 4, 3, 2 or 1 additional amino acids; b) a linker portion comprising the amino acid sequence of SEQ ID NO: 6 and no more than 5, 4, 3, 2 or 1 additional amino acids; c) a heterologous portion comprising the amino acid sequence of SEQ ID NO: 20 or SEQ ID NO: 72 and no more than 25, 20, 15, 10, 5, 4, 3, 2, or 1 additional amino acids; and d) optionally a leader sequence (e.g., SEQ ID NO: 23).

In some embodiments, a fusion protein of the present disclosure comprises: a) an extracellular domain of a TβRII portion; wherein the extracellular domain comprises an amino acid sequence that is at least 85%, 90%, 95%, 97%, or 99% identical to the sequence of SEQ ID NO: 18; b) a heterologous portion, wherein the heterologous portion comprises an amino acid sequence that is at least 85%, 90%, 95%, 97%, or 99% identical to the sequence of SEQ ID NO: 20 or SEQ ID NO: 72; and c) a linker portion connecting the extracellular domain and the heterologous portion; wherein the linker comprises an amino acid sequence that is at least 85%, 90%, 95%, 97%, or 99% identical to the amino acid sequence of SEQ ID NO: 6.

In some embodiments, the fusion protein comprises: a) an extracellular domain of a TβRII portion; wherein the extracellular domain comprises the amino acid sequence of SEQ ID NO: 18; b) a heterologous portion, wherein the heterologous portion comprises the amino acid sequence of SEQ ID NO: 20 or SEQ ID NO: 72; and c) a linker portion connecting the extracellular domain and the heterologous portion; wherein the linker comprises the amino acid sequence of SEQ ID NO: 6.

In some embodiments, the TβRII antagonist comprises an amino acid sequence at least 90% identical to SEQ ID NO: 48. In some embodiments, the TβRII antagonist comprises an amino acid sequence at least 95% identical to SEQ ID NO: 48. In some embodiments, the TβRII antagonist comprises the amino acid sequence of SEQ ID NO: 48. In some embodiments, the TβRII antagonist consists of the amino acid sequence of SEQ ID NO: 48.

In some embodiments, the TβRII antagonist comprises an amino acid sequence at least 90% identical to SEQ ID NO: 67. In some embodiments, the TβRII antagonist comprises an amino acid sequence at least 95% identical to SEQ ID NO: 67. In some embodiments, the TβRII antagonist comprises the amino acid sequence of SEQ ID NO: 67. In some embodiments, the TβRII antagonist consists of the amino acid sequence of SEQ ID NO: 67.

In some embodiments, the antagonist includes one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, the antagonist is glycosylated.

### BRIEF DESCRIPTION OF THE DRAWINGS

FIG. 1 shows the amino acid sequence of native precursor for the B (short) isoform of human TGFβ receptor type II (hTβRII) (NP 003233.4) (SEQ ID NO: 1). Solid underline indicates the mature extracellular domain (ECD) (residues 23-159), and double underline indicates valine that is replaced in the A (long) isoform. Dotted underline denotes leader (residues 1-22).

FIG. 2 shows the amino acid sequence of native precursor for the A (long) isoform of human TβRII (NP_001020018.1) (SEQ ID NO: 2). Solid underline indicates the mature ECD (residues 23-184), and double underline indicates the splice-generated isoleucine substitution. Dotted underline denotes leader (residues 1-22).

FIG. 3 shows a comparison of the linker sequences of five different TβRII constructs (SEQ ID NOs 62-66, respectively, in order of appearance).

FIGS. 4A and 4B show in tabular form the binding affinity between TGFβ1 and TGFβ3 and one of several different TβRII-Fc fusion protein constructs.

FIGS. 5A and 5C graph the results from reporter gene assays testing the affinity of TGFβ1 for one of several different TβRII-Fc fusion protein constructs. FIGS. 5B and 5D graph the results from reporter gene assays testing the affinity of the TGFβ3 for one of several different TβRII-Fc fusion protein constructs. FIGS. 5E and 5F provide IC₅₀ data from these same experiments in tabular form.

FIGS. 6A to 6G shows the anti-fibrotic effects of mTβRII-mFc, a murine fusion protein that selectively binds to TGF-β1 and TGF-β3, in a mouse model of lung fibrosis induced by bleomycin infusion. Male 8-week old C57BL/6J mice were administered either saline or bleomycin by osmotic mini-pumps implanted subcutaneously under the back skin for 7 days, after which pumps were removed. Starting on day 9, mice began receiving treatment twice per week (except for “Baseline” mice), treatment comprising either mTβRII-mFc at a dose of 1 mg/kg, 3 mg/kg, or 10 mg/kg or PBS (10 uL/g, equivolume to treatment groups). At the completion of the study (day 9 after pump implantation for “Baseline” group and day 34, day 35, or day 36 for other groups), mice were euthanized for blood, bronchoalveolar lavage (BAL) and lung tissue collection. The level of SMAD2 phosphorylation, as measured by the ratio of pSMAD2 to tSMAD2 signal, was determined by an MSD assay (FIG. 6A). Levels of SMAD2 phosphorylation were significantly elevated at “Baseline” and day 35 post-bleomycin (“Vehicle” (bleomycin)) (p≤0.01 and 0.0001, respectively). mTβRII-mFc dose-dependently reduced SMAD2 phosphorylation in bleomycin treated mice, with significant effects for the 3 and 10 mg/kg doses of mTβRII-mFc (p≤0.0001). mTβRII-mFc (10 mg/kg) showed no effect on SMAD2 phosphorylation levels in saline-infused mice (FIG. 6A). Expression of fibrosis-related genes and downstream targets of TGF-β signaling were analyzed by qPCR (FIGS. 6B-6E). Expression was normalized to GAPDH and data are shown relative to “Vehicle” (saline). The “Vehicle” (bleomycin) group showed significantly elevated expression levels of SerpinE1 (FIG. 6B), Colla1 (FIG. 6C), FN1 (FIG. 6D) and MMP12 (FIG. 6E) (p<0.0001). mTβRII-mFc significantly and dose-dependently reduced expression of the pro-fibrotic markers SerpinE1, Colla1, and FN1 (p≤0.05 for 10 mg/kg), while showing no effect on MMP12 expression in bleomycin treated mice. Tissue levels of aSMA and collagen-1A in lung tissue were assessed using IHC with quantitative image analysis (FIGS. 6F and 6G, respectively). “Vehicle” (bleomycin) mice showed significantly more positive area of staining for aSMA and collagen-1A in the lung compared to “Vehicle” (saline) mice (p≤0.0001). mTβRII-mFc treatment significantly and dose-dependently inhibited increases in aSMA and collagen-1A staining at both 3 and 10 mg/kg doses in bleomycin treated mice (p≤0.05-0.0001). Statistical analysis (One-way ANOVA with Dunnett's post hoc tests): #p≤0.05, ##p≤0.01, ###p≤0.001, ####p≤0.0001 vs “Vehicle” (saline) and *p≤0.05, **p≤0.01, ***p≤0.001, ****p≤0.0001 vs “Vehicle” (bleomycin).

### DETAILED DESCRIPTION

### 1. Overview

In part, the disclosure relates to TβRII antagonists that can be used to treat chronic liver disease. In particular, the disclosure relates to TβRII antagonists that can be used to treat disorders associated with chronic liver disease, such as portal hypertension, primary sclerosing cholangitis, and hepatopulmonary syndrome. The disclosure also relates to TβRII antagonists that can be used to treat clinical complications associated with chronic liver disease, portal hypertension, primary sclerosing cholangitis, and hepatopulmonary syndrome, particularly clinical complications such as, for example, pulmonary fibrosis or interstitial lung disease (ILD). Accordingly, the disclosure provides methods of treating chronic liver disease, portal hypertension, primary sclerosing cholangitis, and hepatopulmonary syndrome, or a complication thereof (e.g., pulmonary fibrosis, ILD) comprising administering to a subject in need thereof one or more TβRII antagonists as described herein. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Optionally, such methods further comprise administering to the subject one or more additional active agents and/or supportive therapies for treating chronic liver disease or a disorder or complication associated with chronic liver disease (e.g., portal hypertension, primary sclerosing cholangitis, and hepatopulmonary syndrome, ILD, pulmonary fibrosis). In some embodiments, portal hypertension is associated with chronic liver disease. In some embodiments, primary sclerosing cholangitis is associated with chronic liver disease. In some embodiments, hepatopulmonary syndrome is associated with chronic liver disease. In some embodiments, a subject with chronic liver disease, may have complications arising from one or more of portal hypertension, primary sclerosing cholangitis, and/or hepatopulmonary syndrome. In some embodiments, a complication associated with chronic liver disease is interstitial lung disease (ILD). In some embodiments, a complication associated with chronic liver disease is pulmonary fibrosis. In some embodiments, a complication associated with portal hypertension is interstitial lung disease (ILD). In some embodiments, a complication associated with portal hypertension is pulmonary fibrosis. In some embodiments, a complication associated with primary sclerosing cholangitis is interstitial lung disease (ILD). In some embodiments, a complication associated with primary sclerosing cholangitis is pulmonary fibrosis. In some embodiments, a complication associated with hepatopulmonary syndrome is interstitial lung disease (ILD). In some embodiments, a complication associated with hepatopulmonary syndrome is pulmonary fibrosis.

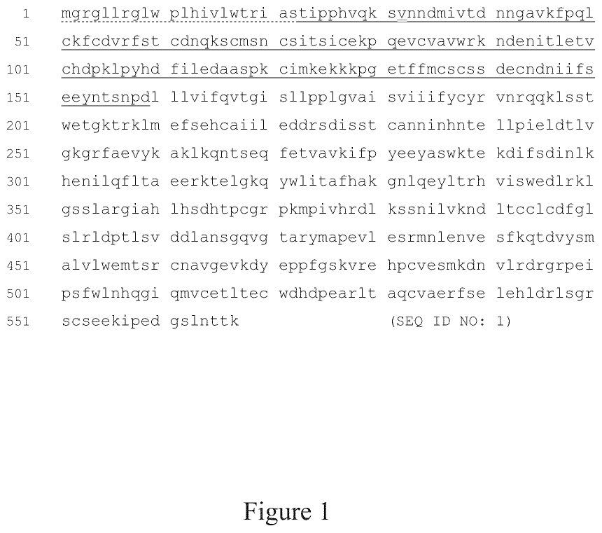

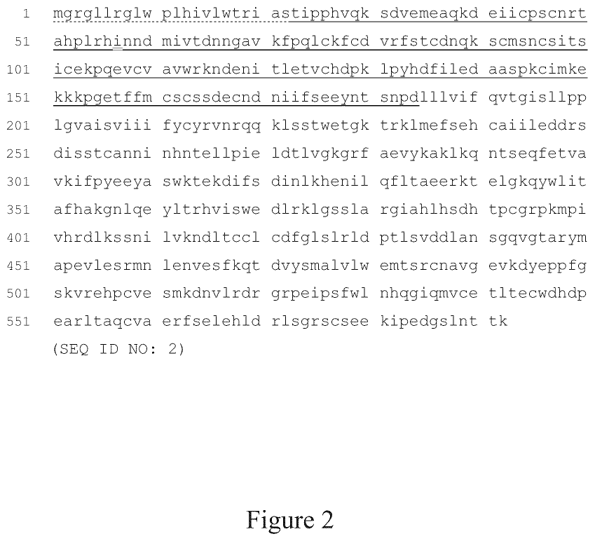

Proteins described herein are the human forms, unless otherwise specified. NCBI references for the proteins are as follows: human TβRII isoform A (hTβRII_{long}), (NP 001020018.1) (SEQ ID NO: 2) and human TβRII isoform B (hTβRIIₛₕₒᵣₜ), (NP 003233.4) (SEQ ID NO: 1). Sequences of native TβRII proteins from human are set forth in FIGS. 1 and 2. In some embodiments, the TβRII proteins are from non-human animals, such as a mouse, rat, cow or monkey.

The TGFβ superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, chondrogenesis, cardiogenesis, hematopoiesis, neurogenesis, and epithelial cell differentiation. By manipulating the activity of a member of the TGFβ family, it is often possible to cause significant physiological changes in an organism. For example, the Piedmontese and Belgian Blue cattle breeds carry a loss-of-function mutation in the GDF8 (also called myostatin) gene that causes a marked increase in muscle mass. Grobet et al., Nat Genet. 1997, 17(1):71-4. Similarly, in humans, inactive alleles of GDF8 are associated with increased muscle mass and, reportedly, exceptional strength. Schuelke et al., N Engl J Med 2004, 350:2682-8.

TGFβ signals are mediated by heteromeric complexes of type I (e.g. TβRI) and type II (e.g. TβRII) serine/threonine kinase receptors, which phosphorylate and activate downstream SMAD proteins upon ligand stimulation (Massague, 2000, Nat. Rev. Mol. Cell Biol. 1:169-178). These type I and type II receptors are transmembrane proteins, composed of a ligand-binding extracellular domain with cysteine-rich region, a transmembrane domain, and a cytoplasmic domain with predicted serine/threonine specificity. Type I receptors are essential for signaling; and type II receptors are required for binding ligands and for expression of type I receptors. Type I and II receptors form a stable complex after ligand binding, resulting in phosphorylation of type I receptors by type II receptors. TGFβ has three mammalian isoforms, TGFβ1, TGFβ2 and TGFβ3, each with distinct functions in vivo. The binding of TGFβs to TβRII is a crucial step in initiating activation of the TGFβ signaling pathway, leading to phosphorylation of SMAD2, and translocation of the activated SMAD2/SMAD4 complex to the nucleus to modulate gene expression.

In part, the disclosure relates to TβRII antagonists that can be used to treat disorders associated with chronic liver disease such as, portal hypertension, primary sclerosing cholangitis, and hepatopulmonary syndrome. In particular, the disclosure relates to TβRII antagonists that can be used to treat clinical complications associated with chronic liver disease, portal hypertension, primary sclerosing cholangitis, and hepatopulmonary syndrome including, for example, pulmonary fibrosis and interstitial lung disease (ILD). In some embodiments, the disclosure provides TβRII polypeptides as antagonists of TGFβ1 or TGFβ3 for use in treating disorders associated with chronic liver disease, such as portal hypertension, primary sclerosing cholangitis, and hepatopulmonary syndrome. In some embodiments, the disclosure provides TβRII polypeptides as antagonists of TGFβ1 or TGFβ3 for use in treating a complication associated with chronic liver disease, portal hypertension, primary sclerosing cholangitis, and hepatopulmonary syndrome, such as pulmonary fibrosis or interstitial lung disease (ILD). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

The present disclosure demonstrates that treatment with TβRII-Fc suppresses lung fibrosis in an animal model, and may be useful in the treatment or prevention of lung fibrosis. Recent studies of liver disease highlight the involvement of several cytokines that are similarly involved in the pathogenesis of interstitial lung disease (ILD) or pulmonary fibrosis. Furthermore, a subpopulation of liver disease patients has been identified with high prevalence (21%) of short telomere syndromes including ILD and pulmonary fibrosis. According to the data presented herein (See, e.g., Example 3), mTβRII-mFc has anti-fibrotic effects in a mouse model of lung fibrosis induced by bleomycin infusion. Such data suggests that a TβRII antagonist of the present disclosure would have effects on lung fibrosis, and also possibly broader effects in diseases and disorders associated with fibrosis in general, including liver fibrosis (e.g., chronic liver disease, portal hypertension, primary sclerosing cholangitis, and hepatopulmonary syndrome). Additionally, methods and uses directed to TβRII-Fc treatment as disclosed herein, may overcome severe complications associated with therapies targeted toward chronic liver disease, portal hypertension, primary sclerosing cholangitis, and/or hepatopulmonary syndrome patients with ILD or pulmonary fibrosis. In particular, these patients currently have severely limited treatment options, since most are contraindicated due to the presence of liver disease. Together, the present disclosure demonstrates that TβRII antagonists (e.g. TβRII-Fc fusion proteins) administered according to the methods and uses disclosed herein, may be useful in the treatment of patients with chronic liver disease, portal hypertension, primary sclerosing cholangitis, and/or hepatopulmonary syndrome, particularly those patients with pulmonary fibrosis or ILD associated with chronic liver disease, portal hypertension, primary sclerosing cholangitis, and/or hepatopulmonary syndrome. In some embodiments, a subject treated by the methods and uses disclosed herein may have chronic liver disease, portal hypertension, primary sclerosing cholangitis, and/or hepatopulmonary syndrome, and a short telomere syndrome. In certain embodiments, the short telomere syndrome is interstitial lung disease (ILD). In certain embodiments, the short telomere syndrome is pulmonary fibrosis. In certain embodiments, the subject has chronic liver disease, portal hypertension, primary sclerosing cholangitis, and/or hepatopulmonary syndrome, and ILD. In certain embodiments, the subject has chronic liver disease, portal hypertension, primary sclerosing cholangitis, and/or hepatopulmonary syndrome, and pulmonary fibrosis. In certain embodiments, the subject has chronic liver disease, portal hypertension, primary sclerosing cholangitis, and/or hepatopulmonary syndrome, and a complication of the lungs. In some embodiments, a complication of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, a complication of the lungs is selected from the group consisting of interstitial lung disease (ILD), pulmonary fibrosis, or idiopathic pulmonary fibrosis. In certain embodiments, the complication of the lungs is pulmonary fibrosis. In certain embodiments, the complication of the lungs is idiopathic pulmonary fibrosis. In certain embodiments, the complication of the lungs is interstitial lung disease (ILD).

The terms used in this specification generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. The scope or meaning of any use of a term will be apparent from the specific context in which the term is used.

The terms “treatment”, “treating”, “alleviation” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition.

“Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. The term “sequence similarity,” in all its grammatical forms, refers to the degree of identity or correspondence between nucleic acid or amino acid sequences that may or may not share a common evolutionary origin. However, in common usage and in the instant application, the term “homologous,” when modified with an adverb such as “highly,” may refer to sequence similarity and may or may not relate to a common evolutionary origin.

“Percent (%) sequence identity” or “percent (%) identical” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers. As used herein, the term “comprises” also encompasses the use of the narrower terms “consisting” and “consisting essentially of”

The term “consisting essentially of” is limited to the specified materials or steps and those that do not materially affect the basic and novel characteristics of the invention(s) disclosed herein.

The term “appreciable affinity” as used herein means binding with a dissociation constant (K D) of less than 50 nM.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

### 2. Chronic Liver Disease

In part, the disclosure relates to TβRII antagonists that can be used to treat chronic liver disease. In particular, the disclosure relates to TβRII antagonists that can be used to treat disorders associated with chronic liver disease, such as portal hypertension, primary sclerosing cholangitis, and hepatopulmonary syndrome. The disclosure also relates to TβRII antagonists that can be used to treat complications associated with chronic liver disease, portal hypertension, primary sclerosing cholangitis, and hepatopulmonary syndrome, particularly clinical complications such as, for example, pulmonary fibrosis or interstitial lung disease (ILD). Accordingly, the disclosure provides methods of treating disorders associated with chronic liver disease, (e.g. portal hypertension, primary sclerosing cholangitis, and hepatopulmonary syndrome) comprising administering to a patient in need thereof one or more TβRII antagonists as described herein. The disclosure also provides methods of treating complications associated with chronic liver disease, portal hypertension, primary sclerosing cholangitis, and hepatopulmonary syndrome (e.g. pulmonary fibrosis or ILD) comprising administering to a patient in need thereof one or more TβRII antagonists as described herein. Optionally, such methods further comprise administering to the patient one or more additional active agents and/or supportive therapies for treating a disorder associated with chronic liver disease or a complication associated with chronic liver disease (e.g., pulmonary fibrosis, ILD).

The disclosure provides methods of treating or preventing a disease or condition associated with a TGFβ superfamily member by administering to a subject one or more TβRII antagonists, including, but not limited to, TβRII polypeptides and fusion proteins comprising the same as described herein. In some embodiments, the disease or condition is associated with dysregulated TGFβ1 or TGFβ3 signaling. In some embodiments, the disease or condition to be treated is chronic liver disease. In some embodiments, a TβRII antagonist for use in treating chronic liver disease is administered to a subject in need thereof. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In part, the disclosure relates to TβRII antagonists that can be used to treat chronic liver disease. Chronic liver disease is a progressive deterioration of liver function leading to hepatic fibrosis and cirrhosis, and can result from a broad spectrum of underlying etiologies. Disorders associated with chronic liver disease may include, for example, portal hypertension, primary sclerosing cholangitis, and hepatopulmonary syndrome. In particular, the disclosure relates to TβRII antagonists that can be used to treat disorders associated with chronic liver disease, such as portal hypertension, primary sclerosing cholangitis, and hepatopulmonary syndrome. Portal hypertension is a major complication of chronic liver disease and results from resistance in portal blood flow occurring due to cirrhotic or noncirrhotic causes. Primary sclerosing cholangitis (PSC) is a chronic progressive disorder of unknown etiology, and is characterized by inflammation, thickening, and abnormal formation of fibrous tissue (fibrosis) within the bile ducts. Hepatopulmonary syndrome (HPS) is a pulmonary disorder of liver disease, and is characterized by a triad of liver disease, arterial hypoxemia, and intrapulmonary vascular abnormalities.

In some embodiments, the disease or condition to be treated is chronic liver disease. In some embodiments, the disease or condition to be treated is a disorder associated with chronic liver disease. In some embodiments, the disorder associated with chronic liver disease is portal hypertension, primary sclerosing cholangitis, or hepatopulmonary syndrome. In some embodiments, the disease or condition to be treated is a complication associated with chronic liver disease. In some embodiments, the complication associated with chronic liver disease is pulmonary fibrosis or interstitial lung disease. In some embodiments, the disease or condition to be treated is pulmonary fibrosis associated with chronic liver disease. In some embodiments, the disease or condition to be treated is interstitial lung disease associated with chronic liver disease. In some embodiments, a TβRII antagonist for use in treating chronic liver disease is administered to a subject in need thereof. In some embodiments, a TβRII antagonist for use in treating a disorder associated with chronic liver disease is administered to a subject in need thereof. In some embodiments, a TβRII antagonist for use in treating a complication associated with chronic liver disease is administered to a subject in need thereof. In some embodiments, a TβRII antagonist for use in treating pulmonary fibrosis associated with chronic liver disease is administered to a subject in need thereof. In some embodiments, a TβRII antagonist for use in treating interstitial lung disease associated with chronic liver disease is administered to a subject in need thereof. In some embodiments, the disclosure provides a method of treating chronic liver disease, comprising administering a TβRII antagonist to a subject in need thereof. In some embodiments, the disease or condition to be treated is pulmonary fibrosis associated with chronic liver disease. In some embodiments, a TβRII antagonist for use in treating pulmonary fibrosis associated with chronic liver disease is administered to a subject in need thereof. In some embodiments, the disclosure provides a method of treating pulmonary fibrosis associated with chronic liver disease, comprising administering a TβRII antagonist to a subject in need thereof. In some embodiments, the disease or condition to be treated is interstitial lung disease (ILD) associated with chronic liver disease. In some embodiments, a TβRII antagonist for use in treating ILD associated with chronic liver disease is administered to a subject in need thereof. In some embodiments, the disclosure provides a method of treating ILD associated with chronic liver disease, comprising administering a TβRII antagonist to a subject in need thereof. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disclosure provides methods of treating chronic liver disease, comprising administering a TβRII antagonist to a subject in need thereof, wherein the treatment increases the subject's length of life when compared to a reference subject that is not receiving treatment. In some embodiments, the disclosure provides methods of treating chronic liver disease, comprising administering a TβRII antagonist to a subject in need thereof, wherein the treatment reduces the subject's risk of death due to chronic liver disease when compared to a reference subject that is not receiving treatment. In some embodiments, the disclosure provides methods of treating chronic liver disease, comprising administering a TβRII antagonist to a subject in need thereof, wherein the treatment reduces the subject's risk of hospitalization due to chronic liver disease when compared to a reference subject that is not receiving treatment. In some embodiments, a reference subject that is not receiving treatment has similar characteristics to the subject (height, sex, age, race, weight) and is not receiving one or more of TβRII antagonists of the present disclosure.

Chronic Liver Disease: Symptoms and Complications

Chronic liver disease is a progressive deterioration of liver function and involves a continuous process of inflammation, destruction, and regeneration of liver parenchyma, leading to hepatic fibrosis and cirrhosis. Chronic liver disease can result from a broad spectrum of underlying etiologies including alcoholic liver disease, non-alcoholic fatty liver disease (NALFD), chronic viral hepatitis, genetics (e.g., Wilson's disease), and autoimmune destruction of liver parenchyma (e.g., primary sclerosing cholangitis). In 2017, there were 2.14 million deaths associated with chronic liver disease worldwide, with cirrhosis accounting for 61.7% of cases (Paik et al., Hepatology. 2020 November; 72(5):1605-1616).

Chronic liver disease manifests as a result of a variety of etiologies. Alcohol induced liver diseases include alcoholic fatty liver disease, alcoholic hepatitis, and alcoholic cirrhosis. Alcoholic liver disease is preventable and treatable, and liver injury may be reversed with abstinence from alcohol consumption. Another cause of chronic liver disease may be non-alcoholic fatty liver disease, which may be simple steatosis, or steatohepatitis. Steatosis involves accumulation of fat in the liver without chronic damage or inflammation. Steatohepatitis involves accumulation of fat in the liver with inflammation and scarring which may progress to cirrhosis. Liver disease may also be caused by genetic abnormalities. For example, hereditary hematochromatosis is a genetically inheritable condition where the body cannot regulate iron, resulting in accumulation of iron in the liver which eventually progresses to cirrhosis. Wilson's disease is another genetically inheritable condition in which abnormal copper transport leads to excess deposition of copper in the liver, resulting in liver injury, hepatitis, and/or liver failure. Finally, liver disease may also stem from viral infections such as hepatitis B or hepatitis C. A subject treated by the methods and uses disclosed herein may have chronic liver disease. In some embodiments, the subject has alcohol induced liver disease. In some embodiments, the subject has alcoholic fatty liver disease. In some embodiments, the subject has alcoholic hepatitis. In some embodiments, the subject has alcoholic cirrhosis. In some embodiments, the subject has non-alcoholic fatty liver disease. In some embodiments, the subject has steatosis. In some embodiments, the subject has steatohepatitis. In some embodiments, the subject has liver disease caused by genetic abnormalities. In some embodiments, the subject has hereditary hematochromatosis. In some embodiments, the subject has Wilson's disease. In some embodiments, the subject has liver disease caused by viral infection. In some embodiments, the subject has liver disease caused by hepatitis B. In some embodiments, the subject has liver disease caused by hepatitis C.

Hepatic cirrhosis may result from a variety of diseases and/or conditions not limited to alcohol abuse, chronic viral hepatitis, fat accumulation in the liver as a result of nonalcoholic fatty liver disease, iron buildup as a result of hemochromatosis, cystic fibrosis, copper accumulation in the liver resulting from Wilson's disease, poor formation of bile ducts resulting from biliary atresia, alpha-1 antitrypsin deficiency, galactosemia, digestive disorders such as alagille syndrome, autoimmune hepatitis, destruction of the bile ducts (primary biliary cirrhosis), hardening of the bile ducts (primary sclerosing cholangitis), infection, and medication. A subject treated by the methods and uses disclosed herein may have chronic liver disease. A subject treated by the methods and uses disclosed herein may have hepatic cirrhosis. In certain embodiments, the subject has a liver disease selected from the group consisting of cryptogenic cirrhosis, postnecrotic cirrhosis, alcoholic cirrhosis, autoimmune cirrhosis, primary biliary cirrhosis, primary biliary cholangitis, chronic active hepatitis, primary sclerosing cholangitis, alpha-1 antitrypsin deficiency, cystic fibrosis, nonalcoholic fatty liver disease, galactosemia, alagille syndrome, autoimmune hepatitis, Wilson's disease, sarcoidosis, hemochromatosis, biliary atresia, portal hypertension, tyrosinemia, Gaucher disease, schistosomiasis, nodular regenerative hyperplasia of the liver, hepatic allograft rejection, Langerhans cell histiocytosis, hepatic graft-versus host disease following hematopoietic stem cell transplantation, short telomere syndrome, Budd Chiari Syndrome, chronic granulomatous hepatitis, some acute liver diseases, and congenital vascular disease (e.g., cavopulmonary shunt, abernethy malformation). In certain embodiments, the subject has primary sclerosing cholangitis. In certain embodiments, the subject has portal hypertension.

Hepatic fibrosis is mediated by hepatic stellate cells, which occupy the space between hepatocytes and sinusoidal endothelial cells, termed the perisinusoidal space. Following hepatic injury by any underlying etiology, hepatic stellate cells become activated and proliferate, producing excessive amounts of fibrillar extracellular matrix (ECM) proteins (e.g., collagen I and III). The changes in ECM composition induce liver sinusoidal endothelial cells (LSEC) to form a basement membrane in a process termed LSEC capillarization. These pathophysiological changes interfere with normal nutrient transport between sinusoidal blood and surrounding cells, especially hepatocytes, which leads to functional disturbance. If the liver damage-inducing agent is not properly removed, liver fibrogenesis perpetuates until the liver architecture is strongly distorted, and can then progress to the late stages of liver disease, liver cirrhosis and hepatocellular carcinoma, and thus could finally lead to liver failure and death (Dewidar et. al., Cells. 2019 November; 8(11): 1419). In some embodiments, a subject treated by the methods and uses disclosed herein may have hepatic fibrosis.

Early-stage chronic liver disease is known as compensated chronic liver disease, and involves nonspecific symptoms such as fatigue and/or weight loss. Late stage chronic liver disease is known as decompensated chronic liver disease. At this stage, the liver is nonfunctional due to extensive hepatic fibrosis. Clinical complications associated with decompensated chronic liver disease may involve portal hypertension, primary sclerosing cholangitis, enlarged blood vessels (e.g., esophageal varices), fatigue, easy bruising and bleeding, itching (pruritus), yellowing of the skin and eyes (jaundice), fluid build-up in the abdomen (ascites), fluid build-up in the ankles and legs, abdominal pain, nausea, fever, weight loss, confusion or memory loss (associated with hepatic encephalopathy), or liver cancer. Chronic liver disease may also impair estrogen catabolism, leading to hyperestrogenism. The resulting excess estrogen in the body may manifest as palmar erythema, spider angiomas, gynecomastia, and testicular atrophy. A subject treated by the methods and uses disclosed herein may have chronic liver disease. In some embodiments, the subject has compensated chronic liver disease. In some embodiments, the subject has decompensated chronic liver disease. In some embodiments, the subject has one or more clinical complications associated with chronic liver disease. In some embodiments, the one or more clinical complications associated with chronic liver disease are selected from the group consisting of portal hypertension, enlarged blood vessels (e.g., esophageal varices), fatigue, easy bruising and bleeding, itching (pruritus), yellowing of the skin and eyes (jaundice), fluid build-up in the abdomen (ascites), fluid build-up in the ankles and legs, abdominal pain, nausea, fever, weight loss, confusion or memory loss (associated with hepatic encephalopathy), or liver cancer. In some embodiments, the one or more clinical complications is associated with decompensated chronic liver disease. In some embodiments, the one or more clinical complications is portal hypertension. In some embodiments, the one or more clinical complications is primary sclerosing cholangitis.

Since hepatic fibrosis is irreversible when the disease has progressed to decompensated chronic liver disease, treatment is focused on halting the progression of the disease and management of symptoms to improve overall quality of life. Patients diagnosed with decompensated chronic liver disease have an average life expectancy between 1 and 3 years. At this stage, treatment is targeted toward the root cause of the disease, such as mitigating risk factors including heavy alcohol use, obesity, type-2 diabetes, drug use, and/or chemical/toxin exposure. In cases involving severe cirrhosis, treatment includes liver transplant. The five year survival rate after liver transplant in decompensated chronic liver disease patients is approximately 75% (Kim et. al., Am J Transplant. 2018 January; 18 Suppl 1:172-253).

A major complication of chronic liver disease is portal hypertension. Portal hypertension is a result of resistance in portal vein blood flow, and may occur due to cirrhotic or non-cirrhotic causes. Portal hypertension often develops in the setting of cirrhosis, schistosomiasis, or extrahepatic portal vein thrombosis, and results from resistance to portal blood flow. In some embodiments, a subject treated by the methods and uses disclosed herein may have chronic liver disease. In some embodiments, the subject has portal hypertension. In some embodiments, the subject has chronic liver disease and portal hypertension.

A major complication of chronic liver disease is primary sclerosing cholangitis (PSC). PSC is a chronic progressive disorder of unknown etiology. It is characterized by inflammation, thickening, and abnormal formation of fibrous tissue (fibrosis) within the bile ducts, and both the bile ducts within the liver (intrahepatic) and outside the liver (extrahepatic) are affected in PSC, and results in obstructed bile flow from the liver, which is known as cholestasis. In some embodiments, a subject treated by the methods and uses disclosed herein may have chronic liver disease. In certain embodiments, the subject has primary sclerosing cholangitis. In some embodiments, the subject has chronic liver disease and primary sclerosing cholangitis. In some embodiments, the subject has obstructed bile flow from the liver.

A major complication of chronic liver disease is Hepatopulmonary syndrome (HPS). HPS is a pulmonary disorder of liver disease, and is characterized by a triad of liver disease, arterial hypoxemia, and intrapulmonary vascular abnormalities. Severe hepatic dysfunction is not required for diagnosis, and HPS can present in patients with mild liver disease. In some embodiments, a subject treated by the methods and uses disclosed herein may have chronic liver disease. In some embodiments, the subject has chronic liver disease and HPS. In some embodiments, the subject has HPS. In some embodiments, the subject has HPS and severe hepatic dysfunction. In some embodiments, the subject has HPS and mild liver disease.

Another complication associated with chronic liver disease is hepatic encephalopathy. Hepatic encephalopathy is a neuropsychiatric syndrome caused by increased levels of ammonia in a patient with impaired liver function due to cirrhosis. Increased levels of ammonia that are normally cleared in the liver instead accumulate in the blood, eventually traveling to and damaging the brain. Eventually, affected individuals may develop symptoms related to progressive dysfunction of the brain, including, for example, personality changes, intellectual impairment, impaired memory, and loss of consciousness (coma). The severity of hepatic encephalopathy follows a graded scale where grade 0/minimal involves normal mental status with minimal changes in memory; grade 1 involves a lack of awareness or shortened attention span; grade 2 involves lethargy, apathy, disorientation, personality change, and asterixis; grade 3 involves somnolence and confusion; grade 4 involves entering into a coma. In some embodiments, a subject treated by the methods and uses disclosed herein may have chronic liver disease. In some embodiments, the subject has hepatic encephalopathy. In some embodiments the subject has chronic liver disease and hepatic encephalopathy. In some embodiments, the subject has grade 0 hepatic encephalopathy. In some embodiments, the subject has grade 1 hepatic encephalopathy. In some embodiments, the subject has grade 2 hepatic encephalopathy. In some embodiments, the subject has grade 3 hepatic encephalopathy. In some embodiments, the subject has grade 4 hepatic encephalopathy. In some embodiments, treatment according to the methods and uses disclosed herein reduces the severity of hepatic encephalopathy. In some embodiments, treatment according to the methods and uses disclosed herein reduces the severity of hepatic encephalopathy from grade 4 to grade 3 hepatic encephalopathy. In some embodiments, treatment according to the methods and uses disclosed herein reduces the severity of hepatic encephalopathy from grade 4 to grade 2 hepatic encephalopathy. In some embodiments, treatment according to the methods and uses disclosed herein reduces the severity of hepatic encephalopathy from grade 4 to grade 1 hepatic encephalopathy. In some embodiments, treatment according to the methods and uses disclosed herein reduces the severity of hepatic encephalopathy from grade 4 to grade 0 hepatic encephalopathy. In some embodiments, treatment according to the methods and uses disclosed herein reduces the severity of hepatic encephalopathy from grade 3 to grade 2 hepatic encephalopathy. In some embodiments, treatment according to the methods and uses disclosed herein reduces the severity of hepatic encephalopathy from grade 3 to grade 1 hepatic encephalopathy. In some embodiments, treatment according to the methods and uses disclosed herein reduces the severity of hepatic encephalopathy from grade 3 to grade 0 hepatic encephalopathy. In some embodiments, treatment according to the methods and uses disclosed herein reduces the severity of hepatic encephalopathy from grade 2 to grade 1 hepatic encephalopathy. In some embodiments, treatment according to the methods and uses disclosed herein reduces the severity of hepatic encephalopathy from grade 2 to grade 0 hepatic encephalopathy. In some embodiments, treatment according to the methods and uses disclosed herein reduces the severity of hepatic encephalopathy from grade 1 to grade 0 hepatic encephalopathy. In some embodiments, treatment according to the methods and uses disclosed herein reduces the severity of hepatic encephalopathy to grade 3 hepatic encephalopathy. In some embodiments, treatment according to the methods and uses disclosed herein reduces the severity of hepatic encephalopathy to grade 2 hepatic encephalopathy. In some embodiments, treatment according to the methods and uses disclosed herein reduces the severity of hepatic encephalopathy to grade 1 hepatic encephalopathy. In some embodiments, treatment according to the methods and uses disclosed herein reduces the severity of hepatic encephalopathy to grade 0 hepatic encephalopathy.

Another complication associated with chronic liver disease is jaundice. Jaundice is a discoloration of the eyes, skin, and mucous membrane due to overproduction or impaired clearance of bilirubin. Hemoglobin and myoglobin metabolism produces bilirubin in the spleen, which then circulates throughout the body while bound to albumin. A functional liver dissociates the bilirubin-albumin complex, producing conjugated bilirubin. A cirrhotic liver cannot dissociate the bilirubin-albumin complex, and unconjugated bilirubin deposits in various tissues of the body, leading to accumulation of bile salts, pruritus, and clinically visible jaundice. In some embodiments a subject treated by the methods and uses disclosed herein may have chronic liver disease. In some embodiments, the subject has jaundice. In some embodiments, the subject has one or more symptoms of jaundice selected from the group consisting of weight gain, fever, and abdominal pain. In some embodiments, the subject has one or more complications associated with jaundice. In some embodiments, the one or more complications associated with jaundice is spontaneous bacterial peritonitis. Another complication of chronic liver disease is ascites. Ascites occurs upon disruption of the pressure gradient (typically due to portal hypertension) between intravascular and extravascular fluid spaces, thus allowing extravascular fluid to accumulate in the anterior peritoneal cavity. A complication associated with ascites is spontaneous bacterial peritonitis (SBP). SBP occurs when bacteria seeps through the gastrointestinal tract and infects the ascetic fluid. This infection spreads to the peritoneal membrane and causes inflammation. Chronic liver disease patients with ascites and/or SBP present with weight gain, fever, and abdominal pain. Treatment may include removal of fluid from the abdomen, administration of diuretic medicines, and/or treatment of SBP with cephalosporins. In some embodiments, a subject treated by the methods and uses disclosed herein may have chronic liver disease. In some embodiments, the subject has ascites. In some embodiments, the subject has chronic liver disease and ascites. In some embodiments, the subject has ascites and spontaneous bacterial peritonitis (SBP). In some embodiments, the spontaneous bacterial peritonitis is treated by removal of fluid from the abdomen. In some embodiments, the spontaneous bacterial peritonitis is treated by administration of diuretic medicines. In some embodiments, the spontaneous bacterial peritonitis is treated by administration of cephalosporins.

Chronic liver disease (CLD) is diagnosed according to the etiology of the disease, as well as the complications presented in the patient. The diagnostic procedure typically involves laboratory diagnostics, imaging technology, and clinical algorithms to determine both the etiology and presence of hepatic fibrosis. A liver biopsy is considered the gold standard diagnostic method to identify cirrhosis, but is invasive compared to the laboratory and imaging diagnostics. Radiologic imaging methods used in the diagnosis of chronic liver disease may include the ultrasound, computed tomography, transient elastography, Doppler scans, and electroencephalograms. Ultrasound detects the size, echogenicity nodularity of the liver, thereby diagnosing liver cirrhosis. Other benefits of ultrasound in CLD include measurement of portal vein diameter as portal vein diameter increases in portal hypertension, and assessment of a clot in the hepatic vein (Budd-Chari syndrome) and portal vein in portal vein thrombosis. Computed tomography scans can precisely reveal lesions in the liver or obstruction of biliary channels. Transient elastography (TE) detects early stages of cirrhosis, and can detect cardiovascular damage in patients with NAFLD. TE is the most effective approach to diagnose cirrhosis in chronic liver disease. Doppler scans are used in the diagnosis of Budd-Chiari and portal vein thrombosis. Electroencephalography (EEG) may be used to monitor electrical activity in the brain in a disease CLD setting involving hepatic encephalopathy. Diagnostic methodologies related to the methods of the present disclosure are enumerated further below. In some embodiments, a subject undergoes one or more of a liver biopsy, ultrasound, computed tomography, transient elastography, Doppler scans, and/or electroencephalograms to determine if the subject has chronic liver disease.

Short telomere syndromes represent an archetype of premature aging syndromes, where accelerated telomere shortening results in organ dysfunction, typically manifested in the form of reduced lung function and ILD. The most common telomere-related mutation-associated ILD is idiopathic pulmonary fibrosis (IPF). Up to one-third of individuals with familial IPF have shortened telomeres and/or carry a telomere-related mutation, and 1 in 10 individuals with sporadic IPF have telomere-related mutations. Regardless of ILD phenotype, individuals with short telomeres and/or known telomere-related mutations have more rapid disease progression and shorter lung transplant-free survival. Although a population of patients with chronic liver disease and concurrent ILD has been identified, the relationship between chronic liver disease and ILD has yet to be elucidated. Both liver cirrhosis and pulmonary fibrosis are fibrotic disorders characterized by epithelial/endothelial injury with dysregulated healing leading to fibroproliferation and tissue remodeling (Rodriguez-Pons et al., Arch Bronconeumol. 2018 November; 54(11):582-584; Mercer and Chambers, Biochim Biophys Acta. 2013 July; 1832(7):1018-27; Shahangian et al., Lung 2014; 192:421-427; Raevens and Fallon, Hepatology, 2018 November 68: 2016-2028). It is thought that there is significant overlap in the pathogenesis of fibrosis, despite the variable causes for different organs. One proposed mechanism suggests that sustained activation of populations of mesenchymal cells leads to the excessive deposition of extracellular matrix proteins and gross tissue distortion, resulting in complete loss of organ function (Mercer and Chambers, Biochim Biophys Acta. 2013 July; 1832(7):1018-27). In the most common and fatal form of lung fibrosis, idiopathic pulmonary fibrosis (IPF), epithelial injury is thought to progress through dysregulated epithelial—mesenchymal interactions to a cycle of aberrant tissue repair and injury.

In some embodiments, a subject treated by the methods and uses disclosed herein may have chronic liver disease and portal hypertension. In some embodiments, a subject treated by the methods and uses disclosed herein may have chronic liver disease and primary sclerosing cholangitis. In some embodiments, a subject treated by the methods and uses disclosed herein may have chronic liver disease and hepatopulmonary syndrome (HPS). In some embodiments, a subject treated by the methods and uses disclosed herein may have chronic liver disease, and a short telomere syndrome. In certain embodiments, the short telomere syndrome is interstitial lung disease (ILD). In certain embodiments, the short telomere syndrome is pulmonary fibrosis. In certain embodiments, the subject has chronic liver disease and ILD. In certain embodiments, the subject has chronic liver disease and pulmonary fibrosis. In certain embodiments, the subject has chronic liver disease and a complication of the lungs. In some embodiments, a complication of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, a complication of the lungs is selected from the group consisting of interstitial lung disease (ILD), pulmonary fibrosis, or idiopathic pulmonary fibrosis. In certain embodiments, the complication of the lungs is pulmonary fibrosis. In certain embodiments, the complication of the lungs is idiopathic pulmonary fibrosis. In certain embodiments, the complication of the lungs is interstitial lung disease (ILD).

### 3. Portal Hypertension

In part, the disclosure relates to TβRII antagonists that can be used to treat a disorder associated with chronic liver disease. In part, the disclosure relates TβRII antagonists that can be used to treat portal hypertension associated with chronic liver disease. In part, the disclosure relates TβRII antagonists that can be used to treat portal hypertension. In part, the disclosure relates TβRII antagonists that can be used to treat a complication associated with portal hypertension, such as, for example, pulmonary fibrosis or interstitial lung disease (ILD). Accordingly, the disclosure provides methods of treating portal hypertension, or a complication associated with portal hypertension (e.g., pulmonary fibrosis or ILD) comprising administering to a patient in need thereof one or more TβRII antagonists as described herein. Optionally, such methods further comprise administering to the patient one or more additional active agents and/or supportive therapies for treating portal hypertension or a complication associated with portal hypertension (e.g., pulmonary fibrosis or ILD).

In some embodiments, the disease or condition to be treated is portal hypertension associated with chronic liver disease. In some embodiments, the disease or condition to be treated is portal hypertension. In some embodiments, a TβRII antagonist for use in treating portal hypertension associated with chronic liver disease is administered to a subject in need thereof. In some embodiments, a TβRII antagonist for use in treating portal hypertension is administered to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disease or condition to be treated is a complication associated with portal hypertension. In some embodiments, the disease or condition to be treated is ILD associated with portal hypertension. In some embodiments, a TβRII antagonist for use in treating ILD associated with portal hypertension is administered to a subject in need thereof. In some embodiments, the disclosure provides a method of treating ILD associated with portal hypertension, comprising administering a TβRII antagonist to a subject in need thereof. In some embodiments, the disease or condition to be treated is pulmonary fibrosis associated with portal hypertension. In some embodiments, a TβRII antagonist for use in treating pulmonary fibrosis associated with portal hypertension is administered to a subject in need thereof. In some embodiments, the disclosure provides a method of treating pulmonary fibrosis associated with portal hypertension, comprising administering a TβRII antagonist to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disclosure provides methods of treating portal hypertension, comprising administering a TβRII antagonist to a subject in need thereof, wherein the treatment increases the subject's length of life when compared to a reference subject that is not receiving treatment. In some embodiments, the disclosure provides methods of treating portal hypertension, comprising administering a TβRII antagonist to a subject in need thereof, wherein the treatment reduces the subject's risk of death due to portal hypertension when compared to a reference subject that is not receiving treatment. In some embodiments, the disclosure provides methods of treating portal hypertension, comprising administering a TβRII antagonist to a subject in need thereof, wherein the treatment reduces the subject's risk of hospitalization due to portal hypertension when compared to a reference subject that is not receiving treatment. In some embodiments, a reference subject that is not receiving treatment has similar characteristics to the subject (height, sex, age, race, weight) and is not receiving one or more of TβRII antagonists of the present disclosure. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Portal Hypertension: Symptoms and Complications

Portal hypertension is a major complication of chronic liver disease and results from resistance in portal blood flow occurring due to cirrhotic or non-cirrhotic causes. Cirrhosis is the most common cause of portal hypertension. The liver receives blood from the hepatic artery and the portal vein, and each runs alongside the bile duct. The hepatic artery delivers highly-oxygenated blood to hepatocytes. The portal vein supplies the majority of blood to the liver from the gastrointestinal tract, gall bladder, spleen, and pancreas. Venous blood from the gastrointestinal tract contains not only digested nutrients, but also toxins and microbes. Once within the liver, blood from the hepatic artery and portal vein empty into hepatic sinusoids, where highly-oxygenated blood (from the hepatic artery) is combined with poorly oxygenated blood (from the portal vein), resulting in delivery of blood with reduced oxygenation to hepatocytes. Hepatic stellate cells occupy the space between hepatocytes and sinusoidal endothelial cells, termed the perisinusoidal space. When hepatic stellate cells become activated and proliferate, the buildup of fibrotic tissue in the perisinusoidal space compresses the central veins and hepatic sinusoids. As central veins and hepatic sinusoids compress, pressure increases in the portal vein, leading to portal hypertension. Normal portal vein pressures range from 5-10 mm Hg. Venous pressure more than 5 mm Hg greater than the inferior vena cava pressure is defined as portal hypertension, and is typically a portal venous pressure above 7 mm Hg. In some embodiments, a subject treated by the methods and uses disclosed herein may have portal hypertension. In some embodiments, the subject has a venous pressure more than 5 mm Hg greater than the inferior vena cava pressure. In some embodiments, the subject has a venous pressure above 7 mm Hg. In some embodiments, administration of a TβRII antagonist reduces the venous pressure. In some embodiments, administration of a TβRII antagonist reduces the venous pressure below 7 mm Hg. In some embodiments, administration of a TβRII antagonist reduces the venous pressure to less than 5 mm Hg greater than the inferior vena cava pressure. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Portal hypertension may also result from prehepatic and post-hepatic causes. The most common cause of prehepatic portal hypertension is portal vein thrombosis, which refers to the complete or partial obstruction of blood flow in the portal vein due to the presence of a thrombus (clot) in the vasal lumen. Post-hepatic causes of portal hypertension include Budd-Chiari syndrome, which occurs when the hepatic vein becomes thrombosed, resulting in marked dilatation and congestion of the central vein. A subject treated by the methods and uses disclosed herein may have portal hypertension. In some embodiments, the subject has prehepatic portal hypertension. In some embodiments, the subject has post-hepatic portal hypertension. In some embodiments, the subject has complete or partial obstruction of blood flow in the portal vein. In some embodiments, the subject has complete or partial obstruction of blood flow in the portal vein due to a thrombus. In some embodiments, administration of a TβRII antagonist relieves the complete or partial obstruction of blood flow in the portal vein. In some embodiments, administration of a TβRII antagonist relieves the complete or partial obstruction of blood flow in the portal vein. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Symptoms associated with portal hypertension are similar to those described above with regard to chronic liver disease. Specifically, symptoms and complications of portal hypertension include gastrointestinal bleeding (variceal bleeding), ascites, swollen abdomen, abdominal discomfort, jaundice, coagulopathy, spider angiomata, splenomegaly, dilated abdominal wall veins, encephalopathy, reduced levels of platelets, and/or decreased white blood cell counts. In some embodiments, a subject treated by the methods and uses disclosed herein may have portal hypertension. In some embodiments, the subject has a symptom or complication associated with portal hypertension selected from the group consisting of gastrointestinal bleeding (variceal bleeding), ascites, swollen abdomen, abdominal discomfort, jaundice, coagulopathy, spider angiomata, splenomegaly, dilated abdominal wall veins, encephalopathy, reduced levels of platelets, and/or decreased white blood cell counts.

In some embodiments, the subject has a symptom or complication associated with portal hypertension selected from the group consisting of gastrointestinal bleeding (variceal bleeding), ascites, swollen abdomen, abdominal discomfort, jaundice, coagulopathy, spider angiomata, splenomegaly, dilated abdominal wall veins, encephalopathy, reduced levels of platelets, and/or decreased white blood cell counts. In some embodiments, administration of a TβRII antagonist relieves one or more symptoms or complications associated with portal hypertension. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Portal hypertension can be diagnosed in several ways. Imaging studies of patients with portal hypertension help define portal venous anatomy. Duplex doppler ultrasonography is used to determine the direction and velocity of portal flow. MRI (magnetic resonance imaging) and computed tomography (CT) may also be used in diagnosis, and are capable of providing similar information. Endoscopy is the standard diagnostic approach in patients with acute gastrointestinal hemorrhage. Gastrointestinal endoscopy allows the physician to visualize and biopsy the mucosa of the upper gastrointestinal tract including the esophagus, stomach, and duodenum. In this procedure, an endoscope is passed through the mouth into the esophagus, and captures images of the esophagus, stomach and duodenum. Diagnostic methodologies related to the methods of the present disclosure are enumerated further below.

Treatment of portal hypertension focuses on treatment and/or prevention of complications associated with portal hypertension, and decreasing portal pressure. Ascites is typically treated by administering a diuretic therapy to the patient to reduce sodium retention in the kidneys. However, abdominal paracentesis may be used to therapeutically remove ascites and is useful in relieving respiratory difficulties, acute oliguria or pain. Use of beta-blockers has been shown to decrease portal pressures, but side effects of the drugs are sometimes prohibitive. Use of vasopressin in the acutely bleeding patient is effective and works by decreasing splanchnic blood flow. The short-term (7 days) administration of antibiotics to patients with cirrhosis and acute variceal hemorrhage is the standard of practice. In these patients, antibiotics have been shown to reduce the rate of re-bleeding and increase overall survival. Antibiotics are typically administered to the portal hypertension patient irrespective of the presence of ascites. In some embodiments, a subject treated by the methods and uses disclosed herein may have portal hypertension. In some embodiments, the subject is treated with a diuretic therapy. In some embodiments, a TβRII antagonist is administered concurrently with a diuretic therapy. In some embodiments, the subject is treated by abdominal paracentesis. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Portal hypertension can be treated by a variety of endoscopic therapies and/or surgical shunting procedures. Acute variceal hemorrhage is managed by variceal ligation with elastic rings, commonly called banding, where elastic rings are placed over a varix. Sclerotherapy involves injection of a sclerosing agent directly into and around the varices. Sclerosing agents include sodium morrhuate, sodium tetradecyl sulfate, ethanolamine oleate, or absolute alcohol. Nonsurgical Transjugular Intrahepatic Portal-Systemic Shunt (TIPSS) is a radiologic procedure contraindicated to severe liver dysfunction, renal failure, and heart failure. In TIPSS, access to the hepatic vein is obtained through the right internal jugular vein. A needle is passed through the liver parenchyma into the portal vein, followed by dilation of the tract and placement of a stent. The stent is then dilated to achieve a portal to hepatic vein gradient of less than 10 mm Hg. Liver transplantation is the only effective treatment for end-stage liver disease. In some embodiments, a subject treated by the methods and uses disclosed herein may have portal hypertension. In some embodiments, the subject is treated with an endoscopic therapy. In some embodiments, the subject is treated with a surgical shunting procedure. In some embodiments, the subject is treated with sclerotherapy. In some embodiments, the subject is treated with one or more sclerosing agents. In some embodiments, the subject is treated with one or more sclerosing agents selected from the group consisting of sodium morrhuate, sodium tetradecyl sulfate, ethanolamine oleate, or absolute alcohol.

As described above, a population of patients with chronic liver disease and concurrent ILD has been identified, however the relationship between chronic liver disease and ILD has yet to be elucidated. In some embodiments, a subject treated by the methods and uses disclosed herein may have portal hypertension and a short telomere syndrome. In certain embodiments, the short telomere syndrome is interstitial lung disease (ILD). In certain embodiments, the short telomere syndrome is pulmonary fibrosis. In certain embodiments, the subject has portal hypertension and ILD. In certain embodiments, the subject has portal hypertension and pulmonary fibrosis. In certain embodiments, the subject has portal hypertension and a complication of the lungs. In some embodiments, a complication of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, a complication of the lungs is selected from the group consisting of interstitial lung disease (ILD), pulmonary fibrosis, or idiopathic pulmonary fibrosis. In certain embodiments, the complication of the lungs is pulmonary fibrosis. In certain embodiments, the complication of the lungs is idiopathic pulmonary fibrosis. In certain embodiments, the complication of the lungs is interstitial lung disease (ILD).

### 4. Primary Sclerosing Cholangitis

In part, the disclosure relates to TβRII antagonists that can be used to treat a disorder associated with chronic liver disease. In part, the disclosure relates TβRII antagonists that can be used to treat primary sclerosing cholangitis associated with chronic liver disease. In part, the disclosure relates TβRII antagonists that can be used to treat primary sclerosing cholangitis. In part, the disclosure relates TβRII antagonists that can be used to treat a complication associated with primary sclerosing cholangitis, such as, for example, pulmonary fibrosis or interstitial lung disease (ILD). Accordingly, the disclosure provides methods of treating primary sclerosing cholangitis, or a complication associated with primary sclerosing cholangitis (e.g., pulmonary fibrosis or ILD) comprising administering to a patient in need thereof one or more TβRII antagonists as described herein. Optionally, such methods further comprise administering to the patient one or more additional active agents and/or supportive therapies for treating primary sclerosing cholangitis or a complication associated with primary sclerosing cholangitis (e.g., pulmonary fibrosis or ILD).

In some embodiments, the disease or condition to be treated is primary sclerosing cholangitis associated with chronic liver disease. In some embodiments, the disease or condition to be treated is primary sclerosing cholangitis. In some embodiments, a TβRII antagonist for use in treating primary sclerosing cholangitis associated with chronic liver disease is administered to a subject in need thereof. In some embodiments, a TβRII antagonist for use in treating primary sclerosing cholangitis is administered to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disease or condition to be treated is a complication associated with primary sclerosing cholangitis. In some embodiments, the disease or condition to be treated is ILD associated with primary sclerosing cholangitis. In some embodiments, a TβRII antagonist for use in treating ILD associated with primary sclerosing cholangitis is administered to a subject in need thereof. In some embodiments, the disclosure provides a method of treating ILD associated with primary sclerosing cholangitis, comprising administering a TβRII antagonist to a subject in need thereof. In some embodiments, the disease or condition to be treated is pulmonary fibrosis associated with primary sclerosing cholangitis. In some embodiments, a TβRII antagonist for use in treating pulmonary fibrosis associated with primary sclerosing cholangitis is administered to a subject in need thereof. In some embodiments, the disclosure provides a method of treating pulmonary fibrosis associated with primary sclerosing cholangitis, comprising administering a TβRII antagonist to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

The disclosure provides methods of treating or preventing a disease or condition associated with a TGFβ superfamily member by administering to a subject one or more TβRII antagonists, including, but not limited to, TβRII polypeptides and fusion proteins comprising the same as described herein. In some embodiments, the disease or condition is associated with dysregulated TGFβ1 or TGFβ3 signaling. In some embodiments, the disease or condition to be treated is primary sclerosing cholangitis. In some embodiments, a TβRII antagonist for use in treating chronic liver disease is administered to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disease or condition to be treated is primary sclerosing cholangitis. In some embodiments, the disease or condition to be treated is a complication associated with primary sclerosing cholangitis. In some embodiments, the disease or condition to be treated is pulmonary fibrosis associated with primary sclerosing cholangitis. In some embodiments, the disease or condition to be treated is ILD associated with primary sclerosing cholangitis. In some embodiments, a TβRII antagonist for use in treating primary sclerosing cholangitis is administered to a subject in need thereof. In some embodiments, a TβRII antagonist for use in treating pulmonary fibrosis associated with primary sclerosing cholangitis is administered to a subject in need thereof. In some embodiments, a TβRII antagonist for use in treating ILD associated with primary sclerosing cholangitis is administered to a subject in need thereof. In some embodiments, the disclosure provides a method of treating pulmonary fibrosis associated with primary sclerosing cholangitis, comprising administering a TβRII antagonist to a subject in need thereof. In some embodiments, a TβRII antagonist for use in treating ILD associated with primary sclerosing cholangitis is administered to a subject in need thereof. In some embodiments, the disclosure provides a method of treating pulmonary fibrosis associated with primary sclerosing cholangitis, comprising administering a TβRII antagonist to a subject in need thereof. In some embodiments, the disclosure provides a method of treating ILD associated with primary sclerosing cholangitis, comprising administering a TβRII antagonist to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disclosure provides methods of treating primary sclerosing cholangitis or a complication associated with primary sclerosing cholangitis, comprising administering a TβRII antagonist to a subject in need thereof, wherein the treatment increases the subject's length of life when compared to a reference subject that is not receiving treatment. In some embodiments, the disclosure provides methods of treating primary sclerosing cholangitis or a complication associated with primary sclerosing cholangitis, comprising administering a TβRII antagonist to a subject in need thereof, wherein the treatment reduces the subject's risk of death due to primary sclerosing cholangitis when compared to a reference subject that is not receiving treatment. In some embodiments, the disclosure provides methods of treating primary sclerosing cholangitis or a complication associated with primary sclerosing cholangitis, comprising administering a TβRII antagonist to a subject in need thereof, wherein the treatment reduces the subject's risk of hospitalization due to primary sclerosing cholangitis when compared to a reference subject that is not receiving treatment. In some embodiments, a reference subject that is not receiving treatment has similar characteristics to the subject (height, sex, age, race, weight) and is not receiving one or more of TβRII antagonists of the present disclosure. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Primary Sclerosing Cholangitis: Symptoms and Complications

Primary sclerosing cholangitis (PSC) is a chronic progressive disorder of unknown etiology. It is characterized by inflammation, thickening, and abnormal formation of fibrous tissue (fibrosis) within the bile ducts. Bile ducts facilitate bile flow to throughout the body. Bile is a fluid that contains water, electrolytes, bile salts, phospholipids, cholesterol, and bilirubin. Bile flow aids in digestion and absorption of dietary fats, vitamins, and other nutrients, and helps eliminate excess cholesterol, bilirubin, waste, and toxins from the body. A problem with normal bile flow often results in malabsorption of vital nutrients and the accumulation of toxic materials in the body. Both the bile ducts within the liver (intrahepatic) and outside the liver (extrahepatic) are affected in PSC, and results in obstructed bile flow from the liver, which is known as cholestasis. In some embodiments, a subject treated by the methods and uses disclosed herein may have primary sclerosing cholangitis. In some embodiments, the subject has obstructed bile flow from the liver. In some embodiments, the subject has obstructed intrahepatic bile flow. In some embodiments, the subject has obstructed extrahepatic bile flow. In some embodiments, the subject has cholestasis. In some embodiments, administration of a TβRII antagonist alleviates intrahepatic bile flow in the subject. In some embodiments, administration of a TβRII antagonist alleviates extrahepatic bile flow in the subject. In some embodiments, administration of a TβRII antagonist alleviates the cholestasis in the subject. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

PSC is characterized by cholestasis resulting from inflammation, thickening, and/or fibrosis within the bile ducts. Symptoms associated with primary sclerosing cholangitis include pruritus, jaundice, abdominal pain, nausea, and hepatomegaly. The majority of patients with primary sclerosing cholangitis have concurrent ulcerative colitis, which is an inflammatory bowel disease (IBD) of unknown cause that is characterized by chronic inflammation and ulceration of the lining of the major portion of the large intestine (colon). PSC also most often occurs in middle-aged males. Numerous autoimmune or immune-mediated disorders have been reported to occur in association with PSC, and include sarcoidosis, thyroid disease, Peyronie's disease, retroperitoneal fibrosis, psoriasis, rheumatoid arthritis, Celiac disease, Sjogren's syndrome, chronic pancreatitis, lupus, diabetes mellitus, Wegener's granulomatosis, pyoderma gangrenosum, Grave's disease, Langerhans cell histiocytosis and certain immunodeficiency disorders. In some embodiments, a subject treated by the methods and uses disclosed herein may have primary sclerosing cholangitis. In some embodiments, a TβRII antagonist may be used in a method for treating a subject with primary sclerosing cholangitis. In some embodiments, the TβRII antagonist may be used in a method for treating one or more diseases associated with primary sclerosing cholangitis. In some embodiments, the one or more diseases associated with primary sclerosing cholangitis are selected from the group consisting of sarcoidosis, thyroid disease, Peyronie's disease, retroperitoneal fibrosis, psoriasis, rheumatoid arthritis, Celiac disease, Sjogren's syndrome, chronic pancreatitis, lupus, diabetes mellitus, Wegener's granulomatosis, pyoderma gangrenosum, Grave's disease, and Langerhans cell histiocytosis.

Tests and procedures used to diagnose PSC include liver function blood tests, MRI imaging of the bile ducts, x-ray endoscopic retrograde cholangiopancreatography (ERCP), and liver biopsy. An MRI is the most frequently used imaging test used to diagnose primary sclerosing cholangitis. Diagnostic methodologies related to the methods of the present disclosure are enumerated further below. There is no specific, universal treatment for individuals with PSC. Treatment is directed toward the symptoms and complications presented in each patient, and focuses on slowing the progression of the disorder. Endoscopic surgery may be performed to remove blockages and enlarge narrowed bile ducts in order to prevent liver deterioration. Antibiotics are typically administered to control inflammation and/or infection. Liver transplantation is the only known proven effective treatment for individuals with PSC, and is generally reserved for individuals with advanced symptoms of PSC, including end-stage liver disease.

As described above, a population of patients with chronic liver disease and concurrent ILD has been identified, however the relationship between chronic liver disease and ILD has yet to be elucidated. In some embodiments, a subject treated by the methods and uses disclosed herein may have primary sclerosing cholangitis, and a short telomere syndrome. In certain embodiments, the short telomere syndrome is interstitial lung disease (ILD). In certain embodiments, the short telomere syndrome is pulmonary fibrosis. In certain embodiments, the subject has primary sclerosing cholangitis and ILD. In certain embodiments, the subject has primary sclerosing cholangitis and pulmonary fibrosis. In certain embodiments, the subject has primary sclerosing cholangitis and a complication of the lungs. In some embodiments, a complication of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, a complication of the lungs is selected from the group consisting of interstitial lung disease (ILD), pulmonary fibrosis, or idiopathic pulmonary fibrosis. In certain embodiments, the complication of the lungs is pulmonary fibrosis. In certain embodiments, the complication of the lungs is idiopathic pulmonary fibrosis. In certain embodiments, the complication of the lungs is interstitial lung disease (ILD).

Hepatopulmonary Syndrome

In part, the disclosure relates to TβRII antagonists that can be used to treat a disorder associated with chronic liver disease. In part, the disclosure relates TβRII antagonists that can be used to treat hepatopulmonary syndrome associated with chronic liver disease. In part, the disclosure relates TβRII antagonists that can be used to treat hepatopulmonary syndrome. In part, the disclosure relates TβRII antagonists that can be used to treat a complication associated with hepatopulmonary syndrome, such as, for example, pulmonary fibrosis or interstitial lung disease (ILD). Accordingly, the disclosure provides methods of treating hepatopulmonary syndrome, or a complication associated with hepatopulmonary syndrome (e.g., pulmonary fibrosis or ILD) comprising administering to a patient in need thereof one or more TβRII antagonists as described herein. Optionally, such methods further comprise administering to the patient one or more additional active agents and/or supportive therapies for treating hepatopulmonary syndrome or a complication associated with hepatopulmonary syndrome (e.g., pulmonary fibrosis or ILD).

In some embodiments, the disease or condition to be treated is hepatopulmonary syndrome associated with chronic liver disease. In some embodiments, the disease or condition to be treated is hepatopulmonary syndrome. In some embodiments, a TβRII antagonist for use in treating hepatopulmonary syndrome associated with chronic liver disease is administered to a subject in need thereof. In some embodiments, a TβRII antagonist for use in treating hepatopulmonary syndrome is administered to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disease or condition to be treated is a complication associated with hepatopulmonary syndrome. In some embodiments, the disease or condition to be treated is ILD associated with hepatopulmonary syndrome. In some embodiments, a TβRII antagonist for use in treating ILD associated with hepatopulmonary syndrome is administered to a subject in need thereof. In some embodiments, the disclosure provides a method of treating ILD associated with hepatopulmonary syndrome, comprising administering a TβRII antagonist to a subject in need thereof. In some embodiments, the disease or condition to be treated is pulmonary fibrosis associated with hepatopulmonary syndrome. In some embodiments, a TβRII antagonist for use in treating pulmonary fibrosis associated with hepatopulmonary syndrome is administered to a subject in need thereof. In some embodiments, the disclosure provides a method of treating pulmonary fibrosis associated with hepatopulmonary syndrome, comprising administering a TβRII antagonist to a subject in need thereof. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

The disclosure provides methods of treating or preventing a disease or condition associated with a TGFβ superfamily member by administering to a subject one or more TβRII antagonists, including, but not limited to, TβRII polypeptides and fusion proteins comprising the same as described herein. In some embodiments, the disease or condition is associated with dysregulated TGFβ1 or TGFβ3 signaling. In some embodiments, the disease or condition to be treated is hepatopulmonary syndrome. In some embodiments, the disease or condition to be treated is pulmonary fibrosis or ILD associated with hepatopulmonary syndrome. In some embodiments, the disease or condition to be treated is pulmonary fibrosis associated with hepatopulmonary syndrome. In some embodiments, the disease or condition to be treated is ILD associated with hepatopulmonary syndrome. In some embodiments, a TβRII antagonist for use in treating hepatopulmonary syndrome or a complication associated with hepatopulmonary syndrome is administered to a subject in need thereof. In some embodiments, hepatopulmonary syndrome is characterized as type I hepatopulmonary syndrome. In some embodiments the disease or condition to be treated is type I hepatopulmonary syndrome. In some embodiments, a TβRII antagonist for use in treating type I hepatopulmonary syndrome is administered to a subject in need thereof. In some embodiments, hepatopulmonary syndrome is characterized as type II hepatopulmonary syndrome. In some embodiments the disease or condition to be treated is type II hepatopulmonary syndrome. In some embodiments, a TβRII antagonist for use in treating type II hepatopulmonary syndrome is administered to a subject in need thereof. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disease or condition to be treated is pulmonary fibrosis associated with hepatopulmonary syndrome. In some embodiments, a TβRII antagonist for use in treating pulmonary fibrosis associated with hepatopulmonary syndrome is administered to a subject in need thereof. In some embodiments, the disclosure provides a method of treating pulmonary fibrosis associated with hepatopulmonary syndrome, comprising administering a TβRII antagonist to a subject in need thereof. In some embodiments, the disclosure provides methods of treating pulmonary fibrosis associated with hepatopulmonary syndrome, comprising administering a TβRII antagonist to a subject in need thereof, wherein the hepatopulmonary syndrome is type I hepatopulmonary syndrome. In some embodiments, the disclosure provides methods of treating pulmonary fibrosis associated with hepatopulmonary syndrome, comprising administering a TβRII antagonist to a subject in need thereof, wherein the hepatopulmonary syndrome is type II hepatopulmonary syndrome. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disease or condition to be treated is ILD associated with hepatopulmonary syndrome. In some embodiments, a TβRII antagonist for use in treating ILD associated with hepatopulmonary syndrome is administered to a subject in need thereof. In some embodiments, the disclosure provides a method of treating ILD associated with hepatopulmonary syndrome, comprising administering a TβRII antagonist to a subject in need thereof. In some embodiments, the disclosure provides methods of treating ILD associated with hepatopulmonary syndrome, comprising administering a TβRII antagonist to a subject in need thereof, wherein the hepatopulmonary syndrome is type I hepatopulmonary syndrome. In some embodiments, the disclosure provides methods of treating ILD associated with hepatopulmonary syndrome, comprising administering a TβRII antagonist to a subject in need thereof, wherein the hepatopulmonary syndrome is type II hepatopulmonary syndrome. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

In some embodiments, the disclosure provides methods of treating hepatopulmonary syndrome or a complication associated with hepatopulmonary syndrome (e.g., pulmonary fibrosis or ILD), comprising administering a TβRII antagonist to a subject in need thereof, wherein the treatment increases the subject's length of life when compared to a reference subject that is not receiving treatment. In some embodiments, the disclosure provides methods of treating hepatopulmonary syndrome, comprising administering a TβRII antagonist to a subject in need thereof, wherein the treatment reduces the subject's risk of death due to hepatopulmonary syndrome when compared to a reference subject that is not receiving treatment. In some embodiments, the disclosure provides methods of treating hepatopulmonary syndrome, comprising administering a TβRII antagonist to a subject in need thereof, wherein the treatment reduces the subject's risk of hospitalization due to hepatopulmonary syndrome when compared to a reference subject that is not receiving treatment. In some embodiments, a reference subject that is not receiving treatment has similar characteristics to the subject (height, sex, age, race, weight) and is not receiving one or more of TβRII antagonists of the present disclosure. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Hepatopulmonary Syndrome: Symptoms and Complications

Hepatopulmonary syndrome (HPS) is a pulmonary disorder of liver disease. HPS is characterized by a triad of liver disease, arterial hypoxemia, and intrapulmonary vascular abnormalities (Rodriguez-Roisin et al, New England Journal of Medicine. 2008; 358(22):2378-2387). Due to the use of varied criteria in the diagnostic methodology for HPS, rates reporting the prevalence of HPS vary widely. Studies evaluating the different diagnostic methodologies for HPS report a prevalence of 4-47% in patients with liver cirrhosis (Rodriguez-Roisin et al, New England Journal of Medicine. 2008; 358(22):2378-2387; Stoller et al, West J Med. 1995 August; 163(2):133-8; Hopkins et al, Am J Cardiol. 1992 Aug. 15; 70(4):516-9). However, severe hepatic dysfunction is not required for diagnosis, and HPS can present in patients with mild liver disease (Abrams et al, Gastroenterology. 1995 Oct; 109(4):1283-8). The vast majority of HPS patients (82%) initially present with liver disease, while a minority (18%) present with lung (pulmonary) complications (Fallon et al, Hepatology. 2000; 32:859-865; Krowka et al, Chest. 1993; 104(2):515-521; Swanson et al, Hepatology. 2005; 41(5):1122-1129). Progressive shortness of breath at rest or upon exertion is reported in 95% of HPS patients and typically develops after years of liver disease. HPS is associated with a poor prognosis, and has a mortality rate more than double the rate of patients with a similar severity of cirrhotic liver disease. The only known effective treatment for HPS is liver transplantation.

Progressive shortness of breath is known as dyspnea and commonly occurs in the form of platypnea in HPS patients. Platypnea, or more specifically, platypnea-orthodeoxia syndrome (POS), manifests as breathlessness experienced in the upright position, which is improved when lying down (supine position). Dyspnea occurs in as many as 88% of HPS patients, and is typically evaluated using chest X-rays and computed tomography (CT) imaging (Krowka et al., Chest. 1993; 104(2):515-521). These methods may be accompanied by the use of electrocardiogram and spirometry tests to detect issues with the heart, and obtain measurements of the subject's airflow and lung capacity. Subjects experiencing dyspnea may be co-treated with bronchodilators, steroids, and/or pain medication to alleviate symptoms. In some embodiments, a subject treated by the methods and uses disclosed herein may have dyspnea. In certain embodiments, the subject has platypnea-orthodeoxia syndrome. In certain embodiments, the subject is administered one or more of bronchodilators, steroids, and/or pain medication. In some embodiments, the subject is administered one or more of bronchodilators, steroids, and/or pain medication prior to administration of a TβRII antagonist of the present disclosure. In some embodiments, the subject is administered one or more bronchodilators prior to administration of a TβRII antagonist of the present disclosure. In some embodiments, the subject is administered one or more steroids prior to administration of a TβRII antagonist of the present disclosure. In some embodiments, the subject is administered one or more pain medications prior to administration of a TβRII antagonist of the present disclosure. In some embodiments, the subject is administered one or more of bronchodilators, steroids, and/or pain medication after administration of a TβRII antagonist of the present disclosure. In some embodiments, the subject is administered one or more bronchodilators after administration of a TβRII antagonist of the present disclosure. In some embodiments, the subject is administered one or more steroids after administration of a TβRII antagonist of the present disclosure. In some embodiments, the subject is administered one or more pain medications after administration of a TβRII antagonist of the present disclosure. In some embodiments, the subject is administered one or more of bronchodilators, steroids, and/or pain medication in combination with administration of a TβRII antagonist of the present disclosure. In some embodiments, the subject is administered one or more bronchodilators in combination with administration of a TβRII antagonist of the present disclosure. In some embodiments, the subject is administered one or more steroids in combination with administration of a TβRII antagonist of the present disclosure. In some embodiments, the subject is administered one or more pain medications in combination with administration of a TβRII antagonist of the present disclosure. In some embodiments, the TβRII antagonist is a TβRII polypeptide or fusion protein consisting of, or comprising, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Hepatopulmonary syndrome is characterized in part by the presence of liver disease, with a prevalence of 4-47% in patients with liver cirrhosis (Rodriguez-Roisin et al, New England Journal of Medicine. 2008; 358(22):2378-2387; Stoller et al, West J Med. 1995 August; 163(2):133-8; Hopkins et al, Am J Cardiol. 1992 Aug. 15; 70(4):516-9). Cirrhosis comprises a late stage of scarring (fibrosis) of the liver. Cirrhosis may result from a variety of diseases and/or conditions not limited to alcohol abuse, chronic viral hepatitis, fat accumulation in the liver as a result of nonalcoholic fatty liver disease, iron buildup as a result of hemochromatosis, cystic fibrosis, copper accumulation in the liver resulting from Wilson's disease, poor formation of bile ducts resulting from biliary atresia, alpha-1 antitrypsin deficiency, galactosemia, digestive disorders such as alagille syndrome, autoimmune hepatitis, destruction of the bile ducts (primary biliary cirrhosis), hardening of the bile ducts (e.g., primary sclerosing cholangitis), infection, and medication. A subject treated by the methods and uses disclosed herein may have hepatopulmonary syndrome and liver disease. In certain embodiments, the subject has liver disease selected from the group consisting of be cryptogenic cirrhosis, postnecrotic cirrhosis, alcoholic cirrhosis, autoimmune cirrhosis, primary biliary cirrhosis, primary biliary cholangitis, chronic active hepatitis, primary sclerosing cholangitis, alpha-1 antitrypsin deficiency, cystic fibrosis, nonalcoholic fatty liver disease, galactosemia, alagille syndrome, autoimmune hepatitis, Wilson's disease, sarcoidosis, hemochromatosis, biliary atresia, portal hypertension, tyrosinemia, Gaucher disease, schistosomiasis, nodular regenerative hyperplasia of the liver, hepatic allograft rejection, Langerhans cell histiocytosis, hepatic graft-versus host disease following hematopoietic stem cell transplantation, short telomere syndrome, Budd Chiari Syndrome, chronic granulomatous hepatitis, some acute liver diseases, and congenital vascular disease (e.g., cavopulmonary shunt, abernethy malformation). In certain embodiments, the subject has primary sclerosing cholangitis. In certain embodiments, the subject has hepatopulmonary syndrome and primary sclerosing cholangitis. In certain embodiments, the subject has portal hypertension. In certain embodiments, the subject has hepatopulmonary syndrome and portal hypertension.

Hepatopulmonary syndrome patients may also present with symptoms and/or complications of chronic liver disease (i.e. weakness, fatigue, anorexia, a history of gastrointestinal bleeding due to esophageal or gastric varices, inability to concentrate, menstrual irregularities, decreased libido, skin fragility, easy bruising, and pruritus). Other symptoms of chronic liver disease include ascites, anasarca, nail changes, splenomegaly, a large or small liver, caput medusae, gynecomastia, palmar erythema, testicular atrophy, jaundice. A subject treated by the methods and uses disclosed herein may present symptoms and/or complications of liver disease. In certain embodiments, the symptoms and/or complications of liver disease are selected from the group consisting of weakness, fatigue, anorexia, gastrointestinal bleeding, inability to concentrate, menstrual irregularities, decreased libido, skin fragility, bruising, pruritus, ascites, anasarca, nail changes, splenomegaly, a large or small liver, caput medusae, gynecomastia, palmar erythema, testicular atrophy, and jaundice.

Patients with hepatopulmonary syndrome often present hemodynamic and/or pulmonary complications associated with liver dysfunction. Hemodynamic complications in patients with hepatopulmonary syndrome may include spider nevi (also known as spider angiomata/angiomas or nevi araneus) and hyperdynamic circulation. Spider angiomas are a common clinical feature of patients with the hepatopulmonary syndrome. Spider angioma lesions are characterized by a central red arteriole surrounded by a radial pattern of thin-walled capillaries. Patients with cutaneous spider angiomas have been found to have more systemic and pulmonary vasodilation, more profound gas exchange abnormalities, and less hypoxic pulmonary vasoconstriction. These lesions are implicated as cutaneous markers of intrapulmonary vascular dilations. Other hemodynamic and/or pulmonary complications in HPS patients may include clubbing of the fingers or toes, hypertrophic osteoarthropathy, orthodeoxia, and/or cyanosis (abnormal bluish discoloration of skin or mucous membranes due to tissues near the skin surface having low oxygen saturation). A subject treated by the methods and uses disclosed herein may have one or more complications associated with hepatopulmonary syndrome. In certain embodiments, the one or more complications associated with hepatopulmonary syndrome are selected from the group consisting of spider nevi (also known as spider angiomata/angiomas or nevi araneus), hyperdynamic circulation, clubbing of the fingers or toes, hypertrophic osteoarthropathy, orthodeoxia, and cyanosis.

Hepatopulmonary syndrome is divided into two subtypes based upon two distinct different patterns of intrapulmonary vascular abnormalities noted in patients (Krowka and Cortese 1990 November; 98:1053-4). Type I HPS is characterized by diffuse intrapulmonary vascular dilatations at the capillary and pre-capillary levels. In type I HPS, dilated blood vessels may reach a diameter of 15-100 μm or greater, resulting in decreased oxygen diffusion into the dilated capillaries. The vasodilatation of alveolar capillaries in type I HPS results in an excessive amount of blood flowing into the normally ventilated alveoli, which results in overperfusion of the capillary bed, thus leading to arterial hypoxemia. In type II HPS, intrapulmonary arteriovenous shunting results in blood flow that bypasses the alveolus and passes directly into central circulation, thus leading to arterial hypoxemia. In some embodiments, a subject treated by the methods and uses disclosed herein may have Type I HPS or Type II HPS. In certain embodiments the subject has type I HPS. In certain embodiments, the subject has type II HPS. In certain embodiments, the subject has diffuse intrapulmonary vascular dilatations at the capillary and pre-capillary levels. In certain embodiments, the subject has dilated blood vessels that reach a diameter of 15-100 μm. In certain embodiments, the subject has dilated blood vessels that reach a diameter greater than 100 μm. In certain embodiments, the subject has intrapulmonary arteriovenous shunts. In certain embodiments, the subject suffers from arterial hypoxemia.

Pre-capillary and capillary dilation in hepatopulmonary syndrome is thought to occur due to decreased hepatic clearance of inflammatory and angiogenic cytokines. Recent studies of HPS have demonstrated the involvement of several cytokines/growth factors, such as TNF-α, IL-1, TGF-β, endothelin-1, and VEGF, all of which are similarly involved in the pathogenesis of interstitial lung disease (ILD) or pulmonary fibrosis (Shahangian et al., Lung 2014; 192:421-427; Raevens and Fallon, Hepatology, 2018 November 68: 2016-2028). HPS patients often have concurrent pulmonary disease. Recent studies have identified a high prevalence (21%) of HPS in subjects with short telomere syndromes including ILD and pulmonary fibrosis (Gorgy et al., Chest 2015; 148:1019-1026). One study identified concurrent ILD in 17% of HPS patients listed for liver transplantation (Shahangian et al., Lung 2014; 192:421-427). Short telomere syndromes represent an archetype of premature aging syndromes, where accelerated telomere shortening results in organ dysfunction, typically manifested in the form of reduced lung function and ILD. The most common telomere-related mutation-associated ILD is idiopathic pulmonary fibrosis (IPF). Up to one-third of individuals with familial IPF have shortened telomeres and/or carry a telomere-related mutation, and 1 in 10 individuals with sporadic IPF have telomere-related mutations. Regardless of ILD phenotype, individuals with short telomeres and/or known telomere-related mutations have more rapid disease progression and shorter lung transplant-free survival. Although a population of HPS patients with concurrent ILD has been identified, the relationship between HPS and ILD has yet to be elucidated. In some embodiments, a subject of the present disclosure has a short telomere syndrome. In some embodiments, a subject of the present disclosure has shortened telomeres. In some embodiments, a subject of the present disclosure has shortened telomeres and ILD. In some embodiments, a subject of the present disclosure carries a telomere-related mutation. In some embodiments, s subject of the present disclosure has accelerated telomere shortening resulting in organ dysfunction, typically manifested in the form of reduced lung function and/or ILD. In some embodiments, a subject of the present disclosure with short telomeres and/or a telomere-related mutation has rapid disease progression and shorter survival time without a lung transplant.

In some embodiments, a subject treated by the methods and uses disclosed herein may have HPS and a short telomere syndrome. In certain embodiments, the short telomere syndrome is interstitial lung disease (ILD). In certain embodiments, the short telomere syndrome is pulmonary fibrosis. In certain embodiments, the subject has HPS and ILD. In certain embodiments, the subject has HPS and pulmonary fibrosis. In certain embodiments, the subject has HPS and a complication of the lungs. In some embodiments, a complication of the lungs is selected from the group consisting of interstitial lung disease (ILD), pulmonary fibrosis, idiopathic pulmonary fibrosis, alveolitis, recurrent aspiration, and pulmonary vasculopathy. In some embodiments, a complication of the lungs is selected from the group consisting of interstitial lung disease (ILD), pulmonary fibrosis, or idiopathic pulmonary fibrosis. In certain embodiments, the complication of the lungs is pulmonary fibrosis. In certain embodiments, the complication of the lungs is idiopathic pulmonary fibrosis. In certain embodiments, the complication of the lungs is interstitial lung disease (ILD).

Typical diagnostic methods for hepatopulmonary syndrome test for impaired oxygenation and the presence of intrapulmonary vascular dilatations and/or shunts. These methods include detection of the presence of one or more of liver disease, portal hypertension, an elevated alveolar-arterial difference for the partial pressure of oxygen (AaO2), reduced partial pressure of arterial oxygen, and/or evidence of intrapulmonary vascular dilatations. Typical diagnostic methodologies include chest radiography, echocardiography, chest computed tomography, contrast pulmonary angiography, and pulmonary function tests. A subject treated by the methods and uses disclosed herein may be tested for impaired oxygenation and the presence of intrapulmonary vascular dilatations and/or shunts.

Patients with hepatopulmonary syndrome are typically screened for intrapulmonary vascular dilations. The presence of intrapulmonary vascular dilations can be confirmed using one of three imaging modalities: contrast-enhanced echocardiography (CEE), technetium 99m-labeled macroaggregated albumin scanning, and pulmonary angiography.

### 6. Diagnostic Methods

Contrast-Enhanced Transthoracic Echocardiography

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using contrast-enhanced transthoracic echocardiography (CTTE). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. CTTE is used to detect intrapulmonary vascular dilation in subjects with hepatopulmonary syndrome. In CTTE, agitated saline contrast solution produces microbubbles greater than 20 μm in diameter, which are larger than the normal 8-15 μm diameter of pulmonary capillary. The agitated saline is administered in a peripheral vein in the patient's arm, where under normal circumstances, the microbubbles are filtered by the pulmonary capillary bed and do not appear in the left side of the heart. In the presence of an intrapulmonary or intracardiac right-to-left shunt, the microbubbles will opacify the left heart chambers. A subject treated by the methods and uses disclosed herein may be tested for the presence of intrapulmonary vascular dilatations and/or shunts. In certain embodiments, contrast-enhanced transthoracic echocardiography is performed to detect the presence of intrapulmonary vascular dilatations and/or shunts.

Technetium 99m-Labeled Macroaggregated Albumin (Tc-99m MAA)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using Technetium 99m-labeled macroaggregated albumin (Tc-99m MAA) scanning. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Technetium 99m-labeled macroaggregated albumin scanning may be used as an alternative method for identifying intrapulmonary vascular dilations. Tc-99m MAA scanning involves intravenous injection of labeled albumin macroaggregates greater than 20 μm in diameter. Similar to contrast-enhanced echocardiography, the macroaggregates exceed the normal 8-15 μm pulmonary capillary diameter. Scans identifying uptake of the radionucleotide by the brain suggest that the macroaggregates passed through either an intrapulmonary or intracardiac shunt. In normal patients 3% to 5% of albumin macroaggregates pass through the pulmonary vasculature; therefore, a calculated shunt fraction (percent of uptake in brain compared to lungs) of greater than or equal to 6% is considered abnormal. A shunt fraction value of less than 5% obviates the need for further evaluation, whereas, a shunt fraction greater than 5% suggests intrapulmonary or extrapulmonary shunting. Tc-99m MAA scanning is unable to distinguish between intrapulmonary or extrapulmonary shunting and is less sensitive than contrast-enhanced echocardiography. A subject treated by the methods and uses disclosed herein may be tested for the presence of intrapulmonary vascular dilatations and/or shunts. In certain embodiments, technetium 99m-labeled macroaggregated albumin (Tc-99m MAA) scanning may be used to detect the presence of intrapulmonary vascular dilatations and/or shunts. In certain embodiments, the subject exhibits intrapulmonary shunting on contrast-enhanced echocardiogram. In some embodiments, the subject has a shunt fraction greater than 5%. In some embodiments, the subject has a shunt fraction greater than 10%. In some embodiments, the subject has a shunt fraction greater than 20%. In some embodiments, administration of a TβRII antagonist according to the methods and uses disclosed herein decreases the shunt fraction to less than 20%. In some embodiments, administration of a TβRII antagonist according to the methods and uses disclosed herein decreases the shunt fraction to less than %. In some embodiments, administration of a TβRII antagonist according to the methods and uses disclosed herein decreases the shunt fraction to less than 5%.

Contrast Transesophageal Echocardiography

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering to a patient in need thereof one or more TβRII antagonists as described herein, wherein the subject is evaluated using contrast transesophageal echocardiography. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Contrast transesophageal echocardiography can also be used to detect intrapulmonary vascular dilations with greater specificity due to the direct visualization of microbubbles in the pulmonary veins as they enter the left atrium. In this method, an endoscope with an ultrasound device is inserted down the esophagus in order to provide an image of the heart without interference from skin, muscle, or bone tissue. This transthoracic methodology is more sensitive than the injection of technetium 99m-labeled macroaggregated albumin and less invasive than pulmonary angiography. The additional benefit is that cardiac function and pulmonary artery pressures can also be evaluated. In some embodiments, a subject treated by the methods and uses disclosed herein may be tested for the presence of intrapulmonary vascular dilatations and/or shunts. In certain embodiments, contrast transesophageal echocardiography is performed to detect the presence of intrapulmonary vascular dilatations and/or shunts.

Pulmonary Angiography

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using pulmonary angiography. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Pulmonary angiography may be used as an alternative method for direct radiographic visualization of intrapulmonary vascular dilations in patients with the hepatopulmonary syndrome. A contrast dye is injected into the blood vessels, and highlights clots or other blockages in blood flow upon X-ray imaging. Pulmonary angiography is capable of distinguishing between the two types of pulmonary vascular dilations noted on type I and type II HPS, where type 1 is characterized by a pattern of finely diffuse infiltrates or blotches, and type II is characterized by discrete, localized arteriovenous communications. Pulmonary angiography is capable of excluding alternative causes of hypoxemia and/or identifying the type of intrapulmonary vascular dilations noted above. Pulmonary angiography is typically performed when hypoxia is severe (i.e., the partial pressure of oxygen is <60 mmHg). In some embodiments, a subject treated by the methods and uses disclosed herein may be tested for the presence of type I or type II hepatopulmonary syndrome. In some embodiments, the subject exhibits type I or type II hepatopulmonary syndrome based on pulmonary angiography. In some embodiments, the subject has type I hepatopulmonary syndrome based on pulmonary angiography. In some embodiments, the subject has type II hepatopulmonary syndrome based on pulmonary angiography. In some embodiments, the subject has a pattern of finely diffuse infiltrates as visualized using pulmonary angiography. In some embodiments, the subject has type I HPS, and has a pattern of finely diffuse infiltrates as visualized using pulmonary angiography. In some embodiments, the subject has localized arteriovenous communications as visualized using pulmonary angiography. In some embodiments, the subject has type II HPS, and has localized arteriovenous communications as visualized using pulmonary angiography.

Chest Radiography

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using chest radiography. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Chest radiography can be used together with other methods of detection described herein to determine if a subject has hepatopulmonary syndrome or complications thereof. Pulmonary complications and lung disease associated with hepatopulmonary syndrome may be detected using chest radiography. The most common radiographic abnormality on a routine chest radiograph is a reticular pattern (e.g., fine or ground glass, medium or irregular, coarse or honeycomb), however, nodular or mixed patterns (e.g., alveolar filling and/or increased interstitial markings) are not unusual. Basic patterns of diffuse lung disease include, but are not limited to, nodular, reticular (fine or ground glass, medium or irregular, coarse or honeycomb), linear (interlobular septal or Kerley lines and/or intralobular septal lines), combined reticular and/or nodular, destructive, alveolar, bronchial, and/or vascular. Although a chest radiograph is useful in suggesting presence of interstitial lung disease (ILD), the correlation between the radiographic pattern and the stage of disease (clinical or histopathologic) is generally poor. A radiographic finding of honeycombing (small cystic spaces) may correlate with pathologic findings and, when present, can portend a poor prognosis. In evaluation of ILD, it is important to review all previous chest films to assess the rate of change in disease activity.

Honeycombing can be a feature of end stage interstitial lung disease. It represents restructuring of the lung parenchyma, with simplification of the lung architecture and subsequent small cyst formation surrounded by fibrotic tissue. Accompanying bronchiolectasis is regularly present. The small subpleural spaces, which usually measure between 3 and 10 mm in diameter may connect with small airway and some collapse on expiratory imaging. Honeycombs are usually stacked and are not separated by intervening normal lung. Bronchiolectasis which can mimic honeycombing are usually separated by interposed lung parenchyma. The expiratory collapse of some of these spaces distinguishes them from the subpleural spaces formed by paraseptal emphysema, which do not decrease in size with expiration. In addition, honeycombs have slightly thicker walls than emphysematous spaces. Honeycombing forms an array of multilayered, stacked spaces, while paraseptal emphysematous spaces are single-layered (i.e., single-tiered). The differentiation of honeycombing, bronchiolectasis and paraseptal emphysema is typically achieved with additional analysis using high resolution computed tomography. Honeycombing can be seen in subjects suffering from, but not limited to IPF/UIP, other interstitial pneumonias, Langerhans cell histiocytosis (previously eosinophilic granuloma), collagen vascular diseases, healed necrotizing infections, end stage pneumoconiosis, and end stage hypersensitivity pneumonitis (i.e., extrinsic allergic alveolitis). Classic radiographic features of established ILD comprise symmetric, reticular (e.g., fine or ground glass, medium or irregular, coarse or honeycomb), opacities. Radiographic features are usually most pronounced at the lung bases.

In some embodiments, a subject has a chest radiograph with one or more radiographic abnormalities. In some embodiments, a subject has a chest radiograph with one or more of a reticular pattern, a modular pattern, or a mixed pattern. In some embodiments, a subject has a chest radiograph with one or more of a nodular pattern, reticular pattern, a linear pattern, a combined reticular and nodular pattern, a destructive pattern, an alveolar pattern, a bronchial pattern, and/or a vascular pattern. In some embodiments, a subject has a chest radiograph with a reticular pattern. In some embodiments, a subject has a chest radiograph with a honeycombing pattern. A chest radiograph can be normal in as many as 10 percent of patients with some forms of ILD, particularly those with hypersensitivity pneumonitis. Thus, a complete evaluation is generally undertaken even if a patient has a normal chest radiograph or radiographic evidence of ILD.

High Resolution-Computer Topography (HRCT)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using high resolution computer topography. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. A more sensitive alternative to chest radiography for diagnosing and/or evaluating pulmonary complications and/or lung disease associated with hepatopulmonary syndrome (e.g., ILD) is High Resolution-Computer Topography (HRCT). HRCT scans thin sections (3 mm or less) of the lung. HRCT can demonstrate character and/or distribution of fine structural abnormalities that are not visible on chest radiographs. Prone (e.g., face down) images are often obtained in addition to the usual supine (e.g., face up) images to differentiate between increased density due to ILD and/or dependent atelectasis at the posterior lung bases. HRCT is generally performed using a conventional CT scanner. However, imaging parameters are chosen to maximize spatial resolution, which include, but are not limited to, a narrow slice width (usually 1-2 mm), a high spatial resolution image reconstruction algorithm, field of view is minimization (e.g., to minimize the size of each pixel), and/or other scan factors (e.g., focal spot). Depending on the suspected diagnosis, an HRCT scan may be performed in both inspiration and expiration. HRCT is conventionally performed by taking thin sections (e.g., less than 3 mm) that are about 10-40 mm apart. The resulting scan is generally a few images that should be representative of the lungs in general, but that cover only approximately one tenth of the lungs.

HRCT patterns found in ILD, for example, may mirror common histopathological patterns of idiopathic interstitial pneumonias, which include but are not limited to fibrotic nonspecific interstitial pneumonia (NSIP), usual interstitial pneumonia (UIP), and/or centrilobular fibrosis. Among patients with ILD, the most common pathologic pattern is typically NSIP, which can be associated with an HRCT finding of ground glass opacities in a peripheral distribution and/or a lower proportion of coarse reticulation, but is not typically associated with a honeycomb pattern. Early HRCT changes are typically a narrow, often ill-defined, subpleural crescent of increased density in the posterior (dependent) segments of the lower lobes. As ILD progresses, there is usually volume loss associated with a reticular appearance and/or traction bronchiectasis. In a subset of patients with a histologic UIP pattern, the HRCT appearance is usually similar to that of fibrotic NSIP. However, sometimes the HRCT pattern is more consistent with that of UIP with bibasilar reticular opacities, associated with traction bronchiectasis and/or the development of subpleural honeycomb air spaces, which ultimately coalesce into large cystic air spaces. A honeycomb pattern can be indicative of a subject that is in advanced stages of disease and will not respond to immunosuppressive therapy. Centrilobular fibrosis is typically a rare pattern that may be associated with patchy ground glass or consolidative opacities with a central distribution on HRCT scanning. Centrilobular nodules and/or “tree-in-bud” patterns are other features of recurrent aspiration. In some embodiments, a subject with ILD has an HRCT scan that reveals lung parenchymal changes including any round glass opacity and fibrotic peripheral reticulations and honey combing without abnormalities. In some embodiments, a subject has undergone a High Resolution Computed Topography scan (HRCT) of the lungs prior to treatment. In some embodiments, a subject has undergone a HRCT scan of the lungs prior to treatment with one or more TβRII antagonists of the present disclosure. In some embodiments, a subject receives a HRCT scan of the lungs during treatment with one or more TβRII antagonists of the present disclosure. In some embodiments, a subject receives a HRCT scan of the lungs after treatment with one or more TβRII antagonists of the present disclosure. In some embodiments, the present disclosure provides methods of treating ILD, comprising administering a TβRII antagonist to a subject in need thereof, wherein an amount of fibrosis or pattern of interstitial pneumonia is determined by HRCT scan of the lungs of the subject.

In some embodiments, an HRCT scan of a subject has determined a presence of at least between about 1% and about 10% fibrosis of the lungs. In some embodiments, an HRCT scan of a subject has determined a presence of at least between about 5% and about 10% fibrosis of the lungs. In some embodiments, an HRCT scan of a subject has determined a presence of at least between about 10% and about 15% fibrosis of the lungs. In some embodiments, an HRCT scan of a subject has determined a presence of at least between about 15% and about 20% fibrosis of the lungs. In some embodiments, an HRCT scan of a subject has determined a presence of at least between about 20% and about 25% fibrosis of the lungs. In some embodiments, an HRCT scan of a subject has determined a presence of at least between about 25% and about 30% fibrosis of the lungs. In some embodiments, an HRCT scan of a subject has determined a presence of at least between about 30% and about 35% fibrosis of the lungs. In some embodiments, an HRCT scan of a subject has determined a presence of at least between about 35% and about 40% fibrosis of the lungs. In some embodiments, an HRCT scan of a subject has determined a presence of at least between about 40% and about 45% fibrosis of the lungs. In some embodiments, an HRCT scan of a subject has determined a presence of at least between about 45% and about 50% fibrosis of the lungs. In some embodiments, an HRCT scan of a subject has determined a presence of at least between about 1% and about 10% fibrosis of the lungs. In some embodiments, an HRCT scan of a subject has determined a presence of at least between about 10% and about 20% fibrosis of the lungs. In some embodiments, an HRCT scan of a subject has determined a presence of at least between about 20% and about 30% fibrosis of the lungs. In some embodiments, an HRCT scan of a subject has determined a presence of at least between about 30% and about 40% fibrosis of the lungs. In some embodiments, an HRCT scan of a subject has determined a presence of at least between about 40% and about 50% fibrosis of the lungs. In some embodiments, an HRCT scan of a subject has determined a presence of at least 10% fibrosis of the lungs. In some embodiments, an HRCT scan of a subject has determined a presence of greater than or equal to 10% fibrosis of the lungs.

In some embodiments, the methods described herein reduce fibrosis of the lungs in a subject by at least between about 1% and about 10% fibrosis of the lungs. In some embodiments, the methods described herein reduce fibrosis of the lungs in a subject by at least between about 5% and about 10% fibrosis of the lungs. In some embodiments, the methods described herein reduce fibrosis of the lungs in a subject by at least between about 10% and about 15% fibrosis of the lungs. In some embodiments, the methods described herein reduce fibrosis of the lungs in a subject by at least between about 15% and about 20% fibrosis of the lungs. In some embodiments, the methods described herein reduce fibrosis of the lungs in a subject by at least between about 20% and about 25% fibrosis of the lungs. In some embodiments, the methods described herein reduce fibrosis of the lungs in a subject by at least between about 25% and about 30% fibrosis of the lungs. In some embodiments, the methods described herein reduce fibrosis of the lungs in a subject by at least between about 30% and about 35% fibrosis of the lungs. In some embodiments, the methods described herein reduce fibrosis of the lungs in a subject by at least between about 35% and about 40% fibrosis of the lungs. In some embodiments, the methods described herein reduce fibrosis of the lungs in a subject by at least between about 40% and about 45% fibrosis of the lungs. In some embodiments, a the methods described herein reduce fibrosis of the lungs in a subject by at least between about 45% and about 50% fibrosis of the lungs. In some embodiments, the methods described herein reduce fibrosis of the lungs in a subject by at least 10% fibrosis of the lungs. In some embodiments, the methods described herein reduce fibrosis of the lungs in a subject by greater than or equal to 10% fibrosis of the lungs.

Functional Respiratory Imaging (FRI)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using functional respiratory imaging. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. HRCT can be paired with a technology called Functional Respiratory Imaging (FRI). FRI is a non-invasive measurement of the patient-specific respiratory system. A set of distinct biomarkers analyzes exposure, structure and function of the lungs and airways. Phase 1 comprises medical imagine, typically using HRCT scans. Phase 2 comprises image processing and measurements. Phase 3 comprises flow simulation, where Computational Fluid Dynamics (CFD) is used to quantify airflow and exposure to inhaled particles. FRI provides regional information on airway resistance, air trapping, ventilation mapping, ventilation/perfusion reserve, lung and lobar volume, emphysema, internal airflow distribution, blood vessel volume, nodule volume, airway (wall) volume, and aerosol deposition. In some embodiments, a subject has undergone FRI of the lungs prior to treatment. In some embodiments, a subject has undergone an FRI analysis of the lungs prior to treatment with one or more TβRII antagonists of the present disclosure. In some embodiments, a subject receives an FRI analysis of the lungs during treatment with one or more TβRII antagonists of the present disclosure. In some embodiments, a subject receives an FRI analysis of the lungs after treatment with one or more TβRII antagonists of the present disclosure. In some embodiments, the present disclosure provides methods of treating lung disease and/or pulmonary complications associated with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein an amount of fibrosis or pattern of interstitial pneumonia is determined by FRI analysis of the lungs of the subject.

In some embodiments, the present disclosure provides methods of treating lung disease and/or pulmonary complications associated with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein an improvement in the amount of fibrosis in the lungs is determined by FRI analysis of the lungs of the subject. In some embodiments, an improvement in the amount of fibrosis in the lungs of a subject comprises a decrease in the amount of fibrosis in the lungs.

In some embodiments, FRI analysis of a subject has determined an improvement of at least between about 1% and about 10% of fibrosis of the lungs. In some embodiments, FRI analysis of a subject has determined an improvement of at least between about 5% and about 10% of fibrosis of the lungs. In some embodiments, FRI analysis of a subject has determined an improvement of at least between about 10% and about 15% of fibrosis of the lungs. In some embodiments, FRI analysis of a subject has determined an improvement of at least between about 15% and about 20% of fibrosis of the lungs. In some embodiments, FRI analysis of a subject has determined an improvement of at least between about 20% and about 25% of fibrosis of the lungs. In some embodiments, FRI analysis of a subject has determined an improvement of at least between about 25% and about 30% of fibrosis of the lungs. In some embodiments, FRI analysis of a subject has determined an improvement of at least between about 30% and about 35% of fibrosis of the lungs. In some embodiments, FRI analysis of a subject has determined an improvement of at least between about 35% and about 40% of fibrosis of the lungs. In some embodiments, FRI analysis of a subject has determined an improvement of at least between about 40% and about 45% of fibrosis of the lungs. In some embodiments, FRI analysis of a subject has determined an improvement of at least between about 45% and about 50% of fibrosis of the lungs. In some embodiments, FRI analysis of a subject has determined an improvement of at least between about 1% and about 10% of fibrosis of the lungs. In some embodiments, FRI analysis of a subject has determined an improvement of at least between about 10% and about 20% of fibrosis of the lungs. In some embodiments, FRI analysis of a subject has determined an improvement of at least between about 20% and about 30% of fibrosis of the lungs. In some embodiments, FRI analysis of a subject has determined an improvement of at least between about 30% and about 40% of fibrosis of the lungs. In some embodiments, FRI analysis of a subject has determined an improvement of at least between about 40% and about 50% of fibrosis of the lungs.

In some embodiments, FRI analysis of a subject has determined a decrease of at least between about 1% and about 10% of fibrosis of the lungs. In some embodiments, FRI analysis of a subject has determined a decrease of at least between about 5% and about 10% of fibrosis of the lungs. In some embodiments, FRI analysis of a subject has determined a decrease of at least between about 10% and about 15% of fibrosis of the lungs. In some embodiments, FRI analysis of a subject has determined a decrease of at least between about 15% and about 20% of fibrosis of the lungs. In some embodiments, FRI analysis of a subject has determined a decrease of at least between about 20% and about 25% of fibrosis of the lungs. In some embodiments, FRI analysis of a subject has determined a decrease of at least between about 25% and about 30% of fibrosis of the lungs. In some embodiments, FRI analysis of a subject has determined a decrease of at least between about 30% and about 35% of fibrosis of the lungs. In some embodiments, FRI analysis of a subject has determined a decrease of at least between about 35% and about 40% of fibrosis of the lungs. In some embodiments, FRI analysis of a subject has determined a decrease of at least between about 40% and about 45% of fibrosis of the lungs. In some embodiments, FRI analysis of a subject has determined a decrease of at least between about 45% and about 50% of fibrosis of the lungs. In some embodiments, FRI analysis of a subject has determined a decrease of at least between about 1% and about 10% of fibrosis of the lungs. In some embodiments, FRI analysis of a subject has determined a decrease of at least between about 10% and about 20% of fibrosis of the lungs. In some embodiments, FRI analysis of a subject has determined a decrease of at least between about 20% and about 30% of fibrosis of the lungs. In some embodiments, FRI analysis of a subject has determined a decrease of at least between about 30% and about 40% of fibrosis of the lungs. In some embodiments, FRI analysis of a subject has determined a decrease of at least between about 40% and about 50% of fibrosis of the lungs.

Arterial Blood Gas Measurements

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using arterial blood gas measurements. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Pulmonary function tests (PFTs) may be performed in HPS patients with or without concurrent pulmonary disease. Pulmonary function tests in HPS patients without concurrent pulmonary disease may reveal normal flows and lung volumes, though patients may have reduced lung volumes.

Arterial blood gas can be measured to assess the severity of chronic liver disease, portal hypertension, primary sclerosing cholangitis, and hepatopulmonary syndrome. Arterial blood gas measurements are made by drawing a small amount of blood from the subject's artery. The oxygen and carbon dioxide levels in the blood are analyzed to determine if a patient's lungs are functioning properly. An arterial blood gas measurement may assess the pressure of oxygen dissolved in the blood (partial pressure of oxygen), the pressure of carbon dioxide dissolved in the blood (partial pressure of carbon dioxide; PaCO2), pH, and oxygen content and saturation in the blood (the amount of oxygen being carried by hemoglobin present in red blood cells). The difference between the oxygen concentration in the alveoli and arterial system represents the alveolar arterial oxygen gradient (AaO2). AaO2 is defined as PAO2-PaO2, where PAO2 represents alveolar oxygen pressure, and PaO2 represents arterial oxygen pressure (Faustini-Pereira et al., Annals of Hepatology; 2015; 14:361-368). Alveolar oxygen pressure (PAO2) is not easily measured directly and is typically estimated using the alveolar gas equation. Normal subjects tend to have AaO2 in the range of about 15 mm Hg or below, and a partial pressure of oxygen in the range of about 80 mm Hg or above. Subjects with mild HPS have an alveolar-arterial oxygen gradient (AaO2) above, or equal to, mmHg, with a partial pressure of arterial oxygen above, or equal to, 80 mmHg. Subjects with moderate HPS have an AaO2 gradient above, or equal to, 15 mmHg, with a partial pressure of oxygen above, or equal to, 60 mm Hg to 80 mm Hg. Subjects with severe HPS have an AaO2 gradient above, or equal to, 15 mmHg, with a partial pressure of oxygen above, or equal to, 50 to 60 mm Hg. Subjects with very severe HPS have an AaO2 gradient above, or equal to, 15 mmHg, with a partial pressure of oxygen below 50 mmHg (<300 mmHg while the patient is breathing 100% oxygen).

A subject treated by the methods and uses disclosed herein may be tested for the presence of mild to very severe hepatopulmonary syndrome. In some embodiments, the subject has mild HPS. In certain embodiments, the subject has moderate HPS. In certain embodiments, the subject has severe HPS. In certain embodiments, the subject has very severe HPS. In each subject (with mild, moderate, severe, and very severe HPS), administration of a TβRII antagonist of the present disclosure decreases the AaO2 gradient to be below 15 mm Hg, while increasing the partial pressure of arterial oxygen above 80 mm Hg. In certain embodiments, the subject has mild HPS with an AaO2 gradient above, or equal to, 15 mmHg, with a partial pressure of arterial oxygen above, or equal to, 80 mmHg. In certain embodiments, the subject has moderate HPS with an AaO2 gradient above, or equal to, 15 mmHg, with a partial pressure of oxygen above, or equal to, 60 mm Hg to 80 mm Hg. In certain embodiments, the subject has severe HPS with an AaO2 gradient above, or equal to, mmHg, with a partial pressure of oxygen above, or equal to, 50 to 60 mm Hg. In certain embodiments, the subject has very severe HPS with an AaO2 gradient above, or equal to, 15 mmHg, with a partial pressure of oxygen below 50 mmHg (<300 mmHg while the patient is breathing 100% oxygen). In some embodiments, administration of a TβRII antagonist reduces the severity of HPS in the subject. In some embodiments, administration of a TβRII antagonist reduces the severity of HPS in the subject from very severe to severe. In some embodiments, administration of a TβRII antagonist reduces the severity of HPS in the subject from severe to moderate. In some embodiments, administration of a TβRII antagonist reduces the severity of HPS in the subject from moderate to mild. In some embodiments, administration of a TβRII antagonist reduces the severity of HPS in a subject with very severe HPS and an AaO2 gradient above, or equal to, 15 mmHg, with a partial pressure of oxygen below 50 mmHg (<300 mmHg while the patient is breathing 100% oxygen), to severe HPS with an AaO2 gradient above, or equal to, 15 mmHg, with a partial pressure of oxygen above, or equal to, 50 to 60 mm Hg. In some embodiments, administration of a TβRII antagonist reduces the severity of HPS in a subject with very severe HPS and an AaO2 gradient above, or equal to, 15 mmHg, with a partial pressure of oxygen below 50 mmHg (<300 mmHg while the patient is breathing 100% oxygen), to moderate HPS with an AaO2 gradient above, or equal to, 15 mmHg, with a partial pressure of oxygen above, or equal to, 60 mm Hg to 80 mm Hg. In some embodiments, administration of a TβRII antagonist reduces the severity of HPS in a subject with very severe HPS and an AaO2 gradient above, or equal to, 15 mmHg, with a partial pressure of oxygen below 50 mmHg (<300 mmHg while the patient is breathing 100% oxygen), to mild HPS with an AaO2 gradient above, or equal to, 15 mmHg, with a partial pressure of arterial oxygen above, or equal to, 80 mmHg. In some embodiments, administration of a TβRII antagonist reduces the severity of HPS in a subject with severe HPS and an AaO2 gradient above, or equal to, 15 mmHg, with a partial pressure of oxygen above, or equal to, 50 to 60 mm Hg, to moderate HPS with an AaO2 gradient above, or equal to, 15 mmHg, with a partial pressure of oxygen above, or equal to, mm Hg to 80 mm Hg. In some embodiments, administration of a TβRII antagonist reduces the severity of HPS in a subject with severe HPS and an AaO2 gradient above, or equal to, 15 mmHg, with a partial pressure of oxygen above, or equal to, 50 to 60 mm Hg, to mild HPS with an AaO2 gradient above, or equal to, 15 mmHg, with a partial pressure of arterial oxygen above, or equal to, 80 mmHg. In some embodiments, administration of a TβRII antagonist reduces the severity of HPS in a subject with moderate HPS and an AaO2 gradient above, or equal to, 15 mmHg, with a partial pressure of oxygen above, or equal to, mm Hg to 80 mm Hg, to mild HPS with an AaO2 gradient above, or equal to, 15 mmHg, with a partial pressure of arterial oxygen above, or equal to, 80 mmHg. In some embodiments, administration of a TβRII antagonist decreases the subject's AaO2 gradient below 15 mm Hg, and increases the subject's partial pressure of arterial oxygen above 80 mm Hg

Spirometry and Forced Vital Capacity (FVC)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using Spirometry and/or Forced Vital Capacity. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Spirometry, or measuring of breath, is a major pulmonary function test that can determine volume and/or speed (flow) of air that is inhaled and exhaled by a subject. A spirometer is used to measure forced vital capacity (FVC) (measured in liters, milliliters, and/or percentage of predicted) in a forced expiratory volume (FEV) test, among other characteristics. In an FEV test, a subject takes a deep breath, and exhales into a sensor as hard and for as long as possible (e.g., at least 6 seconds). Inhalation can also be tested using spirometry. An FEV test is typically repeated at least three times to ensure accuracy. “Normal” ranges for FVC are typically considered to be between 80% and 100% of predicted. “Of predicted” refers to reporting the subject's results as a percentage of the known predicted values for a healthy subject of similar characteristics (e.g., height, sex, age, race, weight). Other measurements that can be taken include, but are not limited to, FEVs, wherein the FVC is measured within the first second of forced exhalation, and/or forced expiratory flow (FEF), which measures the flow of air coming out of the lung during the middle portion of forced expiration. An FEVs/FVC ratio is also typically calculated. Theoretical assumptions regarding HPS patients suggest that both spirometry and static lung volumes would be normal in the absence of concomitant pulmonary diseases. However, recent studies suggest that a reduction in forced vital capacity and maximum forced expiratory volume is more frequent in HPS patients.

In some embodiments, a subject of the present disclosure has an FVC of between about 100% and about 90% of predicted. In some embodiments, a subject of the present disclosure has an FVC of between about 90% and about 80% of predicted. In some embodiments, a subject of the present disclosure has an FVC of between about 80% and about 70% of predicted. In some embodiments, a subject of the present disclosure has an FVC of between about 70% and about 60% of predicted. In some embodiments, a subject of the present disclosure has an FVC of between about 60% and about 50% of predicted. In some embodiments, a subject of the present disclosure has an FVC of between about 50% and about 40% of predicted. In some embodiments, a subject of the present disclosure has an FVC of between about 40% and about 30% of predicted. In some embodiments, a subject of the present disclosure has an FVC of between about 30% and about 20% of predicted. In some embodiments, a subject of the present disclosure has an FVC of greater than or equal to 50% of predicted.

A general measurement of disease progression can be an annual rate of decline in FVC. In some embodiments, an annual rate of decline in forced vital capacity (FVC) of a subject is measured over a time period of at least one year after administration of one or more TβRII antagonists of the present disclosure. In some embodiments, an annual rate of decline in forced vital capacity (FVC) of a subject is measured over a time period of at least 52 weeks after administration of one or more TβRII antagonists of the present disclosure. In some embodiments, an annual rate of decline in forced vital capacity (FVC) of a subject is measured over a time period of at least one year after administration of one or more TβRII antagonists of the present disclosure and is compared to a baseline FVC measurement. In some embodiments, an annual rate of decline in forced vital capacity (FVC) of a subject is measured over a time period of at least 52 weeks after administration of one or more TβRII antagonists of the present disclosure and is compared to a baseline FVC measurement. In some embodiments, administration of one or more TβRII antagonists of the present disclosure results in a decline in an annual rate of forced vital capacity (FVC). In some embodiments, administration of one or more TβRII antagonists of the present disclosure results in a reduction in annual rate of decline of forced vital capacity (FVC). In some embodiments, an annual rate of decline in FVC has been slowed. In some embodiments, a subject is determined to have a slowing in the rate of decline in pulmonary function after administration of one or more TβRII antagonists of the present disclosure.

In some embodiments, administration of one or more TβRII antagonists of the present disclosure slows the annual rate of decline in FVC compared to a baseline measurement. In some embodiments, administration of one or more TβRII antagonists of the present disclosure slows the annual rate of decline in FVC compared to a subject administered standard of care (SOC).

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, steps comprising measuring at least one initial point of lung function in a subject; administering a Transforming Growth Factor-β Receptor II (TβRII) fusion polypeptide to the subject; re-measuring the at least one point of lung function in the subject; and based on the measuring, determining a change in the rate of decline in lung function of the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, steps comprising measuring at least one initial point of lung function in a subject; administering a Transforming Growth Factor-β Receptor II (TβRII) fusion polypeptide to the subject; re-measuring the at least one point of lung function in the subject; and based on the measuring, determining a change in the rate of decline in lung function of the subject.

In some embodiments, the rate of decline in lung function is measured by Forced Vital Capacity (FVC) of the subject. In some embodiments, the rate of decline in lung function is measured as an annual rate of decline in Forced Vital Capacity (FVC) of the subject.

In some embodiments, a subject of the present disclosure has an initial point of lung function comprising an FVC of between about 100% and about 90% of predicted. In some embodiments, a subject of the present disclosure has an initial point of lung function comprising an FVC of between about 90% and about 80% of predicted. In some embodiments, a subject of the present disclosure has an initial point of lung function comprising an FVC of between about 80% and about 70% of predicted. In some embodiments, a subject of the present disclosure has an initial point of lung function comprising an FVC of between about 70% and about 60% of predicted. In some embodiments, a subject of the present disclosure has an initial point of lung function comprising an FVC of between about 60% and about 50% of predicted. In some embodiments, a subject of the present disclosure has an initial point of lung function comprising an FVC of between about 50% and about 40% of predicted. In some embodiments, a subject of the present disclosure has an initial point of lung function comprising an FVC of between about 40% and about 30% of predicted. In some embodiments, a subject of the present disclosure has an initial point of lung function comprising an FVC of between about 30% and about 20% of predicted.

In some embodiments, a subject of the present disclosure has an initial point of lung function comprising an FVC of greater than or equal to 50% of predicted. In some embodiments the present disclosure provides methods of treating hepatopulmonary syndrome with one or more pulmonary complications (e.g., ILD), comprising administering a Transforming Growth Factor-β Receptor II (TβRII) fusion polypeptide to the subject, wherein the subject has an FVC of greater than or equal to 50% of predicted at baseline.

In some embodiments, an annual rate of decline in FVC of a subject is between about 1% and about 10%. In some embodiments, an annual rate of decline in FVC of a subject is between about 5% and about 10%. In some embodiments, an annual rate of decline in FVC of a subject is between about 10% and about 15%. In some embodiments, an annual rate of decline in FVC of a subject is between about 15% and about 20%. In some embodiments, an annual rate of decline in FVC of a subject is between about 20% and about 25%. In some embodiments, an annual rate of decline in FVC of a subject is between about 25% and about 30%. In some embodiments, an annual rate of decline in FVC of a subject is between about 30% and about 35%. In some embodiments, an annual rate of decline in FVC of a subject is between about 35% and about 40%. In some embodiments, an annual rate of decline in FVC of a subject is between about 40% and about 45%. In some embodiments, an annual rate of decline in FVC of a subject is between about 45% and about 50%. In some embodiments, an annual rate of decline in FVC of a subject is between about 50% and about 55%. In some embodiments, an annual rate of decline in FVC of a subject is between about 55% and about 60%. In some embodiments, an annual rate of decline in FVC of a subject is between about 60% and about 65%. In some embodiments, an annual rate of decline in FVC of a subject is between about 65% and about 70%. In some embodiments, an annual rate of decline in FVC of a subject is between about 10% and about 20%. In some embodiments, an annual rate of decline in FVC of a subject is between about 20% and about 30%. In some embodiments, an annual rate of decline in FVC of a subject is between about 30% and about 40%. In some embodiments, an annual rate of decline in FVC of a subject is between about 40% and about 50%. In some embodiments, an annual rate of decline in FVC of a subject is between about 50% and about 60%. In some embodiments, an annual rate of decline in FVC of a subject is between about 60% and about 70%.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein an annual rate of decline of the subject is reduced. In some embodiments, an annual rate of decline in FVC of a subject is reduced by between about 1% and about 10%. In some embodiments, an annual rate of decline in FVC of a subject is reduced by between about 5% and about 10%. In some embodiments, an annual rate of decline in FVC of a subject is reduced by between about 10% and about 15%. In some embodiments, an annual rate of decline in FVC of a subject is reduced by between about 15% and about 20%. In some embodiments, an annual rate of decline in FVC of a subject is reduced by between about 20% and about 25%. In some embodiments, an annual rate of decline in FVC of a subject is reduced by between about 25% and about 30%. In some embodiments, an annual rate of decline in FVC of a subject is reduced by between about 30% and about 35%. In some embodiments, an annual rate of decline in FVC of a subject is reduced by between about 35% and about 40%. In some embodiments, an annual rate of decline in FVC of a subject is reduced by between about 40% and about 45%. In some embodiments, an annual rate of decline in FVC of a subject is reduced by between about 45% and about 50%. In some embodiments, an annual rate of decline in FVC of a subject is reduced by between about 50% and about 55%. In some embodiments, an annual rate of decline in FVC of a subject is reduced by between about 55% and about 60%. In some embodiments, an annual rate of decline in FVC of a subject is reduced by between about 60% and about 65%. In some embodiments, an annual rate of decline in FVC of a subject is reduced by between about 65% and about 70%. In some embodiments, an annual rate of decline in FVC of a subject is reduced by between about 10% and about 20%. In some embodiments, an annual rate of decline in FVC of a subject is reduced by between about 20% and about 30%. In some embodiments, an annual rate of decline in FVC of a subject is reduced by between about 30% and about 40%. In some embodiments, an annual rate of decline in FVC of a subject is reduced by between about 40% and about 50%. In some embodiments, an annual rate of decline in FVC of a subject is reduced by between about 50% and about 60%. In some embodiments, an annual rate of decline in FVC of a subject is reduced by between about 60% and about 70%.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein an annual rate of decline of the subject is reduced relative to a subject treated with standard of care (SOC). In some embodiments, an annual rate of decline in FVC of a subject is reduced by between about 1% and about 10% relative to a subject treated with SOC. In some embodiments, an annual rate of decline in FVC of a subject is reduced by between about 5% and about 10% relative to a subject treated with SOC. In some embodiments, an annual rate of decline in FVC of a subject is reduced by between about 10% and about 15% relative to a subject treated with SOC. In some embodiments, an annual rate of decline in FVC of a subject is reduced by between about 15% and about 20% relative to a subject treated with SOC. In some embodiments, an annual rate of decline in FVC of a subject is reduced by between about 20% and about 25% relative to a subject treated with SOC. In some embodiments, an annual rate of decline in FVC of a subject is reduced by between about 25% and about 30% relative to a subject treated with SOC. In some embodiments, an annual rate of decline in FVC of a subject is reduced by between about 30% and about 35% relative to a subject treated with SOC. In some embodiments, an annual rate of decline in FVC of a subject is reduced by between about 35% and about 40% relative to a subject treated with SOC. In some embodiments, an annual rate of decline in FVC of a subject is reduced by between about 40% and about 45% relative to a subject treated with SOC. In some embodiments, an annual rate of decline in FVC of a subject is reduced by between about 45% and about 50% relative to a subject treated with SOC. In some embodiments, an annual rate of decline in FVC of a subject is reduced by between about 50% and about 55% relative to a subject treated with SOC. In some embodiments, an annual rate of decline in FVC of a subject is reduced by between about 55% and about 60% relative to a subject treated with SOC. In some embodiments, an annual rate of decline in FVC of a subject is reduced by between about 60% and about 65% relative to a subject treated with SOC. In some embodiments, an annual rate of decline in FVC of a subject is reduced by between about 65% and about 70% relative to a subject treated with SOC. In some embodiments, an annual rate of decline in FVC of a subject is reduced by between about 10% and about 20% relative to a subject treated with SOC. In some embodiments, an annual rate of decline in FVC of a subject is reduced by between about 20% and about 30% relative to a subject treated with SOC. In some embodiments, an annual rate of decline in FVC of a subject is reduced by between about 30% and about 40% relative to a subject treated with SOC. In some embodiments, an annual rate of decline in FVC of a subject is reduced by between about 40% and about 50% relative to a subject treated with SOC. In some embodiments, an annual rate of decline in FVC of a subject is reduced by between about 50% and about 60% relative to a subject treated with SOC. In some embodiments, an annual rate of decline in FVC of a subject is reduced by between about 60% and about 70% relative to a subject treated with SOC. In some embodiments, annual rate of decline is measured in milliliters (mL). In some embodiments, an annual rate of decline in FVC of a subject is reduced by between about 1 mL and about 10 mL. In some embodiments, an annual rate of decline in FVC of a subject is reduced by between about 5 mL and about 10 mL. In some embodiments, an annual rate of decline in FVC of a subject is reduced by between about 10 mL and about 15 mL. In some embodiments, an annual rate of decline in FVC of a subject is reduced by between about 15 mL and about 20 mL. In some embodiments, an annual rate of decline in FVC of a subject is reduced by between about 20 mL and about 25 mL. In some embodiments, an annual rate of decline in FVC of a subject is reduced by between about 25 mL and about 30 mL. In some embodiments, an annual rate of decline in FVC of a subject is reduced by between about 30 mL and about 35 mL. In some embodiments, an annual rate of decline in FVC of a subject is reduced by between about 35 mL and about 40 mL. In some embodiments, an annual rate of decline in FVC of a subject is reduced by between about 40 mL and about 45 mL. In some embodiments, an annual rate of decline in FVC of a subject is reduced by between about 45 mL and about 50 mL. In some embodiments, an annual rate of decline in FVC of a subject is reduced by between about 50 mL and about 55 mL. In some embodiments, an annual rate of decline in FVC of a subject is reduced by between about 55 mL and about 60 mL. In some embodiments, an annual rate of decline in FVC of a subject is reduced by between about 60 mL and about 65 mL. In some embodiments, an annual rate of decline in FVC of a subject is reduced by between about 65 mL and about 70 mL. In some embodiments, an annual rate of decline in FVC of a subject is reduced by between about 70 mL and about 75 mL. In some embodiments, an annual rate of decline in FVC of a subject is reduced by between about 75 mL and about 80 mL. In some embodiments, an annual rate of decline in FVC of a subject is reduced by between about 85 mL and about 90 mL. In some embodiments, an annual rate of decline in FVC of a subject is reduced by between about 90 mL and about 95 mL. In some embodiments, an annual rate of decline in FVC of a subject is reduced by between about 95 mL and about 100 mL. In some embodiments, an annual rate of decline in FVC of a subject is reduced by between about 10 mL and about 20 mL. In some embodiments, an annual rate of decline in FVC of a subject is reduced by between about 20 mL and about 30 mL. In some embodiments, an annual rate of decline in FVC of a subject is reduced by between about 30 mL and about 40 mL. In some embodiments, an annual rate of decline in FVC of a subject is reduced by between about 40 mL and about 50 mL. In some embodiments, an annual rate of decline in FVC of a subject is reduced by between about 50 mL and about 60 mL. In some embodiments, an annual rate of decline in FVC of a subject is reduced by between about 60 mL and about 70 mL. In some embodiments, an annual rate of decline in FVC of a subject is reduced by between about 70 mL and about 80 mL. In some embodiments, an annual rate of decline in FVC of a subject is reduced by between about 80 mL and about 90 mL. In some embodiments, an annual rate of decline in FVC of a subject is reduced by between about 90 mL and about 100 mL. In some embodiments, an annual rate of decline in FVC of a subject is reduced by about 100 mL. In some embodiments, an annual rate of decline in FVC of a subject is reduced by more than about 100 mL.

In some embodiments, annual rate of decline is measured in milliliters (mL) relative to a subject treated with standard of care (SOC). In some embodiments, an annual rate of decline in FVC of a subject is reduced by between about 1 mL and about 10 mL relative to a subject treated with SOC. In some embodiments, an annual rate of decline in FVC of a subject is reduced by between about 5 mL and about 10 mL relative to a subject treated with SOC. In some embodiments, an annual rate of decline in FVC of a subject is reduced by between about 10 mL and about 15 mL relative to a subject treated with SOC. In some embodiments, an annual rate of decline in FVC of a subject is reduced by between about 15 mL and about 20 mL relative to a subject treated with SOC. In some embodiments, an annual rate of decline in FVC of a subject is reduced by between about 20 mL and about 25 mL relative to a subject treated with SOC. In some embodiments, an annual rate of decline in FVC of a subject is reduced by between about 25 mL and about 30 mL relative to a subject treated with SOC. In some embodiments, an annual rate of decline in FVC of a subject is reduced by between about 30 mL and about 35 mL relative to a subject treated with SOC. In some embodiments, an annual rate of decline in FVC of a subject is reduced by between about 35 mL and about 40 mL relative to a subject treated with SOC. In some embodiments, an annual rate of decline in FVC of a subject is reduced by between about 40 mL and about mL relative to a subject treated with SOC. In some embodiments, an annual rate of decline in FVC of a subject is reduced by between about 45 mL and about 50 mL relative to a subject treated with SOC. In some embodiments, an annual rate of decline in FVC of a subject is reduced by between about 50 mL and about 55 mL relative to a subject treated with SOC. In some embodiments, an annual rate of decline in FVC of a subject is reduced by between about 55 mL and about 60 mL relative to a subject treated with SOC. In some embodiments, an annual rate of decline in FVC of a subject is reduced by between about 60 mL and about 65 mL relative to a subject treated with SOC. In some embodiments, an annual rate of decline in FVC of a subject is reduced by between about 65 mL and about 70 mL relative to a subject treated with SOC. In some embodiments, an annual rate of decline in FVC of a subject is reduced by between about 70 mL and about 75 mL relative to a subject treated with SOC. In some embodiments, an annual rate of decline in FVC of a subject is reduced by between about 75 mL and about 80 mL relative to a subject treated with SOC. In some embodiments, an annual rate of decline in FVC of a subject is reduced by between about 85 mL and about 90 mL relative to a subject treated with SOC. In some embodiments, an annual rate of decline in FVC of a subject is reduced by between about 90 mL and about mL relative to a subject treated with SOC. In some embodiments, an annual rate of decline in FVC of a subject is reduced by between about 95 mL and about 100 mL relative to a subject treated with SOC. In some embodiments, an annual rate of decline in FVC of a subject is reduced by between about 10 mL and about 20 mL relative to a subject treated with SOC. In some embodiments, an annual rate of decline in FVC of a subject is reduced by between about 20 mL and about 30 mL relative to a subject treated with SOC. In some embodiments, an annual rate of decline in FVC of a subject is reduced by between about 30 mL and about 40 mL relative to a subject treated with SOC. In some embodiments, an annual rate of decline in FVC of a subject is reduced by between about 40 mL and about 50 mL relative to a subject treated with SOC. In some embodiments, an annual rate of decline in FVC of a subject is reduced by between about 50 mL and about 60 mL relative to a subject treated with SOC. In some embodiments, an annual rate of decline in FVC of a subject is reduced by between about 60 mL and about 70 mL relative to a subject treated with SOC. In some embodiments, an annual rate of decline in FVC of a subject is reduced by between about 70 mL and about 80 mL relative to a subject treated with SOC. In some embodiments, an annual rate of decline in FVC of a subject is reduced by between about 80 mL and about 90 mL relative to a subject treated with SOC. In some embodiments, an annual rate of decline in FVC of a subject is reduced by between about 90 mL and about 100 mL relative to a subject treated with SOC. In some embodiments, an annual rate of decline in FVC of a subject is reduced by about 100 mL relative to a subject treated with SOC. In some embodiments, an annual rate of decline in FVC of a subject is reduced by more than about 100 mL relative to a subject treated with SOC.

In some embodiments, administration of one or more TβRII antagonists of the present disclosure results in a reduction in annual rate of decline of forced vital capacity (FVC) relative to a subject treated with standard of care (SOC). In some embodiments, an annual rate of decline in FVC has been slowed relative to a subject treated with standard of care (SOC). In some embodiments, administration of one or more TβRII antagonists of the present disclosure slows the annual rate of decline in FVC relative to a subject treated with standard of care (SOC). In some embodiments the present disclosure provides methods of treating lung disease and/or pulmonary complications associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to the subject; wherein an annual rate of decline in FVC of a subject is reduced by about 100 mL relative to a subject treated with SOC. In some embodiments the present disclosure provides methods of treating lung disease and/or pulmonary complications associated with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) fusion polypeptide to the subject; wherein an annual rate of decline in FVC of a subject is reduced by 100 mL relative to a subject treated with SOC. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, comprising administering a Transforming Growth Factor-β Receptor II (TβRII) fusion polypeptide to the subject; wherein an annual rate of decline in FVC of a subject is reduced by more than about 100 mL relative to a subject treated with SOC.

Diffusing Capacity for Carbon Monoxide (DLCO)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using a measurement of the diffusing capacity of the lungs for carbon monoxide (DLCO). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. DLCO is a medical test that determines how much oxygen travels from the alveoli of the lungs to the blood stream. Testing for DLCO involves measuring the partial pressure difference between inspired and expired carbon monoxide. A DLCO measurement relies on the strong affinity and/or large absorption capacity of red blood cells for carbon monoxide and thus demonstrates gas uptake by the capillaries that are less dependent on cardiac output. A measurement of DLCO can be affected by atmospheric pressure and/or altitude and correction factors can be calculated using the method recommended by the American Thoracic Society. Expected DLCO is also affected by the amount of hemoglobin, carboxyhemoglobin, age and/or sex of the subject. Generally, DLCO is measured in “mL/min/kPa” and can also be presented as a percentage of predicted. Of predicted” refers to reporting the subject's results as a percentage of the known predicted values for a healthy subject of similar characteristics (e.g., height, sex, age, race, and weight). DLCO can be measured by one or more of a rapidly responding gas analyzer (RGA), flow volume analyzer, and/or gas analyzer. Several factors can lead to a decrease in DLCO, which includes, but is not limited to, hindrance in the alveolar wall (e.g., fibrosis, alveolitis, vasculitis), decrease of total lung area (e.g., restrictive lung disease or lung resection (partial or total)), chronic obstructive pulmonary disease (COPD) due to decreased surface area in the alveoli as well as damage to the capillary bed, pulmonary embolism, cardiac insufficiency, pulmonary hypertension (PH), exposure to bleomycin (upon administration of at least about 200 units), chronic heart failure, anemia due to a decrease in blood volume, and/or amiodarone high cumulative dose (e.g., at least about 400 milligrams per day). HPS subjects have been noted to have more profound reductions in diffusion capacity, with a mean DLCO of 55% predicted versus a mean of 72% predicted in people with cirrhosis who do not have HPS (Lima et al., Mayo Clin Proc. 2004; 79(1):42-48).

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein DLCO is measured using a rapidly responding gas analyzer (RGA). In some embodiments, a subject has a DLCO of between about 90% and about 80% of predicted. In some embodiments, a subject has a DLCO of between about 80% and about 70% of predicted. In some embodiments, a subject has a DLCO of between about 70% and about 60% of predicted. In some embodiments, a subject has a DLCO of between about 60% and about 50% of predicted. In some embodiments, a subject has a DLCO of between about 50% and about 40% of predicted. In some embodiments, a subject has a DLCO of between about 40% and about 30% of predicted. In some embodiments, a subject has a DLCO of between about 30% and about 20% of predicted.

In some embodiments, a subject of the present disclosure has a DLCO of greater than or equal to 40% of predicted. In some embodiments, a subject of the present disclosure has a DLCO of greater than or equal to 40% of predicted, corrected by Hgb. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein the subject has a DLCO of greater than or equal to 40% of predicted at baseline. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has a DLCO of greater than or equal to 40% of predicted (corrected by Hgb) at baseline.

In some embodiments, a subject of the present disclosure has a DLCO of between about 40% and about 89% of predicted. In some embodiments, a subject of the present disclosure has a DLCO of between about 40% and about 89% of predicted, corrected by Hgb. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein the subject has a DLCO of between about 40% and about 89% of predicted at baseline. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein the subject has a DLCO of between about 40% and about 89% of predicted (corrected by Hgb) at baseline.

In some embodiments, an annual rate of decline in diffusing capacity of the lungs (DLCO) of a subject is measured over a time period of at least one year after administration of one or more TβRII antagonists of the present disclosure. In some embodiments, an annual rate of decline in diffusing capacity of the lungs (DLCO) of a subject is measured over a time period of at least one year after administration of one or more TβRII antagonists of the present disclosure and is compared to a baseline DLCO measurement. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein an annual rate of DLCO of the subject is reduced.

In some embodiments, an annual rate of decline in DLCO of a subject is reduced by between about 1% and about 10%. In some embodiments, an annual rate of decline in DLCO of a subject is reduced by between about 5% and about 10%. In some embodiments, an annual rate of decline in DLCO of a subject is reduced by between about 10% and about 15%. In some embodiments, an annual rate of decline in DLCO of a subject is reduced by between about 15% and about 20%. In some embodiments, an annual rate of decline in DLCO of a subject is reduced by between about 20% and about 25%. In some embodiments, an annual rate of decline in DLCO of a subject is reduced by between about 25% and about 30%. In some embodiments, an annual rate of decline in DLCO of a subject is reduced by between about 30% and about 35%. In some embodiments, an annual rate of decline in DLCO of a subject is reduced by between about 35% and about 40%. In some embodiments, an annual rate of decline in DLCO of a subject is reduced by between about 40% and about 45%. In some embodiments, an annual rate of decline in DLCO of a subject is reduced by between about 45% and about 50%. In some embodiments, an annual rate of decline in DLCO of a subject is reduced by between about 50% and about 55%. In some embodiments, an annual rate of decline in DLCO of a subject is reduced by between about 55% and about 60%. In some embodiments, an annual rate of decline in DLCO of a subject is reduced by between about 60% and about 65%. In some embodiments, an annual rate of decline in DLCO of a subject is reduced by between about 65% and about 70%. In some embodiments, an annual rate of decline in DLCO of a subject is reduced by between about 10% and about 20%. In some embodiments, an annual rate of decline in DLCO of a subject is reduced by between about 20% and about 30%. In some embodiments, an annual rate of decline in DLCO of a subject is reduced by between about 30% and about 40%. In some embodiments, an annual rate of decline in DLCO of a subject is reduced by between about 40% and about 50%. In some embodiments, an annual rate of decline in DLCO of a subject is reduced by between about 50% and about 60%. In some embodiments, an annual rate of decline in DLCO of a subject is reduced by between about 60% and about 70%.

In some embodiments, annual rate of decline is measured in mL/min/kPa per year. In some embodiments, an annual rate of decline in DLCO of a subject is reduced by between about 1 mL/min/kPa and about 10 mL/min/kPa per year. In some embodiments, an annual rate of decline in DLCO of a subject is reduced by between about 5 mL/min/kPa and about mL/min/kPa per year. In some embodiments, an annual rate of decline in DLCO of a subject is reduced by between about 10 mL/min/kPa and about 15 mL/min/kPa per year. In some embodiments, an annual rate of decline in DLCO of a subject is reduced by between about 15 mL/min/kPa and about 20 mL/min/kPa per year. In some embodiments, an annual rate of decline in DLCO of a subject is reduced by between about 20 mL/min/kPa and about mL/min/kPa per year. In some embodiments, an annual rate of decline in DLCO of a subject is reduced by between about 25 mL/min/kPa and about 30 mL/min/kPa per year. In some embodiments, an annual rate of decline in DLCO of a subject is reduced by between about 30 mL/min/kPa and about 35 mL/min/kPa per year. In some embodiments, an annual rate of decline in DLCO of a subject is reduced by between about 35 mL/min/kPa and about mL/min/kPa per year. In some embodiments, an annual rate of decline in DLCO of a subject is reduced by between about 40 mL/min/kPa and about 45 mL/min/kPa per year. In some embodiments, an annual rate of decline in DLCO of a subject is reduced by between about 45 mL/min/kPa and about 50 mL/min/kPa per year. In some embodiments, an annual rate of decline in DLCO of a subject is reduced by between about 50 mL/min/kPa and about mL/min/kPa per year. In some embodiments, an annual rate of decline in DLCO of a subject is reduced by between about 55 mL/min/kPa and about 60 mL/min/kPa per year. In some embodiments, an annual rate of decline in DLCO of a subject is reduced by between about 60 mL/min/kPa and about 65 mL/min/kPa per year. In some embodiments, an annual rate of decline in DLCO of a subject is reduced by between about 65 mL/min/kPa and about mL/min/kPa per year. In some embodiments, an annual rate of decline in DLCO of a subject is reduced by between about 70 mL/min/kPa and about 75 mL/min/kPa per year. In some embodiments, an annual rate of decline in DLCO of a subject is reduced by between about 75 mL/min/kPa and about 80 mL/min/kPa per year. In some embodiments, an annual rate of decline in DLCO of a subject is reduced by between about 85 mL/min/kPa and about mL/min/kPa per year. In some embodiments, an annual rate of decline in DLCO of a subject is reduced by between about 90 mL/min/kPa and about 95 mL/min/kPa per year. In some embodiments, an annual rate of decline in DLCO of a subject is reduced by between about 95 mL/min/kPa and about 100 mL/min/kPa per year. In some embodiments, an annual rate of decline in DLCO of a subject is reduced by between about 10 mL/min/kPa and about mL/min/kPa per year. In some embodiments, an annual rate of decline in DLCO of a subject is reduced by between about 20 mL/min/kPa and about 30 mL/min/kPa per year. In some embodiments, an annual rate of decline in DLCO of a subject is reduced by between about 30 mL/min/kPa and about 40 mL/min/kPa per year. In some embodiments, an annual rate of decline in DLCO of a subject is reduced by between about 40 mL/min/kPa and about mL/min/kPa per year. In some embodiments, an annual rate of decline in DLCO of a subject is reduced by between about 50 mL/min/kPa and about 60 mL/min/kPa per year. In some embodiments, an annual rate of decline in DLCO of a subject is reduced by between about 60 mL/min/kPa and about 70 mL/min/kPa per year. In some embodiments, an annual rate of decline in DLCO of a subject is reduced by between about 70 mL/min/kPa and about mL/min/kPa per year. In some embodiments, an annual rate of decline in DLCO of a subject is reduced by between about 80 mL/min/kPa and about 90 mL/min/kPa per year. In some embodiments, an annual rate of decline in DLCO of a subject is reduced by between about 90 mL/min/kPa and about 100 mL/min/kPa per year. In some embodiments, an annual rate of decline in DLCO of a subject is reduced by more than about 100 mL/min/kPa per year.

Lung Volume

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using measurement of lung volumes. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Measurement of lung volumes may be important when spirometry shows a decreased forced vital capacity. Lung volume is typically measured by body plethysmography, particularly in the setting of significant airflow obstruction. Alternative methods to measure lung function include, but are not limited to, helium dilution, nitrogen washout, and/or measurements based on chest imaging.

Measurements of total lung capacity (TLC) using chest radiograph and/or high resolution computed tomography (HRCT) generally correlate within 15 percent of those obtained by body plethysmography.

Common lung volume measurements include vital capacity, total lung capacity, functional residual capacity, and/or residual volume. Vital capacity (VC) comprises maximum volume exhaled after maximum inspiration, and can be measured during forced exhalation (FVC) or slow exhalation (SVC). Functional residual capacity (FRC) comprises volume of air remaining in chest at the end of a tidal volume breath. Residual volume (RV) comprises volume of air remaining in chest after maximal exhalation. Expiratory reserve volume (ERV) comprises volume of air exhaled from end-tidal volume (FRC) to point of maximal exhalation (RV), therefore RV plus ERV=FRC. Inspiratory capacity (IC) comprises maximum inspiration from end-tidal volume (FRC) to total lung capacity. Total lung capacity (TLC) comprises volume of air in lungs at end of maximal inspiration (usually calculated by RV plus VC or FRC plus IC)

Combination of an FEV1/FVC and TLC both less than the fifth percentile lower limit of normal is considered a mixed defect. “Mixed defect” ventilatory impairment in majority cases represents airway obstruction with lung hyperinflation. Combination of a normal FEV1/FVC and a normal TLC, and a low FEV1 or FVC, is considered a nonspecific pattern. A pattern in which an FVC is disproportionately reduced relative to TLC has been described as a complex restrictive pattern.

In some embodiments, a subject with hepatopulmonary syndrome has a decreased lung volume. In some embodiments, a subject with hepatopulmonary syndrome has a decreased total lung capacity (TLC). In some embodiments, a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) has a decreased lung volume. In some embodiments, a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) has a decreased total lung capacity (TLC). In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has a decreased TLC.

In some embodiments, a subject has a TLC of between about 100% and about 90% of predicted. In some embodiments, a subject has a TLC of between about 90% and about 80% of predicted. In some embodiments, a subject has a TLC of between about 80% and about 70% of predicted. In some embodiments, a subject has a TLC of between about 70% and about 60% of predicted. In some embodiments, a subject has a TLC of between about 60% and about 50% of predicted. In some embodiments, a subject has a TLC of between about 50% and about 40% of predicted. In some embodiments, a subject has a TLC of between about 40% and about 30% of predicted. In some embodiments, a subject has a TLC of between about 30% and about 20% of predicted. In some embodiments, administration of a TβRII antagonist improves the subject's TLC to between about 100% and about 90% of predicted. In some embodiments, administration of a TβRII antagonist improves the subject's TLC to between about 90% and about 80% of predicted. In some embodiments, administration of a TβRII antagonist improves the subject's TLC to between about 80% and about 70% of predicted. In some embodiments, administration of a TβRII antagonist improves the subject's TLC to between about 70% and about 60% of predicted. In some embodiments, administration of a TβRII antagonist improves the subject's TLC to between about 60% and about 50% of predicted. In some embodiments, administration of a TβRII antagonist improves the subject's TLC to between about 50% and about 40% of predicted. In some embodiments, administration of a TβRII antagonist improves the subject's TLC to between about 40% and about 30% of predicted.

Six Minute Walk Test

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using a six-minute walk test (6MWT). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. A 6MWT assesses both distance walked (meters) and/or degree of oxygen desaturation of a subject. It can be used as a measure of submaximal exercise performance in a variety of pulmonary disease settings. Assessment of pulse oxygen desaturation can be problematic in patients with connective tissue disorders, diseases of the arteries, carpal tunnel syndrome, or patients who smoke due to Raynaud phenomenon and/or poor finger perfusion. If the appropriate attachment is available, measurement of pulse oxygen saturation using an earlobe clip may be more effective for subjects with chronic liver disease or a complication associated with chronic liver disease as disclosed herein. A healthy subject (e.g., a subject without chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD)) typically walks between about 400 to about 700 meters in a six minute walk test. Average distance walked may vary by characteristics such as sex, age, weight, or height, among others. A reference subject for a six minute walk test may be a healthy subject (e.g., no respiratory disease) of similar age, height, weight, and/or same sex. An improvement of at least about 30 meters in distance walked during a six minute walk test typically indicates an improvement in respiratory function.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein the subject has a decrease in distance walked during a six minute walk test. In some embodiments, a subject has a decrease in oxygen desaturation. In some embodiments, administration of one or more TβRII antagonists of the present disclosure results in an increase in distance walked during a six minute walk test. In some embodiments, administration of one or more TβRII antagonists of the present disclosure results in an increase in oxygen desaturation during a six minute walk test.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein the subject has an improved distance walked in a six minute walk test after the treatment. In some embodiments, a subject improves distance walked in a six minute walk test by at least about 30 meters compared to a reference subject. In some embodiments, a reference subject is a healthy person of similar characteristics (e.g., sex, age, height, weight).

In some embodiments, a subject improves distance walked in a six minute walk test by at least between about 10 meters and about 15 meters compared to a reference subject. In some embodiments, a subject improves distance walked in a six minute walk test by at least between about 15 meters and about 20 meters compared to a reference subject. In some embodiments, a subject improves distance walked in a six minute walk test by at least between about 20 meters and about 25 meters compared to a reference subject. In some embodiments, a subject improves distance walked in a six minute walk test by at least between about 25 meters and about 30 meters compared to a reference subject. In some embodiments, a subject improves distance walked in a six minute walk test by at least between about 30 meters and about 35 meters compared to a reference subject. In some embodiments, a subject improves distance walked in a six minute walk test by at least between about 35 meters and about 40 meters compared to a reference subject. In some embodiments, a subject improves distance walked in a six minute walk test by at least between about 40 meters and about 45 meters compared to a reference subject. In some embodiments, a subject improves distance walked in a six minute walk test by at least between about 45 meters and about 50 meters compared to a reference subject. In some embodiments, a subject improves distance walked in a six minute walk test by at least between about 50 meters and about 55 meters compared to a reference subject. In some embodiments, a subject improves distance walked in a six minute walk test by at least between about 55 meters and about 60 meters compared to a reference subject. In some embodiments, a subject improves distance walked in a six minute walk test by at least between about 60 meters and about 65 meters compared to a reference subject. In some embodiments, a subject improves distance walked in a six minute walk test by at least between about 65 meters and about 70 meters compared to a reference subject. In some embodiments, a subject improves distance walked in a six minute walk test by at least between about 70 meters and about 75 meters compared to a reference subject. In some embodiments, a subject improves distance walked in a six minute walk test by at least between about 75 meters and about 80 meters compared to a reference subject. In some embodiments, a subject improves distance walked in a six minute walk test by at least between about 80 meters and about 85 meters compared to a reference subject. In some embodiments, a subject improves distance walked in a six minute walk test by at least between about 85 meters and about 90 meters compared to a reference subject. In some embodiments, a subject improves distance walked in a six minute walk test by at least between about 90 meters and about 95 meters compared to a reference subject. In some embodiments, a subject improves distance walked in a six minute walk test by at least between about 95 meters and about 100 meters compared to a reference subject. In some embodiments, a subject improves distance walked in a six minute walk test by at least more than 100 meters compared to a reference subject.

In some embodiments, treatment of a subject with one or more of a TβRII antagonist of the present disclosure improves distance walked in a six minute walk test of a subject. In some embodiments, treatment of a subject with one or more of a TβRII antagonist of the present disclosure improves distance walked in a six minute walk test of a subject by between about 1% and about 3%. In some embodiments, treatment of a subject with one or more of a TβRII antagonist of the present disclosure improves distance walked in a six minute walk test of a subject by between about 1% and about 5%. In some embodiments, treatment of a subject with one or more of a TβRII antagonist of the present disclosure improves distance walked in a six minute walk test of a subject by between about 1% and about 8%. In some embodiments, treatment of a subject with one or more of a TβRII antagonist of the present disclosure improves distance walked in a six minute walk test of a subject by between about 1% and about 10%. In some embodiments, treatment of a subject with one or more of a TβRII antagonist of the present disclosure improves distance walked in a six minute walk test of a subject by between about 10% and about 15%. In some embodiments, treatment of a subject with one or more of a TβRII antagonist of the present disclosure improves distance walked in a six minute walk test of a subject by between about 15% and about 20%. In some embodiments, treatment of a subject with one or more of a TβRII antagonist of the present disclosure improves distance walked in a six minute walk test of a subject by between about 20% and about 25%. In some embodiments, treatment of a subject with one or more of a TβRII antagonist of the present disclosure improves distance walked in a six minute walk test of a subject by between about 25% and about 30%. In some embodiments, treatment of a subject with one or more of a TβRII antagonist of the present disclosure improves distance walked in a six minute walk test of a subject by between about 30% and about 35%. In some embodiments, treatment of a subject with one or more of a TβRII antagonist of the present disclosure improves distance walked in a six minute walk test of a subject by between about 35% and about 40%. In some embodiments, treatment of a subject with one or more of a TβRII antagonist of the present disclosure improves distance walked in a six minute walk test of a subject by between about 40% and about 45%. In some embodiments, treatment of a subject with one or more of a TβRII antagonist of the present disclosure improves distance walked in a six minute walk test of a subject by between about 45% and about 50%.

Bronchoalveolar Lavage (BAL)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using Bronchoalveolar lavage (BAL). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. BAL is performed during flexible bronchoscopy to obtain samples of cells and/or fluid from distal airways and/or alveoli, and may be used to evaluate chronic liver disease patients with pulmonary fibrosis or ILD. Lavage fluid is sent for cell counts; cultures for mycobacterial, viral, and/or fungal pathogens; and/or cytologic analysis. BAL is particularly useful in the evaluation of patients with interstitial lung disease (ILD) that is associated with hemoptysis, is acute or rapidly progressive, or is likely caused by one or more of the following diseases: sarcoidosis, hypersensitivity pneumonitis, pulmonary Langerhans histiocytosis, or infection. Virtually all patients presenting with hemoptysis and/or radiographic ILD are recommended to undergo BAL with sequential lavages to confirm an alveolar source of bleeding and/or identify any infectious etiologies.

The majority of patients with an acute onset of ILD will undergo BAL to evaluate for acute eosinophilic pneumonia, alveolar hemorrhage, malignancy, and/or opportunistic or atypical infection, which can often be diagnosed on the basis of BAL findings. For patients with a subacute or chronic presentation of ILD, BAL is often performed when sarcoidosis, hypersensitivity pneumonitis, pulmonary Langerhans cell histiocytosis (PLCH), and/or infection are suspected based on the radiographic pattern (e.g., upper lobe predominance of reticular opacities, hilar lymphadenopathy, irregular cystic airspaces), history of exposure (e.g., bird keeping, farming), or concomitant clinical findings (e.g., hemoptysis, renal insufficiency). In these patients, the results of BAL analysis may be used to narrow the differential diagnostic possibilities between various types of ILD, but tissue confirmation is usually required.

Chronic liver disease patients with pulmonary fibrosis or ILD may have elevated numbers of granulocytes in their BAL fluid, particularly neutrophils and/or eosinophils, and/or may sometimes have an increase in lymphocytes and/or mast cells. In some embodiments, a subject with a complication associated with chronic liver disease, portal hypertension, primary sclerosing cholangitis, and/or hepatopulmonary syndrome (e.g., pulmonary fibrosis or ILD) is tested using BAL. In some embodiments, ILD in a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is confirmed using BAL. In some embodiments, a BAL exam of a subject is used to assess the degree of alveolitis and/or predict response to immunosuppressive therapy.

AST to Platelet Ratio Index (APRI)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using the AST (aspartate aminotransferase) to platelet ratio index (APRI). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. APRI was developed as a low cost method for estimating liver fibrosis and cirrhosis severity using information from a patient's AST and platelet count. The liver produces increased levels of AST when liver damage or fibrosis is present. High circulating levels of AST in the blood are typically used as an indicator of cirrhosis. AST values vary based on a person's body mass index, however normal AST values for adults is 8 to 48 international units per liter (IU/L). Patients with cirrhosis often have low platelet counts (below 150,000 platelets per microliter). An APRI score is calculated by applying the patient's AST and platelet count, along with what is considered to be the upper limit of the normal AST range (typically 40 units per liter), as follows: [(AST/upper limit of the normal AST range)×100]/Platelet Count. APRI scores less than or equal to 0.5 suggest the liver is free from fibrosis or may have low levels of scarring. APRI scores greater than or equal to 1.5 suggest that the liver has scarring and cirrhosis. In some embodiments, the subject has an APRI score of less than or equal to 0.5. In some embodiments, the subject has an APRI score greater than or equal to 1.5. In some embodiments, administration of a TβRII antagonist reduces the subjects APRI score to less than 1.5. In some embodiments, administration of a TβRII antagonist reduces the subjects APRI score to 0.5. In some embodiments, administration of a TβRII antagonist reduces the subjects APRI score to less than or equal to 0.5. In some embodiments, administration of a TβRII antagonist reduces the subjects APRI score to less than 0.5.

In certain embodiments, the subject has chronic liver disease and increased levels of AST. In certain embodiments, the subject has pulmonary fibrosis or ILD associated with chronic liver disease and increased levels of AST. In certain embodiments, the subject has chronic liver disease and low platelet counts. In certain embodiments, the subject has chronic liver disease and platelet counts less than or equal to 150,000 platelets per microliter. In certain embodiments, the subject has pulmonary fibrosis or ILD associated with chronic liver disease and low platelet counts. In certain embodiments, the subject has pulmonary fibrosis or ILD associated with chronic liver disease and platelet counts less than or equal to 150,000 platelets per microliter. In certain embodiments, the subject has pulmonary fibrosis or ILD associated with chronic liver disease with low platelet counts and increased levels of AST. In certain embodiments, the subject has pulmonary fibrosis or ILD associated with chronic liver disease, platelet counts less than or equal to 150,000 platelets per microliter, and AST levels greater than 40 units per liter. In certain embodiments, the subject has pulmonary fibrosis or ILD associated with portal hypertension and increased levels of AST. In certain embodiments, the subject has portal hypertension and low platelet counts. In certain embodiments, the subject has pulmonary fibrosis or ILD associated with portal hypertension and low platelet counts. In certain embodiments, the subject has pulmonary fibrosis or ILD associated with portal hypertension and platelet counts less than or equal to 150,000 platelets per microliter. In certain embodiments, the subject has pulmonary fibrosis or ILD associated with portal hypertension, low platelet counts, and increased levels of AST. In certain embodiments, the subject has pulmonary fibrosis or ILD associated with portal hypertension, platelet counts less than or equal to 150,000 platelets per microliter, and AST levels greater than 40 units per liter. In certain embodiments, the subject has pulmonary fibrosis or ILD associated with primary sclerosing cholangitis and increased levels of AST. In certain embodiments, the subject has primary sclerosing cholangitis and low platelet counts. In certain embodiments, the subject has primary sclerosing cholangitis and platelet counts less than or equal to 150,000 platelets per microliter. In certain embodiments, the subject has pulmonary fibrosis or ILD associated with primary sclerosing cholangitis and low platelet counts. In certain embodiments, the subject has pulmonary fibrosis or ILD associated with primary sclerosing cholangitis and platelet counts less than or equal to 150,000 platelets per microliter. In certain embodiments, the subject has pulmonary fibrosis or ILD associated with primary sclerosing cholangitis, low platelet counts, and increased levels of AST. In certain embodiments, the subject has pulmonary fibrosis or ILD associated with primary sclerosing cholangitis, platelet counts less than or equal to 150,000 platelets per microliter, and AST levels greater than 40 units per liter. In certain embodiments, the subject has pulmonary fibrosis or ILD associated with HPS and increased levels of AST. In certain embodiments, the subject has HPS and low platelet counts. In certain embodiments, the subject has HPS and platelet counts less than or equal to 150,000 platelets per microliter. In certain embodiments, the subject has pulmonary fibrosis or ILD associated with HPS and low platelet counts. In certain embodiments, the subject has pulmonary fibrosis or ILD associated with HPS and platelet counts less than or equal to 150,000 platelets per microliter. In certain embodiments, the subject has pulmonary fibrosis or ILD associated with HPS, low platelet counts, and increased levels of AST. In certain embodiments, the subject has pulmonary fibrosis or ILD associated with HPS, platelet counts less than or equal to 150,000 platelets per microliter, and AST levels greater than 40 units per liter. In certain embodiments, the increased levels of AST are greater than 40 units per liter. In certain embodiments, the increased levels of AST are greater than or equal to 50 units per liter. In certain embodiments, the increased levels of AST are greater than or equal to 60 units per liter. In certain embodiments, the increased levels of AST are greater than or equal to 70 units per liter. In certain embodiments, the increased levels of AST are greater than or equal to 80 units per liter. In certain embodiments, the increased levels of AST are greater than or equal to 90 units per liter. In certain embodiments, the increased levels of AST are greater than or equal to 100 units per liter. In certain embodiments, the increased levels of AST are greater than or equal to 120 units per liter. In certain embodiments, the increased levels of AST are greater than or equal to 130 units per liter. In certain embodiments, the increased levels of AST are greater than or equal to 140 units per liter. In certain embodiments, the subject has HPS and low platelet counts. In certain embodiments, the low platelet counts are less than or equal to 150,000 platelets per microliter. In certain embodiments, the low platelet counts are less than or equal to 125,000 platelets per microliter. In certain embodiments, the low platelet counts are less than or equal to 100,000 platelets per microliter. In certain embodiments, the low platelet counts are less than or equal to 90,000 platelets per microliter. In certain embodiments, the low platelet counts are less than or equal to 80,000 platelets per microliter. In certain embodiments, the low platelet counts are less than or equal to 70,000 platelets per microliter. In certain embodiments, the low platelet counts are less than or equal to 60,000 platelets per microliter. In certain embodiments, the low platelet counts are less than or equal to 50,000 platelets per microliter. In certain embodiments, the low platelet counts are less than or equal to 40,000 platelets per microliter. In certain embodiments, the low platelet counts are less than or equal to 30,000 platelets per microliter. In certain embodiments, the low platelet counts are less than or equal to 20,000 platelets per microliter. In certain embodiments, the low platelet counts are less than or equal to 10,000 platelets per microliter. In certain embodiments, the subject has HPS and platelet counts less than or equal to 150,000 platelets per microliter. In certain embodiments, the subject has pulmonary fibrosis or ILD associated with HPS and low platelet counts. In certain embodiments, the subject has pulmonary fibrosis or ILD associated with HPS and platelet counts less than or equal to 150,000 platelets per microliter. In certain embodiments, the subject has pulmonary fibrosis or ILD associated with HPS, low platelet counts, and increased levels of AST. In certain embodiments, the subject has pulmonary fibrosis or ILD associated with HPS, platelet counts less than or equal to 150,000 platelets per microliter, and AST levels greater than 40 units per liter.

Bonacini Index

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using the Bonacini index. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. The Bonacini index was developed as an inexpensive and rapid method for predicting cirrhosis. The Bonacini score has a range of possible values from 0 to 11, and the value of the score is significantly higher in patients with advanced fibrosis or cirrhosis than in patients with histological fibrosis. This score is derived from three laboratory parameters: platelet count, ALT/AST ratio (where ALT is alanine aminotransferase), and PT (prothrombin time). Scores less than or equal to 3 indicate a low probability of fibrosis or cirrhosis. Scores in the range of 4-7 indicate a high probability of fibrosis or cirrhosis. Scores greater than 7 indicate a very high, nearly absolute probability of fibrosis or cirrhosis. In certain embodiments, the subject has a Bonacini index score less than or equal to 3. In certain embodiments, the subject has a Bonacini index score of 4. In certain embodiments, the subject has a Bonacini index score of 5. In certain embodiments, the subject has a Bonacini index score of 6. In certain embodiments, the subject has a Bonacini index score of 7. In certain embodiments, the subject has a Bonacini index score greater than 7. In certain embodiments, the subject has a Bonacini index score of 8. In certain embodiments, the subject has a Bonacini index score of 9. In certain embodiments, the subject has a Bonacini index score of 10. In certain embodiments, the subject has a Bonacini index score of 11. In certain embodiments, the subject has a Bonacini index score of 4. In certain embodiments, the subject has a Bonacini index score of 5. In certain embodiments, the subject has a Bonacini index score of 6. In certain embodiments, the subject has a Bonacini index score of 7. In certain embodiments, the subject has a Bonacini index score greater than 7. In certain embodiments, the subject has a Bonacini index score of 8. In certain embodiments, the subject has a Bonacini index score of 9. In certain embodiments, the subject has a Bonacini index score of 10. In certain embodiments, the subject has a Bonacini index score of 11. In some embodiments, administration of a TβRII antagonist decreases the Bonacini score to less than 7. In some embodiments, administration of a TβRII antagonist decreases the Bonacini score to 4-7. In some embodiments, administration of a TβRII antagonist decreases the Bonacini score to less than 6. In some embodiments, administration of a TβRII antagonist decreases the Bonacini score to less than 5. In some embodiments, administration of a TβRII antagonist decreases the Bonacini score to less than 4. In some embodiments, administration of a TβRII antagonist decreases the Bonacini score to less than 3. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Enhanced Liver Fibrosis Index (ELF)

In some embodiments, the disclosure provides methods of treating chronic liver disease, portal hypertension, primary sclerosing cholangitis, and hepatopulmonary syndrome, or a complication thereof (e.g., pulmonary fibrosis, ILD), comprising administering a TβRII antagonist to a subject in need thereof, wherein the subject is evaluated using the enhanced liver fibrosis index (ELF). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. The ELF index is a non-invasive blood test that measures three biomarkers associated with fibrosis. The ELF score is calculated using an algorithm which combines values for hyaluronic acid, procollagen III amino terminal peptide (PIIINP), and tissue inhibitor of matrix metalloproteinase 1 (TIMP-1). The ELF test is typically used to assess the risk of progression to cirrhosis in patients with liver disease. Three cut-off values have been identified with respect to ELF scoring. A ELF score greater than or equal to 9.8 has been demonstrated to represent clear evidence for a high risk/high probability of progression to advanced fibrosis, whereas an ELF score less than 9.8 has been shown to be associated with less risk/lower probability of progression to advanced fibrosis. A single unit increase in ELF score has also been shown to be associated with a 4.34-fold increase in risk of progression to advanced fibrosis in patients with an ELF score less than 9.8.

In some embodiments of the methods and uses disclosed herein, a subject has chronic liver disease, portal hypertension, primary sclerosing cholangitis, and/or hepatopulmonary syndrome, or a complication associated with chronic liver disease, portal hypertension, primary sclerosing cholangitis, and/or hepatopulmonary syndrome (e.g., pulmonary fibrosis or ILD). In some embodiments of the methods and uses disclosed herein, a subject has ILD associated with chronic liver disease, portal hypertension, primary sclerosing cholangitis, and/or hepatopulmonary syndrome. In some embodiments of the methods and uses disclosed herein, a subject has pulmonary fibrosis associated with chronic liver disease, portal hypertension, primary sclerosing cholangitis, and/or hepatopulmonary syndrome. In some embodiments of the methods and uses disclosed herein, the subject has an ELF score less than or equal to 7. In certain embodiments, the subject has an ELF score less than or equal to 7.1. In certain embodiments, the subject has an ELF score less than or equal to 7.3. In certain embodiments, the subject has an ELF score less than or equal to 7.5. In certain embodiments, the subject an ELF score less than or equal to 7.7. In certain embodiments, the subject has an ELF score of 7.7. In certain embodiments, the subject has an ELF score greater than or equal to 7.7. In certain embodiments, the subject has an ELF score greater than or equal to 8. In certain embodiments, the subject has an ELF score greater than or equal to 8.5. In certain embodiments, the subject has an ELF score greater than or equal to 9. In certain embodiments, the subject has an ELF score greater than or equal to 9.5. In certain embodiments, the subject has an ELF score less than or equal to 9.8. In certain embodiments, the subject has an ELF score of 9.8. In certain embodiments, the subject has an ELF score greater than or equal to 9.8. In certain embodiments, the subject has an ELF score greater than or equal to 10. In certain embodiments, the subject has an ELF score greater than or equal to 11. In certain embodiments, the subject has an ELF score less than or equal to 11.3. In certain embodiments, the subject has an ELF score of 11.3. In certain embodiments, the subject has an ELF score greater than or equal to 11.3. In some embodiments, administration of a TβRII antagonist decreases the subject's ELF score below 9.8. In some embodiments, administration of a TβRII antagonist decreases the subject's ELF score below 11.3.

Fibrosis-4 (FIB-4) Index for Liver Fibrosis

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using the fibrosis-4 index for liver fibrosis (FIB-4). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. FIB-4 is used to estimate the amount of scarring in the liver, and is commonly implemented alongside other fibrosis scoring methods as an alternative to invasive liver biopsy methodologies. The score is determined using the patient's AST and ALT levels (in units per liter), as well as the patient's platelet count. The low cutoff value for FIB-4 scoring is 1.45, which represents a negative predictive value of 90% for advanced fibrosis. A FIB-4 score greater than 3.25 represents a 97% specificity and positive predictive value of 65% for advanced fibrosis. In some embodiments of the methods and uses disclosed herein, a subject has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In certain embodiments, the subject has a FIB-4 score less than or equal to 1.45. In certain embodiments, the subject has a FIB-4 score of 1.45. In certain embodiments, the subject has a FIB-4 score greater than or equal to 1.45. In certain embodiments, the subject has a FIB-4 score less than or equal to 3.25. In certain embodiments, the subject has a FIB-4 score of 3.25. In certain embodiments, the subject has a FIB-4 score greater than or equal to 3.25. In some embodiments, the subject has a FIB-4 score between 1.45 and 3.25. In some embodiments, administration of a TβRII antagonist reduces the subject's FIB-4 score to less than 3.25. In some embodiments, administration of a TβRII antagonist reduces the subject's FIB-4 score to less than 1.45.

FibroIndex

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using the FibroIndex. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. The FibroIndex is an index for predicting fibrosis in chronic hepatitis C and is also used as a surrogate marker during antifibrotic treatment for chronic hepatitis C. The FibroIndex is derived from platelet count, AST, and gamma globulin measurements. Changes in the FibroIndex have been shown to correlate significantly with variation in fibrosis stage. A value greater than or equal to 2.25 represents the high cutoff value for the FibroIndex, and suggests with 95% specificity that significant fibrosis is present. A value less than or equal to 1.25 represents the low cutoff value for the FibroIndex, and suggests with 95% specificity that significant fibrosis is not present. In some embodiments of the methods and uses disclosed herein, a subject has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In certain embodiments, the subject has a FibroIndex less than or equal to 1.25. In certain embodiments, the subject has a FibroIndex of 1.25. In certain embodiments, the subject has a FibroIndex greater than or equal to 1.25. In certain embodiments, the subject has a FibroIndex less than 2.25. In certain embodiments, the subject has a FibroIndex of 2.25. In certain embodiments, the subject has a FibroIndex greater than or equal to 2.25. In some embodiments, administration of a TβRII antagonist reduces the subject's FibroIndex to less than 2.25. In some embodiments, administration of a TβRII antagonist reduces the subject's FibroIndex to between 2.25 and 1.25. In some embodiments, administration of a TβRII antagonist reduces the subject's FibroIndex to less than 1.25.

Forn's Index

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using the Forn's index. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. Forn's index is based upon serum biomarkers used to predict liver fibrosis, and was developed as a means to replace invasive liver biopsy or endoscopy procedures in detection of esophageal varices. Forn's index is determined using a patient's platelet count, age, γGT (gamma glutamyl transferase) levels, and total cholesterol. A Forn's index value of 6.9 differentiates mild fibrosis from severe fibrosis. γGT is an enzyme produced throughout the body, but has the highest concentrations in the liver. Diseases which damage the liver or bile ducts typically result in increased circulating levels of γGT. γGT levels are measured as a nonspecific marker for liver damage, but cannot differentiate between various causes of liver damage or liver disease. Because of this nonspecificity, γGT is typically measured in combination with other biomarkers for liver damage such as alkaline phosphatase (ALP). In some embodiments of the methods and uses disclosed herein, a subject has chronic liver disease, portal hypertension, primary sclerosing cholangitis, and hepatopulmonary syndrome, or a complication thereof (e.g., pulmonary fibrosis, ILD). In certain embodiments, the subject has a Forn's index score less than or equal to 6.9. In certain embodiments, the subject has a Forn's index score of 6.9. In certain embodiments, the subject has a Forn's index score greater than or equal to 6.9. In certain embodiments, the subject has a Forn's index score greater than 6.9. In some embodiments, the subject has a Forn index score less than 6.9. In some embodiments, the subject has a Forn index score less than 6.5. In some embodiments, the subject has a Forn index score less than 6.0. In some embodiments, the subject has a Forn index score less than 5.5. In some embodiments, the subject has a Forn index score less than 5.0. In some embodiments, the subject has a Forn index score less than 4.0. In some embodiments, the subject has a Forn index score less than 3.0. In some embodiments, the subject has a Forn index score less than 2.0. In some embodiments, administration of a TβRII antagonist, decreases the subject's Forn index score. In some embodiments, administration of a TβRII antagonist, decreases the subject's Forn index score to less than 6.9. In some embodiments, administration of a TβRII antagonist, decreases the subject's Forn index score to less than 6.5. In some embodiments, administration of a TβRII antagonist, decreases the subject's Forn index score to less than 6.0. In some embodiments, administration of a TβRII antagonist, decreases the subject's Forn index score to less than 5.5. In some embodiments, administration of a TβRII antagonist, decreases the subject's Forn index score to less than 5.0. In some embodiments, administration of a TβRII antagonist, decreases the subject's Forn index score to less than 4.5. In some embodiments, administration of a TβRII antagonist, decreases the subject's Forn index score to less than 4.0. In some embodiments, administration of a TβRII antagonist, decreases the subject's Forn index score to less than 3. In some embodiments, administration of a TβRII antagonist, decreases the subject's Forn index score to less than 2.0. In some embodiments, administration of a TβRII antagonist, decreases the subject's Forn index score to less than 1.0

St. George's Respiratory Questionnaire (SGRQ)

In some embodiments, a subject of the present disclosure may be evaluated using a respiratory questionnaire. In some embodiments, a subject of the present disclosure may be evaluated using a St. George's Respiratory Questionnaire (SGRQ). In some embodiments, an SGRQ evaluates one or more of overall health, daily life, and/or perceived well-being of a subject with one or more obstructive airway diseases (e.g., a complication associated with chronic liver disease, portal hypertension, primary sclerosing cholangitis, and/or hepatopulmonary syndrome (e.g., pulmonary fibrosis or ILD). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. An SGRQ score ranges from 0 to 100 units, with higher scores indicating more limited respiratory function. Questions in an SGRQ are typically sorted into two categories comprising complications and activities that can cause and/or are limited by breathlessness. Categories can be split into multiple parts. Questions can have one or more methods of scoring, including but not limited to scaling, dichotomous true/false, and/or four-point Likert scale. In some embodiments, a subject's SGRQ score positively correlates with presence of cough, sputum and/or wheeze in a subject. In some embodiments, a subject's SGRQ score correlates with one or more measures of disease activity including but not limited to FEV1, FVC, SaO2 at rest, 6-MWD, MRC dyspnea grade, anxiety score, depression score, Sickness Impact Profile (SIP) total score, and SIP physical and/or psychosocial domains. Generally, a mean change in score of about 4 units indicates slightly efficacious treatment, a mean change in score of about 8 units indicates moderately efficacious treatment, and a mean change in score of about 12 units indicates very efficacious treatment. In some embodiments, efficacious treatment comprises a decrease in an SGRQ score of a subject. In some embodiments, treatment of a subject with one or more of a TβRII antagonist of the present disclosure changes a subject's SGRQ score by about 4 units. In some embodiments, treatment of a subject with one or more of a TβRII antagonist of the present disclosure changes a subject's SGRQ score by about 8 units. In some embodiments, treatment of a subject with one or more of a TβRII antagonist of the present disclosure changes a subject's SGRQ score by about 12 units. In some embodiments, treatment of a subject with one or more of a TβRII antagonist of the present disclosure changes a subject's SGRQ score by between about 0 and about 4 units. In some embodiments, treatment of a subject with one or more of a TβRII antagonist of the present disclosure changes a subject's SGRQ score by between about 1 and about 4 units. In some embodiments, treatment of a subject with one or more of a TβRII antagonist of the present disclosure changes a subject's SGRQ score by between about 5 and about 8 units. In some embodiments, treatment of a subject with one or more of a TβRII anta e (SGRQ) score of a subject by between about 1% and about 5%. In some embodiments, treatment of a subject with one or more of a TβRII antagonist of the present disclosure improves a St. George's Respiratory Questionnaire (SGRQ) score of a subject by between about 1% and about 8%. In some embodiments, treatment of a subject with one or more of a TβRII antagonist of the present disclosure improves a St. George's Respiratory Questionnaire (SGRQ) score of a subject by between about 1% and about 10%. In some embodiments, treatment of a subject with one or more of a TβRII antagonist of the present disclosure improves a St. George's Respiratory Questionnaire (SGRQ) score of a subject by between about 10% and about 15%. In some embodiments, treatment of a subject with one or more of a TβRII antagonist of the present disclosure improves a St. George's Respiratory Questionnaire (SGRQ) score of a subject by between about 15% and about 20%. In some embodiments, treatment of a subject with one or more of a TβRII antagonist of the present disclosure improves a St. George's Respiratory Questionnaire (SGRQ) score of a subject by between about 20% and about 25%. In some embodiments, treatment of a subject with one or more of a TβRII antagonist of the present disclosure improves a St. George's Respiratory Questionnaire (SGRQ) score of a subject by between about 25% and about 30%. In some embodiments, treatment of a subject with one or more of a TβRII antagonist of the present disclosure improves a St. George's Respiratory Questionnaire (SGRQ) score of a subject by between about 30% and about 35%. In some embodiments, treatment of a subject with one or more of a TβRII antagonist of the present disclosure improves a St. George's Respiratory Questionnaire (SGRQ) score of a subject by between about 35% and about 40%. In some embodiments, treatment of a subject with one or more of a TβRII antagonist of the present disclosure improves a St. George's Respiratory Questionnaire (SGRQ) score of a subject by between about 40% and about 45%. In some embodiments, treatment of a subject with one or more of a TβRII antagonist of the present disclosure improves a St. George's Respiratory Questionnaire (SGRQ) score of a subject by between about 45% and about 50%. In some embodiments, treatment of a subject with one or more of a TβRII antagonist of the present disclosure improves a St. George's Respiratory Questionnaire (SGRQ) score of a subject by between about 55% and about 60%. In some embodiments, treatment of a subject with one or more of a TβRII antagonist of the present disclosure improves a St. George's Respiratory Questionnaire (SGRQ) score of a subject by between about 60% and about 65%. In some embodiments, treatment of a subject with one or more of a TβRII antagonist of the present disclosure improves a St. George's Respiratory Questionnaire (SGRQ) score of a subject by between about 65% and about 70%. In some embodiments, treatment of a subject with one or more of a TβRII antagonist of the present disclosure improves a St. George's Respiratory Questionnaire (SGRQ) score of a subject by between about 10% and about 20%. In some embodiments, treatment of a subject with one or more of a TβRII antagonist of the present disclosure improves a St. George's Respiratory Questionnaire (SGRQ) score of a subject by between about 20% and about 30%. In some embodiments, treatment of a subject with one or more of a TβRII antagonist of the present disclosure improves a St. George's Respiratory Questionnaire (SGRQ) score of a subject by between about 30% and about 40%. In some embodiments, treatment of a subject with one or more of a TβRII antagonist of the present disclosure improves a St. George's Respiratory Questionnaire (SGRQ) score of a subject by between about 40% and about 50%. In some embodiments, treatment of a subject with one or more of a TβRII antagonist of the present disclosure improves a St. George's Respiratory Questionnaire (SGRQ) score of a subject by between about 60% and about 70%. In some embodiments, an SGRQ score is improved after the administering of one or more of a TβRII antagonists of the present disclosure, compared to a baseline measurement. In some embodiments, an SGRQ score is decreased after the administering of one or more of a TβRII antagonists of the present disclosure, compared to a baseline measurement.

King's Brief Interstitial Lung Disease (KBILD) Questionnaire

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using the King's Brief Interstitial Lung Disease (KBILD) questionnaire. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69. In some embodiments, the disclosure provides a method of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the administration improves subject's KBILD score.

The King's Brief Interstitial Lung Disease (KBILD) is a 15-item validated health-related quality of life (HRQOL) questionnaire. The KBILD questionnaire consists of three domains (breathlessness and activities, chest symptoms and psychological). The method of scoring the KBILD has recently changed to incorporate a logit-scale transformation from one that used raw item responses, as this is potentially a more linear scale. The KBILD questionnaire help to measure the impact ILD has on a subject's wellbeing and daily life. KBILDs can be used to monitor and assess how well treatment for interstitial lung disease associated conditions (e.g., a complication associated with chronic liver disease, portal hypertension, primary sclerosing cholangitis, and/or hepatopulmonary syndrome (e.g., pulmonary fibrosis or ILD)) is working. Each question in the KBILD can be answered with a rank from 1 to 7, with 1 being “all of the time” and 7 being “none of the time”. The lowest score a subject can obtain is 15 points. The highest score a subject can obtain is 105 points.

In some embodiments, a subject of the present disclosure may be evaluated using a respiratory questionnaire. In some embodiments, a subject of the present disclosure may be evaluated using a King's Brief Interstitial Lung Disease (KBILD) questionnaire. In some embodiments, a KBILD evaluates one or more of overall health, daily life, response to treatment and/or perceived well-being of a subject with interstitial lung disease.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein treatment improves a subject's KBILD score. In some embodiments, treatment of a subject with one or more of a TβRII antagonist of the present disclosure improves a subject's KBILD score by about 1 unit. In some embodiments, treatment of a subject with one or more of a TβRII antagonist of the present disclosure improves a subject's KBILD score by about 3 units. In some embodiments, treatment of a subject with one or more of a TβRII antagonist of the present disclosure improves a subject's KBILD score by about 7 units. In some embodiments, treatment of a subject with one or more of a TβRII antagonist of the present disclosure improves a subject's KBILD score by about 10 units. In some embodiments, treatment of a subject with one or more of a TβRII antagonist of the present disclosure improves a subject's KBILD score by about 14 units. In some embodiments, treatment of a subject with one or more of a TβRII antagonist of the present disclosure improves a subject's KBILD score by about 21 units. In some embodiments, treatment of a subject with one or more of a TβRII antagonist of the present disclosure improves a subject's KBILD score by about 28 units. In some embodiments, treatment of a subject with one or more of a TβRII antagonist of the present disclosure improves a subject's KBILD score by about 35 units. In some embodiments, treatment of a subject with one or more of a TβRII antagonist of the present disclosure improves a subject's KBILD score by about 42 units. In some embodiments, treatment of a subject with one or more of a TβRII antagonist of the present disclosure improves a subject's KBILD score by about 49 units. In some embodiments, treatment of a subject with one or more of a TβRII antagonist of the present disclosure improves a subject's KBILD score by about 56 units. In some embodiments, treatment of a subject with one or more of a TβRII antagonist of the present disclosure improves a subject's KBILD score by about 63 units. In some embodiments, treatment of a subject with one or more of a TβRII antagonist of the present disclosure improves a subject's KBILD score by about 70 units. In some embodiments, treatment of a subject with one or more of a TβRII antagonist of the present disclosure improves a subject's KBILD score by about 77 units. In some embodiments, treatment of a subject with one or more of a TβRII antagonist of the present disclosure improves a subject's KBILD score by about 84 units. In some embodiments, treatment of a subject with one or more of a TβRII antagonist of the present disclosure improves a subject's KBILD score by about 91 units. In some embodiments, treatment of a subject with one or more of a TβRII antagonist of the present disclosure improves a subject's KBILD score by about 98 units. In some embodiments, treatment of a subject with one or more of a TβRII antagonist of the present disclosure improves a subject's KBILD score by about 105 units.

In some embodiments, treatment of a subject with one or more of a TβRII antagonist of the present disclosure improves a subject's KBILD score by between about 0 and about 3 units. In some embodiments, treatment of a subject with one or more of a TβRII antagonist of the present disclosure improves a subject's KBILD score by between about 1 and about 7 units. In some embodiments, treatment of a subject with one or more of a TβRII antagonist of the present disclosure improves a subject's KBILD score by between about 7 and about 14 units. In some embodiments, treatment of a subject with one or more of a TβRII antagonist of the present disclosure improves a subject's KBILD score by between about 14 and about 21 units. In some embodiments, treatment of a subject with one or more of a TβRII antagonist of the present disclosure improves a subject's KBILD score by about more than 21 units.

In some embodiments, treatment of a subject with one or more of a TβRII antagonist of the present disclosure improves a KBILD score of a subject. In some embodiments, treatment of a subject with one or more of a TβRII antagonist of the present disclosure improves a KBILD score of a subject by between about 1% and about 3%. In some embodiments, treatment of a subject with one or more of a TβRII antagonist of the present disclosure improves a KBILD score of a subject by between about 1% and about 5%. In some embodiments, treatment of a subject with one or more of a TβRII antagonist of the present disclosure improves a KBILD score of a subject by between about 1% and about 8%. In some embodiments, treatment of a subject with one or more of a TβRII antagonist of the present disclosure improves a KBILD score of a subject by between about 1% and about 10%. In some embodiments, treatment of a subject with one or more of a TβRII antagonist of the present disclosure improves a KBILD score of a subject by between about 10% and about 15%. In some embodiments, treatment of a subject with one or more of a TβRII antagonist of the present disclosure improves a KBILD score of a subject by between about 15% and about 20%. In some embodiments, treatment of a subject with one or more of a TβRII antagonist of the present disclosure improves a KBILD score of a subject by between about 20% and about 25%. In some embodiments, treatment of a subject with one or more of a TβRII antagonist of the present disclosure improves a KBILD score of a subject by between about 25% and about 30%. In some embodiments, treatment of a subject with one or more of a TβRII antagonist of the present disclosure improves a KBILD score of a subject by between about 30% and about 35%. In some embodiments, treatment of a subject with one or more of a TβRII antagonist of the present disclosure improves a KBILD score of a subject by between about 35% and about 40%. In some embodiments, treatment of a subject with one or more of a TβRII antagonist of the present disclosure improves a KBILD score of a subject by between about 40% and about 45%. In some embodiments, treatment of a subject with one or more of a TβRII antagonist of the present disclosure improves a KBILD score of a subject by between about 45% and about 50%. In some embodiments, treatment of a subject with one or more of a TβRII antagonist of the present disclosure improves a KBILD score of a subject by between about 55% and about 60%. In some embodiments, treatment of a subject with one or more of a TβRII antagonist of the present disclosure improves a KBILD score of a subject by between about 60% and about 65%. In some embodiments, treatment of a subject with one or more of a TβRII antagonist of the present disclosure improves a KBILD score of a subject by between about 65% and about 70%. In some embodiments, treatment of a subject with one or more of a TβRII antagonist of the present disclosure improves a KBILD score of a subject by between about 10% and about 20%. In some embodiments, treatment of a subject with one or more of a TβRII antagonist of the present disclosure improves a KBILD score of a subject by between about 20% and about 30%. In some embodiments, treatment of a subject with one or more of a TβRII antagonist of the present disclosure improves a KBILD score of a subject by between about 30% and about 40%. In some embodiments, treatment of a subject with one or more of a TβRII antagonist of the present disclosure improves a KBILD score of a subject by between about 40% and about 50%. In some embodiments, treatment of a subject with one or more of a TβRII antagonist of the present disclosure improves a KBILD score of a subject by between about 60% and about 70%. In some embodiments, a KBILD score is improved after the administering of one or more of a TβRII antagonists of the present disclosure, compared to a baseline measurement. In some embodiments, a KBILD score is increased after the administering of one or more of a TβRII antagonists of the present disclosure, compared to a baseline measurement.

Health Assessment Questionnaire Disability Index (HAQ-DI)

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using a Health Assessment Questionnaire Disability Index (HAQ-DI). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

The Health Assessment Questionnaire-Disability (HAQ-DI) consists of 20 questions referring to eight component sets: dressing/grooming, arising, eating, walking, hygiene, reach, grip and activities. The total score indicates the patient's self-assessed level of disability. A negative change from baseline indicates improvement.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist (e.g., a TβRII polypeptide or fusion protein) to a subject in need thereof, wherein the antagonist improves the subject's HAQ-DI score compared to a baseline measurement. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist imparts a negative change in HAQ-DI score of a subject compared to a baseline measurement. In some embodiments, the complication associated with chronic liver disease, portal hypertension, primary sclerosing cholangitis, and/or hepatopulmonary syndrome is pulmonary fibrosis. In some embodiments, the complication associated with chronic liver disease, portal hypertension, primary sclerosing cholangitis, and/or hepatopulmonary syndrome is ILD.

Physical/Physician Global Assessment and Patient Global Assessment

In some embodiments, the disclosure provides methods of treating chronic liver disease, portal hypertension, primary sclerosing cholangitis, and hepatopulmonary syndrome, a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using a physical/physician global assessment. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

The physician's global assessment is to be completed on the basis of examination and overall assessment of the patient after all other trial procedures have been completed. The physician's assessment of the patient's status will be scored on a 100-mm horizontal visual analog scale (VAS).

In some embodiments, the disclosure provides methods of treating chronic liver disease, portal hypertension, primary sclerosing cholangitis, and hepatopulmonary syndrome, a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist improves the subject's physical/physician global assessment score compared to a baseline measurement.

In some embodiments, the disclosure provides methods of treating chronic liver disease, portal hypertension, primary sclerosing cholangitis, and hepatopulmonary syndrome, a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using a patient global assessment.

The patient's global assessment represents the patient's (e.g., subject's) overall assessment of his or her current status on a 100-mm horizontal visual analog scale (VAS). The patient's global assessment is self-administered by the patient.

In some embodiments, the disclosure provides methods of treating chronic liver disease, portal hypertension, primary sclerosing cholangitis, and hepatopulmonary syndrome, a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist improves the subject's patient global assessment score compared to a baseline measurement.

Clinical Worsening

In some embodiments, the disclosure provides methods of treating chronic liver disease, portal hypertension, primary sclerosing cholangitis, and/or hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist improves the time to clinical worsening of the subject. In some embodiments, the antagonist lengthens the time to clinical worsening of the subject. In some embodiments, the antagonist slows the rate of decline of pulmonary function of the subject. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist extends the subject's length of life compared to a baseline measurement prior to the administering. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist prevents a decline in FVC of the subject of greater than 10% of predicted relative to a baseline measurement. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist prevents at least a greater than 20% increase in mRSS of the subject. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist prevents at least a greater than 5 point increase in total mRSS of the subject. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the antagonist prevents an occurrence of a chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) in the subject. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

Combination Therapies

In certain aspects, the disclosure contemplates the use of a TβRII antagonist in combination with one or more additional active agents or other supportive therapy for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more TβRII antagonists of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the TβRII antagonist of the present disclosure with the additional active agent or therapy and/or the desired effect.

In some embodiments, a patient has been treated with one or more active agents, which is not a TβRII antagonist, or other supportive therapy for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) prior to administration of a TβRII antagonist of the present disclosure. In some embodiments, a patient was not previously taking one or more other active agents or supportive therapies for treating chronic liver disease, portal hypertension, primary sclerosing cholangitis, and hepatopulmonary syndrome, or a complication thereof (e.g., pulmonary fibrosis, ILD) prior to administration of a TβRII antagonist. In some embodiments a patient is administered a TβRII antagonist in combination with one or more additional active agents and/or supportive therapies for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises abatacept (e.g., orencia). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises abituzumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises ajulemic acid (e.g., anabasum or lenabasum or resunab). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises ambrisentan. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises AVID200. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises AVID300. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises azathioprine (e.g., imuran). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises BCD-089. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises belimumab (e.g., benlysta). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises BG00011. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises BMS-986020. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises bortezomib (e.g., velcade). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises bosentan. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises brentuximab (e.g., adcetris). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises carlumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises CC-90001. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises clazakizumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises COR-001. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises cyclophosphamide or CYC. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises cyclosporine A. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises dectrekumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises EHP-101. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises elzonris or SL-401. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises etanercept. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises FCX-013. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises fresolimumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises GLPG1690. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises GSK2126458. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises GSK2330811. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises GSK3008348. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises IBIO-CFB03. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises ifetroban (e.g., vasculan). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises IFNγ. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises imatinib. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises immune globulin (e.g., privigen). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises IW001. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises lanifibranor. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises lebrikizumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises levilimab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises losartan. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises macitentan. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises MEDI-5117. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises methotrexate. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises MSCs. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises mycophenolate mofetil or MMF. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises NAC. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises nandrolone decanoate. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises nintedanib (e.g., ofev). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises olokizumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises pamrevlumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises pirfenidone (e.g., esbriet). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises pirfenidone and vismodegib. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises pomalidomide (e.g., pomalyst). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises PRM-151. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises riociguat (e.g., adempas). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises rituximab (e.g., rituxan). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises SAR156597. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises sildenafil. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises siltuximab (e.g., sylvant). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises simtuzumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises sirolimus. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises sirukumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises tacrolimus. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises tadalafil. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises tanzisertib. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises TD139. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises tetrathiomolybdate. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises tocilizumab (e.g., actemra). In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises tralokinumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises treprostinil. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises vobarilizumab. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises warfarin. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises zileuton. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises ziltivekimab.

In some embodiments, a subject has been treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of abatacept, abituzumab, ajulemic acid, ambrisentan, AVID200, AVID300, azathioprine, BCD-089, belimumab, BG00011, BMS-986020, bortezomib, bosentan, brentuximab, carlumab, CC-90001, cephalosporin, clazakizumab, COR-001, cyclophosphamide (CYC), cyclosporine A, dectrekumab, EHP-101, elzonris/SL-401, etanercept, FCX-013, fresolimumab, GLPG1690, GAS K2126458, GSK2330811, GSK3008348, IBIO-CFB03, ifetroban, IFNγ, imatinib, immune globulin, IW001, lanifibranor, lebrikizumab, levilimab, losartan, macitentan, MEDI-5117, methotrexate, MSCs, mycophenolate mofetil (MMF), NAC, nandrolone decanoate, nintedanib, olokizumab, pamrevlumab, pirfenidone, pirfenidone and vismodegib, pomalidomide, PRM-151. riociguat, rituximab, SAR156597, sildenafil, siltuximab, simtuzumab, sirolimus, sirukumab, tacrolimus, tadalafil, tanzisertib, TD139, tetrathiomolybdate, tocilizumab, tralokinumab, treprostinil, vobarilizumab, warfarin, zileuton, and ziltivekimab.

In some embodiments, a subject is further treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of abatacept, abituzumab, ajulemic acid, AVID200, AVID300, azathioprine, BCD-089, belimumab, bortezomib, bosentan, brentuximab, cephalosporin, clazakizumab, COR-001, cyclophosphamide (CYC), cyclosporine A, EHP-101, elzonris/SL-401, FCX-013, GLPG1690, GSK2330811, IBIO-CFB03, ifetroban, imatinib, immune globulin, lanifibranor, levilimab, MEDI-5117, mycophenolate mofetil (MMF), olokizumab, pirfenidone, pomalidomide, riociguat, rituximab, SAR156597, siltuximab, sirukumab, tacrolimus, tadalafil, tocilizumab, vobarilizumab, and/or ziltivekimab.

In some embodiments, a subject has been treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of abatacept, abituzumab, ajulemic acid, AVID200, AVID300. azathioprine, belimumab, bortezomib, bosentan, brentuximab, cyclophosphamide (CYC), cyclosporine A, EHP-101, elzonris/SL-401, FCX-013, GLPG1690, GSK2330811, IBIO-CFB03, ifetroban, imatinib, lanifibranor, methotrexate, mycophenolate mofetil (MMF), nintedanib, pirfenidone, pomalidomide, privigen, riociguat, rituximab, SAR156597, tacrolimus, tadalafil, and/or tocilizumab.

In some embodiments, a subject is further treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of abatacept, abituzumab, ajulemic acid, AVID200, AVID300, azathioprine, belimumab, bortezomib, bosentan, brentuximab, cyclophosphamide (CYC), cyclosporine A, EHP-101, elzonris/SL-401, FCX-013, GLPG1690, GSK2330811, IBIO-CFB03, ifetroban, imatinib, lanifibranor, methotrexate, mycophenolate mofetil (MMF), nintedanib, pirfenidone, pomalidomide, privigen, riociguat, rituximab, SAR156597, tacrolimus, tadalafil, and/or tocilizumab.

In some embodiments, a subject has been treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of azathioprine, cyclophosphamide (CYC), methotrexate, mycophenolate mofetil (MMF), nintedanib, and/or tocilizumab. In some embodiments, the disclosure provides methods of treating chronic liver disease, portal hypertension, primary sclerosing cholangitis, and hepatopulmonary syndrome, or a complication thereof (e.g., pulmonary fibrosis, ILD), comprising administering a TβRII antagonist to a subject in need thereof, wherein the method further comprises administration of one or more of azathioprine, cyclophosphamide (CYC), methotrexate, mycophenolate mofetil (MMF), nintedanib, and/or tocilizumab.

In some embodiments, a subject is further treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more cephalosporins.

In some embodiments, a subject is further treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of azathioprine, cyclophosphamide (CYC), methotrexate, mycophenolate mofetil (MMF), and/or nintedanib.

In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises one or more of IL-6 and/or/or IL-6R antibodies. In some embodiments, IL-6 antibodies comprise one or more of clazakizumab, COR-001, MEDI-5117, olokizumab, siltuximab, sirukumab, and/or ziltivekimab. In some embodiments, IL-6R antibodies comprise one or more of BCD-089 (levilimab), tocilizumab, and/or vobarilizumab.

In some embodiments, a subject has been treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of clazakizumab, COR-001, MEDI-5117, olokizumab, siltuximab, sirukumab, and/or ziltivekimab.

In some embodiments, a subject is further treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of clazakizumab, COR-001, MEDI-5117, olokizumab, siltuximab, sirukumab, and/or ziltivekimab

In some embodiments, a subject has been treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of BCD-089 (levilimab), tocilizumab, and/or vobarilizumab.

In some embodiments, a subject is further treated with a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprising one or more of BCD-089 (levilimab), tocilizumab, and/or vobarilizumab.

In some embodiments, a therapeutic agent for treating chronic liver disease, portal hypertension, primary sclerosing cholangitis, and hepatopulmonary syndrome, or a complication thereof (e.g., pulmonary fibrosis, ILD) targets IL-3R. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) comprises an endostatin. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets PPARγ/CB2. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets MMP-1 cells. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets autotaxin. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets sGC. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets IL-4/IL-13. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets IL-6/IL-6R. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets TPR. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets oncostatin M. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets p38. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets FGFR/PDGFR. In some embodiments, a therapeutic agent for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) targets CB1/CB2.

The present disclosure contemplates the use of TβRII antagonists in combination with one or more supportive therapies for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a supportive therapy for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is a hematopoietic stem cell transplantation (HSCT). In some embodiments, one or more TβRII antagonists of the present disclosure are administered prior to a hematopoietic stem cell transplantation. In some embodiments, one or more TβRII antagonists of the present disclosure are administered during a hematopoietic stem cell transplantation. In some embodiments, one or more TβRII antagonists of the present disclosure are administered after a hematopoietic stem cell transplantation.

In some embodiments, a supportive therapy for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is a lung transplant. In some embodiments, one or more TβRII antagonists of the present disclosure are administered prior to a lung transplant. In some embodiments, one or more TβRII antagonists of the present disclosure are administered during a lung transplant. In some embodiments, one or more TβRII antagonists of the present disclosure are administered after a lung transplant. In some embodiments, a supportive therapy for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is a liver transplant. In some embodiments, one or more TβRII antagonists of the present disclosure are administered prior to a liver transplant. In some embodiments, one or more TβRII antagonists of the present disclosure are administered during a liver transplant. In some embodiments, one or more TβRII antagonists of the present disclosure are administered after a liver transplant.

In some embodiments, a subject of the present disclosure may have one or more additional fibrotic diseases or conditions. As used herein, the term “fibrosis” or “fibrotic” refers to the aberrant formation or development of excess fibrous connective tissue by cells in an organ or tissue. Although processes related to fibrosis can occur as part of normal tissue formation or repair, dysregulation of these processes can lead to altered cellular composition and/or excess connective tissue deposition that progressively impairs to tissue or organ function. The formation of fibrous tissue can result from a reparative or reactive process. As used herein, the terms “fibrotic disorder”, “fibrotic condition,” and/or “fibrotic disease,” are used interchangeably to refer to a disorder, condition or disease characterized by fibrosis. Additional fibrotic diseases or conditions include, but are not limited to, fibroproliferative disorders associated with vascular diseases, such as cardiac disease, cerebral disease, and/or peripheral vascular disease, as well as tissues and/or organ systems including the heart, skin, kidney, peritoneum, gut, and/or liver (as disclosed in, e.g., Wynn, 2004, Nat Rev 4:583-594, incorporated herein by reference). Exemplary additional fibrotic diseases or conditions that can be treated include, but are not limited to, renal fibrosis, including nephropathies associated with injury/fibrosis, e.g., chronic nephropathies associated with diabetes (e.g., diabetic nephropathy), lupus, glomerular nephritis, focal segmental glomerular sclerosis, and/or IgA nephropathy; gut fibrosis, e.g., radiation-induced gut fibrosis; liver fibrosis, e.g., cirrhosis, alcohol-induced liver fibrosis, biliary duct injury, primary biliary cirrhosis, infection or viral-induced liver fibrosis, congenital hepatic fibrosis and/or autoimmune hepatitis; and/or other fibrotic conditions, such as cystic fibrosis, endomyocardial fibrosis, mediastinal fibrosis, sarcoidosis, spinal cord injury/fibrosis, myelofibrosis, vascular restenosis, atherosclerosis, injection fibrosis (which can occur as a complication of intramuscular injections, especially in children), endomyocardial fibrosis, retroperitoneal fibrosis, nephrogenic systemic fibrosis, vascular fibrosis, pancreatic fibrosis, liver fibrosis (e.g., cirrhosis), renal fibrosis, musculoskeletal fibrosis, cardiac fibrosis (e.g., endomyocardial fibrosis, idiopathic myocardiopathy), skin fibrosis (e.g., post-traumatic, operative cutaneous scarring, keloids and/or cutaneous keloid formation), eye fibrosis (e.g., glaucoma, sclerosis of the eyes, conjunctival and/or corneal scarring, and/or pterygium), myelofibrosis, chronic graft-versus-host disease, Peyronie's disease, post-cystoscopic urethral stenosis, idiopathic and/or pharmacologically induced retroperitoneal fibrosis, mediastinal fibrosis, proliferative fibrosis, neoplastic fibrosis, Dupuytren's disease, strictures, neural scarring, dermal scarring, idiopathic pulmonary fibrosis and/or radiation induced fibrosis.

In particular, TβRII antagonists may be used, alone or in combination with one or more supportive therapies or active agents, to treat one or more complications of an additional diseases or conditions occurring in a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), myelofibrosis, wherein complications include, for example, ineffective hematopoiesis, anemia, inflammation, fibrosis (e.g., bone marrow fibrosis, spleen fibrosis, and/or liver fibrosis), pancytopenia, thrombocytopenia, extramedullary hematopoiesis (e.g., splenic extramedullary hematopoiesis, hepatic extramedullary hematopoiesis, pulmonary extramedullary hematopoiesis, and/or lymphatic extramedullary hematopoiesis), hepatomegaly, splenomegaly, osteosclerosis, osteomyelofibrosis, poikilocytosis, fatigue, weight loss, night sweats, fever, pruritus, bone pain, early satiety, abdominal pain or discomfort, arthralgias, myalgias, parasthesias, cachexia, splenic infarct, and/or bleeding.

As used herein, inhibition of the fibrotic response of a cell, includes, but is not limited to the inhibition of the fibrotic response of one or more cells within the liver (or liver tissue); one or more cells within the kidney (or renal tissue); one or more cells within muscle tissue; one or more cells within the heart (or cardiac tissue); one or more cells within the pancreas; one or more cells within the skin; one or more cells within the bone, one or more cells within the vasculature, one or more stem cells, or one or more cells within the eye.

In some embodiments, any of the TβRII antagonists of the disclosure may be used for treating one or more additional diseases or conditions occurring in a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising chronic obstructive pulmonary disease (COPD), chronic obstructive airway disorder, idiopathic pulmonary fibrosis and/or asthma. In some embodiments, the TβRII antagonist is a TβRII polypeptide or fusion protein comprising or consisting of, an amino acid sequence that is at least 80% identical, and optionally at least 85%, 90%, 91%, 92%, 93%, 94%, 95%, 96%, 97%, 98%, 99%, or 100% identical to SEQ ID NOs: 13, 18, 48, 67, or 69.

The present invention contemplates the use of TβRII antagonists in combination with one or more other therapeutic modalities in a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). Thus, in addition to the use of TβRII antagonists, one may also administer to the subject one or more “standard” therapies for treating fibrotic disorders in a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). For example, the TβRII antagonists can be administered to a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) in combination with (i.e., together with) cytotoxins, immunosuppressive agents, radiotoxic agents, and/or therapeutic antibodies. Particular co-therapeutics contemplated by the present invention include, but are not limited to, steroids (e.g., corticosteroids, such as Prednisone), immune-suppressing and/or anti-inflammatory agents (e.g., gamma-interferon, cyclophosphamide, azathioprine, methotrexate, penicillamine, cyclosporine, colchicine, antithymocyte globulin, mycophenolate mofetil, and/or hydroxychloroquine), cytotoxic drugs, calcium channel blockers (e.g., nifedipine), angiotensin converting enzyme inhibitors (ACE) inhibitors, para-aminobenzoic acid (PABA), dimethyl sulfoxide, transforming growth factor beta (TGFβ) inhibitors, interleukin-5 (IL-5) inhibitors, and/or pan caspase inhibitors.

Additional anti-fibrotic agents that may be used in combination with TβRII antagonists and administered to a subject with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) include, but are not limited to, lectins (as described in, for example, U.S. Pat. No. 7,026,283, the entire contents of which is incorporated herein by reference), as well as the anti-fibrotic agents described by Wynn et al (2007, J Clin Invest 117:524-529, the entire contents of which is incorporated herein by reference). For example, additional anti-fibrotic agents and/or therapies include, but are not limited to, various anti-inflammatory/immunosuppressive/cytotoxic drugs (including colchicine, azathioprine, cyclophosphamide, prednisone, thalidomide, pentoxifylline and/or theophylline), TGFβ signaling modifiers (including relaxin, SMAD7, HGF, and/or BMP7, as well as TGFβ1, TβRII, EGR-I, and/or CTGF inhibitors), cytokine and/or cytokine receptor antagonists (inhibitors of IL-1β, IL-5, IL-6, IL-13, IL-21, IL-4R, IL-13Rαl, GM-CSF, TNF-α, oncostatin M, W1SP-I, and/or PDGFs), cytokines and/or chemokines (IFN-γ, IFN-α/(3, IL-12, IL-10, HGF, CXCL10, and/or CXCL11), chemokine antagonists (inhibitors of CXCL1, CXCL2, CXCL12, CCL2, CCL3, CCL6, CCL17, and/or CCL18), chemokine receptor antagonists (inhibitors of CCR2, CCR3, CCR5, CCR7, CXCR2, and/or CXCR4), TLR antagonists (inhibitors of TLR3, TLR4, and/or TLR9), angiogenesis antagonists (VEGF-specific antibodies and/or adenosine deaminase replacement therapy), antihypertensive drugs (beta blockers and/or inhibitors of ANG 11, ACE, and/or aldosterone), vasoactive substances (ET-1 receptor antagonists and/or bosentan), inhibitors of the enzymes that synthesize and/or process collagen (inhibitors of prolyl hydroxylase), B cell antagonists (rituximab), integrin/adhesion molecule antagonists (molecules that block a1 β1 and/or αvβ6 integrins, as well as inhibitors of integrin-linked kinase, and/or antibodies specific for ICAM-I and/or VCAM-I), proapoptotic drugs that target myofibroblasts, MMP inhibitors (inhibitors of MMP2, MMP9, and/or MMP12), and/or T1MP inhibitors (antibodies specific for TIMP-1).

Biomarkers

Biomarkers disclosed herein may be defined as characteristics that are objectively measured and/or evaluated as an indicator of normal biologic processes, pathogenic processes (e.g., chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), or pharmacologic responses to a therapeutic intervention (e.g., treatment of chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) with one or more TβRII antagonists of the present disclosure). Biomarkers of liver disease may be measured by way of one or more of blood tests, liver biopsy, imaging, liver function, genetic polymorphisms and/or biochemical molecules identifiable in liver tissue. Biomarkers of pulmonary disease (e.g., a complication associated with chronic liver disease, portal hypertension, primary sclerosing cholangitis, and/or hepatopulmonary syndrome [e.g., pulmonary fibrosis or ILD]) may be measured by way of one or more of imaging, lung function, genetic polymorphisms and/or biochemical molecules identifiable in lung tissue, BAL fluid and/or blood. Serum biomarkers of the present disclosure can be useful in all stages of clinical treatment, including, but not limited to, evaluation for predisposition, diagnosis of liver disease, therapeutic treatment of liver disease, and/or research/clinical studies of liver disease. In some embodiments, the disclosure provides a method of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more biomarkers selected from the group consisting of SerpinE1, FN1, Colla1, aSMA, collagen-1. In some embodiments, the disclosure provides a method of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more biomarkers selected from the group consisting of albumin, bilirubin, AST (aspartate aminotransferase), ALT (alanine aminotransferase), γGT (gamma glutamyl transferase), ALP (alkaline phosphatase), prothrombin time, PICP (procollagen I carboxy peptide), PIIINP (procollagen III amino peptide), collagen type-IV, hyaluronic acid, laminin, YKL-40, MMPs including MMP7 and/or MMP12, TIMPs (tissue inhibitor of metalloproteinase), TGFβ1, PDGF (platelet derived growth factor), and microfibril associated protein-4.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject is evaluated using measurements of biomarkers. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is a biomarker of liver disease and/or liver fibrosis. In some embodiments, a biomarker for liver disease and/or liver fibrosis is selected from the group consisting of albumin, bilirubin, AST (aspartate aminotransferase), ALT (alanine aminotransferase), γGT (gamma glutamyl transferase), ALP (alkaline phosphatase), prothrombin time, PICP (procollagen I carboxy peptide), PIIINP (procollagen III amino peptide), collagen type-IV, hyaluronic acid, laminin, YKL-40, MMPs including MMP7 and/or MMP12, TIMPs (tissue inhibitor of metalloproteinase), TGFβ1, PDGF (platelet derived growth factor), and microfibril associated protein-4. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is AST. The liver produces increased levels of AST when liver damage or fibrosis is present. High circulating levels of AST in the blood are typically used as an indicator of cirrhosis. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is ALT. ALT is one of the oldest biomarkers used to assess liver disease with high sensitivity and specificity, where ALT levels 2.25-fold greater than normal levels predict liver histology. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is γGT. γGT is an enzyme produced throughout the body, but has the highest concentrations in the liver. Diseases which damage the liver or bile ducts typically result in increased circulating levels of γGT. γGT levels are measured as a nonspecific marker for liver damage, but cannot differentiate between various causes of liver damage or liver disease. Because of this nonspecificity, γGT is typically measured in combination with other biomarkers for liver damage such as alkaline phosphatase (ALP). In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is ALP. ALP is a widely used biomarker for liver disease, particularly nonalcoholic fatty liver disease (NAFLD) which is associated with insulin resistance. The highest concentrations of ALP are found in liver and bone, and lower concentrations of ALP are present in kidney tubules, intestinal epithelium, lung, and placenta. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is PIIINP. PIIINP is a major component of connective tissue and is measured in serum to reflect the presence and progression of hepatic fibrosis. Increased PIIINP levels correlate with the severity of liver disease. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is prothrombin time. Prothrombin time is measured in a blood test to determine the length of time required for blood to clot. Increased prothrombin times typically indicate liver damage, and suggest poor production of blood clotting proteins in the liver. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is type IV collagen. Type IV collagen is a component of the extracellular matrix and has been studied extensively in liver diseases of different etiologies. Increased type IV collagen levels (using a cutoff value of 5.0 ng/mL) correlate significantly with the extent of hepatic fibrosis. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is hyaluronic acid. Hyaluronic acid is a glycosaminoglycan component of the extracellular matrix and is produced by hepatic stellate cells. Hyaluronic acid is used as a valuable biomarker for advanced fibrosis. Hyaluronic acid levels show a negative predictive value of 98% for excluding cirrhosis, and a cutoff level of greater than or equal to 50 ng/mL is used to detect severe fibrosis. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is laminin. Laminin is a noncollagenous glycoprotein in the extracellular matrix and is also produced by hepatic stellate cells. Elevated serum levels of laminin indicate liver fibrosis and cirrhosis in patients with chronic liver disease. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is YXL-40. YXL-40 is involved in remodeling of the extracellular matrix and is similarly shown to correlate with the degree of fibrosis in liver disease. YXL-40 is used as a biomarker for all forms of liver disease and cirrhosis with cutoff levels defined at 186.4 and 284.8 μg/L, for each respective condition. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is a matrix metalloproteinase. Matrix metalloproteinase enzymes are involved in degradation of the extracellular matrix, and levels of circulating MMP proteins are inversely correlated with fibrosis. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is TGFβ. TGFβ has been used as a biomarker for fibrosis in patients with liver disease, particularly patients with liver disease resulting from hepatitis C infections. Specifically, TGFβ levels were found to be increased in patients with hepatitis C infections, and were found to correlate with progression of hepatic fibrosis. TGFβ levels less than 75 ng/mL are predictive of stable disease. In some embodiments, the biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is PDGF. PDGF is a mitogen of hepatic stellate cells whose levels correlate with the severity of hepatic fibrosis. PDGF at cutoff values greater than or equal to 40.5 ng/L strongly correlate with fibrosis.

In some embodiments, the disclosure provides a method of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more biomarkers selected from the group consisting of aSMA, ADAM12, Anti-HSP70 IgG, BAFF, BLyS, C3M, C4M, C6M, Ca15.3, CC16, CCL2, CCL18, Colla1, Col3a1, CTGF, CXCL4, E-selectin, ET-1, fibronectin, ICAM, IL-6, IL-8, KL-6, MCP-1, MMP7, MMP12, Muc5B, Osteopontin, PAI-1, periostin, pro-C3, pro-C4, pro-C6, SP-A, SP-D, Tnfa, VCAM, VEGF, WFDC2(HE4), and YKL-40.

In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is selected from the group consisting of KL-6/MUC1, surface protein A (SP-A), surface protein D (SP-D), CC16, and/or Ca15.3. In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) may be involved in one or more alveolar epithelial cell damage pathways. In some embodiments, a subject of the present disclosure has an increased level of one or more biomarkers selected from the group consisting of Ca15.3, CC16, KL-6, SP-A, and SP-D. In some embodiments, a subject of the present disclosure has an increased level of one or more biomarkers selected from the group consisting of Ca15.3, CC16, KL-6, SP-A, and SP-D, when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more biomarkers selected from the group consisting of Ca15.3, CC16, KL-6, SP-A, and SP-D.

In some aspects, levels of KL-6 (encoded by MUC1), a glycoprotein found predominantly on type II pneumocytes and/or alveolar macrophages, may be elevated in the serum of subjects with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). Levels of KL-6 may correlate with the presence of pneumonitis and/or a radiological fibrosis score in subjects with ILD. In some embodiments, levels of KL-6 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of KL-6 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Surfactant proteins A and/or D (SP-A and/or SP-D, respectively) are generally elevated in serum of subjects with chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). Surfactant proteins comprise lipoprotein complexes contained within pulmonary surfactant, and/or are produced by type II AECs and/or Clara cells, which are generally important for innate immune defense mechanisms and/or modulating inflammatory response at the alveolar air-liquid interface. In some embodiments, levels of SP-A and/or SP-D are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of SP-A and/or SP-D when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Clara cells are typically multifunctional cells that are predominantly localized at terminal bronchioles that can secrete 16 kDa Clara cell protein (CC16). Clara cell protein (CC16) generally has important protective, immunosuppressive and/or anti-inflammatory functions. Serum CC16 has been shown to be elevated across several pulmonary conditions including ILD, compared with healthy controls and/or subjects. In some embodiments, levels of CC16 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of CC16 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Cancer associated antigen carbohydrate antigen 15.3 (Ca15.3) is produced from the same MUC1 gene that encodes for KL-6 and/or is expressed on various epithelial cells, including type II AECs. Elevated Ca15.3 has been demonstrated in IPF and/or ILD. In a study comprising over 200 subjects, Ca15.3 level correlated strongly with HRCT scores. Furthermore, Ca15.3 has been shown to outperform FVC in predicting survival. In some embodiments, levels of Ca15.3 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level Cal15.3 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is selected from the group consisting of MMP7, osteopontin, and/or ADAM12. In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) may be involved in one or more aberrant fibrogenesis and/or matrix remodeling pathways. In some embodiments, a subject of the present disclosure has an increased level of one or more biomarkers selected from the group consisting of MMP7, osteopontin, and/or ADAM12. In some embodiments, a subject of the present disclosure has an increased level of one or more biomarkers selected from the group consisting of MMP7, osteopontin, and/or ADAM12, when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Matrix metalloproteinases (MMPs) and/or their inhibitors (tissue inhibitors of metalloproteinases, TIMPs) are proteases important in mediating ECM degradation, activity of inflammatory mediators and/or growth factors in the lung. In ILD, serum MMP7, MMP12 and/or TIMP1 have been shown to be elevated when compared with healthy controls, and/or have been shown to correlate inversely with pulmonary function. In some embodiments, levels of any one of MM7, MMP12, and/or TIMP1 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of any one of MM7, MMP12, and/or TIMP1 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

ADAM proteins (a disintegrin and/or metalloproteinases) are a group of multifunctional proteins that share a metalloprotease domain with MMPs, and/or have a unique, multifunctional domain with both proteolytic and/or adhesive functions, and/or are known to play an important role in cell binding, migration and/or signaling. They are implicated in a variety of diseases including CTD, malignancy, Alzheimer's, Crohn's disease and/or possibly pulmonary fibrosis. Serum levels of ADAM12 have been shown to be elevated in subjects with ILD, compared to subjects that do not have ILD, and/or correlate positively with FVC and/or extent of ground glass opacities on HRCT, and/or correlate negatively with HRCT fibrosis score. In some embodiments, levels of ADAM12 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of ADAM12 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Osteopontin (OPN) is a multifunctional protein that is known to regulate inflammation, cellular immune response and/or T cell function, with a pro-fibrotic effect in ILD through mechanisms that are not well known. Serum OPN levels are elevated in IPF compared with subjects that do not have ILD, but may not differentiate well between ILD subtypes. In some embodiments, levels of OPN are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of OPN when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is selected from the group consisting of CCL18, YKL-40, ICAM, VCAM, E-selectin, Anti-HSP70 IgG, BLyS/BAFF, CCL2/MCP-1, IL-6, and/or CXCL4. In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) may be involved in one or more immune dysregulation and/or inflammation pathways. In some embodiments, a subject of the present disclosure has an increased level of one or more biomarkers selected from the group consisting of CCL18, YKL-40, ICAM, VCAM, E-selectin, Anti-HSP70 IgG, BLyS/BAFF, CCL2/MCP-1, IL-6, and/or CXCL4. In some embodiments, a subject of the present disclosure has an increased level of one or more biomarkers selected from the group consisting of CCL18, YKL-40, ICAM, VCAM, E-selectin, Anti-HSP70 IgG, BLyS/BAFF, CCL2/MCP-1, IL-6, and/or CXCL4, when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

C-C motif chemokine ligand 18 (CCL18) is primarily produced by alveolar macrophages with an important role in stimulating fibroblasts to synthesize collagen in fibrotic lung diseases. Although unable to differentiate between ILD subtypes, longitudinal CCL18 levels have demonstrated correlation with lung function and/or mortality in IPF and/or ILD subjects. In some embodiments, levels of CCL18 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of CCL18 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

YKL-40 is a chitinase-like glycoprotein, thought to have a role in regulating connective tissue cell proliferation and/or angiogenesis. Serum YKL-40 may not be able to distinguish between ILD subtypes, however, elevated levels have been identified across a variety of inflammatory and/or fibrotic diseases including, but not limited to, ILD, liver fibrosis, inflammatory arthropathies, asthma and/or chronic obstructive pulmonary disease (COPD). In some embodiments, levels of YKL-40 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of YKL-40 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Adhesion molecules, including intercellular adhesion molecule (ICAM1), vascular cell adhesion molecule 1 (VCAM1), and/or E-selectin, are typically expressed on leucocytes and/or vascular endothelial cells, and are important in mediating adhesion and/or interaction of these cells. Elevated levels of ICAM1, VCAM1, and/or E-selectin are detectable across a number of inflammatory and/or fibrovascular conditions. Integrins interact with adhesion molecules such as VCAM and/or ICAM, and/or other growth-factor receptors and/or ECM components to activate downstream signaling pathways and/or TGF-β mediated pulmonary fibrosis. In some embodiments, levels of one or more of ICAM1, CAM1, and/or E-selectin are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of one or more of ICAM1, CAM1, and/or E-selectin when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Heat shock protein 70 (HSP70) typically induces T-cell proliferation and/or pro-fibrotic cytokine production. Autoantibodies to HSP70 may have pathogenic potential in ILD by augmenting neutrophil recruitment, complement activation and/or production of inflammatory mediators in target organs.

B lymphocyte stimulator (BLyS), also known as B-cell activating factor (BAFF), is a cytokine belonging to the tumor necrosis factor family, critical to B cell maturation and/or antibody production with a possible pathogenic role in several autoimmune diseases and/or IPF. In some embodiments, levels of BLyS are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of BLyS when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

C-C motif chemokine 2 (CCL2), previously known as monocyte chemoattractant protein-1 (MCP-1), plays an important role in innate immunity and/or inflammation, with a potential pro-fibrotic effect. In some embodiments, levels of CCL2 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of CCL2 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Cytokine IL-6 is involved in the differentiation of CD4+ T cells to pro-fibrotic Th2 type cells, and implicated in the activation of fibroblasts. In some embodiments, levels of IL-6 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of IL-6 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

CXCL4, also known as platelet factor 4, is a potent anti-angiogenic chemokine. In some embodiments, levels of CXCL4 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of CXCL4 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD)

In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) is selected from the group consisting of VEGF, ET-1, and/or IL-8. In some embodiments, a biomarker for chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) may be involved in one or more vascular and/or endothelium pathways. In some embodiments, a subject of the present disclosure has an increased level of one or more biomarkers selected from the group consisting of VEGF, ET-1, and IL-8. In some embodiments, a subject of the present disclosure has an increased level of one or more biomarkers selected from the group consisting of VEGF, ET-1, and/or IL-8, when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Aberrant angiogenesis is generally implicated in the pathogenesis of pulmonary fibrosis and/or fundamental mediators of this process include vascular endothelial growth factor (VEGF), endothelin 1 (ET-1) and/or interleukin-8 (IL-8). VEGF is a central cytokine and growth factor for endothelial and/or type II AECs. Nintedanib, a tyrosine kinase inhibitor that targets VEGF signaling slows disease progression in IPF. IL-8 (also known as CXCL8) is produced by phagocytes when exposed to inflammatory stimuli and/or attracts neutrophils and/or promotes angiogenesis. Endothelin-1 (ET-1) is a potent vasoactive peptide with diverse properties including vasoconstriction, bronchoconstriction, cell growth, turnover and/or fibroblast activation. Both IL-8 and/or ET-1 are elevated in idiopathic ILD compared with controls and/or have demonstrated correlation with HRCT fibrosis scores, pulmonary function decline and/or reduced overall, transplant-free and/or progression-free survival. In some embodiments, levels of one or more of VEGF, IL-8, and/or ET-1 are elevated in a subject that has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a subject of the present disclosure has an increased level of one or more of VEGF, IL-8, and/or ET-1 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Other biomarkers used to determine if a subject has chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD) include, but are not limited to Colla1, Col3a1, CTGF, fibronectin, PAI-1, and/or Tnfa.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more biomarkers selected from the group consisting of ADAM12, Anti-HSP70 IgG, BAFF, BLyS, Ca15.3, CC16, CCL2, CCL18, Colla1, Col3a1, CTGF, CXCL4, E-selectin, ET-1, fibronectin, ICAM, IL-6, IL-8, KL-6, MCP-1, MMP7, Osteopontin, PAI-1, SP-A, SP-D, Tnfa, VCAM, VEGF, and YKL-40 when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more biomarkers selected from the group consisting of Ca15.3, CC16, KL-6, SP-A, and SP-D when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more biomarkers selected from the group consisting of Colla1, Col3a1, CTGF, fibronectin, PAI-1, and Tnfa when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has a change in level of one or more collagen biomarkers compared to a baseline measurement. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increase in levels of one or more collagen biomarkers compared to a baseline measurement. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increase in levels of one or more collagen biomarkers when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, a collagen biomarker is selected from the group consisting of type I (C1M), type III (C3A, C3M), type IV (C4M), type V (C5M) and type VI (C6M) collagen degradation biomarkers. In some embodiments, a collagen biomarker is selected from the group consisting of type I (PRO-C1), II (PRO-C2), III (PRO-C3), IV (PRO-C4), V (PRO-05) and VI (PRO-C6) collagen formation biomarkers. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more collagen degradation biomarkers selected from the group consisting of type I (C1M), type III (C3A, C3M), type IV (C4M), type V (C5M) and type VI (C6M), when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of one or more collagen formation biomarkers selected from the group consisting of type I (PRO-C1), II (PRO-C2), III (PRO-C3), IV (PRO-C4), V (PRO-05) and VI (PRO-C6), when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Alpha-smooth muscle actin (aSMA) is the actin isoform that predominates within vascular smooth-muscle cells and plays an important role in fibrogenesis. Myofibroblasts are metabolically and morphologically distinctive fibroblasts expressing aSMA, and their activation plays a key role in development of the fibrotic response. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of aSMA when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

The Muc5B promoter polymorphism is a known genetic risk factor for Idopathic Pulmonary Fibrosis (IPF), and is likely involved in disease pathogenesis through an increase in Muc5B expression in terminal bronchi and honeycombed cysts. Expression of MUCSB is also highly correlated with expression of cilium genes in IPF lung. Mucociliary dysfunction in the distal airway may play a role in the development of progressive fibroproliferative lung disease (Yang, I. V. et al., Ann Am Thorac Soc., 2015, S193-S199). In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of Muc5B when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Similarly, periostin has been shown to be a biomarker for IPF and related diseases. Monomeric periostin has been identified in diseases such as atopic dermatitis, systemic scleroderma, and asthma. Both monomeric and total periostin have been shown to be correlated with decline of % VC and % DLCO (Ohta, S. et al. PLOS One, 2017, 1-17). In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of periostin when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

The Whey Acidic Protein domain (WFDC2 (HEA)) is an evolutionarily conserved motif found in a number of proteins, the best studied of which are antiproteinases involved in the innate immune defense of multiple epithelia. The WFDC2 gene encodes a two WAP domain-containing protein, initially suggested as a marker for epididymis, and it has been shown that it is highly expressed in the lung and salivary gland. In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a TβRII antagonist to a subject in need thereof, wherein the subject has an increased level of WFDC2 (HEA) when compared with a subject that does not have chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

### 7. TβRII Antagonists

In part, the disclosure relates to TGFβ type II receptor (TβRII) polypeptides that can be used to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). While TβRII polypeptides may affect chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), through a mechanism other that inhibiting TβRII activity, the disclosure nonetheless provides that desirable therapeutic agents may be selected on the basis of TβRII antagonism. Therefore, while not wishing to be bound to a particular mechanism of action, it is expected that additional TβRII antagonists may be useful in the treatment of chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). For example, agents that inhibit the activity and/or expression (e.g., transcription, translation, secretion from a cell, or combinations thereof) of one or more of: i) the TβRII receptor, ii) one or more TβRII-binding ligand (e.g., TGFβ1, TGFβ2, and/or TGFβ3); iii) one or more TβRII-associated type I receptor (e.g., ALK5); iv) one or more TβRII-associated co-receptor (e.g., betaglycan); and/or v) one or more TβRII downstream signaling component (e.g., Smad proteins), as well as combinations thereof, may be useful in the treatment of chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD). Such agents are collectively referred to herein as “TβRII antagonists” or “TβRII inhibitors”.

In certain aspects, a TβRII antagonist to be used in accordance with methods and uses described herein is an agent that inhibits activity and/or expression of at least TGFβ1 (e.g., a TGFβ1 antagonist). Effects on TGFβ1 inhibition may be determined, for example, using a cell-based assay including those described herein (e.g., Smad signaling assay). Therefore, in some embodiments, a TβRII antagonist of the disclosure may bind to at least TGFβ1. Ligand binding activity may be determined, for example, using a binding affinity assay including those described herein. In some embodiments, a TβRII antagonist of the disclosure binds to at least TGFβ1 with a K_{D} of at least 1×10⁻⁷ M (e.g., at least 1×10⁻⁸ M, at least 1×10⁻⁹ M, at least 1×10⁻¹⁰ M, at least 1×10⁻¹¹ M, or at least 1×10⁻¹² M). As described herein, various TβRII antagonists that inhibit TGFβ1 can be used in accordance with the methods and uses described herein including, for example, ligand traps (e.g., TβRII or betaglycan polypeptides as well as variants thereof), antibodies, small molecules, nucleotide sequences, and combinations thereof. In certain embodiments, a TβRII antagonist that inhibits TGFβ1 may further inhibit one or more of: TGFβ2, TGFβ3, ALK5, and betaglycan. In some embodiments, a TβRII antagonist that inhibits TGFβ1 further inhibits TGFβ3. In some embodiments, a TβRII antagonist that inhibits TGFβ1 does not inhibit or does not substantially inhibit TGFβ2. In some embodiments, a TβRII antagonist that inhibits TGFβ1 further inhibits TGFβ3 but does not inhibit or does not substantially inhibit TGFβ2.

In certain aspects, a TβRII antagonist to be used in accordance with methods and uses described herein is an agent that inhibits activity and/or expression of at least TGFβ2 (e.g., a TGFβ2 antagonist). Effects on TGFβ2 inhibition may be determined, for example, using a cell-based assay including those described herein (e.g., Smad signaling assay). Therefore, in some embodiments, a TβRII antagonist of the disclosure may bind to at least TGFβ2. Ligand binding activity may be determined, for example, using a binding affinity assay including those described herein. In some embodiments, a TβRII antagonist of the disclosure binds to at least TGFβ2 with a K_{D} of at least 1×10⁻⁷ M (e.g., at least 1×10⁻⁸ M, at least 1×10⁻⁹ M, at least 1×10⁻¹⁰ M, at least 1×10⁻¹¹ M, or at least 1×10⁻¹² M). As described herein, various TβRII antagonists that inhibit TGFβ2 can be used in accordance with the methods and uses described herein including, for example, ligand traps (e.g., TβRII or betaglycan polypeptides as well as variants thereof), antibodies, small molecules, nucleotide sequences, and combinations thereof. In certain embodiments, a TβRII antagonist that inhibits TGFβ2 may further inhibit one or more of: TGFβ1, TGFβ3, ALK5, and betaglycan.

In certain aspects, a TβRII antagonist to be used in accordance with methods and uses described herein is an agent that inhibits activity and/or expression of at least TGFβ3 (e.g., a TGFβ3 antagonist). Effects on TGFβ3 inhibition may be determined, for example, using a cell-based assay including those described herein (e.g., Smad signaling assay). Therefore, in some embodiments, a TβRII antagonist of the disclosure may bind to at least TGFβ3. Ligand binding activity may be determined, for example, using a binding affinity assay including those described herein. In some embodiments, a TβRII antagonist of the disclosure binds to at least TGFβ3 with a K_{D} of at least 1×10⁻⁷ M (e.g., at least 1×10⁻⁸ M, at least 1×10⁻⁹ M, at least 1×10⁻¹⁰ M, at least 1×10⁻¹¹ M, or at least 1×10⁻¹² M). As described herein, various TβRII antagonists that inhibit TGFβ3 can be used in accordance with the methods and uses described herein including, for example, ligand traps (e.g., TβRII or betaglycan polypeptides as well as variants thereof), antibodies, small molecules, nucleotide sequences, and combinations thereof. In certain embodiments, a TβRII antagonist that inhibits TGFβ3 may further inhibit one or more of: TGFβ1, TGFβ2, ALK5, and betaglycan. In some embodiments, a TβRII antagonist that inhibits TGFβ3 further inhibits TGFβ1. In some embodiments, a TβRII antagonist that inhibits TGFβ3 does not inhibit or does not substantially inhibit TGFβ2. In some embodiments, a TβRII antagonist that inhibits TGFβ3 further inhibits TGFβ1 but does not inhibit or does not substantially inhibit TGFβ2.

In certain aspects, a TβRII antagonist to be used in accordance with methods and uses described herein is an agent that inhibits activity and/or expression of at least a TβRII receptor (e.g., a TβRII receptor antagonist). Effects on TβRII inhibition may be determined, for example, using a cell-based assay including those described herein (e.g., Smad signaling assay). Therefore, in some embodiments, a TβRII antagonist of the disclosure may bind to at least a TβRII receptor. Ligand binding activity may be determined, for example, using a binding affinity assay including those described herein. In some embodiments, a TβRII antagonist of the disclosure binds to at least a TβRII receptor with a K_{D} of at least 1×10″ M (e.g., at least 1×10⁻⁸ M, at least 1×10⁻⁹ M, at least 1×10⁻¹⁰ M, at least 1×10⁻¹¹ M, or at least 1×10⁻¹² M). As described herein, various TβRII antagonists that inhibit a TβRII receptor can be used in accordance with the methods and uses described herein including, for example, ligand traps (e.g., TβRII or betaglycan polypeptides as well as variants thereof), antibodies, small molecules, nucleotide sequences, and combinations thereof. In certain embodiments, a TβRII antagonist that inhibits the TβRII receptor may further inhibit one or more of: TGFβ1, TGFβ2, TGFβ3, ALK5, and betaglycan. In some embodiments, a TβRII antagonist that inhibits the TβRII receptor does not inhibit or does not substantially inhibit TGFβ2.

In certain aspects, a TβRII antagonist to be used in accordance with methods and uses described herein is an agent that inhibits activity and/or expression of at least ALK5 (e.g., an ALK5 antagonist). Effects on ALK5 inhibition may be determined, for example, using a cell-based assay including those described herein (e.g., Smad signaling assay). Therefore, in some embodiments, a TβRII antagonist of the disclosure may bind to at least ALK5. Ligand binding activity may be determined, for example, using a binding affinity assay including those described herein. In some embodiments, an ALK5 antagonist of the disclosure binds to at least ALK5 with a K_{D} of at least 1×10⁻⁷ M (e.g., at least 1×10⁻⁸ M, at least 1×10⁻⁹ M, at least 1×10⁻¹⁰ M, at least 1×10⁻¹¹ M, or at least 1×10⁻¹² M). As described herein, various TβRII antagonists that inhibit ALK5 can be used in accordance with the methods and uses described herein including, for example, ligand traps (e.g., TβRII or betaglycan polypeptides as well as variants thereof), antibodies, small molecules, nucleotide sequences, and combinations thereof. In certain embodiments, a TβRII antagonist that inhibits ALK5 may further inhibit one or more of: TGFβ1, TGFβ2, TGFβ3, TβRII, and betaglycan. In some embodiments, a TβRII antagonist that inhibits ALK5 does not inhibit or does not substantially inhibit TGFβ2.

In certain aspects, a TβRII antagonist to be used in accordance with methods and uses described herein is an agent that inhibits activity and/or expression of at least betaglycan (e.g., a betaglycan antagonist). Effects on betaglycan inhibition may be determined, for example, using a cell-based assay including those described herein (e.g., Smad signaling assay). Therefore, in some embodiments, a TβRII antagonist of the disclosure may bind to at least betaglycan. Ligand binding activity may be determined, for example, using a binding affinity assay including those described herein. In some embodiments, a betaglycan antagonist of the disclosure binds to at least betaglycan with a K_{D} of at least 1×10⁻⁷ M (e.g., at least 1×10⁻⁸ M, at least 1×10⁻⁹ M, at least 1×10⁻¹⁰ M, at least 1×10⁻¹¹ M, or at least 1×10⁻¹² M). As described herein, various TβRII antagonists that inhibit betaglycan can be used in accordance with the methods and uses described herein including, for example, ligand traps (e.g., TβRII or betaglycan polypeptides as well as variants thereof), antibodies, small molecules, nucleotide sequences, and combinations thereof. In certain embodiments, a TβRII antagonist that inhibits betaglycan may further inhibit one or more of: TGFβ1, TGFβ2, TGFβ3, TβRII, and ALK5. In some embodiments, a TβRII antagonist that inhibits betaglycan does not inhibit or does not substantially inhibit TGFβ2.

### 8. TβRII Polypeptides

In certain aspects, a TβRII antagonist to be used in accordance with the methods and uses disclosed herein is a TβRII polypeptide. A TβRII polypeptide may inhibit and/or bind to, for example, one or more TβRII ligands (e.g., TGFβ1 and/or TGFβ3). In some embodiments, the ability for a TβRII polypeptide to inhibit activity (e.g., Smad signaling) and/or bind to a target is determined in an in vitro or cell-based assay including, for example, those disclosed herein. As described herein, a TβRII polypeptide may be used alone or in combination with one or more additional active agents or supportive therapies to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

Naturally occurring TβRII proteins are transmembrane proteins, with a portion of the protein positioned outside the cell (the extracellular portion) and a portion of the protein positioned inside the cell (the intracellular portion). Aspects of the present disclosure encompass variant TβRII polypeptides comprising mutations within the extracellular domain and/or truncated portions of the extracellular domain of TβRII. As described above, human TβRII occurs naturally in at least two isoforms—B (short) and A (long)—generated by alternative splicing in the extracellular domain (ECD) (FIG. 1, SEQ ID NO: 1, and FIG. 2, SEQ ID NO: 2, respectively). SEQ ID NO: 27, which corresponds to residues 23-159 of SEQ ID NO: 1, depicts the native full-length extracellular domain of the short isoform of SEQ ID NO: 18, which corresponds to residues 23-184 of SEQ ID NO: 2, depicts the native full-length extracellular domain of the long isoform of TβRII. Unless noted otherwise, amino acid position numbering with regard to variants based on the TβRII short and long isoforms refers to the corresponding position in the native precursors, SEQ ID NO: 1 and SEQ ID NO: 2, respectively.

In certain embodiments, the disclosure provides variant TβRII polypeptides. A TβRII polypeptide of the disclosure may bind to and inhibit the function of a TGFβ superfamily member, such as but not limited to, TGFβ1 or TGFβ3. TβRII polypeptides may include a polypeptide consisting of, or comprising, an amino acid sequence at least 80% identical, and optionally at least 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 153-159 of SEQ ID NO: 1. TβRII polypeptides may include a polypeptide consisting of, or comprising, an amino acid sequence at least 80% identical, and optionally at least 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 178-184 of SEQ ID NO: 2. In particular embodiments, the TβRII polypeptides comprise an amino acid sequence at least 80% identical, and optionally at least 85%, 90%, 91%, 92%, 93%, 94%, 95%, 96%, 97%, 98%, 99%, or 100% identical to the amino acid sequence of SEQ ID NO: 18. Optionally, a TβRII polypeptide does not include more than 5 consecutive amino acids, or more than 10, 20, 30, 40, 50, 52, 60, 70, 80, 90, 100, 150 or 200 or more consecutive amino acids from a sequence consisting of amino acids 160-567 of SEQ ID NO: 1 or from a sequence consisting of amino acids 185-592 of SEQ ID NO: 2. In some embodiments, the TβRII polypeptide does not include amino acids 160-567 of SEQ ID NO: 1. In some embodiments, the TβRII polypeptide does not include amino acids 1-22 of SEQ ID NO: 1. In some embodiments, the TβRII polypeptide does not include amino acids 1-22 and 160-567 of SEQ ID NO: 1. In some embodiments, the TβRII polypeptide does not include amino acids 185-592 of SEQ ID NO: 2. In some embodiments, the TβRII polypeptide does not include amino acids 1-22 of SEQ ID NO: 2. In some embodiments, the TβRII polypeptide does not include amino acids 1-22 and 185-592 of SEQ ID NO: 2. The unprocessed TβRII polypeptide may either include or exclude any signal sequence, as well as any sequence N-terminal to the signal sequence. As elaborated herein, the N-terminus of the mature (processed) TβRII polypeptide may occur at any of amino acids 23-35 of SEQ ID NO: 1 or 23-60 of SEQ ID NO: 2. Examples of mature TβRII polypeptides include, but are not limited to, amino acids 23-159 of SEQ ID NO: 1 (set forth in SEQ ID NO: 27), amino acids 29-159 of SEQ ID NO: 1 (set forth in SEQ ID NO: 28), amino acids 35-159 of SEQ ID NO: 1 (set forth in SEQ ID NO: 29), amino acids 23-153 of SEQ ID NO: 1 (set forth in SEQ ID NO: 30), amino acids 29-153 of SEQ ID NO: 1 (set forth in SEQ ID NO: 31), amino acids 35-153 of SEQ ID NO: 1 (set forth in SEQ ID NO: 32), amino acids 23-184 of SEQ ID NO: 2 (set forth in SEQ ID NO: 18), amino acids 29-184 of SEQ ID NO: 2 (set forth in SEQ ID NO: 33), amino acids 60-184 of SEQ ID NO: 2 (set forth in SEQ ID NO: 29), amino acids 23-178 of SEQ ID NO: 2 (set forth in SEQ ID NO: 34), amino acids 29-178 of SEQ ID NO: 2 (set forth in SEQ ID NO: 35), and amino acids 60-178 of SEQ ID NO: 2 (set forth in SEQ ID NO: 32). It will be understood by one of skill in the art that corresponding variants based on the long isoform of TβRII will include nucleotide sequences encoding the 25-amino acid insertion along with a conservative Val-Ile substitution at the flanking position C-terminal to the insertion. The TβRII polypeptides accordingly may include isolated extracellular portions of TβRII polypeptides, including both the short and the long isoforms, variants thereof (including variants that comprise, for example, no more than 2, 3, 4, 5, 10, 15, 20, 25, 30, or 35 amino acid substitutions in the sequence corresponding to amino acids 23-159 of SEQ ID NO: 1 or amino acids 23-184 of SEQ ID NO: 2), fragments thereof, and fusion proteins comprising any of the foregoing, but in each case preferably any of the foregoing TβRII polypeptides will retain substantial affinity for at least one of, or both of, TGFβ1 or TGFβ3. Generally, a TβRII polypeptide will be designed to be soluble in aqueous solutions at biologically relevant temperatures, pH levels, and osmolarity.

In some embodiments, the variant TβRII polypeptides of the disclosure comprise one or more mutations in the extracellular domain that confer an altered ligand binding profile. A TβRII polypeptide may include one, two, five or more alterations in the amino acid sequence relative to the corresponding portion of a naturally occurring TβRII polypeptide. In some embodiments, the mutation results in a substitution, insertion, or deletion at the position corresponding to position 70 of SEQ ID NO: 1. In some embodiments, the mutation results in a substitution, insertion, or deletion at the position corresponding to position 110 of SEQ ID NO: 1. Examples include, but are not limited to, an N to D substitution or a D to K substitution in the positions corresponding to positions 70 and 110, respectively, of SEQ ID NO: 1. Examples of such variant TβRII polypeptides include, but are not limited to, the sequences set forth in SEQ ID NOs: 36-39. A TβRII polypeptide may comprise a polypeptide or portion thereof that is encoded by any one of SEQ ID NOs: 10, 12, 14 or 16, or silent variants thereof or nucleic acids that hybridize to the complement thereof under stringent hybridization conditions. In particular embodiments, a TβRII polypeptide may comprise a polypeptide or portion thereof that is encoded by any one of SEQ ID NO: 12, or silent variants thereof or nucleic acids that hybridize to the complement thereof under stringent hybridization conditions.

In some embodiments, the variant TβRII polypeptides of the disclosure further comprise an insertion of 36 amino acids (SEQ ID NO: 41) between the pair of glutamate residues (positions 151 and 152 of SEQ ID NO: 1, or positions 176 and 177 of SEQ ID NO: 2) located near the C-terminus of the human TβRII ECD, as occurs naturally in the human TβRII isoform C (Konrad et al., BMC Genomics 8:318, 2007).

The disclosure further demonstrates that TβRII polypeptides can be modified to selectively antagonize TβRII ligands. The N70 residue represents a potential glycosylation site. In some embodiments, the TβRII polypeptides are aglycosylated. In some embodiments, the TβRII polypeptides are aglycosylated or have reduced glycosylation at position Asn157. In some embodiments, the TβRII polypeptides are aglycosylated or have reduced glycosylation at position Asn73.

In certain embodiments, a TβRII polypeptide binds to TGFβ1, and the TβRII polypeptide does not show substantial binding to TGFβ3. In certain embodiments, a TβRII polypeptide binds to TGFβ3, and the TβRII polypeptide does not show substantial binding to TGFβ1. Binding may be assessed using purified proteins in solution or in a surface plasmon resonance system, such as a Biacore™ system.

In certain embodiments, a TβRII polypeptide inhibits TGFβ1 cellular signaling, and the TβRII polypeptide has an intermediate or limited inhibitory effect on TGFβ3 signaling. In certain embodiments, a TβRII polypeptide inhibits TGFβ3 cellular signaling, and the TβRII polypeptide has an intermediate or limited inhibitory effect on TGFβ1 signaling. Inhibitory effect on cell signaling can be assayed by methods known in the art.

Taken together, an active portion of a TβRII polypeptide may comprise amino acid residues 23-153, 23-154, 23-155, 23-156, 23-157, or 23-158 of SEQ ID NO: 1, as well as variants of these amino acid residues starting at any of amino acids 24-35 of SEQ ID NO: 1. Similarly, an active portion of a TβRII polypeptide may comprise amino acid residues 23-178, 23-179, 23-180, 23-181, 23-182, or 23-183 of SEQ ID NO: 2, as well as variants of these amino acid residues starting at any of amino acids 24-60 of SEQ ID NO: 2. Exemplary TβRII polypeptides comprise amino acid residues 29-159, 35-159, 23-153, 29-153 and 35-153 of SEQ ID NO: 1 or amino acid residues 29-184, 60-184, 23-178, 29-178 and 60-178 of SEQ ID NO: 2. Variants within these ranges are also contemplated, particularly those having at least 80%, 85%, 90%, 95%, or 99% identity to the corresponding portion of SEQ ID NO: 1 or SEQ ID NO: 2. A TβRII polypeptide may be selected that does not include the sequence consisting of amino acid residues 160-567 of SEQ ID NO: 1 or amino acid residues 185-592 of SEQ ID NO: 2. In particular embodiments, the TβRII polypeptides comprise an amino acid sequence at least 80% identical, and optionally at least 85%, 90%, 91%, 92%, 93%, 94%, 95%, 96%, 97%, 98%, 99%, or 100% identical to the amino acid sequence of SEQ ID NO: 18.

As described above, the disclosure provides TβRII polypeptides sharing a specified degree of sequence identity or similarity to a naturally occurring TβRII polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In one embodiment, the percent identity between two amino acid sequences is determined using the Needleman and Wunsch (J Mol. Biol. (48):444-453 (1970)) algorithm which has been incorporated into the GAP program in the GCG software package (available at http://www.gcg.com). In a specific embodiment, the following parameters are used in the GAP program: either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux, J., et al., Nucleic Acids Res. 12(1):387 (1984)) (available at http://www.gcg.com). Exemplary parameters include using a NWSgapdna.CMP matrix and a gap weight of 40, 50, 60, 70, or 80 and a length weight of 1, 2, 3, 4, 5, or 6. Unless otherwise specified, percent identity between two amino acid sequences is to be determined using the GAP program using a Blosum 62 matrix, a GAP weight of 10 and a length weight of 3, and if such algorithm cannot compute the desired percent identity, a suitable alternative disclosed herein should be selected.

In another embodiment, the percent identity between two amino acid sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

Another embodiment for determining the best overall alignment between two amino acid sequences can be determined using the FASTDB computer program based on the algorithm of Brutlag et al. (Comp. App. Biosci., 6:237-245 (1990)). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty=0.05.

TβRII polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native TβRII signal sequence may be used to effect extrusion from the cell. Possible leader sequences include native leaders, tissue plasminogen activator (TPA) and honeybee mellitin (SEQ ID NOs: 22-24, respectively). Examples of TβRII-Fc fusion proteins incorporating a TPA leader sequence include SEQ ID NOs: 11, 13, 15, 17, 68, 69, 70, and 71. Processing of signal peptides may vary depending on the leader sequence chosen, the cell type used and culture conditions, among other variables, and therefore actual N-terminal start sites for mature TβRII polypeptides may shift by 1, 2, 3, 4 or 5 amino acids in either the N-terminal or C-terminal direction. Examples of TβRII-Fc fusion proteins include SEQ ID NOs: 11, 13, 15, 17, 68, 69, 70, and 71. It will be understood by one of skill in the art that corresponding variants based on the long isoform of TβRII will include the 25-amino acid insertion along with a conservative Val-Ile substitution at the flanking position C-terminal to the insertion.

In some embodiments, any of the TβRII polypeptides disclosed herein are at least 80%, 85%, 90%, 91%, 92%, 93%, 94%, 95%, 97%, 99% or 100% identical to the amino acid sequence of any one of SEQ ID NOs: 18, 27, 30, 34, 36, 37, 38, 39, 48, 49, 51, 67, or 78, but lack one or more N-terminal amino acids as compared to the amino acid sequences of SEQ ID NO: 18, 27, 30, 34, 36, 37, 38, 39, 48, 49, 51, 67, or 78. In some embodiments, the TβRII polypeptide lacks the amino acid corresponding to the first amino acid (threonine) of any one of SEQ ID NOs: 18, 27, 30, 34, 36, 37, 38, 39, 48, 49, 51, 67, or 78. In some embodiments, the TβRII polypeptide lacks the amino acids corresponding to the first and second amino acids (threonine and isoleucine, respectively) of any one of SEQ ID NOs: 18, 27, 30, 34, 36, 37, 38, 39, 48, 49, 51, 67, or 78. In some embodiments, the TβRII polypeptide lacks the amino acids corresponding to the first, second and third amino acids (threonine, isoleucine, and proline, respectively) of any one of SEQ ID NOs: 18, 27, 30, 34, 36, 37, 38, 39, 48, 49, 51, 67, or 78. In some embodiments, the TβRII polypeptide lacks the amino acids corresponding to the first, second, third and fourth amino acids (threonine, isoleucine, proline, proline, respectively) of any one of SEQ ID NOs: 18, 27, 30, 34, 36, 37, 38, 39, 48, 49, 51, 67, or 78.

In some embodiments, any of the TβRII polypeptides disclosed herein are at least 80%, 85%, 90%, 91%, 92%, 93%, 94%, 95%, 97%, 99% or 100% identical to the amino acid sequence of any one of SEQ ID NOs: 18, 48, 51, 67, or 78, but lack the amino acid corresponding to the first amino acid (threonine) of SEQ ID NO: 18, 48, 51, 67, or 78. In some embodiments, the TβRII polypeptide lacks the amino acids corresponding to the first and second amino acids (threonine and isoleucine, respectively) of SEQ ID NO: 18, 48, 51, 67, or 78. In some embodiments, the TβRII polypeptide lacks the amino acids corresponding to the first, second and third amino acids (threonine, isoleucine, and proline, respectively) of SEQ ID NO: 18, 48, 51, 67, or 78. In some embodiments, the TβRII polypeptide lacks the amino acids corresponding to the first, second, third and fourth amino acids (threonine, isoleucine, proline, proline, respectively) of SEQ ID NO: 18, 48, 51, 67, or 78.

In some embodiments, the disclosure provides for a composition comprising a mixture of TβRII polypeptides, wherein the TβRII polypeptides in the composition each comprise an amino acid sequence that is at least 80%, 85%, 90%, 91%, 92%, 93%, 94%, 95%, 97%, 99% or 100% identical to the amino acid sequence of any one of SEQ ID NOs: 18, 27, 30, 34, 36, 37, 38, 39, 48, 49, 51, 67, or 78; but wherein at least a portion of the TβRII polypeptides (e.g., at least 1%, 3%, 4%, 5%, 10%, 15%, 20%, 25%, 30%, 40%, 50%, 60%, 70%, 80%, 90%, 91%, 92%, 93%, 94%, 95%) in the composition include the amino acids corresponding to the first, second, third and fourth amino acids (threonine, isoleucine, proline and proline, respectively) of any one of SEQ ID NOs: 18, 27, 30, 34, 36, 37, 38, 39, 48, 49, 51, 67, or 78; and wherein at least a portion of the TβRII polypeptides (e.g., at least 1%, 3%, 4%, 5%, 10%, 15%, 20%, 25%, 30%, 40%, 50%, 60%, 70%, 80%, 90%, 91%, 92%, 93%, 94%, 95%) in the composition lack one or more of the amino acids corresponding to the first, second, third and fourth amino acids (threonine, isoleucine, proline and proline, respectively) of any one of SEQ ID NOs: 18, 27, 30, 34, 36, 37, 38, 39, 48, 49, 51, 67, or 78. In some embodiments, the disclosure provides for a composition comprising a mixture of TβRII polypeptides, wherein the TβRII polypeptides are at least 80%, 85%, 90%, 91%, 92%, 93%, 94%, 95%, 97%, 98%, 99% or 100% identical to the amino acid sequence of any one of SEQ ID NOs: 18, 48, 51, 67, or 78, but wherein at least 30% to 80% of the TβRII polypeptides in the composition lack the amino acid corresponding to the first amino acid (threonine) of SEQ ID NO: 18, 48, 51, 67, or 78.

In certain embodiments, the present disclosure contemplates specific mutations of the TβRII polypeptides so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (or asparagine-X-serine) (where “X” is any amino acid) which is specifically recognized by appropriate cellular glycosylation enzymes. The alteration may also be made by the addition of, or substitution by, one or more serine or threonine residues to the sequence of the wild-type TβRII polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a TβRII polypeptide is by chemical or enzymatic coupling of glycosides to the TβRII polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on a TβRII polypeptide may be accomplished chemically and/or enzymatically. Chemical deglycosylation may involve, for example, exposure of the TβRII polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on TβRII polypeptides can be achieved by the use of a variety of endo- and exo-glycosidases as described by Thotakura et al. (1987) Meth. Enzymol. 138:350. the sequence of a TβRII polypeptide may be adjusted, as appropriate, depending on the type of expression system used, as mammalian, yeast, insect and plant cells may all introduce differing glycosylation patterns that can be affected by the amino acid sequence of the peptide. In general, TβRII polypeptides for use in humans will be expressed in a mammalian cell line that provides proper glycosylation, such as HEK293 or CHO cell lines, although other mammalian expression cell lines, yeast cell lines with engineered glycosylation enzymes, and insect cells are expected to be useful as well.

This disclosure further contemplates a method of generating mutants, particularly sets of combinatorial mutants of a TβRII polypeptide, as well as truncation mutants; pools of combinatorial mutants are especially useful for identifying functional variant sequences. The purpose of screening such combinatorial libraries may be to generate, for example, TβRII polypeptide variants which can act as either agonists or antagonist, or alternatively, which possess novel activities all together. A variety of screening assays are provided below, and such assays may be used to evaluate variants. For example, a TβRII polypeptide variant may be screened for ability to bind to a TβRII ligand, to prevent binding of a TβRII ligand to a TβRII polypeptide or to interfere with signaling caused by a TβRII ligand. The activity of a TβRII polypeptide or its variants may also be tested in a cell-based or in vivo assay, particularly any of the assays disclosed in the Examples.

Combinatorically-derived variants can be generated which have a selective or generally increased potency relative to a TβRII polypeptide comprising an extracellular domain of a naturally occurring TβRII polypeptide. Likewise, mutagenesis can give rise to variants which have serum half-lives dramatically different than the corresponding wild-type TβRII polypeptide. For example, the altered protein can be rendered either more stable or less stable to proteolytic degradation or other processes which result in destruction of, or otherwise elimination or inactivation of, a native TβRII polypeptide. Such variants, and the genes which encode them, can be utilized to alter TβRII polypeptide levels by modulating the half-life of the TβRII polypeptides. For instance, a short half-life can give rise to more transient biological effects and can allow tighter control of recombinant TβRII polypeptide levels within the patient. In an Fc fusion protein, mutations may be made in the linker (if any) and/or the Fc portion to alter the half-life of the protein.

A combinatorial library may be produced by way of a degenerate library of genes encoding a library of polypeptides which each include at least a portion of potential TβRII polypeptide sequences. For instance, a mixture of synthetic oligonucleotides can be enzymatically ligated into gene sequences such that the degenerate set of potential TβRII polypeptide nucleotide sequences are expressible as individual polypeptides, or alternatively, as a set of larger fusion proteins (e.g., for phage display).

There are many ways by which the library of potential TβRII polypeptide variants can be generated from a degenerate oligonucleotide sequence. Chemical synthesis of a degenerate gene sequence can be carried out in an automatic DNA synthesizer, and the synthetic genes then be ligated into an appropriate vector for expression. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, TβRII polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, NY; and Greener et al., (1994) Strategies in Mol Biol 7:32-34). Linker scanning mutagenesis, particularly in a combinatorial setting, is an attractive method for identifying truncated (bioactive) forms of TβRII polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of TβRII polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include TβRII ligand binding assays and ligand-mediated cell signaling assays.

In certain embodiments, the TβRII polypeptides of the disclosure may further comprise post-translational modifications in addition to any that are naturally present in the TβRII polypeptides. Such modifications include, but are not limited to, acetylation, carboxylation, glycosylation, phosphorylation, lipidation, pegylation (polyethylene glycol) and acylation. As a result, the modified TβRII polypeptides may contain non-amino acid elements, such as polyethylene glycols, lipids, mono- or poly-saccharides, and phosphates. Effects of such non-amino acid elements on the functionality of a TβRII polypeptide may be tested as described herein for other TβRII polypeptide variants. When a TβRII polypeptide is produced in cells by cleaving a nascent form of the TβRII polypeptide, post-translational processing may also be important for correct folding and/or function of the protein. Different cells (such as CHO, HeLa, MDCK, 293, WI38, NIH-3T3 or HEK-293) have specific cellular machinery and characteristic mechanisms for such post-translational activities and may be chosen to ensure the correct modification and processing of the TβRII polypeptides.

### 9. Linkers

The disclosure provides for TβRII fusion proteins, and in these embodiments, the TβRII portion is connected to the heterologous portion (e.g., Fc portion) by means of a linker. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 19), or repetitions thereof (GGGGS)n, where n≥2 (SEQ ID NO: 57). In particular embodiments n≥3, or n=3-10. The application teaches the surprising finding that proteins comprising a TβRII portion and a heterologous portion fused together by means of a (GGGGS)₄ linker (SEQ ID NO: 59) were associated with a stronger affinity for TGFβ1 and TGFβ3 as compared to a TβRII fusion protein where n<4. As such, in preferred embodiments, n≥4, or n=4-10. The application also teaches that proteins comprising (GGGGS)n linkers (‘GGGGS’ disclosed as SEQ ID NO: 19) in which n>4 had similar inhibitory properties as proteins having the (GGGGS) 4 linker (SEQ ID NO: 59). As such, in some embodiments, n is not greater than 4 in a (GGGGS)n linker (SEQ ID NO: 19). In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)n sequence (SEQ ID NO: 19) also comprises an N-terminal threonine. In some embodiments, the linker is any one of the following:

(SEQ ID NO: 21)
GGGGSGGGGS
(SEQ ID NO: 4)
TGGGGSGGGGS
(SEQ ID NO: 5)
TGGGGSGGGGSGGGGS
(SEQ ID NO: 6)
TGGGGSGGGGSGGGGSGGGGS
(SEQ ID NO: 25)
TGGGGSGGGGSGGGGSGGGGSGGGGS
(SEQ ID NO: 26)
TGGGGSGGGGSGGGGSGGGGSGGGGSGGGGS
or
(SEQ ID NO: 40)
TGGGGSGGGGSGGGGSGGGGSGGGGSGGGGSGGGGS

In some embodiments, the linker comprises the amino acid sequence of TGGGPKSCDK (SEQ ID NO: 7). In some embodiments, the linker is any one of SEQ ID NOs: 21, 4-7, 25-26 or 40 lacking the N-terminal threonine. In some embodiments, the linker does not comprise the amino acid sequence of SEQ ID NO: 26 or 40.

### 10. Heterologous Domains

In certain aspects, functional variants or modified forms of the TβRII polypeptides include fusion proteins having at least a portion of the TβRII polypeptides and one or more heterologous portions. Well-known examples of such heterologous portions include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A heterologous portion may be selected so as to confer a desired property. For example, some heterologous portions are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS6 (SEQ ID NO: 61)) fusion partners. As another example, a heterologous portion may be selected so as to facilitate detection of the TβRII polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the heterologous portions have a protease cleavage site, such as for Factor Xa or Thrombin, which allows the relevant protease to partially digest the fusion proteins and thereby liberate the recombinant proteins therefrom. The liberated proteins can then be isolated from the heterologous portion by subsequent chromatographic separation. In certain preferred embodiments, a TβRII polypeptide is fused with a domain that stabilizes the TβRII polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of heterologous portions that may be selected include multimerizing (e.g., dimerizing, tetramerizing) domains and functional domains.

As specific examples, the present disclosure provides fusion proteins comprising variants of TβRII polypeptides fused to an Fc domain sequence of SEQ ID NO: 20. Optionally, the Fc domain has one or more mutations at residues such as Asp-265, Lys-322, and Asn-434 (numbered in accordance with the corresponding full-length IgG). In certain cases, the mutant Fc domain having one or more of these mutations (e.g., Asp-265 mutation) has reduced ability of binding to the Fcγ receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain. In some embodiments, the C-terminal lysine residue of the Fc domain can be deleted. The amino acid sequence of SEQ ID NO: 20, 42, 43, and 73 may optionally be provided with the lysine removed from the C-terminus (SEQ ID NO: 72, 73, and 74).

It is understood that different elements of the fusion proteins may be arranged in any manner that is consistent with the desired functionality. For example, a TβRII polypeptide may be placed C-terminal to a heterologous domain, or, alternatively, a heterologous domain may be placed C-terminal to a TβRII polypeptide. The TβRII polypeptide domain and the heterologous domain need not be adjacent in a fusion protein, and additional domains or amino acid sequences may be included C- or N-terminal to either domain or between the domains.

As used herein, the term “immunoglobulin Fc domain” or simply “Fe” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain. In some embodiments, the immunoglobulin Fc region is a human immunoglobulin Fc region.

In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Iv), may be used. The choice of appropriate immunoglobulin heavy chain constant region is discussed in detail in U.S. Pat. Nos. 5,541,087 and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc gamma or the homologous domains in any of IgA, IgD, Igε, or IgM.

Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al. (1997) J. Immunol. 159:3613).

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus, in certain embodiments, Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. While alanine substitutions at these sites are effective in reducing ADCC in both human and murine antibodies, these substitutions are less effective at reducing CDC activity. Another single variant P329A, identified by a random mutagenesis approach to map the Clq binding site of the Fc, is highly effective at reducing CDC activity while retaining ADCC activity. A combination of L234A, L235A, and P329A (LALA-PG, Kabat positions) substitutions have been shown to effectively silence the effector function of human IgG1 antibodies. For a detailed discussion of LALA, LALA-PG, and other mutations, see Lo et al. (2017) 1 Biol. Chem. 292:3900-3908, the contents of which are hereby incorporated herein by reference in their entirety. In some embodiments, Fc fusion proteins of the disclosure comprise L234A, L235A, and P329G mutations (LALA-PG; Kabat positions) in the Fc region of the heavy chain. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively, or additionally, cysteine residue(s) may be introduced in the Fc region, thereby allowing interchain disulfide bond formation in this region. The homodimeric antibody or Fc fusion protein thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

In some embodiments, the disclosure provides for TβRII polypeptides fusion proteins comprising an amino acid sequence that is at least 80%, 85%, 90%, 91%, 92%, 93%, 94%, 95%, 96%, 97%, 98%, or 99% identical to the amino acid sequence of any one of SEQ ID NOs: 11, 13, 15, 17, 68, 69, 70, and 71, or biologically active fragments thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NOs: 11, 13, 15, 17, 68, 69, 70, and 71 or biologically active fragments thereof. In some embodiments, the C-terminal lysine residue of an Fc domain can be deleted. The amino acid sequence of SEQ ID NO: 11 may optionally be provided with the lysine removed from the C-terminus (SEQ ID NO: 68). The amino acid sequence of SEQ ID NO: 13 may optionally be provided with the lysine removed from the C-terminus (SEQ ID NO: 69). The amino acid sequence of SEQ ID NO: 15 may optionally be provided with the lysine removed from the C-terminus (SEQ ID NO: 70). The amino acid sequence of SEQ ID NO: 17 may optionally be provided with the lysine removed from the C-terminus (SEQ ID NO: 71).

In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 13, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 69, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 50, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 77, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 51, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 78, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 52, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 79, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 53, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 80, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 54, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 81, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 55, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 82, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 56, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 83, or a biologically active fragment thereof. In some embodiments, the TβRII polypeptides fusion protein comprises an amino acid sequence that is at least 80%, 85%, 90%, 91%, 92%, 93%, 94%, 95%, 96%, 97%, 98%, or 99% identical to the amino acid sequence of SEQ ID NO: 20, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 72, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 48, or a biologically active fragment thereof. In some embodiments, the TβRII polypeptides fusion proteins comprise an amino acid sequence that is at least 80%, 85%, 90%, 91%, 92%, 93%, 94%, 95%, 96%, 97%, 98%, or 99% identical to the amino acid sequence of any one of SEQ ID NO: 67, or a biologically active fragment thereof.

In some embodiments, the fusion proteins described herein have improved binding affinity for TGFβ1 and TGFβ3. In some embodiments, a fusion protein comprising a linker at least 10 amino acids in length (e.g., a fusion protein having the amino acid sequence of any one of SEQ ID NOs: 11, 13, 15, 50-56, 68-70, and 77-83) has improved binding affinity for TGFβ1 and TGFβ3 as compared to a reference fusion protein (e.g., a fusion protein having the amino acid sequence of SEQ ID NO: 9 or SEQ ID NO: 88). In some embodiments, the fusion protein binds to TGFβ1 with a K_{D} of less than 200 pM, less than 150 pM, less than 100 pM, less than 75 pM, less than 50 pM or less than 25 pM. In some embodiments, the fusion protein binds to TGFβ3 with a K_{D} of less than 75 pM, less than 70 pM, less than 60 pM, less than 50 pM, less than 40 pM, less than 35 pM, less than 25 pM, less than 15, less than 10, or less than 5 pM.

In some embodiments any of the polypeptides disclosed herein inhibits TGFβ1 and/or TGFβ3 in a measurable assay. In some embodiments, the polypeptide inhibits TGFβ1 with an IC₅₀ of less than 1.0, 0.9, 0.8, 0.7, 0.6, 0.5, 0.4, 0.3, 0.2, 0.1, 0.08, 0.09, 0.07, 0.06, 0.05, 0.03, or 0.02 nM, as determined using a reporter gene assay. In some embodiments, the polypeptide inhibits TGFβ3 with an IC₅₀ of less than 1.0, 0.9, 0.8, 0.7, 0.6, 0.5, 0.4, 0.3, 0.2, 0.09, 0.08, 0.07, 0.06, 0.05, 0.04, 0.03, or 0.02 nM, as determined using a reporter gene assay. In some embodiments, the reporter gene assay is a CAGA reporter assay. In some embodiments, the CAGA assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3. See, e.g., Example 2.

### 11. Fusion Polypeptides

In some embodiments, the disclosure provides for TβRII-containing fusion polypeptides. The fusion polypeptides may be prepared according to any of the methods disclosed herein or that are known in the art.

In some embodiments, any of the fusion polypeptides disclosed herein comprises the following components: a) any of the TβRII polypeptides disclosed herein (“A”), b) any of the linkers disclosed herein (“B”), c) any of the heterologous portions disclosed herein (“C”), and optionally a linker (“X”). In such embodiments, the fusion polypeptide may be arranged in a manner as follows (N-terminus to C-terminus): A-B-C or C-B-A. In such embodiments, the fusion polypeptide may be arranged in a manner as follows (N-terminus to C-terminus): X-A-B-C or X—C-B-A. In some embodiments, the fusion polypeptide comprises each of A, B and C (and optionally a leader sequence such as the amino acid sequence of SEQ ID NO: 23), and comprises no more than 100, 90, 80, 70, 60, 50, 40, 30, 20, 10, 5, 4, 3, 2 or 1 additional amino acids (but which may include further post-translational modifications, such as PEGylation).

In some embodiments, the fusion polypeptide comprises a leader sequence (e.g., SEQ ID NO: 23) positioned in a manner as follows (N-terminus to C-terminus): X-A-B-C, and the fusion polypeptide comprises 1, 2, 3, 4, or 5 amino acids between X and A. In some embodiments, the fusion polypeptide comprises a leader sequence (e.g., SEQ ID NO: 23) positioned in a manner as follows (N-terminus to C-terminus): X—C-B-A, and the fusion polypeptide comprises 1, 2, 3, 4, or 5 amino acids between X and C. In some embodiments, the fusion polypeptide comprises a leader sequence (e.g., SEQ ID NO: 23) positioned in a manner as follows (N-terminus to C-terminus): X-A-B-C, and the fusion polypeptide comprises an alanine between X and A. In some embodiments, the fusion polypeptide comprises a leader sequence (e.g., SEQ ID NO: 23) positioned in a manner as follows (N-terminus to C-terminus): X—C-B-A, and the fusion polypeptide comprises an alanine between X and C. In some embodiments, the fusion polypeptide comprises a leader sequence (e.g., SEQ ID NO: 23) positioned in a manner as follows (N-terminus to C-terminus): X-A-B-C, and the fusion polypeptide comprises a glycine and an alanine between X and A. In some embodiments, the fusion polypeptide comprises a leader sequence (e.g., SEQ ID NO: 23) positioned in a manner as follows (N-terminus to C-terminus): X—C-B-A, and the fusion polypeptide comprises a glycine and an alanine between X and C. In some embodiments, the fusion polypeptide comprises a leader sequence (e.g., SEQ ID NO: 23) positioned in a manner as follows (N-terminus to C-terminus): X-A-B-C, and the fusion polypeptide comprises a threonine between X and A. In some embodiments, the fusion polypeptide comprises a leader sequence (e.g., SEQ ID NO: 23) positioned in a manner as follows (N-terminus to C-terminus): X—C-B-A, and the fusion polypeptide comprises a threonine between X and C.

In some embodiments, the fusion polypeptide comprises an amino acid sequence that is at least 85%, 90%, 91%, 92%, 93%, 94%, 95%, 97%, 98% or 99% identical to any of the TβRII polypeptide amino acid sequences disclosed herein (e.g., SEQ ID NO: 18), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation). In some embodiments, the fusion polypeptide comprises an amino acid sequence that is at least 85%, 90%, 91%, 92%, 93%, 94%, 95%, 97%, 98%, or 99% identical to any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation). In some embodiments, the fusion polypeptide comprises an amino acid sequence that is at least 85%, 90%, 91%, 92%, 93%, 94%, 95%, 97%, 98%, or 99% identical to any of the heterologous portion sequences disclosed herein (e.g., SEQ ID NO: 20), wherein the heterologous portion of the fusion polypeptide comprises no more than 25, 20, 15, 10, 5, 4, 3, 2, or 1 additional amino acids (but which may include further post-translational modifications, such as PEGylation). In some embodiments, the fusion polypeptide comprises any of the TβRII polypeptide amino acid sequences disclosed herein (e.g., SEQ ID NO: 18), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation). In some embodiments, the fusion polypeptide comprises any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation). In some embodiments, the fusion polypeptide comprises any of the heterologous portion sequences disclosed herein (e.g., SEQ ID NO: 20 or 72), wherein the heterologous portion of the fusion polypeptide comprises no more than 25, 20, 15, 10, 5, 4, 3, 2, or 1 additional amino acids (but which may include further post-translational modifications, such as PEGylation).

In some embodiments, the disclosure provides for a fusion polypeptide, wherein the fusion polypeptide consists or consists essentially of (and not necessarily in the following order): a) an amino acid sequence that is at least 85%, 90%, 91%, 92%, 93%, 94%, 95%, 97%, 98%, or 99% identical to any of the TβRII polypeptide amino acid sequences disclosed herein (e.g., SEQ ID NO: 18), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation); b) an amino acid sequence that is at least 85%, 90%, 91%, 92%, 93%, 94%, 95%, 97%, 98%, or 99% identical to any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation); and c) an amino acid sequence that is at least 85%, 90%, 91%, 92%, 93%, 94%, 95%, 97%, 98%, or 99% identical to any of the heterologous portion sequences disclosed herein (e.g., SEQ ID NO: 20 or 72), wherein the heterologous portion of the fusion polypeptide comprises no more than 25, 20, 15, 10, 5, 4, 3, 2, or 1 additional amino acids (but which may include further post-translational modifications, such as PEGylation); and d) optionally a leader sequence (e.g., SEQ ID NO: 23). In some embodiments, the disclosure provides for a fusion polypeptide, wherein the fusion polypeptide consists or consists essentially of (and not necessarily in the following order): a) any of the TβRII polypeptide amino acid sequences disclosed herein (e.g., SEQ ID NO: 18), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation); b) any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation); and c) any of the heterologous portion sequences disclosed herein (e.g., SEQ ID NO: 20 or 72), wherein the heterologous portion of the fusion polypeptide comprises no more than 25, 20, 15, 10, 5, 4, 3, 2, or 1 additional amino acids (but which may include further post-translational modifications, such as PEGylation); and d) optionally a leader sequence (e.g., SEQ ID NO: 23).

In some embodiments, the disclosure provides for a fusion polypeptide consisting of or consisting essentially of (and not necessarily in the following order): a) a TβRII polypeptide portion consisting of an amino acid sequence that is at least 85%, 90%, 91%, 92%, 93%, 94%, 95%, 97%, 98%, or 99% identical to the amino acid sequence of SEQ ID NO: 18 and no more than 10, 9, 8, 7, 6, 5, 4, 3, 2 or 1 additional amino acids (but which may include further post-translational modifications, such as PEGylation); b) a linker portion consisting of an amino acid sequence that is at least 85%, 90%, 91%, 92%, 93%, 94%, 95%, 97%, 98%, or 99% identical to the amino acid sequence of SEQ ID NO: 6 and no more than 4, 3, 2 or 1 additional amino acids (but which may include further post-translational modifications, such as PEGylation); and c) a heterologous portion consisting of an amino acid sequence that is at least 85%, 90%, 91%, 92%, 93%, 94%, 95%, 97%, 98%, or 99% identical to the amino acid sequence of SEQ ID NO: 20 or SEQ ID NO: 72 and no more than 20, 15, 10, 5, 4, 3, 2, or 1 additional amino acids (but which may include further post-translational modifications, such as PEGylation); and d) optionally a leader sequence (e.g., SEQ ID NO: 23). In some embodiments, the disclosure provides for a fusion polypeptide consisting or consisting essentially of (and not necessarily in the following order): a) a TβRII polypeptide portion consisting of the amino acid sequence of SEQ ID NO: 18 and no more than 10, 9, 8, 7, 6, 5, 4, 3, 2 or 1 additional amino acids (but which may include further post-translational modifications, such as PEGylation); b) a linker portion consisting of the amino acid sequence of SEQ ID NO: 6 and no more than 5, 4, 3, 2 or 1 additional amino acids (but which may include further post-translational modifications, such as PEGylation); and c) a heterologous portion consisting of the amino acid sequence of SEQ ID NO: 20 or SEQ ID NO: 72 and no more than 25, 20, 15, 10, 5, 4, 3, 2, or 1 additional amino acids (but which may include further post-translational modifications, such as PEGylation); and d) optionally a leader sequence (e.g., SEQ ID NO: 23).

In some embodiments, the fusion protein does not comprise a leader sequence. In some embodiments, the fusion protein comprises an amino acid sequence that is at least 85%, 90%, 91%, 92%, 93%, 94%, 95%, 96%, 97%, 98%, 99%, or 100% identical to the amino acid sequence of SEQ ID NO: 48.

(SEQ ID NO: 48)
TIPPHVQKSDVEMEAQKDEIICPSCNRTAHPLRHINNDMIVTDNNGAVK
FPQLCKFCDVRESTCDNQKSCMSNCSITSICEKPQEVCVAVWRKNDENI
TLETVCHDPKLPYHDFILEDAASPKCIMKEKKKPGETFFMCSCSSDECN
DNIIFSEEYNTSNPDTGGGGSGGGGSGGGGSGGGGSTHTCPPCPAPELL
GGPSVELEPPKPKDTLMISRTPEVTCVVVDVSHEDPEVKENWYVDGVEV
HNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEA
LHNHYTQKSLSLSPGK.

In some embodiments, the C-terminal lysine residue of the Fc domain can be deleted. In some embodiments, the amino acid sequence of SEQ ID NO: 48 may optionally be provided with the lysine removed from the C-terminus (SEQ ID NO: 67):

(SEQ ID NO: 67)
TIPPHVQKSDVEMEAQKDEIICPSCNRTAHPLRHINNDMIVTDNNGAVK
FPQLCKFCDVRESTCDNQKSCMSNCSITSICEKPQEVCVAVWRKNDENI
TLETVCHDPKLPYHDFILEDAASPKCIMKEKKKPGETEFMCSCSSDECN
DNIIFSEEYNTSNPDTGGGGSGGGGSGGGGSGGGGSTHTCPPCPAPELL
GGPSVELEPPKPKDTLMISRTPEVTCVVVDVSHEDPEVKENWYVDGVEV
HNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEA
LHNHYTQKSLSLSPG.

In some embodiments, the fusion protein does not comprise a leader sequence. In some embodiments, the C-terminal lysine residue of the Fc domain is deleted. In some embodiments, the fusion protein comprises an amino acid sequence that is at least 85%, 90%, 91%, 92%, 93%, 94%, 95%, 96%, 97%, 98%, 99%, or 100% identical to the amino acid sequence of SEQ ID NO: 67.

In some embodiments, the disclosure provides for a TβRII fusion polypeptide wherein the polypeptide does not comprise an antibody or antigen-binding portion thereof. In some embodiments, the polypeptide does not bind with appreciable affinity to a cytokine other than a transforming growth factor beta superfamily ligand (e.g., TGFβ1, TGFβ2 and/or TGFβ3). In some embodiments, the polypeptide does not bind with appreciable affinity to a cytokine other than TGFβ1, TGFβ2 and/or TGFβ3. In some embodiments, the polypeptide does not bind with appreciable affinity to a cytokine other than TGFβ1 and/or TGFβ3. In some embodiments, the polypeptide does not bind with appreciable affinity to CD4, CD8, CD25, CTLA-4, IL-10, TGFβ Receptor, PD-1, PD-L1, PD-L2, RANK, RANKL, HER2/neu, EGFR1, CD20, VEGF, TNF-α, TNFR2, FoxP3, CD80, CD86, IFN-α, IFN-β, IFN-γ, GITR, 4-1BB, OX-40, TLR1-10, ErbB-1, HER1, ErbB-3/HER3, ErbB-4/HER4, IGFR, IGFBP, IGF-1R, PDGFR, FGFR, VEGFR, HGFR, TRK receptor, ephrin receptors, AXL receptors, LTK receptors, TIE receptors, angiopoietinl, 2, ROR receptor, DDR receptor, RET receptor, KLG receptor, RYK receptor, MuSK receptor, ILβR, IlαR, TNTRSF, TRAIL receptor, ARTC1, alpha-actinin-4, Bcr-abl, B-RAF, caspases, beta-catenin, fibronectin, GPNMB, GDP-L, LDLR, HLA-A2, MLA-A11, HSP70, KIAA205, MART2, MUM-1, 2, 3, PAP, neo-PAP, NFYC, OGT, OS-9, pml-RARalpha fusion protein, PRDXS, PTPRK, KRAS2, NRAS, HRAS, RBAF600, SIRT2. SNRPD1, SYT-SSX1 or -SSX2 fusion protein, Triosephosphate Isomerase, BAGE, BAGE-1. BAGE-2, 3, 4, 5, GAGE-1, 2, 3, 4, 5, 6, 7, 8, GnT-V, HERV-K MEL, KK-LC, KM-HN-1, LAGE, LAGE-1, CAMEL, MAGE-1, MAGE-A2, MAGE-A3, MAGE-A4, MAGE-AS, MAGE-A6, MAGE-A8, MAGE-A9, MAGE-A10. MAGE-A11, MAGE-A12, MAGE-3, MAGE-B1, MAGE-B2, MAGE-B5. MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17. SSX-1, 2, 3, 4, TRP2-1NT2, carcino-embryonic antigen (CEA), Kallikfein 4, mammaglobm-A, OA1, prostate specific antigen (PSA), prostate specific membrane antigen, TRP-1/, 75. TRP-2, AIM-2. BING-4, CPSF, cyclin D1, Ep-CAM, EpbA3, FGF-5, gp250, iCE), AFP, M-CSF, mdm-2, MUCI, p53 (TP53), PBF, FRAME, PSMA, RAGE-1. RNF43, RU2AS, SOX10, STEAP1, survivin (BIRCS), hTERT, telomerase, WT1, SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SP011, TPX1, NY-SAR-35, FTHLI7, NXF2 TDRD1, TEX 15, FATE, TPTE, estrogen receptors (ER), androgen receptors (AR), CD40, CD30, CD20, CD19, CD33, CD4, CD25, CD3, CA 72-4, CA 15-3, CA 27-29, CA 125, CA 19-9, beta-human chorionic gonadotropin, 1-2 microglobulin, squamous cell carcinoma antigen, neuron-specific enoJase, heat shock protein gp96, GM2, sargramostim, CTLA-4, 707-AP, ART-4, CAP-1, CLCA2, Cyp-B, HST-2, HPV proteins, EBV proteins, Hepatitis B or C virus proteins, and/or HIV proteins.

In some embodiments, the disclosure provides for a TβRII fusion polypeptide wherein the polypeptide does not comprise an additional ligand binding domain in addition to the TβRII domain. In some embodiments, the polypeptide comprises a linear amino acid sequence comprising a TβRII domain and a heterologous portion (e.g., an Fc portion), but the linear amino acid sequence does not comprise any additional ligand binding domains. In some embodiments, the polypeptide comprises a linear amino acid sequence comprising a TβRII domain and an Fc portion, but the linear amino acid sequence does not comprise any additional ligand binding domains. In some embodiments, the disclosure provides for a TβRII fusion polypeptide wherein the polypeptide does not comprise multiple ligand binding domains in a single linear amino acid sequence. In some embodiments, the disclosure provides for a TβRII fusion polypeptide wherein the polypeptide does not comprise more than one continuous linker sequence in a single linear amino acid sequence. In some embodiments, the polypeptide does not comprise multiple continuous glycine and/or serine linkers (e.g., a linker comprising (GGGGS)n, wherein n=≥4 (SEQ ID NO: 59)) in a single linear amino acid sequence. In some embodiments, the disclosure provides for a TβRII fusion polypeptide wherein the heterologous portion is an Fc domain, and wherein only one continuous linker is covalently bound to the Fc domain. In some embodiments, the only one continuous linker comprises or consists of a (GGGGS)n linker, wherein n=≥4 (SEQ ID NO: 59).

### 12. Nucleic Acids and Methods of Manufacture

In certain embodiments, the present disclosure makes available isolated and/or purified forms of the TβRII polypeptides fusion proteins, which are isolated from, or otherwise substantially free of (e.g., at least 80%, 90%, 91%, 92%, 93%, 94%, 95%, 96%, 97%, 98%, or 99% free of), other proteins and/or other TβRII polypeptide species. TβRII polypeptides will generally be produced by expression from recombinant nucleic acids.

In certain embodiments, the disclosure includes nucleic acids encoding soluble TβRII polypeptides comprising the coding sequence for an extracellular portion of a TβRII protein. In further embodiments, this disclosure also pertains to a host cell comprising such nucleic acids. The host cell may be any prokaryotic or eukaryotic cell. For example, a polypeptide of the present disclosure may be expressed in bacterial cells such as E. coli, insect cells (e.g., using a baculovirus expression system), yeast, or mammalian cells. Other suitable host cells are known to those skilled in the art. Accordingly, some embodiments of the present disclosure further pertain to methods of producing the TβRII polypeptides.

In certain aspects, the disclosure provides isolated and/or recombinant nucleic acids encoding any of the TβRII polypeptides, including fragments, functional variants and fusion proteins disclosed herein. SEQ ID NOs: 8, 10, 12, 14, 16, 46, 47, and 87 encode variants of TβRII extracellular domain fused to an IgG Fc domain. The subject nucleic acids may be single-stranded or double stranded. Such nucleic acids may be DNA or RNA molecules. These nucleic acids may be used, for example, in methods for making TβRII polypeptides or as direct therapeutic agents (e.g., in an antisense, RNAi or gene therapy approach).

In certain aspects, the subject nucleic acids encoding TβRII polypeptides are further understood to include nucleic acids that are variants of SEQ ID NOs: 8, 10, 12, 14, 16, 46, 47, and 87. Variant nucleotide sequences include sequences that differ by one or more nucleotide substitutions, additions or deletions, such as allelic variants.

In certain embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NOs: 8, 10, 12, 14, 16, 46, 47, and 87. In particular embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 12, or fragments thereof. In some embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 8, or fragments thereof. In some embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 10, or fragments thereof. In some embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 14, or fragments thereof. In some embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 16, or fragments thereof. In some embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 46, or fragments thereof. In some embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 47, or fragments thereof. In some embodiments, the disclosure provides isolated or recombinant nucleic acid sequences that are at least 80%, 85%, 90%, 91%, 92%, 93%, 94%, 95%, 96%, 97%, 98%, 99% or 100% identical to SEQ ID NO: 87, or fragments thereof. One of ordinary skill in the art will appreciate that nucleic acid sequences complementary to SEQ ID NOs: 8, 10, 12, 14, 16, 46, 47, and 87, and variants of SEQ ID NOs: 8, 10, 12, 14, 16, 46, 47, and 87 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to the nucleotide sequences designated in SEQ ID NOs: 8, 10, 12, 14, 16, 46, 47, and 87 complement sequences of SEQ ID NOs: 8, 10, 12, 14, 16, 46, 47, and 87, or fragments thereof. As discussed above, one of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about Both temperature and salt may be varied, or temperature or salt concentration may be held constant while the other variable is changed. In some embodiments, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 8, 10, 12, 14, 16, 46, 47, and 87 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

It will be appreciated by one of skill in the art that corresponding variants based on the long isoform of TβRII will include nucleotide sequences encoding the 25-amino acid insertion along with a conservative Val-Ile substitution at the flanking position C-terminal to the insertion. It will also be appreciated that corresponding variants based on either the long (A) or short (B) isoforms of TβRII will include variant nucleotide sequences comprising an insertion of 108 nucleotides, encoding a 36-amino-acid insertion (SEQ ID NO: 41), at the same location described for naturally occurring TβRII isoform C.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects disclosed herein, the subject nucleic acid is provided in an expression vector comprising a nucleotide sequence encoding a TβRII polypeptide and operably linked to at least one regulatory sequence. Regulatory sequences are art-recognized and are selected to direct expression of the TβRII polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding a TβRII polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid included in the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant TβRII polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In certain embodiments, a vector will be designed for production of the subject TβRII polypeptides in CHO cells, such as a Pcmv-Script vector (Stratagene, La Jolla, Calif.), pcDN4 vectors (Invitrogen, Carlsbad, Calif.) and pCI-neo vectors (Promega, Madison, Wisc.). In a preferred embodiment, a vector will be designed for production of the subject TβRII polypeptides in HEK-293 cells. As will be apparent, the subject gene constructs can be used to cause expression of the subject TβRII polypeptides in cells propagated in culture, e.g., to produce proteins, including fusion proteins or variant proteins, for purification.

This disclosure also pertains to a host cell transfected with a recombinant gene including a coding sequence (e.g., SEQ ID NOs: 8, 10, 12, 14, 16, 46, 47, and 87) for one or more of the subject TβRII polypeptides. The host cell may be any prokaryotic or eukaryotic cell. For example, a TβRII polypeptide disclosed herein may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject TβRII polypeptides. For example, a host cell transfected with an expression vector encoding a TβRII polypeptide can be cultured under appropriate conditions to allow expression of the TβRII polypeptide to occur. The TβRII polypeptide may be secreted and isolated from a mixture of cells and medium containing the TβRII polypeptide. Alternatively, the TβRII polypeptide may be retained cytoplasmically or in a membrane fraction and the cells harvested, lysed and the protein isolated. A cell culture includes host cells, and media. Suitable media for cell culture are well known in the art. The subject TβRII polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the TβRII polypeptides and affinity purification with an agent that binds to a domain fused to the TβRII polypeptide (e.g., a protein A column may be used to purify an TβRII-Fc fusion). In a preferred embodiment, the TβRII polypeptide is a fusion protein containing a domain which facilitates its purification. As an example, purification may be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

In another embodiment, a fusion gene coding for a purification leader sequence, such as a poly-(His)/enterokinase cleavage site sequence at the N-terminus of the desired portion of the recombinant TβRII polypeptide, can allow purification of the expressed fusion protein by affinity chromatography using a Ni²⁺ metal resin. The purification leader sequence can then be subsequently removed by treatment with enterokinase to provide the purified TβRII polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 13. Alterations in Fc-Fusion Proteins

The application further provides TβRII-Fc fusion proteins with engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus, in certain embodiments, Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively, or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

### 14. Antibody Antagonists

In certain aspects, a TβRII antagonist to be used in accordance with the methods and uses disclosed herein is an antibody, or combination of antibodies. An antibody TβRII antagonist may inhibit and/or bind to, for example, one or more TβRII ligands (e.g., TGFβ1, TGFβ2, and/or TGFβ3), the TβRII receptor, TβRII-associated type I receptor (e.g., ALK5), and/or TβRII co-receptor (e.g., betaglycan). In some embodiments, the ability for an antibody TβRII antagonist antibody to inhibit activity (e.g., Smad signaling) and/or bind to a target is determined in an in vitro or cell-based assay including, for example, those disclosed herein. As described herein, an antibody TβRII antagonist may be used alone or in combination with one or more additional active agents or supportive therapies to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In certain embodiments, a TβRII antagonist is an antibody that inhibits at least TGFβ1. Therefore, in some embodiments, an antibody TβRII antagonist binds to at least TGFβ1. As used herein, a TGFβ1 antibody (anti-TGFβ1 antibody) generally refers to an antibody that is capable of binding to TGFβ1 with sufficient affinity such that the antibody is useful as a diagnostic and/or therapeutic agent in targeting TGFβ1. In certain embodiments, the extent of binding of an anti-TGFβ1 antibody to an unrelated, non-TGFβ1 protein is less than about 10%, 9%, 8%, 7%, 6%, 5%, 4%, 3%, 2%, or less than 1% of the binding of the antibody to TGFβ1 as measured, for example, by a radioimmunoassay (RIA). In certain embodiments, an anti-TGFβ1 antibody binds to an epitope of TGFβ1 that is conserved among TGFβ1 from different species. In certain preferred embodiments, an anti-TGFβ1 antibody binds to human TGFβ1. In some embodiments, a TGFβ1 antibody may inhibit TGFβ1 from binding to a type I, type II, and/or co-receptor (e.g., TβRII, ALK5, and/or betaglycan) and thus inhibit TGFβ1-mediated signaling (e.g., Smad signaling). It should be noted that TGFβ1 shares some sequence homology to TGFβ2 and TGFβ3. Therefore, antibodies that bind TGFβ1, in some embodiments, may also bind to TGFβ2 and/or TGFβ3. In some embodiments, the disclosure relates to a multispecific antibody (e.g., bi-specific antibody), and uses thereof, that binds to TGFβ1 and further binds to, for example, one or more additional TβRII ligands (e.g., TGFβ2, TGFβ3, or TGFβ2 and TGFβ3), one or more type I and/or type II receptors (e.g., TβRII and ALK5), and/or one or more co-receptors (e.g., betaglycan). In some embodiments, a multispecific antibody that binds to TGFβ1 does not bind or does not substantially bind to TGFβ2 (e.g., binds to TGFβ2 with a K_{D} of greater than 1×10⁻⁷ M or has relatively modest binding, e.g., about 1×10⁻⁸ M or about 1×10⁻⁹ M). In some embodiments, a multispecific antibody that binds to TGFβ1 further binds to TGFβ3 but does not bind or does not substantially bind to TGFβ2 (e.g., binds to TGFβ2 with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises a TGFβ1 antibody and one or more additional antibodies that bind to, for example, one or more additional TβRII ligands (e.g., TGFβ2, TGFβ3, or TGFβ2 and TGFβ3), one or more type I and/or type II receptors (e.g., TβRII and ALK5), and/or one or more co-receptors (e.g., betaglycan). In some embodiments, a combination of antibodies that comprises a TGFβ1 antibody does not comprise a TGFβ2 antibody. In some embodiments, a combination of antibodies that comprises a TGFβ1 antibody further comprises a TGFβ3 antibody but does not comprise a TGFβ2 antibody.

In certain embodiments, an antibody TβRII antagonist is an antibody that inhibits at least TGFβ2. Therefore, in some embodiments, an antibody TβRII antagonist antibody binds to at least TGFβ2. As used herein, a TGFβ2 antibody (anti-TGFβ2 antibody) generally refers to an antibody that is capable of binding to TGFβ2 with sufficient affinity such that the antibody is useful as a diagnostic and/or therapeutic agent in targeting TGFβ2. In certain embodiments, the extent of binding of an anti-TGFβ2 antibody to an unrelated, non-TGFβ2 protein is less than about 10%, 9%, 8%, 7%, 6%, 5%, 4%, 3%, 2%, or less than 1% of the binding of the antibody to TGFβ2 as measured, for example, by a radioimmunoassay (RIA). In certain embodiments, an anti-TGFβ2 antibody binds to an epitope of TGFβ2 that is conserved among TGFβ2 from different species. In certain preferred embodiments, an anti-TGFβ2 antibody binds to human TGFβ2. In some embodiments, a TGFβ2 antibody may inhibit TGFβ2 from binding to a type I, type II, and/or co-receptor (e.g., TβRII, ALK5, and/or betaglycan) and thus inhibit TGFβ2 activity (e.g., Smad signaling). It should be noted that TGFβ2 shares some sequence homology to TGFβ1 and TGFβ3. Therefore, antibodies that bind TGFβ2, in some embodiments, may also bind to TGFβ1 and/or TGFβ3. In some embodiments, the disclosure relates to a multispecific antibody (e.g., bi-specific antibody), and uses thereof, that binds to TGFβ2 and further binds to, for example, one or more additional TβRII ligands (e.g., TGFβ1, TGFβ3, or TGFβ1 and TGFβ3), one or more type I and/or type II receptors (e.g., TβRII and ALK5), and/or one or more co-receptors (e.g., betaglycan) In some embodiments, the disclosure relates to combinations of antibodies, and uses thereof, wherein the combination of antibodies comprises a TGFβ2 antibody and one or more additional antibodies that bind to, for example, one or more additional TβRII ligands (e.g., TGFβ1, TGFβ3, or TGFβ1 and TGFβ3), one or more type I and/or type II receptors (e.g., TβRII and ALK5), and/or one or more co-receptors (e.g., betaglycan).

In certain embodiments, an antibody TβRII antagonist is an antibody that inhibits at least TGFβ3. Therefore, in some embodiments, an antibody TβRII antagonist antibody binds to at least TGFβ3. As used herein, a TGFβ3 antibody (anti-TGFβ3 antibody) generally refers to an antibody that is capable of binding to TGFβ3 with sufficient affinity such that the antibody is useful as a diagnostic and/or therapeutic agent in targeting TGFβ3. In certain embodiments, the extent of binding of an anti-TGFβ3 antibody to an unrelated, non-TGFβ3 protein is less than about 10%, 9%, 8%, 7%, 6%, 5%, 4%, 3%, 2%, or less than 1% of the binding of the antibody to TGFβ3 as measured, for example, by a radioimmunoassay (RIA). In certain embodiments, an anti-TGFβ3 antibody binds to an epitope of TGFβ3 that is conserved among TGFβ3 from different species. In certain preferred embodiments, an anti-TGFβ3 antibody binds to human TGFβ3. In some embodiments, a TGFβ3 antibody may inhibit TGFβ3 from binding to a type I, type II, and/or co-receptor (e.g., TβRII, ALK5, and/or betaglycan) and thus inhibit TGFβ3 activity (e.g., Smad signaling). It should be noted that TGFβ3 shares some sequence homology to TGFβ2 and TGFβ1. Therefore, antibodies that bind TGFβ3, in some embodiments, may also bind to TGFβ2 and/or TGFβ1. In some embodiments, the disclosure relates to a multispecific antibody (e.g., bi-specific antibody), and uses thereof, that binds to TGFβ3 and further binds to, for example, one or more additional TβRII ligands (e.g., TGFβ2, TGFβ1, or TGFβ2 and TGFβ1), one or more type I and/or type II receptors (e.g., TβRII and ALK5), and/or one or more co-receptors (e.g., betaglycan). In some embodiments, a multispecific antibody that binds to TGFβ3 does not bind or does not substantially bind to TGFβ2 (e.g., binds to TGFβ2 with a K_{D} of greater than 1×10⁻⁷ M or has relatively modest binding, e.g., about 1×10⁻⁸ M or about 1×10⁻⁹ M). In some embodiments, a multispecific antibody that binds to TGFβ3 further binds to TGFβ1 but does not bind or does not substantially bind to TGFβ2 (e.g., binds to TGFβ2 with a K_{D} of greater than 1×10⁻⁷ M or has relatively modest binding, e.g., about 1×10⁻⁸ M or about 1×10⁻⁹ M). In some embodiments, the disclosure relates to combinations of antibodies, and uses thereof, wherein the combination of antibodies comprises a TGFβ3 antibody and one or more additional antibodies that bind to, for example, one or more additional TβRII ligands (e.g., TGFβ2, TGFβ1, or TGFβ2 and TGFβ1), one or more type I and/or type II receptors (e.g., TβRII and ALK5), and/or one or more co-receptors (e.g., betaglycan). In some embodiments, a combination of antibodies that comprises a TGFβ3 antibody does not comprise a TGFβ2 antibody. In some embodiments, a combination of antibodies that comprises a TGFβ3 antibody further comprises a TGFβ1 antibody but does not comprise a TGFβ2 antibody.

In certain aspects, an antibody TβRII antagonist is an antibody that inhibits at least the TβRII receptor. Therefore, in some embodiments, an antibody TβRII antagonist binds to at least the TβRII receptor. As used herein, a TβRII receptor antibody generally refers to an antibody that binds to a TβRII receptor with sufficient affinity such that the antibody is useful as a diagnostic and/or therapeutic agent in targeting a TβRII receptor. In certain embodiments, the extent of binding of an anti-TβRII receptor antibody to an unrelated, non-TβRII receptor protein is less than about 10%, 9%, 8%, 7%, 6%, 5%, 4%, 3%, 2%, or less than about 1% of the binding of the antibody to the TβRII receptor as measured, for example, by a radioimmunoassay (RIA), Biacore™, or other protein-protein interaction or binding affinity assay. In certain embodiments, an anti-TβRII receptor antibody binds to an epitope of a TβRII receptor that is conserved among TβRII receptors from different species. In certain preferred embodiments, an anti-TβRII receptor antibody binds to a human TβRII receptor. In some embodiments, an anti-TβRII receptor antibody may inhibit one or more TβRII ligands [e.g., TGFβ1; TGFβ2; TGFβ3; TGFβ1 and TGFβ3; TGFβ1 and TGFβ2; TGFβ2 and TGFβ3; or TGFβ1, TGFβ2, and TGFβ3] from binding to a TβRII receptor. In some embodiments, an anti-TβRII receptor antibody is a multispecific antibody (e.g., bi-specific antibody) that binds to a TβRII receptor and one or more TβRII ligands [e.g., TGFβ1, TGFβ2, and TGFβ3], type I receptor (e.g., ALK5), and/or co-receptor (e.g., betaglycan). In some embodiments, the disclosure relates to combinations of antibodies, and uses thereof, wherein the combination of antibodies comprises an anti-TβRII receptor antibody and one or more additional antibodies that bind to, for example, one or more TβRII ligands [e.g., TGFβ1, TGFβ2, and TGFβ3], type I receptors (e.g., ALK5), and/or co-receptor (e.g., betaglycan).

In certain aspects, an antibody TβRII antagonist is an antibody that inhibits at least ALK5. Therefore, in some embodiments, an antibody TβRII antagonist antibody binds to at least ALK5. As used herein, an ALK5 antibody (anti-ALK5antibody) generally refers to an antibody that binds to ALK5 with sufficient affinity such that the antibody is useful as a diagnostic and/or therapeutic agent in targeting ALK5. In certain embodiments, the extent of binding of an anti-ALK5 antibody to an unrelated, non-ALK5 protein is less than about 10%, 9%, 8%, 7%, 6%, 5%, 4%, 3%, 2%, or less than about 1% of the binding of the antibody to ALK5 as measured, for example, by a radioimmunoassay (RIA), Biacore, or other protein-protein interaction or binding affinity assay. In certain embodiments, an anti-ALK5 antibody binds to an epitope of ALK5 that is conserved among ALK5 from different species. In certain preferred embodiments, an anti-ALK5 antibody binds to human ALK5. In some embodiments, an anti-ALK5 antibody may inhibit one or more TβRII ligands [e.g., TGFβ1; TGFβ2; TGFβ3; TGFβ1 and TGFβ3; TGFβ1 and TGFβ2; TGFβ2 and TGFβ3; or TGFβ1, TGFβ2, and TGFβ3] from binding to ALK5. In some embodiments, an anti-ALK5 antibody is a multispecific antibody (e.g., bi-specific antibody) that binds to ALK5 and one or more TβRII ligands [e.g., TGFβ1, TGFβ2, and TGFβ3], type II receptor (e.g., TβRII), and/or co-receptor (e.g., betaglycan). In some embodiments, the disclosure relates to combinations of antibodies, and uses thereof, wherein the combination of antibodies comprises an anti-ALK5 antibody and one or more additional antibodies that bind to, for example, one or more TβRII ligands [e.g., TGFβ1, TGFβ2, and TGFβ3], type II receptors (e.g., TβRII), and/or co-receptor (e.g., betaglycan).

In certain aspects, an antibody TβRII antagonist is an antibody that inhibits at least betaglycan. Therefore, in some embodiments, an antibody TβRII antagonist binds to at least betaglycan. As used herein, a betaglycan antibody (anti-betaglycan antibody) generally refers to an antibody that binds to betaglycan with sufficient affinity such that the antibody is useful as a diagnostic and/or therapeutic agent in targeting betaglycan. In certain embodiments, the extent of binding of an anti-betaglycan antibody to an unrelated, non-betaglycan protein is less than about 10%, 9%, 8%, 7%, 6%, 5%, 4%, 3%, 2%, or less than about 1% of the binding of the antibody to betaglycan as measured, for example, by a radioimmunoassay (RIA), Biacore, or other protein-protein interaction or binding affinity assay. In certain embodiments, an anti-betaglycan antibody binds to an epitope of betaglycan that is conserved among betaglycan from different species. In certain preferred embodiments, an anti-betaglycan antibody binds to human betaglycan. In some embodiments, an anti-betaglycan antibody may inhibit one or more TβRII ligands [e.g., TGFβ1; TGFβ2; TGFβ3; TGFβ1 and TGFβ3; TGFβ1 and TGFβ2; TGFβ2 and TGFβ3; or TGFβ1, TGFβ2, and TGFβ3] from binding to betaglycan. In some embodiments, an anti-betaglycan antibody is a multispecific antibody (e.g., bi-specific antibody) that binds to betaglycan and one or more TβRII ligands [e.g., TGFβ1, TGFβ2, and TGFβ3], type I receptor (e.g., ALK5), and/or type II receptors (e.g., TβRII). In some embodiments, the disclosure relates to combinations of antibodies, and uses thereof, wherein the combination of antibodies comprises an anti-betaglycan antibody and one or more additional antibodies that bind to, for example, one or more TβRII ligands [e.g., TGFβ1, TGFβ2, and TGFβ3], type I receptors (e.g., ALK5), and/or type II receptors (e.g., TβRII).

The term antibody is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody fragment refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments. See, e.g., Hudson et al. (2003) Nat. Med. 9:129-134; Pluckthun, in The Pharmacology of Monoclonal Antibodies, vol. 113, Rosenburg and Moore eds., (Springer-Verlag, New York), pp. 269-315 (1994); WO 93/16185; and U.S. Pat. Nos. 5,571,894, and 5,869,046. Antibodies disclosed herein may be polyclonal antibodies or monoclonal antibodies. In certain embodiments, the antibodies of the present disclosure comprise a label attached thereto and able to be detected (e.g., the label can be a radioisotope, fluorescent compound, enzyme, or enzyme co-factor). In preferred embodiments, the antibodies of the present disclosure are isolated antibodies. Diabodies are antibody fragments with two antigen-binding sites that may be bivalent or bispecific. See, e.g., EP 404,097; WO 1993/01161; Hudson et al. (2003) Nat. Med. 9:129-134 (2003); and Hollinger et al. (1993) Proc. Natl. Acad. Sci. USA 90: 6444-6448. Triabodies and tetrabodies are also described in Hudson et al. (2003) Nat. Med. 9:129-134. Single-domain antibodies are antibody fragments comprising all or a portion of the heavy-chain variable domain or all or a portion of the light-chain variable domain of an antibody. In certain embodiments, a single-domain antibody is a human single-domain antibody. See, e.g., U.S. Pat. No. 6,248,516. Antibody fragments can be made by various techniques, including but not limited to proteolytic digestion of an intact antibody as well as production by recombinant host cells (e.g., E. coli or phage), as described herein.

The antibodies herein may be of any class. The class of an antibody refers to the type of constant domain or constant region possessed by its heavy chain. There are five major classes of antibodies: IgA, IgD, IgE, IgG, and IgM, and several of these may be further divided into subclasses (isotypes), for example, IgG₁, IgG₂, IgG₃, IgG₄, IgA₁, and IgA₂. The heavy-chain constant domains that correspond to the different classes of immunoglobulins are called alpha, delta, epsilon, gamma, and mu.

In general, an antibody for use in the methods disclosed herein specifically binds to its target antigen, preferably with high binding affinity. Affinity may be expressed as a K_{D} value and reflects the intrinsic binding affinity (e.g., with minimized avidity effects). Typically, binding affinity is measured in vitro, whether in a cell-free or cell-associated setting. Any of a number of assays known in the art, including those disclosed herein, can be used to obtain binding affinity measurements including, for example, surface plasmon resonance (Biacore™ assay), radiolabeled antigen binding assay (RIA), and ELISA. In some embodiments, antibodies of the present disclosure bind to their target antigens (e.g. TGFβ1, TGFβ2, TGFβ2, ALK5, betaglycan, and TβRII.) with at least a K_{D} of 1×10⁻⁷ or stronger, 1×10⁻⁸ or stronger, 1×10⁻⁹ or stronger, 1×10⁻¹⁰ or stronger, 1×10⁻¹¹ or stronger, 1×10⁻¹² or stronger, 1×10⁻¹³ or stronger, or 1×10⁻¹⁴ or stronger.

In certain embodiments, K_{D} is measured by RIA performed with the Fab version of an antibody of interest and its target antigen as described by the following assay. Solution binding affinity of Fabs for the antigen is measured by equilibrating Fab with a minimal concentration of radiolabeled antigen (e.g., 125 I-labeled) in the presence of a titration series of unlabeled antigen, then capturing bound antigen with an anti-Fab antibody-coated plate [see, e.g., Chen et al. (1999) J. Mol. Biol. 293:865-881]. To establish conditions for the assay, multi-well plates (e.g., MICROTITER® from Thermo Scientific) are coated (e.g., overnight) with a capturing anti-Fab antibody (e.g., from Cappel Labs) and subsequently blocked with bovine serum albumin, preferably at room temperature (e.g., approximately 23° C.). In a non-adsorbent plate, radiolabeled antigen are mixed with serial dilutions of a Fab of interest [e.g., consistent with assessment of the anti-VEGF antibody, Fab-12, in Presta et al., (1997) Cancer Res. 57:4593-4599]. The Fab of interest is then incubated, preferably overnight but the incubation may continue for a longer period (e.g., about 65 hours) to ensure that equilibrium is reached. Thereafter, the mixtures are transferred to the capture plate for incubation, preferably at room temperature for about one hour. The solution is then removed and the plate is washed times several times, preferably with polysorbate 20 and PBS mixture. When the plates have dried, scintillant (e.g., MICROSCINT® from Packard) is added, and the plates are counted on a gamma counter (e.g., TOPCOUNT® from Packard).

According to another embodiment, K_{D} is measured using surface plasmon resonance assays using, for example a BIACORE® 2000 or a BIACORE® 3000 (Biacore, Inc., Piscataway, N.J.) with immobilized antigen CMS chips at about 10 response units (RU). Briefly, carboxymethylated dextran biosensor chips (CMS, Biacore, Inc.) are activated with N-ethyl-N′-(3-dimethylaminopropyl)-carbodiimide hydrochloride (EDC) and N-hydroxysuccinimide (NHS) according to the supplier's instructions. For example, an antigen can be diluted with 10 mM sodium acetate, pH 4.8, to 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20 ®) surfactant (PBST) at a flow rate of approximately 25 μl/min. Association rates (kₒₙ) and dissociation rates (k_{off}) are calculated using, for example, a simple one-to-one Langmuir binding model (BIACORE® Evaluation Software version 3.2) by simultaneously fitting the association and dissociation sensorgrams. The equilibrium dissociation constant (K D) is calculated as the ratio k_{off}/kₒₙ. [see, e.g., Chen et al., (1999) J. Mol. Biol. 293:865-881]. If the on-rate exceeds, for example, 10⁶M⁻¹ s⁻¹ by the surface plasmon resonance assay above, then the on-rate can be determined by using a fluorescent quenching technique that measures the increase or decrease in fluorescence emission intensity (e.g., excitation=295 nm; emission=340 nm, 16 nm band-pass) of a 20 nM anti-antigen antibody (Fab form) in PBS in the presence of increasing concentrations of antigen as measured in a spectrometer, such as a stop-flow equipped spectrophotometer (Aviv Instruments) or a 8000-series SLM-AMINCO® spectrophotometer (ThermoSpectronic) with a stirred cuvette.

The nucleic acid and amino acid sequences of TβRII, ALK5, betaglycan, TGFβ1, TGFβ2, and TGFβ3, particularly human sequences, are well known in the art and thus antibody antagonists for use in accordance with this disclosure may be routinely made by the skilled artisan based on the knowledge in the art and teachings provided herein.

In certain embodiments, an antibody provided herein is a chimeric antibody. A chimeric antibody refers to an antibody in which a portion of the heavy and/or light chain is derived from a particular source or species, while the remainder of the heavy and/or light chain is derived from a different source or species. Certain chimeric antibodies are described, for example, in U.S. Pat. No. 4,816,567; and Morrison et al., (1984) Proc. Natl. Acad. Sci. USA, 81:6851-6855. In some embodiments, a chimeric antibody comprises a non-human variable region (e.g., a variable region derived from a mouse, rat, hamster, rabbit, or non-human primate, such as a monkey) and a human constant region. In some embodiments, a chimeric antibody is a “class switched” antibody in which the class or subclass has been changed from that of the parent antibody. In general, chimeric antibodies include antigen-binding fragments thereof.

In certain embodiments, a chimeric antibody provided herein is a humanized antibody. A humanized antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Humanized antibodies and methods of making them are reviewed, for example, in Almagro and Fransson (2008) Front. Biosci. 13:1619-1633 and are further described, for example, in Riechmann et al., (1988) Nature 332:323-329; Queen et al. (1989) Proc. Nat'l Acad. Sci. USA 86:10029-10033; U.S. Pat. Nos. 7,527,791, 6,982,321, and 7,087,409; Kashmiri et al., (2005) Methods 36:25-34 [describing SDR (a-CDR) grafting]; Padlan, Mol. Immunol. (1991) 28:489-498 (describing “resurfacing”); Dall'Acqua et al. (2005) Methods 36:43-60 (describing “FR shuffling”); Osbourn et al. (2005) Methods 36:61-68; and Klimka et al. Br. J. Cancer (2000) 83:252-260 (describing the “guided selection” approach to FR shuffling).

Human framework regions that may be used for humanization include but are not limited to: framework regions selected using the “best-fit” method [see, e.g., Sims et al. (1993) J. Immunol. 151:2296]; framework regions derived from the consensus sequence of human antibodies of a particular subgroup of light-chain or heavy-chain variable regions [see, e.g., Carter et al. (1992) Proc. Natl. Acad. Sci. USA, 89:4285; and Presta et al. (1993) J. Immunol., 151:2623]; human mature (somatically mutated) framework regions or human germline framework regions [see, e.g., Almagro and Fransson (2008) Front. Biosci. 13:1619-1633]; and framework regions derived from screening FR libraries [see, e.g., Baca et cd., (1997) J. Biol. Chem. 272:10678-10684; and Rosok et cd., (1996) J. Biol. Chem. 271:22611-22618].

In certain embodiments, an antibody provided herein is a human antibody. Human antibodies can be produced using various techniques known in the art. Human antibodies are described generally in van Dijk and van de Winkel (2001) Curr. Opin. Pharmacol. 5: 368-74 and Lonberg (2008) Curr. Opin. Immunol. 20:450-459.

In some embodiments, human antibodies may be prepared by administering an immunogen (e.g., a TβRII, ALK5, betaglycan, TGFβ1, TGFβ2, or TGFβ3 polypeptide) to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. For a review of methods for obtaining human antibodies from transgenic animals, see, for example, Lonberg (2005) Nat. Biotechnol. 23:1117-1125; U.S. Pat. Nos. 6,075,181 and 6,150,584 (describing XENOMOUSE™ technology); U.S. Pat. No. 5,770,429 (describing HuMab® technology); U.S. Pat. No. 7,041,870 (describing K-M MOUSE® technology); and U.S. Patent Application Publication No. 2007/0061900 (describing VelociMouse® technology). Human variable regions from intact antibodies generated by such animals may be further modified, for example, by combining with a different human constant region.

Human antibodies provided herein can also be made by hybridoma-based methods. Human myeloma and mouse-human heteromyeloma cell lines for the production of human monoclonal antibodies have been described [see, e.g., Kozbor J. Immunol., (1984) 133: 3001; Brodeur et al. (1987) Monoclonal Antibody Production Techniques and Applications, pp. 51-63, Marcel Dekker, Inc., New York; and Boerner et al. (1991) J. Immunol., 147: 86]. Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, Xiandai Mianyixue (2006) 26(4):265-268 (2006) (describing human-human hybridomas). Human hybridoma technology (Trioma technology) is also described in Vollmers and Brandlein (2005) Histol. Histopathol., 20(3):927-937 (2005) and Vollmers and Brandlein (2005) Methods Find Exp. Clin. Pharmacol., 27(3):185-91.

Human antibodies provided herein may also be generated by isolating Fv clone variable-domain sequences selected from human-derived phage display libraries. Such variable-domain sequences may then be combined with a desired human constant domain. Techniques for selecting human antibodies from antibody libraries are described herein.

For example, antibodies of the present disclosure may be isolated by screening combinatorial libraries for antibodies with the desired activity or activities. A variety of methods are known in the art for generating phage-display libraries and screening such libraries for antibodies possessing the desired binding characteristics. Such methods are reviewed, for example, in Hoogenboom et al. (2001) in Methods in Molecular Biology 178:1-37, O'Brien et al., ed., Human Press, Totowa, N.J. and further described, for example, in the McCafferty et al. (1991) Nature 348:552-554; Clackson et al., (1991) Nature 352: 624-628; Marks et al. (1992) J. Mol. Biol. 222:581-597; Marks and Bradbury (2003) in Methods in Molecular Biology 248:161-175, Lo, ed., Human Press, Totowa, N.J.; Sidhu et al. (2004) J. Mol. Biol. 338(2):299-310; Lee et al. (2004) J. Mol. Biol. 340(5):1073-1093; Fellouse (2004) Proc. Natl. Acad. Sci. USA 101(34):12467-12472; and Lee et al. (2004) J. Immunol. Methods 284(1-2): 119-132.

In certain phage display methods, repertoires of VH and VL genes are separately cloned by polymerase chain reaction (PCR) and recombined randomly in phage libraries, which can then be screened for antigen-binding phage as described in Winter et al. (1994) Ann. Rev. Immunol., 12: 433-455. Phage typically display antibody fragments, either as single-chain Fv (scFv) fragments or as Fab fragments. Libraries from immunized sources provide high-affinity antibodies to the immunogen (e.g., a betaglycan, TGFβ1, TGFβ2, or TGFβ3 polypeptide) without the requirement of constructing hybridomas. Alternatively, the naive repertoire can be cloned (e.g., from human) to provide a single source of antibodies directed against a wide range of non-self and also self-antigens without any immunization as described by Griffiths et al. (1993) EMBO J, 12: 725-734. Finally, naive libraries can also be made synthetically by cloning un-rearranged V-gene segments from stem cells and using PCR primers containing random sequence to encode the highly variable CDR3 regions and to accomplish rearrangement in vitro, as described by Hoogenboom and Winter (1992) J. Mol. Biol., 227: 381-388. Patent publications describing human antibody phage libraries include, for example: U.S. Pat. No. 5,750,373, and U.S. Patent Publication Nos. 2005/0079574, 2005/0119455, 2005/0266000, 2007/0117126, 2007/0160598, 2007/0237764, 2007/0292936, and 2009/0002360.

In certain embodiments, an antibody provided herein is a multispecific antibody, for example, a bispecific antibody. Multispecific antibodies (typically monoclonal antibodies) have binding specificities for at least two different epitopes (e.g., two, three, four, five, or six or more) on one or more (e.g., two, three, four, five, six or more) antigens. Engineered antibodies with three or more functional antigen binding sites, including “octopus antibodies,” are also included herein (see, e.g., US 2006/0025576A1).

In certain embodiments, the antibodies disclosed herein are monoclonal antibodies. Monoclonal antibody refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present methods may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

For example, by using immunogens derived from a TβRII receptor polypeptide, anti-protein/anti-peptide antisera or monoclonal antibodies can be made by standard protocols [see, e.g., Antibodies: A Laboratory Manual (1988) ed. by Harlow and Lane, Cold Spring Harbor Press]. A mammal, such as a mouse, hamster, or rabbit can be immunized with an immunogenic form of the TβRII polypeptide, an antigenic fragment which is capable of eliciting an antibody response, or a fusion protein. Techniques for conferring immunogenicity on a protein or peptide include conjugation to carriers or other techniques well known in the art. An immunogenic portion of a TβRII polypeptide can be administered in the presence of adjuvant. The progress of immunization can be monitored by detection of antibody titers in plasma or serum. Standard ELISA or other immunoassays can be used with the immunogen as antigen to assess the levels of antibody production and/or level of binding affinity.

Following immunization of an animal with an antigenic preparation of TβRII polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique [see, e.g., Kohler and Milstein (1975) Nature, 256: 495-497], the human B cell hybridoma technique [see, e.g., Kozbar et al. (1983) Immunology Today, 4:72], and the EBV-hybridoma technique to produce human monoclonal antibodies [Cole et al. (1985) Monoclonal Antibodies and Cancer Therapy, Alan R. Liss, Inc. pp. 77-96]. Hybridoma cells can be screened immunochemically for production of antibodies specifically reactive with a TβRII polypeptide, and monoclonal antibodies isolated from a culture comprising such hybridoma cells.

In certain embodiments, one or more amino acid modifications may be introduced into the Fc region of an antibody provided herein thereby generating an Fc-region variant. The Fc-region variant may comprise a human Fc-region sequence (e.g., a human IgG1, IgG2, IgG3 or IgG4 Fc region) comprising an amino acid modification (e.g., a substitution, deletion, and/or addition) at one or more amino acid positions.

For example, the present disclosure contemplates an antibody variant that possesses some but not all effector functions, which make it a desirable candidate for applications in which the half-life of the antibody in vivo is important yet for which certain effector functions [e.g., complement-dependent cytotoxicity (CDC) and antibody-dependent cellular cytotoxicity (ADCC)] are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII FcR expression on hematopoietic cells is summarized in, for example, Ravetch and Kinet (1991) Annu. Rev. Immunol. 9:457-492. Non-limiting examples of in vitro assays to assess ADCC activity of a molecule of interest are described in U.S. Pat. No. 5,500,362; Hellstrom, I. et al. (1986) Proc. Nat'l Acad. Sci. USA 83:7059-7063; Hellstrom, I et al. (1985) Proc. Nat'l Acad. Sci. USA 82:1499-1502; U.S. Pat. No. 5,821,337; and Bruggemann, M. et al. (1987) J. Exp. Med. 166:1351-1361. Alternatively, non-radioactive assay methods may be employed (e.g., ACTI™, non-radioactive cytotoxicity assay for flow cytometry; CellTechnology, Inc. Mountain View, Calif.; and CytoTox 96 ® non-radioactive cytotoxicity assay, Promega, Madison, Wis.). Useful effector cells for such assays include peripheral blood mononuclear cells (PB MC) and natural killer (NK) cells. Alternatively, or additionally, ADCC activity of the molecule of interest may be assessed in vivo, for example, in an animal model such as that disclosed in Clynes et al. (1998) Proc. Nat'l Acad. Sci. USA 95:652-656. Clq binding assays may also be carried out to confirm that the antibody is unable to bind Clq and hence lacks CDC activity [see, e.g., Clq and C3c binding ELISA in WO 2006/029879 and WO 2005/100402]. To assess complement activation, a CDC assay may be performed [see, e.g., Gazzano-Santoro et al. (1996) J. Immunol. Methods 202:163; Cragg, M. S. et al. (2003) Blood 101:1045-1052; and Cragg, M. S, and M. J. Glennie (2004) Blood 103:2738-2743]. FcRn binding and in vivo clearance/half-life determinations can also be performed using methods known in the art [see, e.g., Petkova, S. B. et al. (2006) Int. Immunol. 18(12):1759-1769].

Antibodies of the present disclosure with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, it may be desirable to create cysteine-engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy-chain Fc region. Cysteine engineered antibodies may be generated as described, for example, in U.S. Pat. No. 7,521,541.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore™ binding assay, Biacore AB, Uppsala, Sweden), sandwich assays (e.g., the paramagnetic bead system of IGEN International, Inc., Gaithersburg, Maryland), western blots, immunoprecipitation assays, and immunohistochemistry.

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into, and/or substitutions of residues within, the amino acid sequences of the antibody and/or binding polypeptide. Any combination of deletion, insertion, and substitution can be made to arrive at the final construct, provided that the final construct possesses the desired characteristics, e.g., target-binding (TβRII, ALK5, betaglycan, TGFβ1, TGFβ2, and/or TGFβ3).

Alterations (e.g., substitutions) may be made in HVRs, for example, to improve antibody affinity. Such alterations may be made in HVR “hotspots,” i.e., residues encoded by codons that undergo mutation at high frequency during the somatic maturation process (see, e.g., Chowdhury (2008) Methods Mol. Biol. 207:179-196 (2008)), and/or SDRs (a-CDRs), with the resulting variant VH or VL being tested for binding affinity. Affinity maturation by constructing and reselecting from secondary libraries has been described in the art [see, e.g., Hoogenboom et al., in Methods in Molecular Biology 178:1-37, O'Brien et al., ed., Human Press, Totowa, N.J., (2001)]. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind to the antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two, or three amino acid substitutions.

A useful method for identification of residues or regions of the antibody and/or the binding polypeptide that may be targeted for mutagenesis is called “alanine scanning mutagenesis”, as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or binding polypeptide with antigen is affected. Further substitutions may be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues may be targeted or eliminated as candidates for substitution. Variants may be screened to determine whether they contain the desired properties.

Amino-acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include fusion of the N- or C-terminus of the antibody to an enzyme (e.g., for ADEPT) or a polypeptide which increases the serum half-life of the antibody.

In certain embodiments, an antibody and/or binding polypeptide provided herein may be further modified to contain additional non-proteinaceous moieties that are known in the art and readily available. The moieties suitable for derivatization of the antibody and/or binding polypeptide include but are not limited to water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions.

### 15. Small Molecule Antagonists

In certain aspects, a TβRII antagonist to be used in accordance with the methods and uses disclosed herein is a small molecule (a small molecule TβRII antagonist), or combination of small molecules. A small molecule TβRII antagonist may inhibit, for example, one or more TβRII ligands (e.g., TGFβ1, TGFβ2, and TGFβ3), TβRII receptor, TβRII-associated type I receptor (e.g., ALK5), TβRII-associated co-receptor (e.g., betaglycan), and/or downstream signaling component (e.g., Smad proteins). In some embodiments, the ability for a small molecule TβRII antagonist to inhibit signaling (e.g., Smad signaling) is determined in a cell-based assay including, for example, those described herein. A small molecule TβRII antagonist may be used alone or in combination with one or more additional active agents and/or supportive therapies to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In certain aspects, a small molecule TβRII antagonist inhibits at least TGFβ1 (e.g., inhibition of Smad signaling). Therefore, in some embodiments, a small molecule inhibitor of TGFβ1 binds to TGFβ1. In some embodiments, a small molecule inhibitor of TGFβ1 inhibits expression (e.g., transcription, translation, secretion, or combinations thereof) of TGFβ1. In some embodiments, a small molecule inhibitor of TGFβ1 further inhibits one or more of TGFβ2, TGFβ3, ALK5, and betaglycan. In some embodiments, a small molecule inhibitor of TGFβ1 does not inhibit or does not substantially inhibit TGFβ2. In some embodiments, a small molecule inhibitor of TGFβ1 further inhibits TGFβ3 but does not inhibit or does not substantially inhibit TGFβ2. In certain aspects, a small molecule TβRII antagonist inhibits at least TGFβ2. Therefore, in some embodiments, a small molecule inhibitor of TGFβ2 binds to TGFβ2. In some embodiments, a small molecule inhibitor of TGFβ2 inhibits expression (e.g., transcription, translation, secretion, or combinations thereof) of TGFβ2. In some embodiments, a small molecule inhibitor of TGFβ2 further inhibits one or more of TGFβ3, TGFβ1, ALK5, and betaglycan. In certain aspects, a small molecule TβRII antagonist inhibits at least TGFβ3. Therefore, in some embodiments, a small molecule inhibitor of TGFβ3 binds to TGFβ3. In some embodiments, a small molecule inhibitor of TGFβ3 inhibits expression (e.g., transcription, translation, secretion, or combinations thereof) of TGFβ3. In some embodiments, a small molecule inhibitor of TGFβ3 further inhibits one or more of TGFβ2, TGFβ1, ALK5, and betaglycan. In some embodiments, a small molecule inhibitor of TGFβ3 does not inhibit or does not substantially inhibit TGFβ2. In some embodiments, a small molecule inhibitor of TGFβ3 further inhibits TGFβ1 but does not inhibit or does not substantially inhibit TGFβ2. In certain aspects, a small molecule TβRII antagonist inhibits at least a TβRII receptor. Therefore, in some embodiments, a small molecule inhibitor of TβRII binds to a TβRII receptor. In some embodiments, a small molecule inhibitor of TβRII inhibits expression (e.g., transcription, translation, secretion, or combinations thereof) of a TβRII receptor. In some embodiments, a small molecule inhibitor of a TβRII receptor further inhibits one or more of TGFβ1, TGFβ2, TGFβ3, ALK5, and betaglycan. In certain aspects, a small molecule TβRII antagonist inhibits TGFβ1 from binding to a TβRII receptor. In certain aspects, a small molecule TβRII antagonist small molecule inhibits TGFβ2 from binding to a TβRII receptor. In certain aspects, a small molecule TβRII antagonist inhibits TGFβ3 from binding to a TβRII receptor. In certain aspects, a small molecule TβRII antagonist inhibits TGFβ1 and TGFβ3 from binding to a TβRII receptor. In certain aspects, a small molecule TβRII antagonist inhibits TGFβ1, TGFβ2, and TGFβ3 from binding to a TβRII receptor. In certain aspects, a small molecule TβRII antagonist inhibits TGFβ1 from binding to a TβRII receptor but does not inhibit or does not substantially inhibit TGFβ2 from binding to a TβRII receptor. In certain aspects, a small molecule TβRII antagonist inhibits TGFβ3 from binding to a TβRII receptor but does not inhibit or does not substantially inhibit TGFβ2 from binding to a TβRII receptor. In certain aspects, a small molecule TβRII antagonist inhibits TGFβ1 and TGFβ3 from binding to a TβRII receptor but does not inhibit or does not substantially inhibit TGFβ2 from binding to a TβRII receptor. In certain aspects, a small molecule TβRII antagonist inhibits at least ALK5. Therefore, in some embodiments, a small molecule inhibitor of ALK5 binds to ALK5. In some embodiments, a small molecule inhibitor of ALK5 inhibits expression (e.g., transcription, translation, secretion, or combinations thereof) of ALK5. In some embodiments, a small molecule inhibitor of ALK5 further inhibits one or more of TGFβ1, TGFβ2, TGFβ3, TβRII, and betaglycan. In certain aspects, a small molecule TβRII antagonist inhibits TGFβ1 from binding to ALK5. In certain aspects, a small molecule TβRII antagonist inhibits TGFβ2 from binding to ALK5. In certain aspects, a small molecule TβRII antagonist inhibits TGFβ3 from binding to ALK5. In certain aspects, a small molecule TβRII antagonist inhibits TGFβ1 and TGFβ3 from binding to ALK5. In certain aspects, a small molecule TβRII antagonist inhibits TGFβ1, TGFβ2, and TGFβ3 from binding to ALK5. In certain aspects, a small molecule TβRII antagonist inhibits TGFβ1 from binding to ALK5 but does not inhibit or does not substantially inhibit TGFβ2 from binding to ALK5. In certain aspects, a small molecule TβRII antagonist inhibits TGFβ3 from binding to ALK5 but does not inhibit or does not substantially inhibit TGFβ2 from binding to ALK5. In certain aspects, a small molecule TβRII antagonist inhibits TGFβ1 and TGFβ3 from binding to ALK5 but does not inhibit or does not substantially inhibit TGFβ2 from binding to ALK5. In certain aspects, a small molecule TβRII antagonist inhibits at least betaglycan. Therefore, in some embodiments, a small molecule inhibitor of betaglycan binds to betaglycan. In some embodiments, a small molecule inhibitor of betaglycan inhibits expression (e.g., transcription, translation, secretion, or combinations thereof) of betaglycan. In some embodiments, a small molecule inhibitor of betaglycan further inhibits one or more of TGFβ1, TGFβ2, TGFβ3, TβRII, and ALK5. In certain aspects, a small molecule TβRII antagonist inhibits TGFβ1 from binding to betaglycan. In certain aspects, a small molecule TβRII antagonist inhibits TGFβ2 from binding to betaglycan. In certain aspects, a small molecule TβRII antagonist small molecule inhibits TGFβ3 from binding to betaglycan. In certain aspects, a small molecule TβRII antagonist inhibits TGFβ1 and TGFβ3 from binding to betaglycan. In certain aspects, a small molecule TβRII antagonist inhibits TGFβ1, TGFβ2, and TGFβ3 from binding to betaglycan. In certain aspects, a small molecule TβRII antagonist inhibits TGFβ1 from binding to betaglycan but does not inhibit or does not substantially inhibit TGFβ2 from binding to betaglycan. In certain aspects, a small molecule TβRII antagonist inhibits TGFβ3 from binding to betaglycan but does not inhibit or does not substantially inhibit TGFβ2 from binding to betaglycan. In certain aspects, a small molecule TβRII antagonist inhibits TGFβ1 and TGFβ3 from binding to betaglycan but does not inhibit or does not substantially inhibit TGFβ2 from binding to betaglycan.

Small molecule TβRII antagonist can be direct or indirect inhibitors. For example, a small molecule TβRII antagonist, or combination of small molecules, may inhibit the expression (e.g., transcription, translation, cellular secretion, or combinations thereof) of at least one or more of TβRII, ALK5, betaglycan, TGFβ1, TGFβ2, TGFβ3, and/or one or more downstream TβRII signaling factors (Smads). Alternatively, a direct small molecule TβRII antagonist, or combination of small molecules, may directly bind to, for example, one or more of TβRII, ALK5, betaglycan, TGFβ1, TGFβ2, and TGFβ3 or one or more downstream TβRII signaling factors. Combinations of one or more indirect and one or more direct small molecule TβRII antagonist may be used in accordance with the methods and uses disclosed herein.

Binding organic small molecule antagonists of the present disclosure may be identified and chemically synthesized using known methodology (see, e.g., PCT Publication Nos. WO 00/00823 and WO 00/39585). In general, small molecule antagonists of the disclosure are usually less than about 2000 daltons in size, alternatively less than about 1500, 750, 500, 250 or 200 daltons in size, wherein such organic small molecules that are capable of binding, preferably specifically, to a polypeptide as described herein (e.g., TβRII, ALK5, betaglycan, TGFβ1, TGFβ2, and TGFβ3). Such small molecule antagonists may be identified without undue experimentation using well-known techniques. In this regard, it is noted that techniques for screening organic small molecule libraries for molecules that are capable of binding to a polypeptide target are well-known in the art (see, e.g., international patent publication Nos. WO00/00823 and WO00/39585).

Binding organic small molecules of the present disclosure may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, and acid chlorides.

### 16. Antagonist Polynucleotides

In certain aspects, a TβRII antagonist to be used in accordance with the methods and uses disclosed herein is a polynucleotide (a polynucleotide TβRII antagonist), or combination of polynucleotides. A polynucleotide TβRII antagonist may inhibit, for example, one or more TβRII ligands (e.g., TGFβ1, TGFβ2, and TGFβ3), TβRII receptor, TβRII-associated type I receptor (e.g., ALK5), TβRII-associated co-receptor (e.g., betaglycan), and/or downstream signaling component (e.g., Smad proteins). In some embodiments, the ability for a polynucleotide TβRII antagonist to inhibit signaling (e.g., Smad signaling) is determined in a cell-based assay including, for example, those described herein. A polynucleotide TβRII antagonist may be used alone or in combination with one or more additional active agents and/or supportive therapies to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

In certain aspects, a polynucleotide TβRII antagonist inhibits at least TGFβ1 (e.g., inhibition of Smad signaling). Therefore, in some embodiments, a polynucleotide inhibitor of TGFβ1 binds to TGFβ1. In some embodiments, a polynucleotide inhibitor of TGFβ1 inhibits expression (e.g., transcription, translation, secretion, or combinations thereof) of TGFβ1. In some embodiments, a polynucleotide inhibitor of TGFβ1 further inhibits one or more of TGFβ2, TGFβ3, ALK5, and betaglycan. In some embodiments, a polynucleotide inhibitor of TGFβ1 does not inhibit or does not substantially inhibit TGFβ2. In some embodiments, a polynucleotide inhibitor of TGFβ1 further inhibits TGFβ3 but does not inhibit or does not substantially inhibit TGFβ2. In certain aspects, a polynucleotide TβRII antagonist inhibits at least TGFβ2. Therefore, in some embodiments, a polynucleotide inhibitor of TGFβ2 binds to TGFβ2. In some embodiments, a polynucleotide inhibitor of TGFβ2 inhibits expression (e.g., transcription, translation, secretion, or combinations thereof) of TGFβ2. In some embodiments, a polynucleotide inhibitor of TGFβ2 further inhibits one or more of TGFβ3, TGFβ1, ALK5, and betaglycan. In certain aspects, a polynucleotide TβRII antagonist inhibits at least TGFβ3. Therefore, in some embodiments, a polynucleotide inhibitor of TGFβ3 binds to TGFβ3. In some embodiments, a polynucleotide inhibitor of TGFβ3 inhibits expression (e.g., transcription, translation, secretion, or combinations thereof) of TGFβ3. In some embodiments, a polynucleotide inhibitor of TGFβ3 further inhibits one or more of TGFβ2, TGFβ1, ALK5, and betaglycan. In some embodiments, a polynucleotide inhibitor of TGFβ3 does not inhibit or does not substantially inhibit TGFβ2. In some embodiments, a polynucleotide inhibitor of TGFβ3 further inhibits TGFβ1 but does not inhibit or does not substantially inhibit TGFβ2. In certain aspects, a polynucleotide TβRII antagonist inhibits at least a TβRII receptor. Therefore, in some embodiments, a polynucleotide inhibitor of TβRII binds to a TβRII receptor. In some embodiments, a polynucleotide inhibitor of TβRII inhibits expression (e.g., transcription, translation, secretion, or combinations thereof) of a TβRII receptor. In some embodiments, a polynucleotide inhibitor of a TβRII receptor further inhibits one or more of TGFβ1, TGFβ2, TGFβ3, ALK5, and betaglycan. In some embodiments, a polynucleotide TβRII antagonist inhibits TGFβ1 from binding to a TβRII receptor. In some embodiments, a polynucleotide TβRII antagonist inhibits TGFβ2 from binding to a TβRII receptor. In some embodiments, a polynucleotide TβRII antagonist inhibits TGFβ3 from binding to a TβRII receptor. In some embodiments, a polynucleotide TβRII antagonist inhibits TGFβ1 and TGFβ3 from binding to a TβRII receptor. In some embodiments, a polynucleotide TβRII antagonist inhibits TGFβ1, TGFβ2, and TGFβ3 from binding to a TβRII receptor. In some embodiments, a polynucleotide TβRII antagonist inhibits TGFβ1 from binding to a TβRII receptor but does not inhibit or does not substantially inhibit TGFβ2 from binding to a TβRII receptor. In some embodiments, a polynucleotide TβRII antagonist inhibits TGFβ3 from binding to a TβRII receptor but does not inhibit or does not substantially inhibit TGFβ2 from binding to a TβRII receptor. In some embodiments, a polynucleotide TβRII antagonist inhibits TGFβ1 and TGFβ3 from binding to a TβRII receptor but does not inhibit or does not substantially inhibit TGFβ2 from binding to a TβRII receptor. In certain aspects, a polynucleotide TβRII antagonist inhibits at least ALK5. Therefore, in some embodiments, a polynucleotide inhibitor of ALK5 binds to ALK5. In some embodiments, a polynucleotide inhibitor of ALK5 inhibits expression (e.g., transcription, translation, secretion, or combinations thereof) of ALK5. In some embodiments, a polynucleotide inhibitor of ALK5 further inhibits one or more of TGFβ1, TGFβ2, TGFβ3, TβRII, and betaglycan. In some embodiments, a polynucleotide TβRII antagonist inhibits TGFβ1 from binding to ALK5. In some embodiments, a polynucleotide TβRII antagonist inhibits TGFβ2 from binding to ALK5. In some embodiments, a polynucleotide TβRII antagonist inhibits TGFβ3 from binding to ALK5. In some embodiments, a polynucleotide TβRII antagonist inhibits TGFβ1 and TGFβ3 from binding to ALK5. In some embodiments, a polynucleotide TβRII antagonist inhibits TGFβ1, TGFβ2, and TGFβ3 from binding to ALK5. In some embodiments, a polynucleotide TβRII antagonist inhibits TGFβ1 from binding to ALK5 but does not inhibit or does not substantially inhibit TGFβ2 from binding to ALK5. In some embodiments, a polynucleotide TβRII antagonist inhibits TGFβ3 from binding to ALK5 but does not inhibit or does not substantially inhibit TGFβ2 from binding to ALK5. In some embodiments, a polynucleotide TβRII antagonist inhibits TGFβ1 and TGFβ3 from binding to ALK5 but does not inhibit or does not substantially inhibit TGFβ2 from binding to ALK5. In certain aspects, a polynucleotide TβRII antagonist inhibits at least betaglycan. Therefore, in some embodiments, a polynucleotide inhibitor of betaglycan binds to betaglycan. In some embodiments, a polynucleotide inhibitor of betaglycan inhibits expression (e.g., transcription, translation, secretion, or combinations thereof) of betaglycan. In some embodiments, a polynucleotide inhibitor of betaglycan further inhibits one or more of TGFβ1, TGFβ2, TGFβ3, TβRII, and ALK5. In some embodiments, a polynucleotide TβRII antagonist inhibits TGFβ1 from binding to betaglycan. In some embodiments, a polynucleotide TβRII antagonist inhibits TGFβ2 from binding to betaglycan. In some embodiments, a polynucleotide TβRII antagonist inhibits TGFβ3 from binding to betaglycan. In some embodiments, a polynucleotide TβRII antagonist inhibits TGFβ1 and TGFβ3 from binding to betaglycan. In some embodiments, a polynucleotide TβRII antagonist inhibits TGFβ1, TGFβ2, and TGFβ3 from binding to betaglycan. In some embodiments, a polynucleotide TβRII antagonist inhibits TGFβ1 from binding to betaglycan but does not inhibit or does not substantially inhibit TGFβ2 from binding to betaglycan. In some embodiments, a polynucleotide TβRII antagonist inhibits TGFβ3 from binding to betaglycan but does not inhibit or does not substantially inhibit TGFβ2 from binding to betaglycan. In some embodiments, a polynucleotide TβRII antagonist inhibits TGFβ1 and TGFβ3 from binding to betaglycan but does not inhibit or does not substantially inhibit TGFβ2 from binding to betaglycan.

The polynucleotide antagonists of the present disclosure may be an antisense nucleic acid, an RNAi molecule [e.g., small interfering RNA (siRNA), small-hairpin RNA (shRNA), microRNA (miRNA)], an aptamer and/or a ribozyme. The nucleic acid and amino acid sequences of human TβRII, ALK5, betaglycan, TGFβ1, TGFβ2, and TGFβ3 are known in the art and thus polynucleotide antagonists for use in accordance with methods of the present disclosure may be routinely made by the skilled artisan based on the knowledge in the art and teachings provided herein.

For example, antisense technology can be used to control gene expression through antisense DNA or RNA, or through triple-helix formation. Antisense techniques are discussed, for example, in Okano (1991) J. Neurochem. 56:560; Oligodeoxynucleotides as Antisense Inhibitors of Gene Expression, CRC Press, Boca Raton, Fla. (1988). Triple helix formation is discussed in, for instance, Cooney et al. (1988) Science 241:456; and Dervan et al., (1991) Science 251:1300. The methods are based on binding of a polynucleotide to a complementary DNA or RNA. In some embodiments, the antisense nucleic acids comprise a single-stranded RNA or DNA sequence that is complementary to at least a portion of an RNA transcript of a desired gene. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double-stranded antisense nucleic acids of a gene disclosed herein, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with an RNA it may contain, and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, for example, the 5′-untranslated sequence up to and including the AUG initiation codon, should work most efficiently at inhibiting translation. However, sequences complementary to the 3′-untranslated sequences of mRNAs have been shown to be effective at inhibiting translation of mRNAs as well [see, e.g., Wagner, R., (1994) Nature 372:333-335]. Thus, oligonucleotides complementary to either the 5′- or 3′-untranslated, noncoding regions of a gene of the disclosure, could be used in an antisense approach to inhibit translation of an endogenous mRNA. Polynucleotides complementary to the 5′-untranslated region of the mRNA should include the complement of the AUG start codon. Antisense polynucleotides complementary to mRNA coding regions are less efficient inhibitors of translation but could be used in accordance with the methods of the present disclosure. Whether designed to hybridize to the 5′-untranslated, 3′-untranslated, or coding regions of an mRNA of the disclosure, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides.

In one embodiment, the antisense nucleic acid of the present disclosure is produced intracellularly by transcription from an exogenous sequence. For example, a vector or a portion thereof, is transcribed, producing an antisense nucleic acid (RNA) of a gene of the disclosure. Such a vector would contain a sequence encoding the desired antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding desired genes of the instant disclosure, or fragments thereof, can be by any promoter known in the art to act in vertebrate, preferably human cells. Such promoters can be inducible or constitutive. Such promoters include, but are not limited to, the SV40 early promoter region [see, e.g., Benoist and Chambon (1981) Nature 29:304-310], the promoter contained in the 3′ long terminal repeat of Rous sarcoma virus [see, e.g., Yamamoto et al. (1980) Cell 22:787-797], the herpes thymidine promoter [see, e.g., Wagner et al. (1981) Proc. Natl. Acad. Sci. U.S.A. 78:1441-1445], and the regulatory sequences of the metallothionein gene [see, e.g., Brinster, et al. (1982) Nature 296:39-42].

In some embodiments, the polynucleotide antagonists are interfering RNA or RNAi molecules that target the expression of one or more genes. RNAi refers to the expression of an RNA which interferes with the expression of the targeted mRNA. Specifically, RNAi silences a targeted gene via interacting with the specific mRNA through a siRNA (small interfering RNA). The ds RNA complex is then targeted for degradation by the cell. An siRNA molecule is a double-stranded RNA duplex of 10 to 50 nucleotides in length, which interferes with the expression of a target gene which is sufficiently complementary (e.g. at least 80% identity to the gene). In some embodiments, the siRNA molecule comprises a nucleotide sequence that is at least 85, 90, 95, 96, 97, 98, 99, or 100% identical to the nucleotide sequence of the target gene.

Additional RNAi molecules include short-hairpin RNA (shRNA); also short-interfering hairpin and microRNA (miRNA). The shRNA molecule contains sense and antisense sequences from a target gene connected by a loop. The shRNA is transported from the nucleus into the cytoplasm, and it is degraded along with the mRNA. Pol III or U6 promoters can be used to express RNAs for RNAi. Paddison et al. [Genes & Dev. (2002) 16:948-958, 2002] have used small RNA molecules folded into hairpins as a means to effect RNAi. Accordingly, such short hairpin RNA (shRNA) molecules are also advantageously used in the methods described herein. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the double-stranded RNA (dsRNA) products of the DICER RNase and, in any event, have the same capacity for inhibiting expression of a specific gene. The shRNA can be expressed from a lentiviral vector. An miRNA is a single-stranded RNA of about 10 to 70 nucleotides in length that are initially transcribed as pre-miRNA characterized by a “stem-loop” structure and which are subsequently processed into mature miRNA after further processing through the RISC.

Molecules that mediate RNAi, including without limitation siRNA, can be produced in vitro by chemical synthesis (Hohjoh, FEBS Lett 521:195-199, 2002), hydrolysis of dsRNA (Yang et al., Proc Natl Acad Sci USA 99:9942-9947, 2002), by in vitro transcription with T7 RNA polymerase (Donzeet et al., Nucleic Acids Res 30: e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947, 2002).

According to another aspect, the disclosure provides polynucleotide antagonists including but not limited to, a decoy DNA, a double-stranded DNA, a single-stranded DNA, a complexed DNA, an encapsulated DNA, a viral DNA, a plasmid DNA, a naked RNA, an encapsulated RNA, a viral RNA, a double-stranded RNA, a molecule capable of generating RNA interference, or combinations thereof.

In some embodiments, the polynucleotide antagonists of the disclosure are aptamers. Aptamers are nucleic acid molecules, including double-stranded DNA and single-stranded RNA molecules, which bind to and form tertiary structures that specifically bind to a target molecule, such as a TβRII, betaglycan, TGFβ1, TGFβ2, and TGFβ3 polypeptide. The generation and therapeutic use of aptamers are well established in the art. See, e.g., U.S. Pat. No. 5,475,096. Additional information on aptamers can be found in U.S. Patent Application Publication No. 20060148748. Nucleic acid aptamers are selected using methods known in the art, for example via the Systematic Evolution of Ligands by Exponential Enrichment (SELEX) process. SELEX is a method for the in vitro evolution of nucleic acid molecules with highly specific binding to target molecules as described in, e.g., U.S. Pat. Nos. 5,580,737, 5,567,588, 5,707,796, 5,763,177, 6,011,577, and 6,699,843. Another screening method to identify aptamers is described in U.S. Pat. No. 5,270,163. The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets. The SELEX method involves selection from a mixture of candidate oligonucleotides and step-wise iterations of binding, partitioning and amplification, using the same general selection scheme, to achieve desired binding affinity and selectivity. Starting from a mixture of nucleic acids, which can comprise a segment of randomized sequence, the SELEX method includes steps of contacting the mixture with the target under conditions favorable for binding; partitioning unbound nucleic acids from those nucleic acids which have bound specifically to target molecules; dissociating the nucleic acid-target complexes; amplifying the nucleic acids dissociated from the nucleic acid-target complexes to yield a ligand enriched mixture of nucleic acids. The steps of binding, partitioning, dissociating and amplifying are repeated through as many cycles as desired to yield highly specific high affinity nucleic acid ligands to the target molecule.

Typically, such binding molecules are separately administered to the animal [see, e.g., O'Connor (1991) J. Neurochem. 56:560], but such binding molecules can also be expressed in vivo from polynucleotides taken up by a host cell and expressed in vivo [see, e.g., Oligodeoxynucleotides as Antisense Inhibitors of Gene Expression, CRC Press, Boca Raton, Fla. (1988)].

### 17. Screening Assays

In certain aspects, the present invention relates to the use of TβRII polypeptides (e.g., soluble TβRII polypeptides) to identify compounds (agents) which are agonist or antagonists of the TGFβ1, TGFβ3 and TβRII signaling pathway. Compounds identified through this screening can be tested to assess their ability to modulate TGFβ1 and TGFβ3 signaling activity in vitro. Specifically, compounds identified through this screening can be tested to assess their ability to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), in a subject in need thereof. Accordingly, these compounds can further be tested in animal models to assess their ability to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD).

There are numerous approaches to screening for therapeutic agents for treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), by targeting TGFβ1, TGFβ3 and TβRII polypeptides. In certain embodiments, high-throughput screening of compounds can be carried out to identify agents that perturb TGFβ1, TGFβ3 or TβRII-mediated cell signaling. In certain embodiments, the assay is carried out to screen and identify compounds that specifically inhibit or reduce binding of a TβRII polypeptide to TGFβ1 or TGFβ3. Alternatively, the assay can be used to identify compounds that enhance binding of a TβRII polypeptide to TGFβ1 or TGFβ3. In a further embodiment, the compounds can be identified by their ability to interact with a TGFβ1, TGFβ3 or TβRII polypeptide.

A variety of assay formats will suffice, and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as therapeutic agents to treat chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds of the invention can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatable crosslinkers or any combinations thereof.

In many drug screening programs, which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TβRII polypeptide and TGFβ1 or TGFβ3.

Merely to illustrate, in an exemplary screening assay of the present invention, the compound of interest is contacted with an isolated and purified TβRII polypeptide which is ordinarily capable of binding to TGFβ1 or TGFβ3. To the mixture of the compound and TβRII polypeptide is then added a composition containing a TβRII ligand. Detection and quantification of TPβII/TGFβ1 or TPβII/TGFβ3 complexes provides a means for determining the compound's efficacy at inhibiting (or potentiating) complex formation between the TβRII polypeptide and TGFβ1 or TGFβ3. The efficacy of the compound can be assessed by generating dose response curves from data obtained using various concentrations of the test compound. Moreover, a control assay can also be performed to provide a baseline for comparison. For example, in a control assay, isolated and a purified TGFβ1 or TGFβ3 is added to a composition containing the TβRII polypeptide, and the formation of TPβII/TGFβ1 or TPβII/TGFβ3 complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between the TβRII polypeptide and TGFβ1 or TGFβ3 may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled TβRII polypeptide or TGFβ1 or TGFβ3, by immunoassay, or by chromatographic detection.

In certain embodiments, the present invention contemplates the use of fluorescence polarization assays and fluorescence resonance energy transfer (FRET) assays in measuring, either directly or indirectly, the degree of interaction between a TβRII polypeptide and its binding protein. Further, other modes of detection, such as those based on optical waveguides (PCT Publication WO 96/26432 and U.S. Pat. No. 5,677,196), surface plasmon resonance (SPR), surface charge sensors, and surface force sensors, are compatible with many embodiments of the invention.

Moreover, the present invention contemplates the use of an interaction trap assay, also known as the “two hybrid assay,” for identifying agents that disrupt or potentiate interaction between a TβRII polypeptide and its binding protein. See for example, U.S. Pat. No. 5,283,317 Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present invention contemplates the use of reverse two hybrid systems to identify compounds (e.g., small molecules or peptides) that dissociate interactions between a TβRII polypeptide and its binding protein. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

In certain embodiments, the subject compounds are identified by their ability to interact with a TβRII or TGFβ1 or TGFβ3 polypeptide of the invention. The interaction between the compound and the TβRII or TGFβ1 or TGFβ3 polypeptide may be covalent or non-covalent. For example, such interaction can be identified at the protein level using in vitro biochemical methods, including photo-crosslinking, radiolabeled ligand binding, and affinity chromatography (Jakoby W B et al., 1974, Methods in Enzymology 46: 1). In certain cases, the compounds may be screened in a mechanism based assay, such as an assay to detect compounds which bind to a TGFβ1 or TGFβ3 or TβRII polypeptide. This may include a solid-phase or fluid-phase binding event. Alternatively, the gene encoding a TGFβ1 or TGFβ3 or TβRII polypeptide can be transfected with a reporter system (e.g., β-galactosidase, luciferase, or green fluorescent protein) into a cell and screened against the library preferably by a high-throughput screening or with individual members of the library. Other mechanism-based binding assays may be used, for example, binding assays which detect changes in free energy. Binding assays can be performed with the target fixed to a well, bead or chip or captured by an immobilized antibody or resolved by capillary electrophoresis. The bound compounds may be detected usually using colorimetric or fluorescence or surface plasmon resonance.

In certain aspects, the present invention provides methods and agents for modulating (stimulating or inhibiting) TGFβ1- or TGFβ3-mediated cell signaling. Therefore, any compound identified can be tested in whole cells or tissues, in vitro or in vivo, to confirm their ability to modulate TGFβ1 or TGFβ3 signaling. Various methods known in the art can be utilized for this purpose.

### 18. Pharmaceutical Compositions

The therapeutic agents described herein (e.g., TβRII fusion antagonists including but not limited to TβRII polypeptides) may be formulated into pharmaceutical compositions. Pharmaceutical compositions for use in accordance with the present disclosure may be formulated in conventional manner using one or more physiologically acceptable carriers or excipients. Such formulations will generally be substantially pyrogen-free, in compliance with most regulatory requirements.

In some embodiments, the therapeutic method of the disclosure includes reconstituting a lyophilized powder of a TβRII antagonist of the present disclosure. In some embodiments, the lyophilized TβRII antagonist is reconstituted in sterile water. In some embodiments, a single use vial of lyophilized TβRII antagonist comprises 50 mg of the TβRII antagonist. In some embodiments, the lyophilized TβRII antagonist is reconstituted for administration by subcutaneous injection.

In certain embodiments, the therapeutic method of the disclosure includes administering the composition systemically, or locally as an implant or device. When administered, the therapeutic composition for use in this disclosure is in a pyrogen-free, physiologically acceptable form. Therapeutically useful agents other than the TβRII antagonists which may also optionally be included in the composition as described above, may be administered simultaneously or sequentially with the subject compounds (e.g., TβRII antagonists) in the methods disclosed herein.

Typically, protein therapeutic agents disclosed herein will be administered parentally, and/or particularly intravenously or subcutaneously. In some embodiments, TβRII antagonists of the present disclosure are administered subcutaneously. Pharmaceutical compositions suitable for parenteral administration may comprise one or more TβRII antagonists in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and/or nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and/or injectable organic esters, such as ethyl oleate. Proper fluidity can be maintained, for example, by the use of coating materials, such as lecithin, by the maintenance of the required particle size in the case of dispersions, and by the use of surfactants.

The compositions and/or formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., TβRII antagonists) to a target tissue site, providing a structure for the developing tissue and/or optimally capable of being resorbed into the body. For example, the matrix may provide slow release of the TβRII antagonists. Such matrices may be formed of materials presently in use for other implanted medical applications.

The choice of matrix material is based on biocompatibility, biodegradability, mechanical properties, cosmetic appearance and/or interface properties. The particular application of the subject compositions will define the appropriate formulation. Potential matrices for the compositions may be biodegradable and/or chemically defined calcium sulfate, tricalcium phosphate, hydroxyapatite, polylactic acid and/or polyanhydrides. Other potential materials are biodegradable and/or biologically well defined, such as bone or dermal collagen. Further matrices are comprised of pure proteins or extracellular matrix components. Other potential matrices are non-biodegradable and/or chemically defined, such as sintered hydroxyapatite, bioglass, aluminates, or other ceramics. Matrices may be comprised of combinations of any of the above mentioned types of material, such as polylactic acid and/or hydroxyapatite or collagen and/or tricalcium phosphate. The bioceramics may be altered in composition, such as in calcium-aluminate-phosphate and processing to alter pore size, particle size, particle shape, and/or biodegradability.

In certain embodiments, methods of the disclosure can be administered for orally, e.g., in the form of capsules, cachets, pills, tablets, lozenges (using a flavored basis, usually sucrose and/or acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and/or glycerin, or sucrose and/or acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, and/or/or any of the following: (1) fillers or extenders, such as starches, lactose, sucrose, glucose, mannitol, and/or/or silicic acid; (2) binders, such as, for example, carboxymethylcellulose, alginates, gelatin, polyvinyl pyrrolidone, sucrose, and/or/or acacia; (3) humectants, such as glycerol; (4) disintegrating agents, such as agar-agar, calcium carbonate, potato or tapioca starch, alginic acid, certain silicates, and/or sodium carbonate; (5) solution retarding agents, such as paraffin; (6) absorption accelerators, such as quaternary ammonium compounds; (7) wetting agents, such as, for example, cetyl alcohol and/or glycerol monostearate; (8) absorbents, such as kaolin and/or bentonite clay; (9) lubricants, such a talc, calcium stearate, magnesium stearate, solid polyethylene glycols, sodium lauryl sulfate, and/or mixtures thereof; and/or (10) coloring agents. In the case of capsules, tablets and/or pills, the pharmaceutical compositions may also comprise buffering agents. Solid compositions of a similar type may also be employed as fillers in soft and/or hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and/or elixirs. In addition to the active ingredient, the liquid dosage forms may contain inert diluents commonly used in the art, such as water or other solvents, solubilizing agents and/or emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and/or sesame oils), glycerol, tetrahydrofuryl alcohol, polyethylene glycols and/or fatty acid esters of sorbitan, and/or mixtures thereof. Besides inert diluents, the oral compositions can also include adjuvants such as wetting agents, emulsifying and/or suspending agents, sweetening, flavoring, coloring, perfuming, and/or preservative agents.

Suspensions, in addition to the active compounds, may contain suspending agents such as ethoxylated isostearyl alcohols, polyoxyethylene sorbitol, and/or sorbitan esters, microcrystalline cellulose, aluminum metahydroxide, bentonite, agar-agar and/or tragacanth, and/or mixtures thereof.

The compositions of the invention may also contain adjuvants, such as preservatives, wetting agents, emulsifying agents and/or dispersing agents. Prevention of the action of microorganisms may be ensured by the inclusion of various antibacterial and/or antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and/or gelatin.

It is understood that the dosage regimen will be determined by the attending physician considering various factors which modify the action of the subject compounds of the disclosure (e.g., TβRII antagonists). The various factors include, but are not limited to, the patient's age, sex, and/or diet, the severity disease, time of administration, and/or other clinical factors. Optionally, the dosage may vary with the type of matrix used in the reconstitution and/or the types of compounds in the composition. The addition of other known growth factors to the final composition, may also affect the dosage. Progress can be monitored by periodic assessment of bone growth and/or repair, for example, X-rays (including DEXA), histomorphometric determinations, and/or tetracycline labeling.

In some embodiments, one or more TβRII antagonists of the present disclosure are administered in one or more doses. In some embodiments, a dose of one or more TβRII antagonists comprises 0.05 mg/kg of the antagonists. In some embodiments, a dose of one or more TβRII antagonists comprises 0.10 mg/kg of the antagonists. In some embodiments, a dose of one or more TβRII antagonists comprises 0.15 mg/kg of the antagonists. In some embodiments, a dose of one or more TβRII antagonists comprises 0.20 mg/kg of the antagonists. In some embodiments, a dose of one or more TβRII antagonists comprises 0.25 mg/kg of the antagonists. In some embodiments, a dose of one or more TβRII antagonists comprises 0.50 mg/kg of the antagonists. In some embodiments, a dose of one or more TβRII antagonists comprises 0.75 mg/kg of the antagonists. In some embodiments, a dose of one or more TβRII antagonists comprises 1.00 mg/kg of the antagonists. In some embodiments, a dose of one or more TβRII antagonists comprises 1.25 mg/kg of the antagonists. In some embodiments, a dose of one or more TβRII antagonists comprises 1.50 mg/kg of the antagonists. In some embodiments, a dose of one or more TβRII antagonists comprises 1.75 mg/kg of the antagonists. In some embodiments, a dose of one or more TβRII antagonists comprises 2.00 mg/kg of the antagonists. In some embodiments, a dose of one or more TβRII antagonists comprises 2.25 mg/kg of the antagonists. In some embodiments, a dose of one or more TβRII antagonists comprises 2.50 mg/kg of the antagonists. In some embodiments, a dose of one or more TβRII antagonists comprises 2.75 mg/kg of the antagonists. In some embodiments, a dose of one or more TβRII antagonists comprises 3.00 mg/kg of the antagonists. In some embodiments, a dose of one or more TβRII antagonists comprises 3.25 mg/kg of the antagonists. In some embodiments, a dose of one or more TβRII antagonists comprises 3.50 mg/kg of the antagonists. In some embodiments, a dose of one or more TβRII antagonists comprises 3.75 mg/kg of the antagonists. In some embodiments, a dose of one or more TβRII antagonists comprises 4.00 mg/kg of the antagonists. In some embodiments, a dose of one or more TβRII antagonists comprises 4.25 mg/kg of the antagonists. In some embodiments, a dose of one or more TβRII antagonists comprises 4.50 mg/kg of the antagonists. In some embodiments, a dose of one or more TβRII antagonists comprises 4.75 mg/kg of the antagonists. In some embodiments, a dose of one or more TβRII antagonists comprises 5.00 mg/kg of the antagonists. In some embodiments, a dose of one or more TβRII antagonists comprises 5.25 mg/kg of the antagonists. In some embodiments, a dose of one or more TβRII antagonists comprises 5.50 mg/kg of the antagonists. In some embodiments, a dose of one or more TβRII antagonists comprises 5.75 mg/kg of the antagonists. In some embodiments, a dose of one or more TβRII antagonists comprises 6.0 mg/kg of the antagonists. In some embodiments, a dose of one or more TβRII antagonists comprises 6.25 mg/kg of the antagonists. In some embodiments, a dose of one or more TβRII antagonists comprises 6.50 mg/kg of the antagonists. In some embodiments, a dose of one or more TβRII antagonists comprises 6.75 mg/kg of the antagonists. In some embodiments, a dose of one or more TβRII antagonists comprises 7.0 mg/kg of the antagonists.

In some embodiments, a dose of one or more TβRII antagonists comprises no more than 0.05 mg/kg of the antagonists. In some embodiments, a dose of one or more TβRII antagonists comprises no more than 0.10 mg/kg of the antagonists. In some embodiments, a dose of one or more TβRII antagonists comprises no more than 0.15 mg/kg of the antagonists. In some embodiments, a dose of one or more TβRII antagonists comprises no more than 0.20 mg/kg of the antagonists. In some embodiments, a dose of one or more TβRII antagonists comprises no more than 0.25 mg/kg of the antagonists. In some embodiments, a dose of one or more TβRII antagonists comprises no more than 0.50 mg/kg of the antagonists. In some embodiments, a dose of one or more TβRII antagonists comprises no more than 0.75 mg/kg of the antagonists. In some embodiments, a dose of one or more TβRII antagonists comprises no more than 1.00 mg/kg of the antagonists. In some embodiments, a dose of one or more TβRII antagonists comprises no more than 1.25 mg/kg of the antagonists. In some embodiments, a dose of one or more TβRII antagonists comprises no more than 1.50 mg/kg of the antagonists. In some embodiments, a dose of one or more TβRII antagonists comprises no more than 1.75 mg/kg of the antagonists. In some embodiments, a dose of one or more TβRII antagonists comprises no more than 2.0 mg/kg of the antagonists. In some embodiments, a dose of one or more TβRII antagonists comprises no more than 2.25 mg/kg of the antagonists. In some embodiments, a dose of one or more TβRII antagonists comprises no more than 2.50 mg/kg of the antagonists. In some embodiments, a dose of one or more TβRII antagonists comprises no more than 2.75 mg/kg of the antagonists. In some embodiments, a dose of one or more TβRII antagonists comprises no more than 3.0 mg/kg of the antagonists. In some embodiments, a dose of one or more TβRII antagonists comprises no more than 3.5 mg/kg of the antagonists. In some embodiments, a dose of one or more TβRII antagonists comprises no more than 4.0 mg/kg of the antagonists. In some embodiments, a dose of one or more TβRII antagonists comprises no more than 4.50 mg/kg of the antagonists. In some embodiments, a dose of one or more TβRII antagonists comprises no more than 5.0 mg/kg of the antagonists. In some embodiments, a dose of one or more TβRII antagonists comprises no more than 5.50 mg/kg of the antagonists. In some embodiments, a dose of one or more TβRII antagonists comprises no more than 6.0 mg/kg of the antagonists. In some embodiments, a dose of one or more TβRII antagonists comprises no more than 6.50 mg/kg of the antagonists. In some embodiments, a dose of one or more TβRII antagonists comprises no more than 7.0 mg/kg of the antagonists.

In some embodiments, a dose of one or more TβRII antagonists comprises at least mg/kg of the antagonists. In some embodiments, a dose of one or more TβRII antagonists comprises at least 0.10 mg/kg of the antagonists. In some embodiments, a dose of one or more TβRII antagonists comprises at least 0.15 mg/kg of the antagonists. In some embodiments, a dose of one or more TβRII antagonists comprises at least 0.20 mg/kg of the antagonists. In some embodiments, a dose of one or more TβRII antagonists comprises at least 0.25 mg/kg of the antagonists. In some embodiments, a dose of one or more TβRII fusion antagonists comprises between about 0.25 mg/to about 4.50 mg/kg of the antagonists. In some embodiments, a dose of one or more TβRII fusion antagonists comprises between about 0.05 mg/to about 6.0 mg/kg of the antagonists. In some embodiments, a dose of one or more TβRII fusion antagonists comprises between about 0.25 mg/to about 6.0 mg/kg of the antagonists. In some embodiments, a dose of one or more TβRII fusion antagonists comprises between about 0.75 mg/to about 6.0 mg/kg of the antagonists. In some embodiments, a dose of one or more TβRII fusion antagonists comprises between about 0.75 mg/to about 2.25 mg/kg of the antagonists. In some embodiments, a dose of one or more TβRII fusion antagonists comprises between about 1.25 mg/to about 6.0 mg/kg of the antagonists. In some embodiments, a dose of one or more TβRII fusion antagonists comprises between about 1.25 mg/to about 2.0 mg/kg of the antagonists. In some embodiments, a dose of one or more TβRII fusion antagonists comprises between about 1.50 mg/to about 4.50 mg/kg of the antagonists. In some embodiments, a dose of one or more TβRII fusion antagonists comprises between about 2.5 mg/to about 4.0 mg/kg of the antagonists. In some embodiments, a dose of one or more TβRII fusion antagonists comprises between about 3.0 mg/to about 6.0 mg/kg of the antagonists. In some embodiments, a dose of one or more TβRII fusion antagonists comprises between about 4.0 mg/to about 6.0 mg/kg of the antagonists.

In some embodiments, a dose of one or more TβRII antagonists of the present disclosure is administered once every day. In some embodiments, a dose of one or more TβRII antagonists of the present disclosure is administered once every two days. In some embodiments, a dose of one or more TβRII antagonists of the present disclosure is administered once every three days. In some embodiments, a dose of one or more TβRII antagonists of the present disclosure is administered once every four days. In some embodiments, a dose of one or more TβRII antagonists of the present disclosure is administered once every five days. In some embodiments, a dose of one or more TβRII antagonists of the present disclosure is administered once every six days. In some embodiments, a dose of one or more TβRII antagonists of the present disclosure is administered once every week. In some embodiments, a dose of one or more TβRII antagonists of the present disclosure is administered once every two weeks. In some embodiments, a dose of one or more TβRII antagonists of the present disclosure is administered once every three weeks. In some embodiments, a dose of one or more TβRII antagonists of the present disclosure is administered once every four weeks. In some embodiments, a dose of one or more TβRII antagonists of the present disclosure is administered once every five weeks. In some embodiments, a dose of one or more TβRII antagonists of the present disclosure is administered once every six weeks. In some embodiments, a dose of one or more TβRII antagonists of the present disclosure is administered once every seven weeks. In some embodiments, a dose of one or more TβRII antagonists of the present disclosure is administered once every eight weeks. In some embodiments, a dose of one or more TβRII antagonists of the present disclosure is administered once every other week. In some embodiments, a dose of one or more TβRII antagonists of the present disclosure is administered once every month. In some embodiments, a dose of one or more TβRII antagonists of the present disclosure is administered once every two months. In some embodiments, a dose of one or more TβRII antagonists of the present disclosure is administered once every three months. In some embodiments, a dose of one or more TβRII antagonists of the present disclosure is administered once every four months. In some embodiments, a dose of one or more TβRII antagonists of the present disclosure is administered once every five months. In some embodiments, a dose of one or more TβRII antagonists of the present disclosure is administered once every six months. In some embodiments, a dose of one or more TβRII antagonists of the present disclosure is administered once every year.

In some embodiments, a dose of one or more TβRII antagonists of the present disclosure is administered twice every day. In some embodiments, a dose of one or more TβRII antagonists of the present disclosure is administered twice every two days. In some embodiments, a dose of one or more TβRII antagonists of the present disclosure is administered twice every three days. In some embodiments, a dose of one or more TβRII antagonists of the present disclosure is administered twice every four days. In some embodiments, a dose of one or more TβRII antagonists of the present disclosure is administered twice every five days. In some embodiments, a dose of one or more TβRII antagonists of the present disclosure is administered twice every six days. In some embodiments, a dose of one or more TβRII antagonists of the present disclosure is administered twice every week. In some embodiments, a dose of one or more TβRII antagonists of the present disclosure is administered twice every two weeks. In some embodiments, a dose of one or more TβRII antagonists of the present disclosure is administered twice every three weeks. In some embodiments, a dose of one or more TβRII antagonists of the present disclosure is administered twice every four weeks. In some embodiments, a dose of one or more TβRII antagonists of the present disclosure is administered twice every five weeks. In some embodiments, a dose of one or more TβRII antagonists of the present disclosure is administered twice every six weeks. In some embodiments, a dose of one or more TβRII antagonists of the present disclosure is administered twice every seven weeks. In some embodiments, a dose of one or more TβRII antagonists of the present disclosure is administered twice every eight weeks. In some embodiments, a dose of one or more TβRII antagonists of the present disclosure is administered twice every other week. In some embodiments, a dose of one or more TβRII antagonists of the present disclosure is administered twice every month. In some embodiments, a dose of one or more TβRII antagonists of the present disclosure is administered twice every two months. In some embodiments, a dose of one or more TβRII antagonists of the present disclosure is administered twice every three months. In some embodiments, a dose of one or more TβRII antagonists of the present disclosure is administered twice every four months. In some embodiments, a dose of one or more TβRII antagonists of the present disclosure is administered twice every five months. In some embodiments, a dose of one or more TβRII antagonists of the present disclosure is administered twice every six months. In some embodiments, a dose of one or more TβRII antagonists of the present disclosure is administered twice every year.

In some embodiments, a dose of one or more TβRII antagonists of the present disclosure is administered three times every day. In some embodiments, a dose of one or more TβRII antagonists of the present disclosure is administered three times every two days. In some embodiments, a dose of one or more TβRII antagonists of the present disclosure is administered three times every three days. In some embodiments, a dose of one or more TβRII antagonists of the present disclosure is administered three times every four days. In some embodiments, a dose of one or more TβRII antagonists of the present disclosure is administered three times every five days. In some embodiments, a dose of one or more TβRII antagonists of the present disclosure is administered three times every six days. In some embodiments, a dose of one or more TβRII antagonists of the present disclosure is administered three times every week. In some embodiments, a dose of one or more TβRII antagonists of the present disclosure is administered three times every two weeks. In some embodiments, a dose of one or more TβRII antagonists of the present disclosure is administered three times every three weeks. In some embodiments, a dose of one or more TβRII antagonists of the present disclosure is administered three times every four weeks. In some embodiments, a dose of one or more TβRII antagonists of the present disclosure is administered three times every five weeks. In some embodiments, a dose of one or more TβRII antagonists of the present disclosure is administered three times every six weeks. In some embodiments, a dose of one or more TβRII antagonists of the present disclosure is administered three times every seven weeks. In some embodiments, a dose of one or more TβRII antagonists of the present disclosure is administered three times every eight weeks. In some embodiments, a dose of one or more TβRII antagonists of the present disclosure is administered three times every other week. In some embodiments, a dose of one or more TβRII antagonists of the present disclosure is administered three times every month. In some embodiments, a dose of one or more TβRII antagonists of the present disclosure is administered three times every two months. In some embodiments, a dose of one or more TβRII antagonists of the present disclosure is administered three times every three months. In some embodiments, a dose of one or more TβRII antagonists of the present disclosure is administered three times every four months. In some embodiments, a dose of one or more TβRII antagonists of the present disclosure is administered three times every five months. In some embodiments, a dose of one or more TβRII antagonists of the present disclosure is administered three times every six months. In some embodiments, a dose of one or more TβRII antagonists of the present disclosure is administered three times every year.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered in a dose of between about 0.05 mg/kg and about 6.00 mg/kg to a subject in need thereof. In some embodiments, the TβRII antagonist is administered at least once every week. In some embodiments, the TβRII antagonist is administered at least once every three weeks. In some embodiments, the TβRII antagonist is administered at least once every four weeks. In some embodiments, the TβRII antagonist is administered at least once every five weeks. In some embodiments, the TβRII antagonist is administered at least once every six weeks. In some embodiments, the TβRII antagonist is administered subcutaneously.

In some embodiments, the TβRII antagonist is administered by a parenteral route of administration. In some embodiments, a parenteral route of administration is selected from the group consisting of intramuscular, intraperitoneal, intradermal, intravitreal, epidural, intracerebral, intra-arterial, intraarticular, intra-cavernous, intra-lesional, intraosseous, intraocular, intrathecal, intravenous, transdermal, trans-mucosal, extra-amniotic administration, subcutaneous, and combinations thereof. In some embodiments, a parenteral route of administration is subcutaneous. In some embodiments, a parenteral route of administration is a subcutaneous injection. In some embodiments, compositions of the present disclosure are administered by subcutaneous injection.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of at least between about 10 and about 20 μg/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 10 and about 20 μg/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 20 and about 30 μg/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 30 and about 40 μg/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 40 and about 50 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 10 and about 50 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 30 and about 50 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 50 and about 60 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 60 and about 70 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 70 and about 80 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a serum concentration of antagonist of between about 10 and about 80 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of at least between about 30 and about 50 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 30 and about 50 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 10 and about 20 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 20 and about 30 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 30 and about 40 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 40 and about 50 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 50 and about 60 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 60 and about 70 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 70 and about 80 ug/mL in the subject.

In some embodiments, the disclosure provides methods of treating chronic liver disease, portal hypertension, primary sclerosing cholangitis, and hepatopulmonary syndrome, or a complication associated with chronic liver disease, portal hypertension, primary sclerosing cholangitis, and/or hepatopulmonary syndrome (e.g., pulmonary fibrosis or ILD), comprising administering a Transforming Growth Factor-β Receptor II (TβRII) antagonist to a subject in need thereof, wherein the TβRII antagonist is administered at a dose that achieves a concentration of antagonist in the lung of between about 10 and about 80 ug/mL in the subject.

In certain embodiments, the present invention also provides gene therapy for the in vivo production of TβRII antagonists. Such therapy would achieve its therapeutic effect by introduction of the TβRII antagonist polynucleotide sequences into cells or tissues having the disorders as listed above. Delivery of TβRII antagonist polynucleotide sequences can be achieved using a recombinant expression vector such as a chimeric virus or a colloidal dispersion system. Preferred for therapeutic delivery of TβRII antagonist polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include but are not limited to: Moloney murine leukemia virus (MoMuLV), Harvey murine sarcoma virus (HaMuSV), murine mammary tumor virus (MuMTV), and/or Rous Sarcoma Virus (RSV). A number of additional retroviral vectors can incorporate multiple genes. All of these vectors can transfer or incorporate a gene for a selectable marker so that transduced cells can be identified and/or generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the TβRII antagonist polynucleotide. In a preferred embodiment, the vector is targeted to bone or cartilage.

Alternatively, tissue culture cells can be directly transfected with plasmids encoding the retroviral structural genes gag, pol and/or env, by conventional calcium phosphate transfection. These cells are then transfected with the vector plasmid containing the genes of interest. The resulting cells release the retroviral vector into the culture medium.

Another targeted delivery system for TβRII antagonist polynucleotides is a colloidal dispersion system. Colloidal dispersion systems include macromolecule complexes, nanocapsules, microspheres, beads, and/or lipid-based systems including oil-in-water emulsions, micelles, mixed micelles, and/or liposomes. The preferred colloidal system of this invention is a liposome. Liposomes are artificial membrane vesicles which are useful as delivery vehicles in vitro and/or in vivo. RNA, DNA and/or intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and/or the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and/or gangliosides. Illustrative phospholipids include egg phosphatidylcholine, dipalmitoylphosphatidylcholine, and/or distearoylphosphatidylcholine. The targeting of liposomes is also possible based on, for example, organ-specificity, cell-specificity, and/or organelle-specificity and is known in the art.

The disclosure provides formulations that may be varied to include acids and/or bases to adjust the pH; and/or buffering agents to keep the pH within a narrow range.

### EXEMPLIFICATION

The invention now being generally described, it will be more readily understood by reference to the following examples, which are included merely for purposes of illustration of certain embodiments of the present invention, and are not intended to limit the invention.

### Example 1. Generation of Receptor Fusion Protein Variants

### TβRII ECD Variants

TβRII fusion proteins comprising a soluble extracellular portion of human TβRII and a human Fc portion were generated. For each fusion protein, a TβRII amino acid sequence having the amino acid sequence of SEQ ID NO: 18 was fused to an IgG Fc portion having the amino acid sequence of SEQ ID NO: 20 by means of one of several different linkers. Each of the fusion proteins also included a TPA leader sequence having the amino acid sequence of SEQ ID NO: 23 (below).

Tissue plasminogen activator (TPA):
(SEQ ID NO: 23)
MDAMKRGLCCVLLLCGAVEVSP

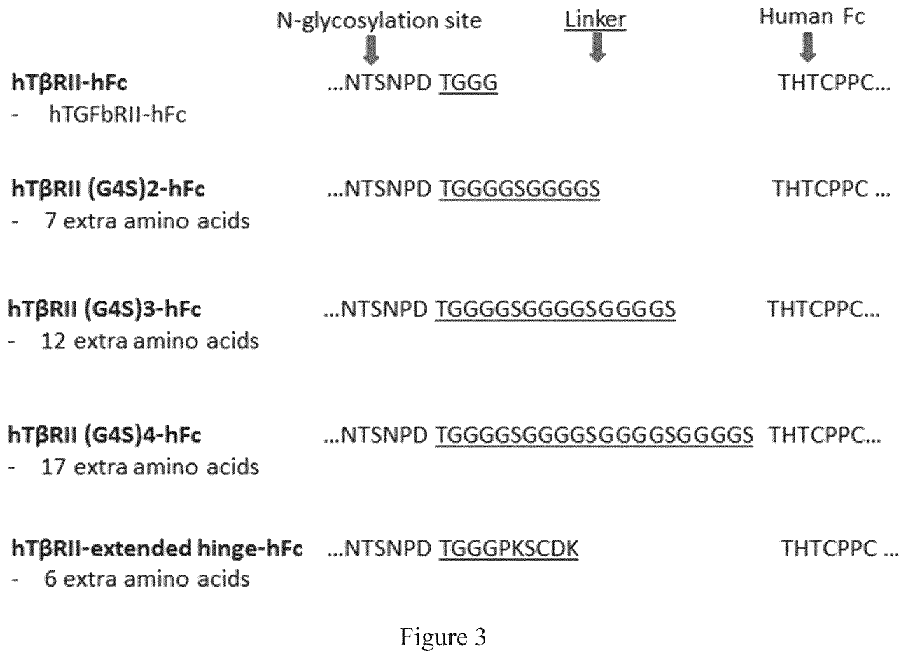

An illustration summary of several of the constructs designed is provided as FIG. 3. A table detailing the sequences for the different constructs tested in the Exemplification section is provided below:

| | Construct | |
|---|---|---|
| Construct | Amino Acid | |
| Name | Sequence | Linker Sequence |
| hTβRII-hFc | SEQ ID NO: 9 | TGGG (SEQ ID NO: 3) |
| | or 88 | |
| hTβRII (G4S)2- | SEQ ID NO: 15 | TGGGGSGGGGS (SEQ ID NO: 4) |
| hFc | or 70 | |
| hTβRII (G4S)3- | SEQ ID NO: 11 | TGGGGSGGGGSGGGGS (SEQ ID NO: 5) |
| hFc | or 68 | |
| hTβRII (G4S)4- | SEQ ID NO: 13 | TGGGGSGGGGSGGGGSGGGGS (SEQ ID NO: 6) |
| hFc | or 69 | |
| hTβRII | SEQ ID NO: 17 | TGGGPKSCDK (SEQ ID NO: 7) |
| extended hinge- | or 71 | |
| hFc | | |
| hTβRII (G4S)5- | SEQ ID NO: 44 | TGGGGSGGGGSGGGGSGGGGSGGGGS (SEQ |
| hFc | or 75 | ID NO: 25) |
| hTβRII (G4S)6- | SEQ ID NO: 45 | TGGGGSGGGGSGGGGSGGGGSGGGGSGGGGS |
| hFc | or 76 | (SEQ ID NO: 26) |

The amino acid sequences for the construct components and each of the constructs, along with the nucleic acid sequence used to express these constructs, are provided below.

| TβRII Portion: Amino Acid Sequence | |
|---|---|
| (SEQ ID NO: 18) | |
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PD |
| Fc Portion: Amino Acid Sequence | |
| (SEQ ID NO: 20) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

The C-terminal lysine residue of the Fc domain can be deleted. The amino acid sequence of SEQ ID NO: 20 may optionally be provided with the lysine removed from the C-terminus (SEQ ID NO: 72)

| (SEQ ID NO: 72) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPG |
| hTβRII-hFc: Nucleic Acid Sequence | |
| (SEQ ID NO: 8) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 10 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA ACTCACACAT GCCCACCGTG CCCAGCACCT |
| 601 | GAACTCCTGG GGGGACCGTC AGTCTTCCTC TTCCCCCCAA AACCCAAGGA |
| 651 | CACCCTCATG ATCTCCCGGA CCCCTGAGGT CACATGCGTG GTGGTGGACG |
| 701 | TGAGCCACGA AGACCCTGAG GTCAAGTTCA ACTGGTACGT GGACGGCGTG |
| 751 | GAGGTGCATA ATGCCAAGAC AAAGCCGCGG GAGGAGCAGT ACAACAGCAC |
| 801 | GTACCGTGTG GTCAGCGTCC TCACCGTCCT GCACCAGGAC TGGCTGAATG |
| 851 | GCAAGGAGTA CAAGTGCAAG GTCTCCAACA AAGCCCTCCC AGCCCCCATC |
| 901 | GAGAAAACCA TCTCCAAAGC CAAAGGGCAG CCCCGAGAAC CACAGGTGTA |
| 951 | CACCCTGCCC CCATCCCGGG AGGAGATGAC CAAGAACCAG GTCAGCCTGA |
| 1001 | CCTGCCTGGT CAAAGGCTTC TATCCCAGCG ACATCGCCGT GGAGTGGGAG |
| 1051 | AGCAATGGGC AGCCGGAGAA CAACTACAAG ACCACGCCTC CCGTGCTGGA |
| 1101 | CTCCGACGGC TCCTTCTTCC TCTATAGCAA GCTCACCGTG GACAAGAGCA |
| 1151 | GGTGGCAGCA GGGGAACGTC TTCTCATGCT CCGTGATGCA TGAGGCTCTG |
| 1201 | CACAACCACT ACACGCAGAA GAGCCTCTCC CTGTCTCCGG GTAAATGA |
| hTβRII-hFc: Amino Acid Sequence | |
| (SEQ ID NO: 9) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG THTCPPCPAP |
| 201 | ELLGGPSVFL FPPKPKDTLM ISRTPEVTCV VVDVSHEDPE VKFNWYVDGV |
| 251 | EVHNAKTKPR EEQYNSTYRV VSVLTVLHQD WLNGKEYKCK VSNKALPAPI |
| 301 | EKTISKAKGQ PREPQVYTLP PSREEMTKNQ VSLTCLVKGF YPSDIAVEWE |
| 351 | SNGQPENNYK TTPPVLDSDG SFFLYSKLTV DKSRWQQGNV FSCSVMHEAL |
| 401 | HNHYTQKSLS LSPGK |

The C-terminal lysine residue of the Fc domain can be deleted. The amino acid sequence of SEQ ID NO: 9 may optionally be provided with the lysine removed from the C-terminus (SEQ ID NO: 88):

| (SEQ ID NO: 88) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG THTCPPCPAP |
| 201 | ELLGGPSVFL FPPKPKDTLM ISRTPEVTCV VVDVSHEDPE VKFNWYVDGV |
| 251 | EVHNAKTKPR EEQYNSTYRV VSVLTVLHQD WLNGKEYKCK VSNKALPAPI |
| 301 | EKTISKAKGQ PREPQVYTLP PSREEMTKNQ VSLTCLVKGF YPSDIAVEWE |
| 351 | SNGQPENNYK TTPPVLDSDG SFFLYSKLTV DKSRWQQGNV FSCSVMHEAL |
| 401 | HNHYTQKSLS LSPG |
| hTβRII (G4S)3-hFc: Nucleic Acid Sequence | |
| (SEQ ID NO: 10) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA GGAAGTGGTG GAGGTGGTTC TGGAGGTGGT |
| 601 | GGAAGTACTC ACACATGCCC ACCGTGCCCA GCACCTGAAC TCCTGGGGGG |
| 651 | ACCGTCAGTC TTCCTCTTCC CCCCAAAACC CAAGGACACC CTCATGATCT |
| 701 | CCCGGACCCC TGAGGTCACA TGCGTGGTGG TGGACGTGAG CCACGAAGAC |
| 751 | CCTGAGGTCA AGTTCAACTG GTACGTGGAC GGCGTGGAGG TGCATAATGC |
| 801 | CAAGACAAAG CCGCGGGAGG AGCAGTACAA CAGCACGTAC CGTGTGGTCA |
| 851 | GCGTCCTCAC CGTCCTGCAC CAGGACTGGC TGAATGGCAA GGAGTACAAG |
| 901 | TGCAAGGTCT CCAACAAAGC CCTCCCAGCC CCCATCGAGA AAACCATCTC |
| 951 | CAAAGCCAAA GGGCAGCCCC GAGAACCACA GGTGTACACC CTGCCCCCAT |
| 1001 | CCCGGGAGGA GATGACCAAG AACCAGGTCA GCCTGACCTG CCTGGTCAAA |
| 1051 | GGCTTCTATC CCAGCGACAT CGCCGTGGAG TGGGAGAGCA ATGGGCAGCC |
| 1101 | GGAGAACAAC TACAAGACCA CGCCTCCCGT GCTGGACTCC GACGGCTCCT |
| 1151 | TCTTCCTCTA TAGCAAGCTC ACCGTGGACA AGAGCAGGTG GCAGCAGGGG |
| 1201 | AACGTCTTCT CATGCTCCGT GATGCATGAG GCTCTGCACA ACCACTACAC |
| 1251 | GCAGAAGAGC CTCTCCCTGT CTCCGGGTAA ATGA |
| hTβRII (G4S)3-hFc: Amino Acid Sequence | |
| (SEQ ID NO: 11) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSTHTCPPCP APELLGGPSV FLFPPKPKDT LMISRTPEVT CVVVDVSHED |
| 251 | PEVKFNWYVD GVEVHNAKTK PREEQYNSTY RVVSVLTVLH QDWLNGKEYK |
| 301 | CKVSNKALPA PIEKTISKAK GQPREPQVYT LPPSREEMTK NQVSLTCLVK |
| 351 | GFYPSDIAVE WESNGQPENN YKTTPPVLDS DGSFFLYSKL TVDKSRWQQG |
| 401 | NVFSCSVMHE ALHNHYTQKS LSLSPGK |

The C-terminal lysine residue of the Fc domain can be deleted. The amino acid sequence of SEQ ID NO: 11 may optionally be provided with the lysine removed from the C-terminus (SEQ ID NO: 68):

| (SEQ ID NO: 68) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSTHTCPPCP APELLGGPSV FLFPPKPKDT LMISRTPEVT CVVVDVSHED |
| 251 | PEVKFNWYVD GVEVHNAKTK PREEQYNSTY RVVSVLTVLH QDWLNGKEYK |
| 301 | CKVSNKALPA PIEKTISKAK GQPREPQVYT LPPSREEMTK NQVSLTCLVK |
| 351 | GFYPSDIAVE WESNGQPENN YKTTPPVLDS DGSFFLYSKL TVDKSRWQQG |
| 401 | NVFSCSVMHE ALHNHYTQKS LSLSPG |
| hTβRII (G4S)4-hFc: Nucleic Acid Sequence | |
| (SEQ ID NO: 12) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA GGTTCTGGAG GTGGAGGAAG TGGTGGAGGT |
| 601 | GGTTCTGGAG GTGGTGGAAG TACTCACACA TGCCCACCGT GCCCAGCACC |
| 651 | TGAACTCCTG GGGGGACCGT CAGTCTTCCT CTTCCCCCCA AAACCCAAGG |
| 701 | ACACCCTCAT GATCTCCCGG ACCCCTGAGG TCACATGCGT GGTGGTGGAC |
| 751 | GTGAGCCACG AAGACCCTGA GGTCAAGTTC AACTGGTACG TGGACGGCGT |
| 801 | GGAGGTGCAT AATGCCAAGA CAAAGCCGCG GGAGGAGCAG TACAACAGCA |
| 851 | CGTACCGTGT GGTCAGCGTC CTCACCGTCC TGCACCAGGA CTGGCTGAAT |
| 901 | GGCAAGGAGT ACAAGTGCAA GGTCTCCAAC AAAGCCCTCC CAGCCCCCAT |
| 951 | CGAGAAAACC ATCTCCAAAG CCAAAGGGCA GCCCCGAGAA CCACAGGTGT |
| 1001 | ACACCCTGCC CCCATCCCGG GAGGAGATGA CCAAGAACCA GGTCAGCCTG |
| 1051 | ACCTGCCTGG TCAAAGGCTT CTATCCCAGC GACATCGCCG TGGAGTGGGA |
| 1101 | GAGCAATGGG CAGCCGGAGA ACAACTACAA GACCACGCCT CCCGTGCTGG |
| 1151 | ACTCCGACGG CTCCTTCTTC CTCTATAGCA AGCTCACCGT GGACAAGAGC |
| 1201 | AGGTGGCAGC AGGGGAACGT CTTCTCATGC TCCGTGATGC ATGAGGCTCT |
| 1251 | GCACAACCAC TACACGCAGA AGAGCCTCTC CCTGTCTCCG GGTAAATGA |
| hTβRII (G4S)4-hFc: Amino Acid Sequence | |
| (SEQ ID NO: 13) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSTHT CPPCPAPELL GGPSVFLFPP KPKDTLMISR TPEVTCVVVD |
| 251 | VSHEDPEVKF NWYVDGVEVH NAKTKPREEQ YNSTYRVVSV LTVLHQDWLN |
| 301 | GKEYKCKVSN KALPAPIEKT ISKAKGQPRE PQVYTLPPSR EEMTKNQVSL |
| 351 | TCLVKGFYPS DIAVEWESNG QPENNYKTTP PVLDSDGSFF LYSKLTVDKS |
| 401 | RWQQGNVFSC SVMHEALHNH YTQKSLSLSP GK |

The C-terminal lysine residue of the Fc domain can be deleted. The amino acid sequence of SEQ ID NO: 13 may optionally be provided with the lysine removed from the C-terminus (SEQ ID NO: 69):

| (SEQ ID NO: 69) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSTHT CPPCPAPELL GGPSVFLFPP KPKDTLMISR TPEVTCVVVD |
| 251 | VSHEDPEVKF NWYVDGVEVH NAKTKPREEQ YNSTYRVVSV LTVLHQDWLN |
| 301 | GKEYKCKVSN KALPAPIEKT ISKAKGQPRE PQVYTLPPSR EEMTKNQVSL |
| 351 | TCLVKGFYPS DIAVEWESNG QPENNYKTTP PVLDSDGSFF LYSKLTVDKS |
| 401 | RWQQGNVFSC SVMHEALHNH YTQKSLSLSP G |
| hTβRII (G4S)4-hFc: Amino Acid Sequence lacking leader sequence | |
| (SEQ ID NO: 50) | |
| 1 | GATIPPHVQK SDVEMEAQKD EIICPSCNRT AHPLRHINND MIVTDNNGAV |
| 51 | KFPQLCKFCD VRFSTCDNQK SCMSNCSITS ICEKPQEVCV AVWRKNDENI |
| 101 | TLETVCHDPK LPYHDFILED AASPKCIMKE KKKPGETFFM CSCSSDECND |
| 151 | NIIFSEEYNT SNPDTGGGGS GGGGSGGGGS GGGGSTHTCP PCPAPELLGG |
| 201 | PSVFLFPPKP KDTLMISRTP EVTCVVVDVS HEDPEVKFNW YVDGVEVHNA |
| 251 | KTKPREEQYN STYRVVSVLT VLHQDWLNGK EYKCKVSNKA LPAPIEKTIS |
| 301 | KAKGQPREPQ VYTLPPSREE MTKNQVSLTC LVKGFYPSDI AVEWESNGQP |
| 351 | ENNYKTTPPV LDSDGSFFLY SKLTVDKSRW QQGNVFSCSV MHEALHNHYT |
| 401 | QKSLSLSPGK |

The C-terminal lysine residue of the Fc domain can be deleted. The amino acid sequence of SEQ ID NO: 50 may optionally be provided with the lysine removed from the C-terminus (SEQ ID NO: 77):

| (SEQ ID NO: 77) | |
|---|---|
| 1 | GATIPPHVQK SDVEMEAQKD EIICPSCNRT AHPLRHINND MIVTDNNGAV |
| 51 | KFPQLCKFCD VRFSTCDNQK SCMSNCSITS ICEKPQEVCV AVWRKNDENI |
| 101 | TLETVCHDPK LPYHDFILED AASPKCIMKE KKKPGETFFM CSCSSDECND |
| 151 | NIIFSEEYNT SNPDTGGGGS GGGGSGGGGS GGGGSTHTCP PCPAPELLGG |
| 201 | PSVFLFPPKP KDTLMISRTP EVTCVVVDVS HEDPEVKFNW YVDGVEVHNA |
| 251 | KTKPREEQYN STYRVVSVLT VLHQDWLNGK EYKCKVSNKA LPAPIEKTIS |
| 301 | KAKGQPREPQ VYTLPPSREE MTKNQVSLTC LVKGFYPSDI AVEWESNGQP |
| 351 | ENNYKTTPPV LDSDGSFFLY SKLTVDKSRW QQGNVFSCSV MHEALHNHYT |
| 401 | QKSLSLSPG |
| hTβRII (G4S)4-hFc: Amino Acid Sequence lacking leader sequence | |
| and lacking glycine prior to hTβRII portion | |
| (SEQ ID NO: 52) | |
| 1 | ATIPPHVQKS DVEMEAQKDE IICPSCNRTA HPLRHINNDM IVTDNNGAVK |
| 51 | FPQLCKFCDV RESTCDNQKS CMSNCSITSI CEKPQEVCVA VWRKNDENIT |
| 101 | LETVCHDPKL PYHDFILEDA ASPKCIMKEK KKPGETFFMC SCSSDECNDN |
| 151 | IIFSEEYNTS NPDTGGGGSG GGGSGGGGSG GGGSTHTCPP CPAPELLGGP |
| 201 | SVFLFPPKPK DTLMISRTPE VTCVVVDVSH EDPEVKENWY VDGVEVHNAK |
| 251 | TKPREEQYNS TYRVVSVLTV LHQDWLNGKE YKCKVSNKAL PAPIEKTISK |
| 301 | AKGQPREPQV YTLPPSREEM TKNQVSLTCL VKGFYPSDIA VEWESNGQPE |
| 351 | NNYKTTPPVL DSDGSFFLYS KLTVDKSRWQ QGNVFSCSVM HEALHNHYTQ |
| 401 | KSLSLSPGK |

The C-terminal lysine residue of the Fc domain can be deleted. The amino acid sequence of SEQ ID NO: 52 may optionally be provided with the lysine removed from the C-terminus (SEQ ID NO: 79):

| (SEQ ID NO: 79) | |
|---|---|
| 1 | ATIPPHVQKS DVEMEAQKDE IICPSCNRTA HPLRHINNDM IVTDNNGAVK |
| 51 | FPQLCKFCDV RESTCDNQKS CMSNCSITSI CEKPQEVCVA VWRKNDENIT |
| 101 | LETVCHDPKL PYHDFILEDA ASPKCIMKEK KKPGETFFMC SCSSDECNDN |
| 151 | IIFSEEYNTS NPDTGGGGSG GGGSGGGGSG GGGSTHTCPP CPAPELLGGP |
| 201 | SVFLFPPKPK DTLMISRTPE VTCVVVDVSH EDPEVKENWY VDGVEVHNAK |
| 251 | TKPREEQYNS TYRVVSVLTV LHQDWLNGKE YKCKVSNKAL PAPIEKTISK |
| 301 | AKGQPREPQV YTLPPSREEM TKNQVSLTCL VKGFYPSDIA VEWESNGQPE |
| 351 | NNYKTTPPVL DSDGSFFLYS KLTVDKSRWQ QGNVFSCSVM HEALHNHYTQ |
| 401 | KSLSLSPG |
| hTβRII (G4S)4-hFc: Amino Acid Sequence lacking leader sequence | |
| and lacking glycine and alanine prior to hTβRII portion | |
| (SEQ ID NO: 51) | |
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PDTGGGGSGG GGSGGGGSGG GGSTHTCPPC PAPELLGGPS |
| 201 | VFLFPPKPKD TLMISRTPEV TCVVVDVSHE DPEVKFNWYV DGVEVHNAKT |
| 251 | KPREEQYNST YRVVSVLTVL HQDWLNGKEY KCKVSNKALP APIEKTISKA |
| 301 | KGQPREPQVY TLPPSREEMT KNQVSLTCLV KGFYPSDIAV EWESNGQPEN |
| 351 | NYKTTPPVLD SDGSFFLYSK LTVDKSRWQQ GNVFSCSVMH EALHNHYTQK |
| 401 | SLSLSPGK |

The C-terminal lysine residue of the Fc domain can be deleted. The amino acid sequence of SEQ ID NO: 51 may optionally be provided with the lysine removed from the C-terminus (SEQ ID NO: 78):

| (SEQ ID NO: 78) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PDTGGGGSGG GGSGGGGSGG GGSTHTCPPC PAPELLGGPS |
| 201 | VFLFPPKPKD TLMISRTPEV TCVVVDVSHE DPEVKFNWYV DGVEVHNAKT |
| 251 | KPREEQYNST YRVVSVLTVL HQDWLNGKEY KCKVSNKALP APIEKTISKA |
| 301 | KGQPREPQVY TLPPSREEMT KNQVSLTCLV KGFYPSDIAV EWESNGQPEN |
| 351 | NYKTTPPVLD SDGSFFLYSK LTVDKSRWQQ GNVFSCSVMH EALHNHYTQK |
| 401 | SLSLSPG |
| hTβRII (G4S)4-hFc: Amino Acid Sequence lacking leader sequence and | |
| lacking glycine, alanine, and threonine prior to hTβRII portion | |
| (SEQ ID NO: 53) | |
| 1 | IPPHVQKSDV EMEAQKDEII CPSCNRTAHP LRHINNDMIV TDNNGAVKFP |
| 51 | QLCKFCDVRF STCDNQKSCM SNCSITSICE KPQEVCVAVW RKNDENITLE |
| 101 | TVCHDPKLPY HDFILEDAAS PKCIMKEKKK PGETFFMCSC SSDECNDNII |
| 151 | FSEEYNTSNP DTGGGGSGGG GSGGGGSGGG GSTHTCPPCP APELLGGPSV |
| 201 | FLFPPKPKDT LMISRTPEVT CVVVDVSHED PEVKFNWYVD GVEVHNAKTK |
| 251 | PREEQYNSTY RVVSVLTVLH QDWLNGKEYK CKVSNKALPA PIEKTISKAK |
| 301 | GQPREPQVYT LPPSREEMTK NQVSLTCLVK GFYPSDIAVE WESNGQPENN |
| 351 | YKTTPPVLDS DGSFFLYSKL TVDKSRWQQG NVFSCSVMHE ALHNHYTQKS |
| 401 | LSLSPGK |

The C-terminal lysine residue of the Fc domain can be deleted. The amino acid sequence of SEQ ID NO: 53 may optionally be provided with the lysine removed from the C-terminus (SEQ ID NO: 80):

| (SEQ ID NO: 80) | |
|---|---|
| 1 | IPPHVQKSDV EMEAQKDEII CPSCNRTAHP LRHINNDMIV TDNNGAVKFP |
| 51 | QLCKFCDVRF STCDNQKSCM SNCSITSICE KPQEVCVAVW RKNDENITLE |
| 101 | TVCHDPKLPY HDFILEDAAS PKCIMKEKKK PGETFFMCSC SSDECNDNII |
| 151 | FSEEYNTSNP DTGGGGSGGG GSGGGGSGGG GSTHTCPPCP APELLGGPSV |
| 201 | FLFPPKPKDT LMISRTPEVT CVVVDVSHED PEVKFNWYVD GVEVHNAKTK |
| 251 | PREEQYNSTY RVVSVLTVLH QDWLNGKEYK CKVSNKALPA PIEKTISKAK |
| 301 | GQPREPQVYT LPPSREEMTK NQVSLTCLVK GFYPSDIAVE WESNGQPENN |
| 351 | YKTTPPVLDS DGSFFLYSKL TVDKSRWQQG NVFSCSVMHE ALHNHYTQKS |
| 401 | LSLSPG |
| hTβRII (G4S)4-hFc: Amino Acid Sequence lacking leader sequence and | |
| lacking glycine, alanine, threonine, and isoleucine prior to hTβRII | |
| portion | |
| (SEQ ID NO: 54) | |
| 1 | PPHVQKSDVE MEAQKDEIIC PSCNRTAHPL RHINNDMIVT DNNGAVKFPQ |
| 51 | LCKFCDVRFS TCDNQKSCMS NCSITSICEK PQEVCVAVWR KNDENITLET |
| 101 | VCHDPKLPYH DFILEDAASP KCIMKEKKKP GETFFMCSCS SDECNDNIIF |
| 151 | SEEYNTSNPD TGGGGSGGGG SGGGGSGGGG STHTCPPCPA PELLGGPSVF |
| 201 | LFPPKPKDTL MISRTPEVTC VVVDVSHEDP EVKFNWYVDG VEVHNAKTKP |
| 251 | REEQYNSTYR VVSVLTVLHQ DWLNGKEYKC KVSNKALPAP IEKTISKAKG |
| 301 | QPREPQVYTL PPSREEMTKN QVSLTCLVKG FYPSDIAVEW ESNGQPENNY |
| 351 | KTTPPVLDSD GSFFLYSKLT VDKSRWQQGN VFSCSVMHEA LHNHYTQKSL |
| 401 | SLSPGK |

The C-terminal lysine residue of the Fc domain can be deleted. The amino acid sequence of SEQ ID NO: 54 may optionally be provided with the lysine removed from the C-terminus (SEQ ID NO: 81):

| (SEQ ID NO: 81) | |
|---|---|
| 1 | PPHVQKSDVE MEAQKDEIIC PSCNRTAHPL RHINNDMIVT DNNGAVKFPQ |
| 51 | LCKFCDVRFS TCDNQKSCMS NCSITSICEK PQEVCVAVWR KNDENITLET |
| 101 | VCHDPKLPYH DFILEDAASP KCIMKEKKKP GETFFMCSCS SDECNDNIIF |
| 151 | SEEYNTSNPD TGGGGSGGGG SGGGGSGGGG STHTCPPCPA PELLGGPSVF |
| 201 | LFPPKPKDTL MISRTPEVTC VVVDVSHEDP EVKFNWYVDG VEVHNAKTKP |
| 251 | REEQYNSTYR VVSVLTVLHQ DWLNGKEYKC KVSNKALPAP IEKTISKAKG |
| 301 | QPREPQVYTL PPSREEMTKN QVSLTCLVKG FYPSDIAVEW ESNGQPENNY |
| 351 | KTTPPVLDSD GSFFLYSKLT VDKSRWQQGN VFSCSVMHEA LHNHYTQKSL |
| 401 | SLSPG |
| | hTβRII (G4S)4-hFc: Amino Acid Sequence lacking leader |
| | sequence and lacking glycine, alanine, threonine, |
| | isoleucine, and proline prior to hTβRII portion |
| (SEQ ID NO: 55) | |
| 1 | PHVQKSDVEM EAQKDEIICP SCNRTAHPLR HINNDMIVTD NNGAVKFPQL |
| 51 | CKFCDVRFST CDNQKSCMSN CSITSICEKP QEVCVAVWRK NDENITLETV |
| 101 | CHDPKLPYHD FILEDAASPK CIMKEKKKPG ETFFMCSCSS DECNDNIIFS |
| 151 | EEYNTSNPDT GGGGSGGGGS GGGGSGGGGS THTCPPCPAP ELLGGPSVFL |
| 201 | FPPKPKDTLM ISRTPEVTCV VVDVSHEDPE VKFNWYVDGV EVHNAKTKPR |
| 251 | EEQYNSTYRV VSVLTVLHQD WLNGKEYKCK VSNKALPAPI EKTISKAKGQ |
| 301 | PREPQVYTLP PSREEMTKNQ VSLTCLVKGF YPSDIAVEWE SNGQPENNYK |
| 351 | TTPPVLDSDG SFFLYSKLTV DKSRWQQGNV FSCSVMHEAL HNHYTQKSLS |
| 401 | LSPGK |

The C-terminal lysine residue of the Fc domain can be deleted. The amino acid sequence of SEQ ID NO: 55 may optionally be provided with the lysine removed from the C-terminus (SEQ ID NO: 82):

| (SEQ ID NO: 82) | |
|---|---|
| 1 | PHVQKSDVEM EAQKDEIICP SCNRTAHPLR HINNDMIVTD NNGAVKFPQL |
| 51 | CKFCDVRFST CDNQKSCMSN CSITSICEKP QEVCVAVWRK NDENITLETV |
| 101 | CHDPKLPYHD FILEDAASPK CIMKEKKKPG ETFFMCSCSS DECNDNIIFS |
| 151 | EEYNTSNPDT GGGGSGGGGS GGGGSGGGGS THTCPPCPAP ELLGGPSVFL |
| 201 | FPPKPKDTLM ISRTPEVTCV VVDVSHEDPE VKFNWYVDGV EVHNAKTKPR |
| 251 | EEQYNSTYRV VSVLTVLHQD WLNGKEYKCK VSNKALPAPI EKTISKAKGQ |
| 301 | PREPQVYTLP PSREEMTKNQ VSLTCLVKGF YPSDIAVEWE SNGQPENNYK |
| 351 | TTPPVLDSDG SFFLYSKLTV DKSRWQQGNV FSCSVMHEAL HNHYTQKSLS |
| 401 | LSPG |
| | hTβRII (G4S)4-hFc: Amino Acid Sequence lacking leader |
| | sequence and lacking glycine, alanine, threonine, |
| | isoleucine, proline, and proline prior to hTβRII |
| | portion |
| (SEQ ID NO: 56) | |
| 1 | HVQKSDVEME AQKDEIICPS CNRTAHPLRH INNDMIVTDN NGAVKFPQLC |
| 51 | KFCDVRFSTC DNQKSCMSNC SITSICEKPQ EVCVAVWRKN DENITLETVC |
| 101 | HDPKLPYHDF ILEDAASPKC IMKEKKKPGE TFFMCSCSSD ECNDNIIFSE |
| 151 | EYNTSNPDTG GGGSGGGGSG GGGSGGGGST HTCPPCPAPE LLGGPSVFLF |
| 201 | PPKPKDTLMI SRTPEVTCVV VDVSHEDPEV KFNWYVDGVE VHNAKTKPRE |
| 251 | EQYNSTYRVV SVLTVLHQDW LNGKEYKCKV SNKALPAPIE KTISKAKGQP |
| 301 | REPQVYTLPP SREEMTKNQV SLTCLVKGFY PSDIAVEWES NGQPENNYKT |
| 351 | TPPVLDSDGS FFLYSKLTVD KSRWQQGNVF SCSVMHEALH NHYTQKSLSL |
| 401 | SPGK |

The C-terminal lysine residue of the Fc domain can be deleted. The amino acid sequence of SEQ ID NO: 56 may optionally be provided with the lysine removed from the C-terminus (SEQ ID NO: 83):

| (SEQ ID NO: 83) | |
|---|---|
| 1 | HVQKSDVEME AQKDEIICPS CNRTAHPLRH INNDMIVTDN NGAVKFPQLC |
| 51 | KFCDVRFSTC DNQKSCMSNC SITSICEKPQ EVCVAVWRKN DENITLETVC |
| 101 | HDPKLPYHDF ILEDAASPKC IMKEKKKPGE TFFMCSCSSD ECNDNIIFSE |
| 151 | EYNTSNPDTG GGGSGGGGSG GGGSGGGGST HTCPPCPAPE LLGGPSVFLF |
| 201 | PPKPKDTLMI SRTPEVTCVV VDVSHEDPEV KFNWYVDGVE VHNAKTKPRE |
| 251 | EQYNSTYRVV SVLTVLHQDW LNGKEYKCKV SNKALPAPIE KTISKAKGQP |
| 301 | REPQVYTLPP SREEMTKNQV SLTCLVKGFY PSDIAVEWES NGQPENNYKT |
| 351 | TPPVLDSDGS FFLYSKLTVD KSRWQQGNVF SCSVMHEALH NHYTQKSLSL |
| 401 | SPG |
| | hTβRII (G4S)2-hFc: Nucleic Acid Sequence |
| (SEQ ID NO: 14) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGAGGT GGTTCTGGAG GTGGTGGAAG TACTCACACA |
| 601 | TGCCCACCGT GCCCAGCACC TGAACTCCTG GGGGGACCGT CAGTCTTCCT |
| 651 | CTTCCCCCCA AAACCCAAGG ACACCCTCAT GATCTCCCGG ACCCCTGAGG |
| 701 | TCACATGCGT GGTGGTGGAC GTGAGCCACG AAGACCCTGA GGTCAAGTTC |
| 751 | AACTGGTACG TGGACGGCGT GGAGGTGCAT AATGCCAAGA CAAAGCCGCG |
| 801 | GGAGGAGCAG TACAACAGCA CGTACCGTGT GGTCAGCGTC CTCACCGTCC |
| 851 | TGCACCAGGA CTGGCTGAAT GGCAAGGAGT ACAAGTGCAA GGTCTCCAAC |
| 901 | AAAGCCCTCC CAGCCCCCAT CGAGAAAACC ATCTCCAAAG CCAAAGGGCA |
| 951 | GCCCCGAGAA CCACAGGTGT ACACCCTGCC CCCATCCCGG GAGGAGATGA |
| 1001 | CCAAGAACCA GGTCAGCCTG ACCTGCCTGG TCAAAGGCTT CTATCCCAGC |
| 1051 | GACATCGCCG TGGAGTGGGA GAGCAATGGG CAGCCGGAGA ACAACTACAA |
| 1101 | GACCACGCCT CCCGTGCTGG ACTCCGACGG CTCCTTCTTC CTCTATAGCA |
| 1151 | AGCTCACCGT GGACAAGAGC AGGTGGCAGC AGGGGAACGT CTTCTCATGC |
| 1201 | TCCGTGATGC ATGAGGCTCT GCACAACCAC TACACGCAGA AGAGCCTCTC |
| 1251 | CCTGTCTCCG GGTAAATGA |
| | hTβRII (G4S)2-hFc: Amino Acid Sequence |
| (SEQ ID NO: 15) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSTHT |
| 201 | CPPCPAPELL GGPSVFLFPP KPKDTLMISR TPEVTCVVVD VSHEDPEVKF |
| 251 | NWYVDGVEVH NAKTKPREEQ YNSTYRVVSV LTVLHQDWLN GKEYKCKVSN |
| 301 | KALPAPIEKT ISKAKGQPRE PQVYTLPPSR EEMTKNQVSL TCLVKGFYPS |
| 351 | DIAVEWESNG QPENNYKTTP PVLDSDGSFF LYSKLTVDKS RWQQGNVFSC |
| 401 | SVMHEALHNH YTQKSLSLSP GK |

The C-terminal lysine residue of the Fc domain can be deleted. The amino acid sequence of SEQ ID NO: 15 may optionally be provided with the lysine removed from the C-terminus (SEQ ID NO: 70):

| (SEQ ID NO: 70) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSTHT |
| 201 | CPPCPAPELL GGPSVFLFPP KPKDTLMISR TPEVTCVVVD VSHEDPEVKF |
| 251 | NWYVDGVEVH NAKTKPREEQ YNSTYRVVSV LTVLHQDWLN GKEYKCKVSN |
| 301 | KALPAPIEKT ISKAKGQPRE PQVYTLPPSR EEMTKNQVSL TCLVKGFYPS |
| 351 | DIAVEWESNG QPENNYKTTP PVLDSDGSFF LYSKLTVDKS RWQQGNVFSC |
| 401 | SVMHEALHNH YTQKSLSLSP G |
| | hTβRII extended hinge-hFc: Nucleic Acid Sequence |
| (SEQ ID NO: 16) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGTGGA CCCAAATCTT GTGACAAAAC TCACACATGC |
| 601 | CCACCGTGCC CAGCACCTGA ACTCCTGGGG GGACCGTCAG TCTTCCTCTT |
| 651 | CCCCCCAAAA CCCAAGGACA CCCTCATGAT CTCCCGGACC CCTGAGGTCA |
| 701 | CATGCGTGGT GGTGGACGTG AGCCACGAAG ACCCTGAGGT CAAGTTCAAC |
| 751 | TGGTACGTGG ACGGCGTGGA GGTGCATAAT GCCAAGACAA AGCCGCGGGA |
| 801 | GGAGCAGTAC AACAGCACGT ACCGTGTGGT CAGCGTCCTC ACCGTCCTGC |
| 851 | ACCAGGACTG GCTGAATGGC AAGGAGTACA AGTGCAAGGT CTCCAACAAA |
| 901 | GCCCTCCCAG CCCCCATCGA GAAAACCATC TCCAAAGCCA AAGGGCAGCC |
| 951 | CCGAGAACCA CAGGTGTACA CCCTGCCCCC ATCCCGGGAG GAGATGACCA |
| 1001 | AGAACCAGGT CAGCCTGACC TGCCTGGTCA AAGGCTTCTA TCCCAGCGAC |
| 1051 | ATCGCCGTGG AGTGGGAGAG CAATGGGCAG CCGGAGAACA ACTACAAGAC |
| 1101 | CACGCCTCCC GTGCTGGACT CCGACGGCTC CTTCTTCCTC TATAGCAAGC |
| 1151 | TCACCGTGGA CAAGAGCAGG TGGCAGCAGG GGAACGTCTT CTCATGCTCC |
| 1201 | GTGATGCATG AGGCTCTGCA CAACCACTAC ACGCAGAAGA GCCTCTCCCT |
| 1251 | GTCCCCGGGT AAATGA |
| | hTβRII extended hinge-hFc: Amino Acid Sequence |
| (SEQ ID NO: 17) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG PKSCDKTHTC |
| 201 | PPCPAPELLG GPSVFLFPPK PKDTLMISRT PEVTCVVVDV SHEDPEVKFN |
| 251 | WYVDGVEVHN AKTKPREEQY NSTYRVVSVL TVLHQDWLNG KEYKCKVSNK |
| 301 | ALPAPIEKTI SKAKGQPREP QVYTLPPSRE EMTKNQVSLT CLVKGFYPSD |
| 351 | IAVEWESNGQ PENNYKTTPP VLDSDGSFFL YSKLTVDKSR WQQGNVFSCS |
| 401 | VMHEALHNHY TQKSLSLSPG K |

The C-terminal lysine residue of the Fc domain can be deleted. The amino acid sequence of SEQ ID NO: 17 may optionally be provided with the lysine removed from the C-terminus (SEQ ID NO: 71):

| (SEQ ID NO: 71) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG PKSCDKTHTC |
| 201 | PPCPAPELLG GPSVFLFPPK PKDTLMISRT PEVTCVVVDV SHEDPEVKFN |
| 251 | WYVDGVEVHN AKTKPREEQY NSTYRVVSVL TVLHQDWLNG KEYKCKVSNK |
| 301 | ALPAPIEKTI SKAKGQPREP QVYTLPPSRE EMTKNQVSLT CLVKGFYPSD |
| 351 | IAVEWESNGQ PENNYKTTPP VLDSDGSFFL YSKLTVDKSR WQQGNVFSCS |
| 401 | VMHEALHNHY TQKSLSLSPG |
| | hTβRII (G4S)5-hFc: Amino Acid Sequence |
| (SEQ ID NO: 44) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSGGG GSTHTCPPCP APELLGGPSV FLFPPKPKDT LMISRTPEVT |
| 251 | CVVVDVSHED PEVKFNWYVD GVEVHNAKTK PREEQYNSTY RVVSVLTVLH |
| 301 | QDWLNGKEYK CKVSNKALPA PIEKTISKAK GQPREPQVYT LPPSREEMTK |
| 351 | NQVSLTCLVK GFYPSDIAVE WESNGQPENN YKTTPPVLDS DGSFFLYSKL |
| 401 | TVDKSRWQQG NVFSCSVMHE ALHNHYTQKS LSLSPGK |

The C-terminal lysine residue of the Fc domain can be deleted. The amino acid sequence of SEQ ID NO: 44 may optionally be provided with the lysine removed from the C-terminus (SEQ ID NO: 75):

| (SEQ ID NO: 75) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSGGG GSTHTCPPCP APELLGGPSV FLFPPKPKDT LMISRTPEVT |
| 251 | CVVVDVSHED PEVKFNWYVD GVEVHNAKTK PREEQYNSTY RVVSVLTVLH |
| 301 | QDWLNGKEYK CKVSNKALPA PIEKTISKAK GQPREPQVYT LPPSREEMTK |
| 351 | NQVSLTCLVK GFYPSDIAVE WESNGQPENN YKTTPPVLDS DGSFFLYSKL |
| 401 | TVDKSRWQQG NVFSCSVMHE ALHNHYTQKS LSLSPG |
| | hTβRII (G4S)6-hFc: Amino Acid Sequence |
| (SEQ ID NO: 45) | |
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRFSTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSGGG GSGGGGSTHT CPPCPAPELL GGPSVFLFPP KPKDTLMISR |
| 251 | TPEVTCVVVD VSHEDPEVKF NWYVDGVEVH NAKTKPREEQ YNSTYRVVSV |
| 301 | LTVLHQDWLN GKEYKCKVSN KALPAPIEKT ISKAKGQPRE PQVYTLPPSR |
| 351 | EEMTKNQVSL TCLVKGFYPS DIAVEWESNG QPENNYKTTP PVLDSDGSFF |
| 401 | LYSKLTVDKS RWQQGNVFSC SVMHEALHNH YTQKSLSLSP GK |

The C-terminal lysine residue of the Fc domain can be deleted. The amino acid sequence of SEQ ID NO: 45 may optionally be provided with the lysine removed from the C-terminus (SEQ ID NO: 76):

| (SEQ ID NO: 76) | |
|---|---|
| 1 | MDAMKRGLCC VLLLCGAVFV SPGATIPPHV QKSDVEMEAQ KDEIICPSCN |
| 51 | RTAHPLRHIN NDMIVTDNNG AVKFPQLCKF CDVRESTCDN QKSCMSNCSI |
| 101 | TSICEKPQEV CVAVWRKNDE NITLETVCHD PKLPYHDFIL EDAASPKCIM |
| 151 | KEKKKPGETF FMCSCSSDEC NDNIIFSEEY NTSNPDTGGG GSGGGGSGGG |
| 201 | GSGGGGSGGG GSGGGGSTHT CPPCPAPELL GGPSVFLFPP KPKDTLMISR |
| 251 | TPEVTCVVVD VSHEDPEVKF NWYVDGVEVH NAKTKPREEQ YNSTYRVVSV |
| 301 | LTVLHQDWLN GKEYKCKVSN KALPAPIEKT ISKAKGQPRE PQVYTLPPSR |
| 351 | EEMTKNQVSL TCLVKGFYPS DIAVEWESNG QPENNYKTTP PVLDSDGSFF |
| 401 | LYSKLTVDKS RWQQGNVFSC SVMHEALHNH YTQKSLSLSP G |
| | hTβRII (G4S)5-hFc: Nucleotide Sequence |
| (SEQ ID NO: 46) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGAGGA GGTTCTGGTG GTGGAGGTTC TGGAGGTGGA |
| 601 | GGAAGTGGTG GAGGTGGTTC TGGAGGTGGT GGAAGTACTC ACACATGCCC |
| 651 | ACCGTGCCCA GCACCTGAAC TCCTGGGGGG ACCGTCAGTC TTCCTCTTCC |
| 701 | CCCCAAAACC CAAGGACACC CTCATGATCT CCCGGACCCC TGAGGTCACA |
| 751 | TGCGTGGTGG TGGACGTGAG CCACGAAGAC CCTGAGGTCA AGTTCAACTG |
| 801 | GTACGTGGAC GGCGTGGAGG TGCATAATGC CAAGACAAAG CCGCGGGAGG |
| 851 | AGCAGTACAA CAGCACGTAC CGTGTGGTCA GCGTCCTCAC CGTCCTGCAC |
| 901 | CAGGACTGGC TGAATGGCAA GGAGTACAAG TGCAAGGTCT CCAACAAAGC |
| 951 | CCTCCCAGCC CCCATCGAGA AAACCATCTC CAAAGCCAAA GGGCAGCCCC |
| 1001 | GAGAACCACA GGTGTACACC CTGCCCCCAT CCCGGGAGGA GATGACCAAG |
| 1051 | AACCAGGTCA GCCTGACCTG CCTGGTCAAA GGCTTCTATC CCAGCGACAT |
| 1101 | CGCCGTGGAG TGGGAGAGCA ATGGGCAGCC GGAGAACAAC TACAAGACCA |
| 1151 | CGCCTCCCGT GCTGGACTCC GACGGCTCCT TCTTCCTCTA TAGCAAGCTC |
| 1201 | ACCGTGGACA AGAGCAGGTG GCAGCAGGGG AACGTCTTCT CATGCTCCGT |
| 1251 | GATGCATGAG GCTCTGCACA ACCACTACAC GCAGAAGAGC CTCTCCCTGT |
| 1301 | CTCCGGGTAA ATGA |
| | hTβRII (G4S)6-hFc: Nucleotide Sequence |
| (SEQ ID NO: 47) | |
| 1 | ATGGATGCAA TGAAGAGAGG GCTCTGCTGT GTGCTGCTGC TGTGTGGAGC |
| 51 | AGTCTTCGTT TCGCCCGGCG CCACGATCCC ACCGCACGTT CAGAAGTCGG |
| 101 | ATGTGGAAAT GGAGGCCCAG AAAGATGAAA TCATCTGCCC CAGCTGTAAT |
| 151 | AGGACTGCCC ATCCACTGAG ACATATTAAT AACGACATGA TAGTCACTGA |
| 201 | CAACAACGGT GCAGTCAAGT TTCCACAACT GTGTAAATTT TGTGATGTGA |
| 251 | GATTTTCCAC CTGTGACAAC CAGAAATCCT GCATGAGCAA CTGCAGCATC |
| 301 | ACCTCCATCT GTGAGAAGCC ACAGGAAGTC TGTGTGGCTG TATGGAGAAA |
| 351 | GAATGACGAG AACATAACAC TAGAGACAGT TTGCCATGAC CCCAAGCTCC |
| 401 | CCTACCATGA CTTTATTCTG GAAGATGCTG CTTCTCCAAA GTGCATTATG |
| 451 | AAGGAAAAAA AAAAGCCTGG TGAGACTTTC TTCATGTGTT CCTGTAGCTC |
| 501 | TGATGAGTGC AATGACAACA TCATCTTCTC AGAAGAATAT AACACCAGCA |
| 551 | ATCCTGACAC CGGTGGAGGT GGAAGTGGTG GAGGAGGTTC TGGTGGTGGA |
| 601 | GGTTCTGGAG GTGGAGGAAG TGGTGGAGGT GGTTCTGGAG GTGGTGGAAG |
| 651 | TACTCACACA TGCCCACCGT GCCCAGCACC TGAACTCCTG GGGGGACCGT |
| 701 | CAGTCTTCCT CTTCCCCCCA AAACCCAAGG ACACCCTCAT GATCTCCCGG |
| 751 | ACCCCTGAGG TCACATGCGT GGTGGTGGAC GTGAGCCACG AAGACCCTGA |
| 801 | GGTCAAGTTC AACTGGTACG TGGACGGCGT GGAGGTGCAT AATGCCAAGA |
| 851 | CAAAGCCGCG GGAGGAGCAG TACAACAGCA CGTACCGTGT GGTCAGCGTC |
| 901 | CTCACCGTCC TGCACCAGGA CTGGCTGAAT GGCAAGGAGT ACAAGTGCAA |
| 951 | GGTCTCCAAC AAAGCCCTCC CAGCCCCCAT CGAGAAAACC ATCTCCAAAG |
| 1001 | CCAAAGGGCA GCCCCGAGAA CCACAGGTGT ACACCCTGCC CCCATCCCGG |
| 1051 | GAGGAGATGA CCAAGAACCA GGTCAGCCTG ACCTGCCTGG TCAAAGGCTT |
| 1101 | CTATCCCAGC GACATCGCCG TGGAGTGGGA GAGCAATGGG CAGCCGGAGA |
| 1151 | ACAACTACAA GACCACGCCT CCCGTGCTGG ACTCCGACGG CTCCTTCTTC |
| 1201 | CTCTATAGCA AGCTCACCGT GGACAAGAGC AGGTGGCAGC AGGGGAACGT |
| 1251 | CTTCTCATGC TCCGTGATGC ATGAGGCTCT GCACAACCAC TACACGCAGA |
| 1301 | AGAGCCTCTC CCTGTCTCCG GGTAAATGA |

The various constructs were successfully expressed in CHO cells and were purified to a high degree of purity as determined by analytical size-exclusion chromatography and SDS-PAGE. The hTβRII (G4S)2-hFc, hTβRII (G4S)3-hFc, hTβRII (G4S)₄-hFc, hTβRII (G4S)5-hFc and hTβRII (G4S)6-hFc proteins displayed similarly strong stability as determined by SDS-PAGE analysis when maintained in PBS for 13 days at 37° C. The hTβRII (G45)2-hFc, hTβRII (G45)3-hFc, hTβRII (G45)4-hFc proteins were also maintained in rat, mouse or human serum and displayed similarly strong stability.

### TβRII ECD Variants

In addition to the TβRII domains included in the fusion proteins described above (e.g., SEQ ID NO: 18), the disclosure also contemplates fusion proteins comprising alternative TβRII domains. For example, the fusion protein may comprise the wild-type hTβRII short (23-159) sequence shown below (SEQ ID NO: 27) or any of the other TβRII polypeptides disclosed below:

| (SEQ ID NO: 27) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE YNTSNPD |

(1) The hTβRIIₛₕₒᵣₜ (23-159/D110K) amino acid sequence shown below (SEQ ID NO: 36), in which the substituted residue is underlined.

| (SEQ ID NO: 36) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHKFI LEDAASPKCI |
| 101 | MKEKKKPGET FFMCSCSSDE CNDNIIFSEE YNTSNPD |

(2) The N-terminally truncated hTβRIIₛₕₒᵣₜ(29-159) amino acid sequence shown below (SEQ ID NO: 28).

| (SEQ ID NO: 28) | |
|---|---|
| 1 | QKSVNNDMIV TDNNGAVKFP QLCKFCDVRF STCDNQKSCM SNCSITSICE |
| 51 | KPQEVCVAVW RKNDENITLE TVCHDPKLPY HDFILEDAAS PKCIMKEKKK |
| 101 | PGETFFMCSC SSDECNDNII FSEEYNTSNP D |

(3) The N-terminally truncated hTβRIIₛₕₒᵣₜ(35-159) amino acid sequence shown below (SEQ ID NO: 29).

| (SEQ ID NO: 29) | |
|---|---|
| 1 | DMIVTDNNGA VKFPQLCKFC DVRFSTCDNQ KSCMSNCSIT SICEKPQEVC |
| 51 | VAVWRKNDEN ITLETVCHDP KLPYHDFILE DAASPKCIMK EKKKPGETFF |
| 101 | MCSCSSDECN DNIIFSEEYN TSNPD |

(4) The C-terminally truncated hTβRIIₛₕₒᵣₜ(23-153) amino acid sequence shown below (SEQ ID NO: 30).

| (SEQ ID NO: 30) | |
|---|---|
| 1 | TIPPHVQKSV NNDMIVTDNN GAVKFPQLCK FCDVRFSTCD NQKSCMSNCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE Y |

(5) The C-terminally truncated hTβRIIₛₕₒᵣₜ(23-153/N70D) amino acid sequence shown below (SEQ ID NO: 38), in which the substituted residue is underlined.

| (SEQ ID NO: 38) | |
|---|---|
| 1 | TIPPHVQKSV NNDMIVTDNN GAVKFPQLCK FCDVRFSTCD NQKSCMSDCS |
| 51 | ITSICEKPQE VCVAVWRKND ENITLETVCH DPKLPYHDFI LEDAASPKCI |
| 101 | MKEKKKPGET FFMCSCSSDE CNDNIIFSEE Y |

Applicants also envision five corresponding variants (SEQ ID NOs: 37, 33, 34, 39) based on the wild-type hTβRII_{long} (23-184) sequence shown above and below (SEQ ID NO: 49), in which the 25 amino-acid insertion is underlined. Note that splicing results in a conservative amino acid substitution (Val-41e) at the flanking position C-terminal to the insertion.

| (SEQ ID NO: 49) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEYNTSN PD |

(1) The hTβRII_{long} (23-184/D135K) amino acid sequence shown below (SEQ ID NO: 37), in which the substituted residue is double underlined.

| (SEQ ID NO: 37) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHKFILEDAA SPKCIMKEKK KPGETFFMCS CSSDECNDNI |
| 151 | IFSEEYNTSN PD |

(2) The N-terminally truncated hTβRII_{long} (29-184) amino acid sequence shown below (SEQ ID NO: 33).

| (SEQ ID NO: 33) | |
|---|---|
| 1 | QKSDVEMEAQ KDEIICPSCN RTAHPLRHIN NDMIVTDNNG AVKFPQLCKF |
| 51 | CDVRFSTCDN QKSCMSNCSI TSICEKPQEV CVAVWRKNDE NITLETVCHD |
| 101 | PKLPYHDFIL EDAASPKCIM KEKKKPGETF FMCSCSSDEC NDNIIFSEEY |
| 151 | NTSNPD |

(3) The N-terminally truncated hTβRII_{long} (60-184) amino acid sequence shown below (same as SEQ ID NO: 29).

| (same as SEQ INO: 29) | |
|---|---|
| 1 | DMIVTDNNGA VKFPQLCKFC DVRFSTCDNQ KSCMSNCSIT SICEKPQEVC |
| 51 | VAVWRKNDEN ITLETVCHDP KLPYHDFILE DAASPKCIMK EKKKPGETFF |
| 101 | MCSCSSDECN DNIIFSEEYN TSNPD |

(4) The C-terminally truncated hTβRII_{long} (23-178) amino acid sequence shown below (SEQ ID NO: 34).

| (SEQ ID NO: 34) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSNCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEY |

(5) The C-terminally truncated hTβRII_{long} (23-178/N95D) amino acid sequence shown below (SEQ ID NO: 39), in which the substituted residue is double underlined.

| (SEQ ID NO: 39) | |
|---|---|
| 1 | TIPPHVQKSD VEMEAQKDEI ICPSCNRTAH PLRHINNDMI VTDNNGAVKF |
| 51 | PQLCKFCDVR FSTCDNQKSC MSDCSITSIC EKPQEVCVAV WRKNDENITL |
| 101 | ETVCHDPKLP YHDFILEDAA SPKCIMKEKK KPGETFFMCS CSSDECNDNI |
| 151 | IFSEEY |

Additional TβRII ECD variants include:
(A) The N- and C-terminally truncated hTβRIIₛₕₒᵣₜ(35-153) or hTβRII_{long} (60-178) amino acid sequence shown below (SEQ ID NO: 32).

| (SEQ ID NO: 32) | |
|---|---|
| 1 | DMIVTDNNGA VKFPQLCKFC DVRFSTCDNQ KSCMSNCSIT SICEKPQEVC |
| 51 | VAVWRKNDEN ITLETVCHDP KLPYHDFILE DAASPKCIMK EKKKPGETFF |
| 101 | MCSCSSDECN DNIIFSEEY |

(B) The N- and C-terminally truncated hTβRIIₛₕₒᵣₜ(29-153) amino acid sequence shown below (SEQ ID NO: 31).

| (SEQ ID NO: 31) | |
|---|---|
| 1 | QKSVNNDMIV TDNNGAVKFP QLCKFCDVRF STCDNQKSCM SNCSITSICE |
| 51 | KPQEVCVAVW RKNDENITLE TVCHDPKLPY HDFILEDAAS PKCIMKEKKK |
| 101 | PGETFFMCSC SSDECNDNII FSEEY |

(C) The N- and C-terminally truncated hTβRII_{long} (29-178) amino acid sequence shown below (SEQ ID NO: 35).

| (SEQ ID NO: 35) | |
|---|---|
| 1 | QKSDVEMEAQ KDEIICPSCN RTAHPLRHIN NDMIVTDNNG AVKFPQLCKF |
| 51 | CDVRFSTCDN QKSCMSNCSI TSICEKPQEV CVAVWRKNDE NITLETVCHD |
| 101 | PKLPYHDFIL EDAASPKCIM KEKKKPGETF FMCSCSSDEC NDNIIFSEEY |

Any of the above variants (SEQ ID NO: 36, 28, 29, 30, 38, 37, 33, 34, 39, 32, 31, and 35) could incorporate an insertion of 36 amino acids (SEQ ID NO: 41) between the pair of glutamate residues (positions 151 and 152 of SEQ ID NO: 1, or positions 176 and 177 of SEQ ID NO: 2) located near the C-terminus of the hTβRII ECD, as occurs naturally in the hTβRII isoform C (Konrad et al., BMC Genomics 8:318, 2007).

(SEQ ID NO: 41)
GRCKIRHIGS NNRLQRSTCQ NTGWESAHVM KTPGFR

As an example, the paired glutamate residues flanking the optional insertion site are denoted below (underlined) for the hTβRIIₛₕₒᵣₜ (29-159) variant (SEQ ID NO: 28).

| (SEQ ID NO: 28) | |
|---|---|
| 1 | QKSVNNDMIV TDNNGAVKFP QLCKFCDVRF STCDNQKSCM SNCSITSICE |
| 51 | KPQEVCVAVW RKNDENITLE TVCHDPKLPY HDFILEDAAS PKCIMKEKKK |
| 101 | PGETFFMCSC SSDECNDNII FSEEYNTSNP D |

### Fc Domain Variants

While the constructs described above were generated with an Fc domain having the amino acid sequence of SEQ ID NO: 20, the disclosure contemplates hTβRII-hFc fusion proteins comprising alternative Fc domains, including a human IgG2 Fc domain (SEQ ID NO: 42, below) or full-length human IgG1 Fc (hG1Fc) (SEQ ID NO: 43, below). Optionally, a polypeptide unrelated to an Fc domain could be attached in place of the Fc domain.

| (SEQ ID NO: 42) | |
|---|---|
| 1 | VECPPCPAPP VAGPSVFLFP PKPKDTLMIS RTPEVTCVVV DVSHEDPEVQ |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

The C-terminal lysine residue of the Fc domain can be deleted. The amino acid sequence of SEQ ID NO: 42 may optionally be provided with the lysine removed from the C-terminus (SEQ ID NO: 73):

| (SEQ ID NO: 73) | |
|---|---|
| 1 | VECPPCPAPP VAGPSVFLFP PKPKDTLMIS RTPEVTCVVV DVSHEDPEVQ |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PG |
| (SEQ ID NO: 43) | |
| 1 | GGPKSCDKTH TCPPCPAPEL LGGPSVFLFP PKPKDTLMIS RTPEVTCVVV |
| 51 | DVSHEDPEVK FNWYVDGVEV HNAKTKPREE QYNSTYRVVS VLTVLHQDWL |
| 101 | NGKEYKCKVS NKALPAPIEK TISKAKGQPR EPQVYTLPPS REEMTKNQVS |
| 151 | LTCLVKGFYP SDIAVEWESN GQPENNYKTT PPVLDSDGSF FLYSKLTVDK |
| 201 | SRWQQGNVFS CSVMHEALHN HYTQKSLSLS PGK |

The C-terminal lysine residue of the Fc domain can be deleted. The amino acid sequence of SEQ ID NO: 43 may optionally be provided with the lysine removed from the C-terminus (SEQ ID NO: 74):

| (SEQ ID NO: 74) | |
|---|---|
| 1 | GGPKSCDKTH TCPPCPAPEL LGGPSVFLFP PKPKDTLMIS RTPEVTCVVV |
| 51 | DVSHEDPEVK FNWYVDGVEV HNAKTKPREE QYNSTYRVVS VLTVLHQDWL |
| 101 | NGKEYKCKVS NKALPAPIEK TISKAKGQPR EPQVYTLPPS REEMTKNQVS |
| 151 | LTCLVKGFYP SDIAVEWESN GQPENNYKTT PPVLDSDGSF FLYSKLTVDK |
| 201 | SRWQQGNVFS CSVMHEALHN HYTQKSLSLS PG |

### Leader Sequence Variants

While the generated constructs described above included the TPA leader sequence, alternative leader sequences may be used, such as the native leader sequence (SEQ ID NO: 22—below) or the honey bee melittin (SEQ ID NO: 24—below) leader sequences.

Native:
(SEQ ID NO: 22)
MGRGLLRGLWPLHIVLWTRIAS
Honey bee melittin (HBML):
(SEQ ID NO: 24)
MKFLVNVALVFMVVYISYIYA

mTβRII-mFc comprises murine TβRII extracellular domain and murine IgG2a Fc. The signal sequence is underlined. The linker in mTβRII-mFc is TGGG (SEQ ID NO: 9), bolded and underlined below. The human version (SEQ ID NO: 48) has a longer linker (SEQ ID NO: 6). The unprocessed sequence (SEQ ID NO: 84) is below:

| (SEQ ID NO: 84) | |
|---|---|
| 1 | MDAMKRGLCC VLLLCGAVFV SPGAIPPHVP KSDVEMEAQK DASIHLSCNR |
| 51 | TIHPLKHENS DVMASDNGGA VKLPQLCKFC DVRLSTCDNQ KSCMSNCSIT |
| 101 | AICEKPHEVC VAVWRKNDKN ITLETVCHDP KLTYHGFTLE DAASPKCVMK |
| 151 | EKKRAGETFF MCACNMEECN DYIIFSEEYT TSSPDTGGGE PRVPITQNPC |
| 201 | PPLKECPPCA APDLLGGPSV FIFPPKIKDV LMISLSPMVT CVVVDVSEDD |
| 251 | PDVQISWFVN NVEVHTAQTQ THREDYNSTL RVVSALPIQH QDWMSGKEFK |
| 301 | CKVNNRALPS PIEKTISKPR GPVRAPQVYV LPPPAEEMTK KEFSLTCMIT |
| 351 | GFLPAEIAVD WTSNGRTEQN YKNTATVLDS DGSYFMYSKL RVQKSTWERG |
| 401 | SLFACSVVHE GLHNHLTTKT ISRSLGK* |
| The mature version of mTβRII-mFc is shown as SEQ ID NO: 85: | |
| (SEQ ID NO: 85) | |
| 1 | IPPHVPKSDV EMEAQKDASI HLSCNRTIHP LKHFNSDVMA SDNGGAVKLP |
| 51 | QLCKFCDVRL STCDNQKSCM SNCSITAICE KPHEVCVAVW RKNDKNITLE |
| 101 | TVCHDPKLTY HGFTLEDAAS PKCVMKEKKR AGETFFMCAC NMEECNDYII |
| 151 | FSEEYTTSSP DTGGGEPRVP ITQNPCPPLK ECPPCAAPDL LGGPSVFIFP |
| 201 | PKIKDVLMIS LSPMVTCVVV DVSEDDPDVQ ISWFVNNVEV HTAQTQTHRE |
| 251 | DYNSTLRVVS ALPIQHQDWM SGKEFKCKVN NRALPSPIEK TISKPRGPVR |
| 301 | APQVYVLPPP AEEMTKKEFS LTCMITGFLP AEIAVDWTSN GRTEQNYKNT |
| 351 | ATVLDSDGSY FMYSKLRVQK STWERGSLFA CSVVHEGLHN HLTTKTISRS |
| 401 | LGK |

Furthermore, the C-terminal lysine residue of the Fc domain can be deleted. The amino acid sequence of SEQ ID NO: 84 may optionally be provided with the lysine removed from the C-terminus (SEQ ID NO: 86)

| (SEQ ID NO: 86) | |
|---|---|
| 1 | IPPHVPKSDV EMEAQKDASI HLSCNRTIHP LKHFNSDVMA SDNGGAVKLP |
| 51 | QLCKFCDVRL STCDNQKSCM SNCSITAICE KPHEVCVAVW RKNDKNITLE |
| 101 | TVCHDPKLTY HGFTLEDAAS PKCVMKEKKR AGETFFMCAC NMEECNDYII |
| 151 | FSEEYTTSSP DTGGGEPRVP ITQNPCPPLK ECPPCAAPDL LGGPSVFIFP |
| 201 | PKIKDVLMIS LSPMVTCVVV DVSEDDPDVQ ISWFVNNVEV HTAQTQTHRE |
| 251 | DYNSTLRVVS ALPIQHODWM SGKEFKCKVN NRALPSPIEK TISKPRGPVR |
| 301 | APQVYVLPPP AEEMTKKEFS LTCMITGFLP AEIAVDWTSN GRTEQNYKNT |
| 351 | ATVLDSDGSY FMYSKLRVQK STWERGSLFA CSVVHEGLHN HLTTKTISRS |
| 401 | LG |
| mTβRII-mFc nucleic acid sequence (SEQ ID NO: 87): | |
| (SEQ ID NO: 87) | |
| 1 | ATGGATGCAA TGAAGAGAGG GCTCTGCTGT GTGCTGCTGC TGTGTGGAGC |
| 51 | AGTCTTCGTT TCGCCCGGCG CCATCCCGCC GCACGTTCCC AAGTCGGATG |
| 101 | TGGAAATGGA AGCCCAGAAA GATGCATCCA TCCACCTAAG CTGTAATAGG |
| 151 | ACCATCCATC CACTGAAACA TTTTAACAGT GATGTCATGG CCAGCGACAA |
| 201 | TGGCGGTGCG GTCAAGCTTC CACAGCTGTG CAAGTTTTGC GATGTGAGAC |
| 251 | TGTCCACTTG CGACAACCAG AAGTCCTGCA TGAGCAACTG CAGCATCACG |
| 301 | GCCATCTGTG AGAAGCCGCA TGAAGTCTGC GTGGCCGTGT GGAGGAAGAA |
| 351 | CGACAAGAAC ATTACTCTGG AGACGGTTTG CCACGACCCC AAGCTCACCT |
| 401 | ACCACGGCTT CACTCTGGAA GATGCCGCTT CTCCCAAGTG TGTCATGAAG |
| 451 | GAAAAGAAAA GGGCGGGCGA GACTTTCTTC ATGTGTGCCT GTAACATGGA |
| 501 | AGAGTGCAAC GATTACATCA TCTTTTCGGA AGAATACACC ACCAGCAGTC |
| 551 | CCGACACCGG TGGGGGTGAG CCCAGAGTGC CCATAACACA GAACCCCTGT |
| 601 | CCTCCACTCA AAGAGTGTCC CCCATGCGCA GCTCCAGACC TCTTGGGTGG |
| 651 | ACCATCCGTC TTCATCTTCC CTCCAAAGAT CAAGGATGTA CTCATGATCT |
| 701 | CCCTGAGCCC CATGGTCACA TGTGTGGTGG TGGATGTGAG CGAGGATGAC |
| 751 | CCAGACGTCC AGATCAGCTG GTTTGTGAAC AACGTGGAAG TACACACAGC |
| 801 | TCAGACACAA ACCCATAGAG AGGATTACAA CAGTACTCTC CGGGTGGTCA |
| 851 | GTGCCCTCCC CATCCAGCAC CAGGACTGGA TGAGTGGCAA GGAGTTCAAA |
| 901 | TGCAAGGTCA ACAACAGAGC CCTCCCATCC CCCATCGAGA AAACCATCTC |
| 951 | AAAACCCAGA GGGCCAGTAA GAGCTCCACA GGTATATGTC TTGCCTCCAC |
| 1001 | CAGCAGAAGA GATGACTAAG AAAGAGTTCA GTCTGACCTG CATGATCACA |
| 1051 | GGCTTCTTAC CTGCCGAAAT TGCTGTGGAC TGGACCAGCA ATGGGCGTAC |
| 1101 | AGAGCAAAAC TACAAGAACA CCGCAACAGT CCTGGACTCT GATGGTTCTT |
| 1151 | ACTTCATGTA CAGCAAGCTC AGAGTACAAA AGAGCACTTG GGAAAGAGGA |
| 1201 | AGTCTTTTCG CCTGCTCAGT GGTCCACGAG GGTCTGCACA ATCACCTTAC |
| 1251 | GACTAAGACC ATCTCCCGGT CTCTGGGTAA ATGA |

### Example 2. Differential Ligand Inhibition by Receptor Fusion Protein Variants in Cell-Based Assay

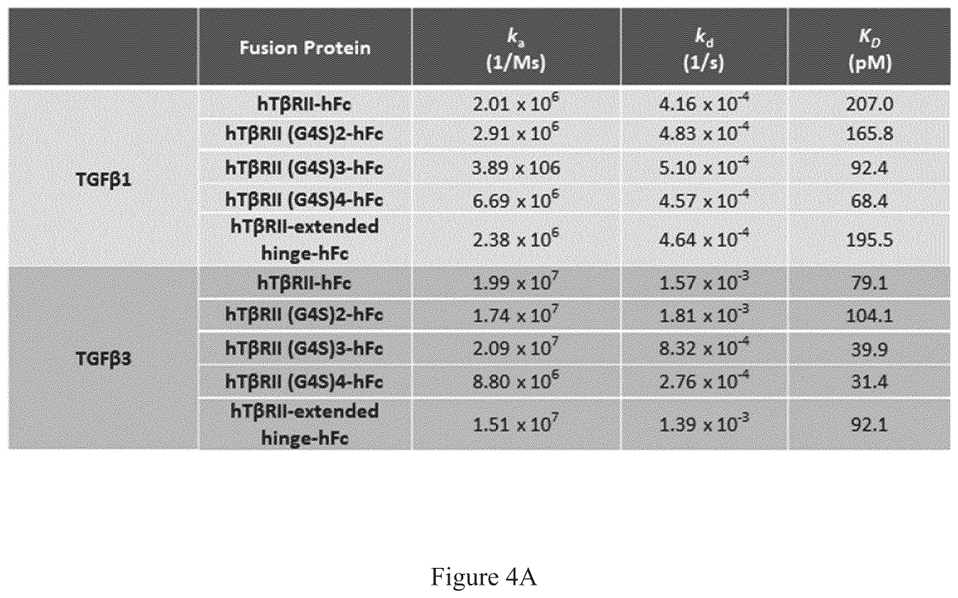

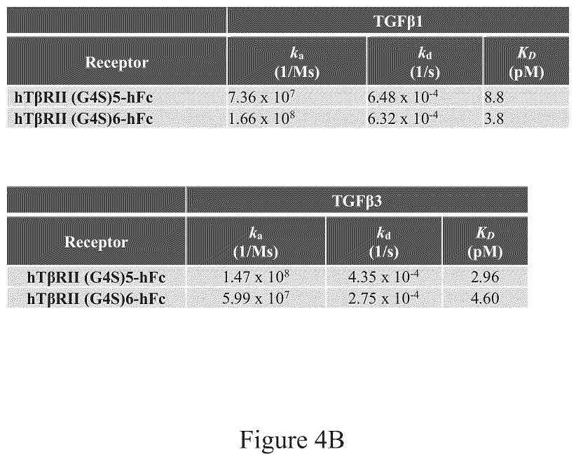

Affinities of TGFβ1, TGFβ2 and TGFβ3 for hTβRII (G4S)2-hFc; hTβRII (G4S)3-hFc; hTβRII (G4S)₄-hFc; hTβRII-hFc; and hTβRII extended hinge-hFc proteins were evaluated in vitro with a Biacore™ instrument, and the results are summarized in FIGS. 4A and 4B. Each of the fusion proteins was capable of binding TGFβ1 and TGFβ3 with high affinity, but the constructs having linker lengths longer than or equal to (G4S)4 (SEQ ID NO: 59) were surprisingly capable of binding to both TGFβ1 and TGFβ3 with higher affinity than constructs having linker lengths shorter than (G45)4 (SEQ ID NO: 59). Binding between TGFβ2 and any of the constructs was low or transient. Deglycosylation of the constructs did not change binding.

A reporter gene assay in A549 cells was used to determine the ability of hTβRII-hFc variants to inhibit activity of TGFβ1, TGFβ2 and TGFβ3. This assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates. On the second day, a solution containing pGL3(CAGA)12, pRLCMV, X-tremeGENE 9 (Roche Applied Science), and OptiMEM (Invitrogen) was preincubated, then added to Eagle's minimum essential medium (EMEM, ATCC®) supplemented with 0.1% BSA, which was applied to the plated cells for incubation overnight at 37° C., 5% CO₂. On the third day, medium was removed, and cells were incubated overnight at 37° C., 5% CO₂ with a mixture of ligands and inhibitors prepared as described below.

Serial dilutions of test articles were made in a 48-well plate in assay buffer (EMEM+% BSA). An equal volume of assay buffer containing the test ligand was added to obtain a final ligand concentration equal to the EC50 determined previously. Human TGFβ1, human TGFβ2, and human TGFβ3 were obtained from PeproTech. Test solutions were incubated at 37° C. for 30 minutes, then a portion of the mixture was added to all wells. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred in duplicate to a chemiluminescence plate (96-well) and analyzed in a luminometer with reagents from a Dual-Luciferase Reporter Assay system (Promega E1980) to determine normalized luciferase activity.

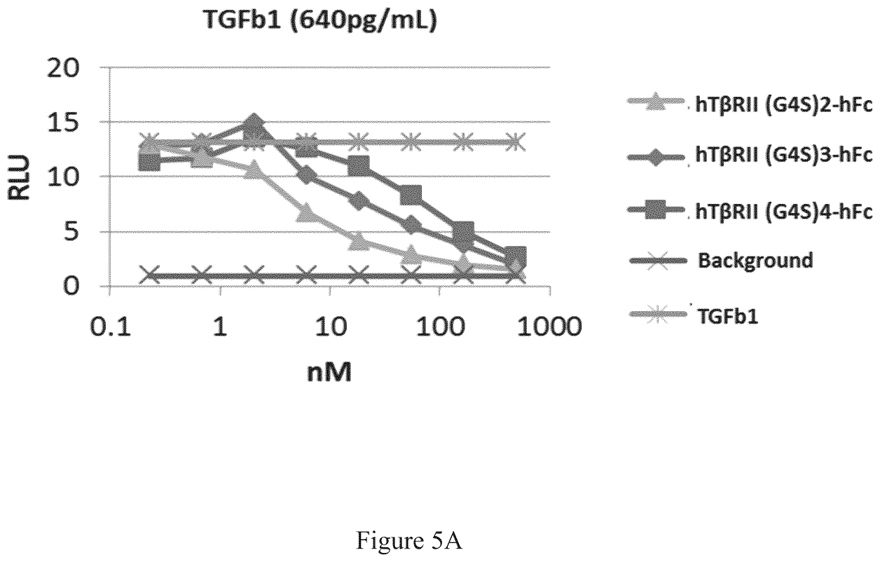

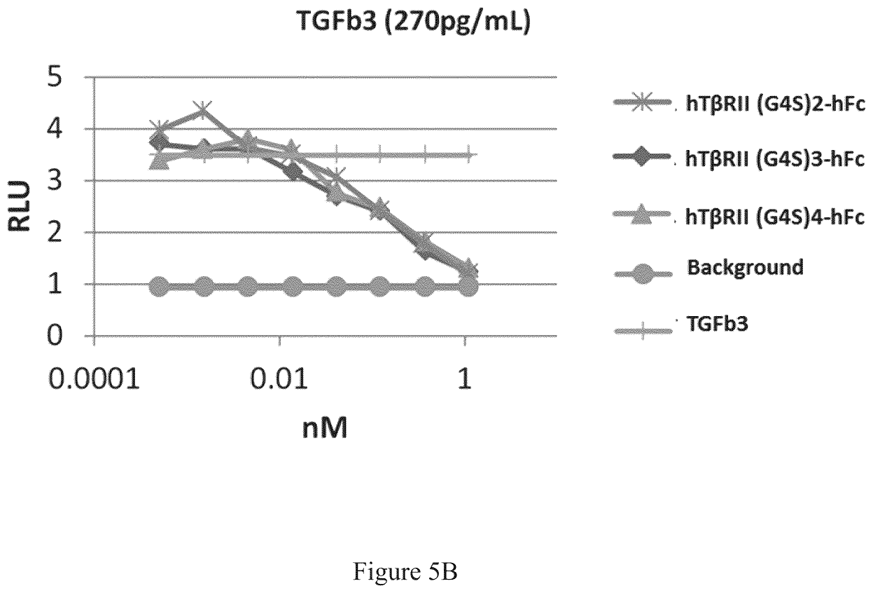

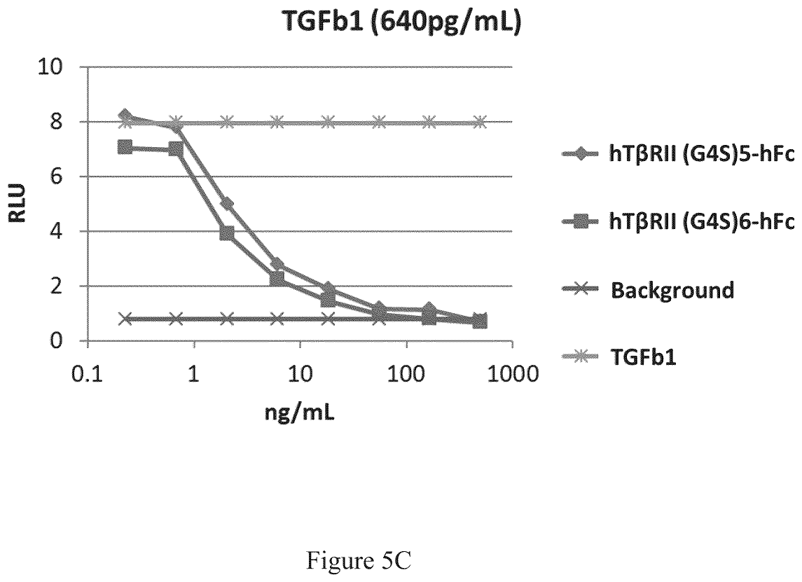

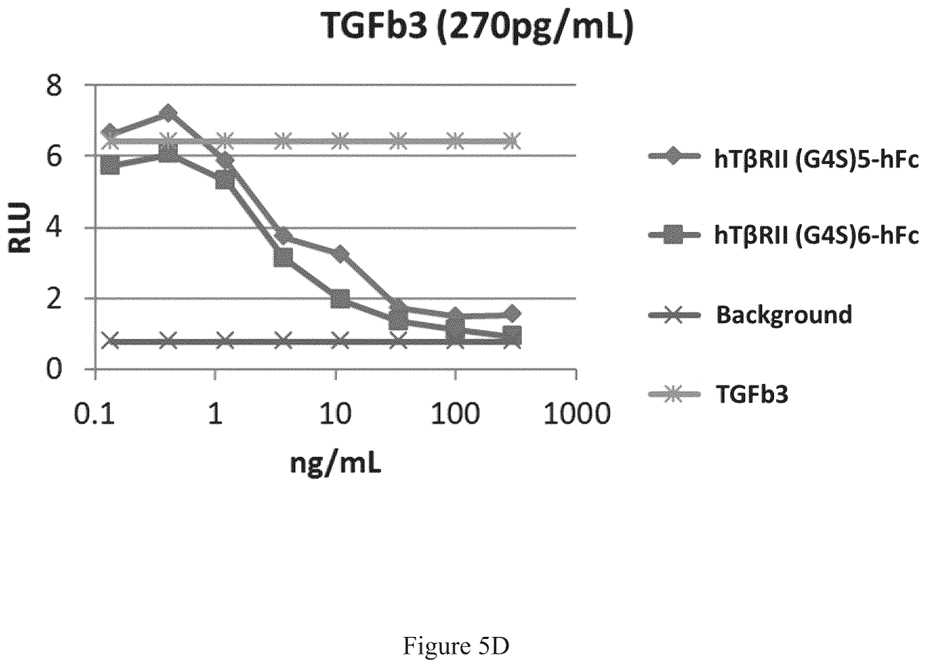

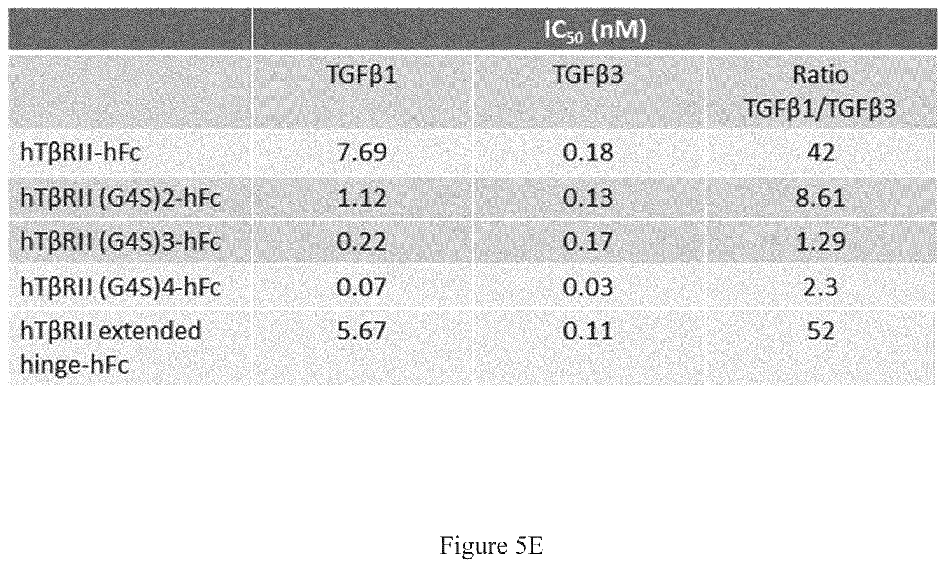

As illustrated in FIGS. 5A-5F, the hTβRII (G4S)2-hFc; hTβRII (G4S)3-hFc; hTβRII (G4S)₄-hFc; hTβRII (G4S)5-hFc; hTβRII (G4S)6-hFc; hTβRII-hFc; and hTβRII extended hinge-hFc proteins all were capable of inhibiting both TGFβ1 and TGFβ3. Interestingly, while there was a correlation between improved TGFβ1 and TGFβ3 inhibition and linker length for the hTβRII (G4S)2-hFc; hTβRII (G4S)3-hFc and hTβRII (G4S)₄-hFc constructs (FIG. 5E), this improvement trend appeared to have plateaued for hTβRII (G4S)5-hFc and hTβRII (G4S)6-hFc constructs (FIG. 5F).

### Example 3. mTβRII-mFc Treatment Suppresses Lung Fibrosis in a Bleomycin Mouse Model

Anti-fibrotic effects of mTβRII-mFc, a murine fusion protein that selectively binds to TGF-β1 and TGF-β3, were assessed in a bleomycin mouse model of lung fibrosis. Lung fibrosis in this model is induced by bleomycin infused through subcutaneously implanted mini-pumps. Bleomycin was administered at a dose of 100 U/kg over seven days at a rate of 1 uL/hr. This bleomycin infusion causes infiltration of inflammatory cells reminiscent of early-stage SSc (systemic sclerosis), leading to release of pro-fibrotic molecules (including TGF-β isoforms) that induce fibrotic lesions with the same subpleural localization found in human patients with lung fibrosis (e.g., SSc-ILD). This model was developed by Lee et al (2014) Am J Physiol Lung Cell Mol Physiol 15:306 (8): L736-48.

Male 8-week old C57BL/6J mice were obtained from Jackson Laboratories and divided into study groups (Table 2). Mice were administered saline (Groups 1 and 2) or bleomycin (Group 3-7) by osmotic mini-pumps implanted subcutaneously under the back skin for 7 days, after which pumps were removed. Starting on day 9, mice began receiving treatment twice per week, treatment comprising either mTβRII-mFc at a dose of 1 mg/kg, 3 mg/kg, or 10 mg/kg or PBS vehicle. At the completion of the study (day 9 after pump implantation for “Baseline” group and day 34, day 35, or day 36 for other groups), mice were euthanized for blood, bronchoalveolar lavage (BAL) and lung tissue collection.

TABLE 2
Mouse Lung Fibrosis Study Design

| | | | | Dosing | | Dosing | |
|---|---|---|---|---|---|---|---|
| Group | N | Injury | Treatment | Schedule | Dose | Conc. | Route |
| 1 | 10 | Saline | PBS | Twice per | 10 uL/g | PBS | s.c. |
| “Vehicle” (Saline) | | | | week for a | | | |
| | | | | duration of | | | |
| | | | | 24, 25, or 26 | | | |
| | | | | days | | | |
| 2 | 10 | Saline | mTβRII-mFc | Twice per | 10 mg/kg | 2 mg/mL | s.c. |
| “mTβRII-mFc | | | | week for a | | | |
| (10 mg/kg)” | | | | duration of | | | |
| (Saline) | | | | 24, 25, or 26 | | | |
| | | | | days | | | |
| 3 | 10 | Bleo | — | — | — | — | — |
| “Baseline” | | | | | | | |
| (Bleomycin) | | | | | | | |
| 4 | 10 | Bleo | PBS | Twice per | 10 uL/g | PBS | s.c. |
| “Vehicle” | | | | week for a | | | |
| (Bleomycin) | | | | duration of | | | |
| | | | | 24, 25, or 26 | | | |
| | | | | days | | | |
| 5 | 10 | Bleo | mTβRII-mFc | Twice per | 1 mg/kg | 0.2 mg/mL | s.c. |
| “mTβRII-mFc | | | | week for a | | | |
| (1 mg/kg)” | | | | duration of | | | |
| (Bleomycin) | | | | 24, 25, or 26 | | | |
| | | | | days | | | |
| 6 | 10 | Bleo | mTβRII-mFc | Twice per | 3 mg/kg | 0.6 mg/mL | s.c. |
| “mTβRII-mFc | | | | week for a | | | |
| (3 mg/kg)” | | | | duration of | | | |
| (Bleomycin) | | | | 24, 25, or 26 | | | |
| | | | | days | | | |
| 7 | 10 | Bleo | mTβRII-mFc | Twice per | 10 mg/kg | 2 mg/mL | s.c. |
| “mTβRII-mFc | | | | week for a | | | |
| (10 mg/kg)” | | | | duration of | | | |
| (Bleomycin) | | | | 24, 25, or 26 | | | |
| | | | | days | | | |

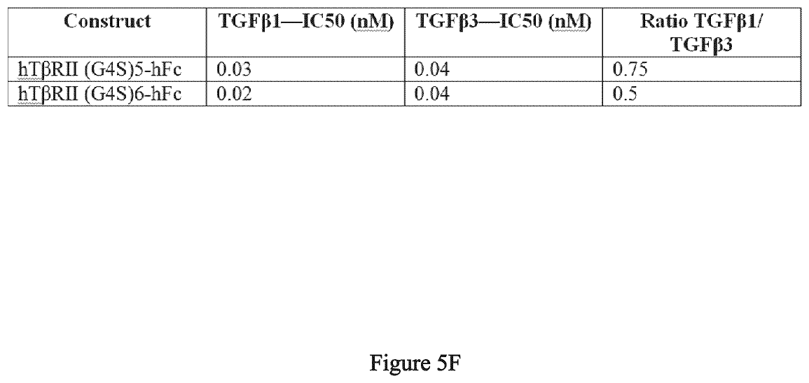

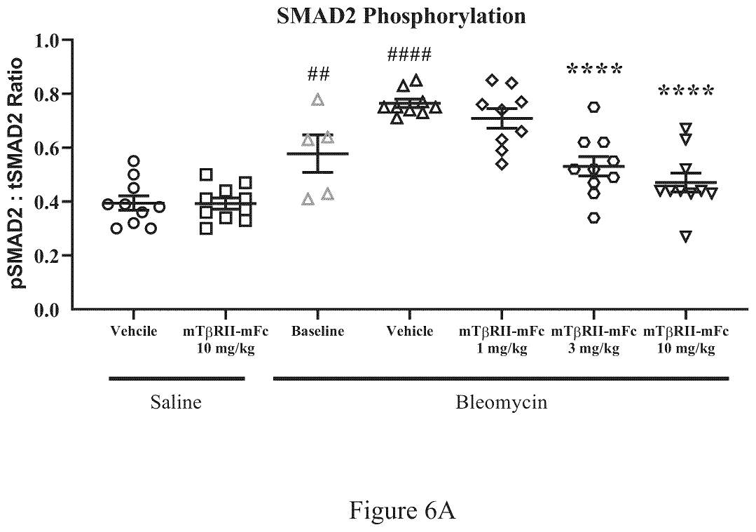

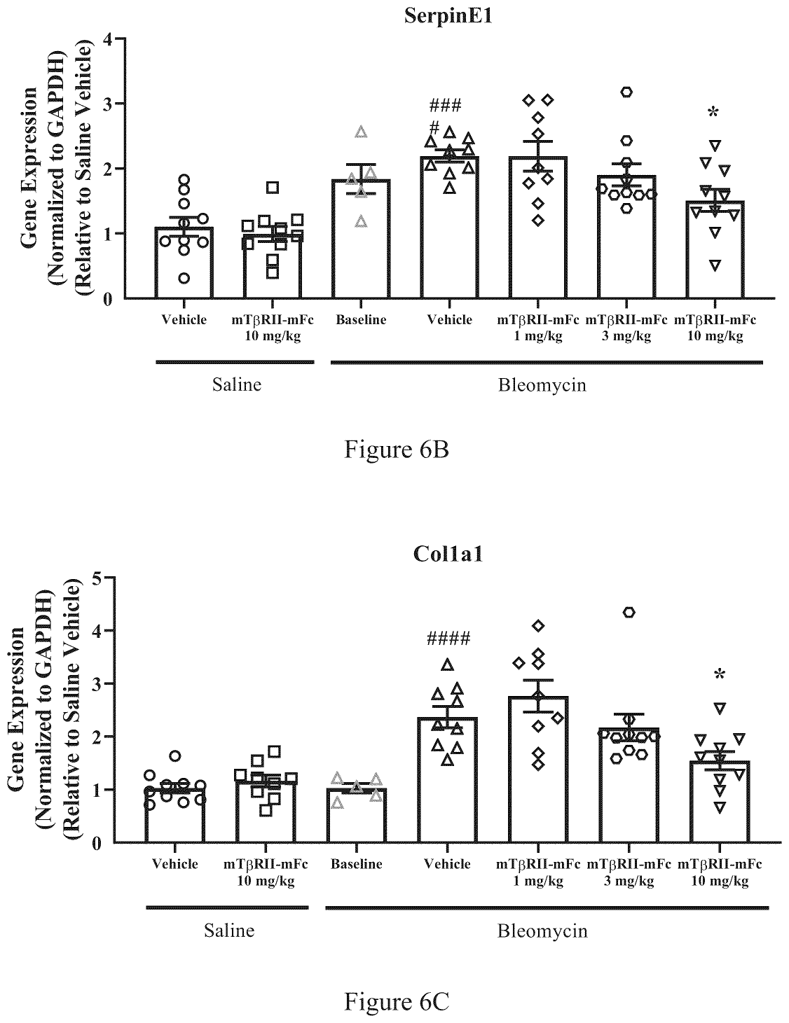

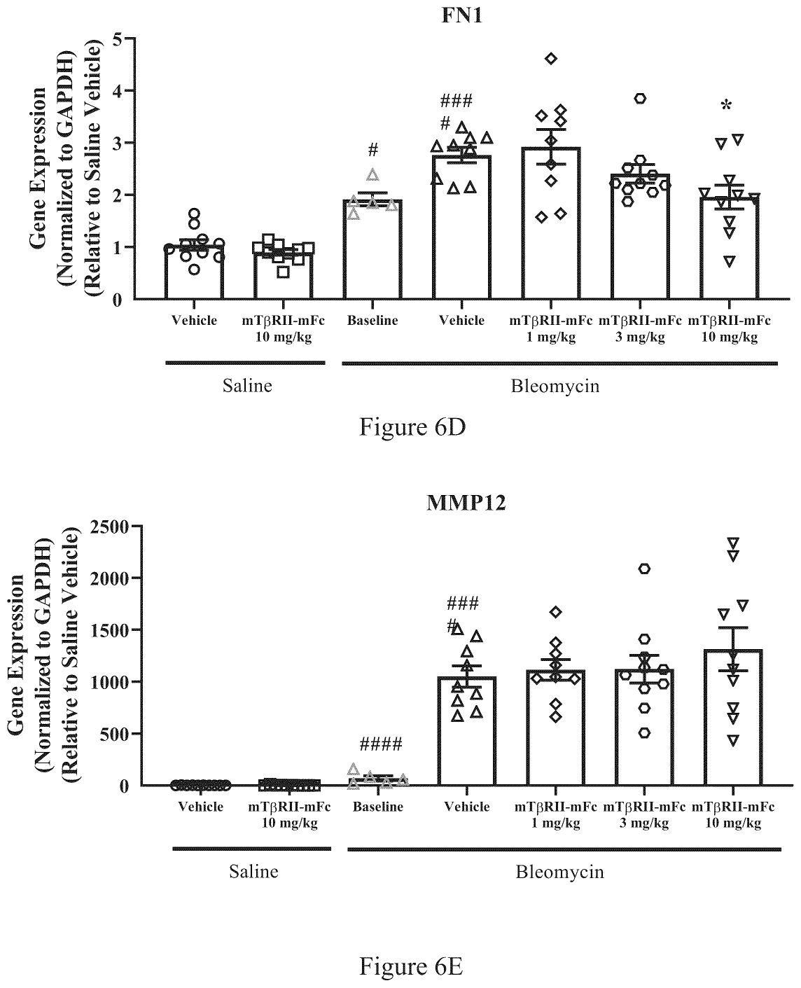

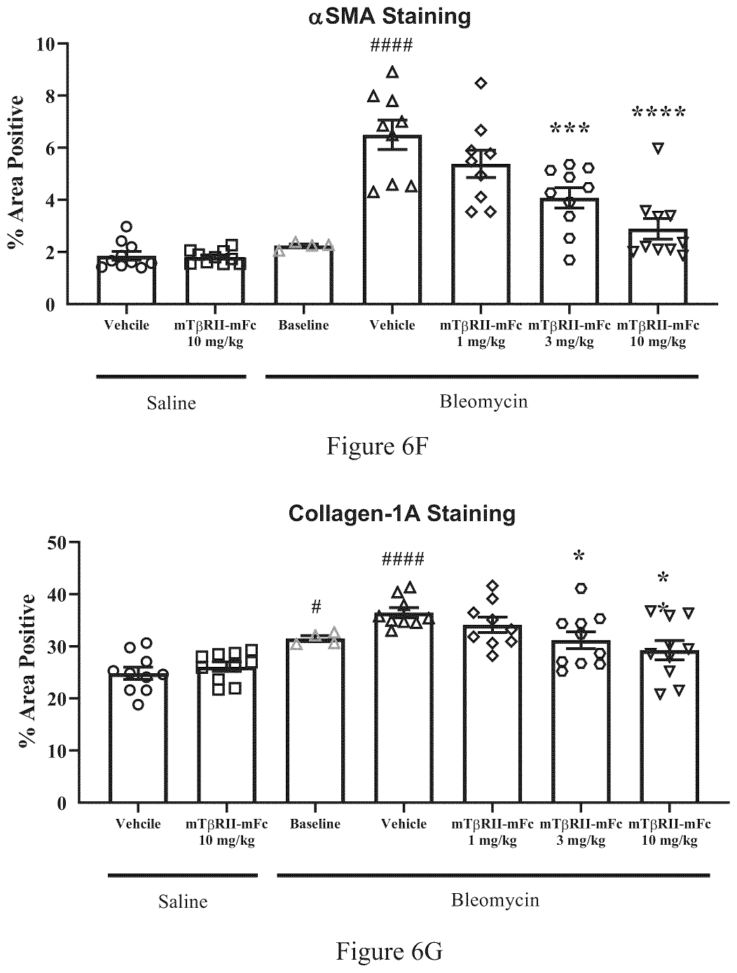

Activation of the TGFβRII receptor by TGF-β triggers phosphorylation of SMAD2, thus the extent of SMAD2 phosphorylation serves as a surrogate for TGF-β signaling. Levels of pSMAD2 and tSMAD2 were measured by an MSD assay with SMAD2 phosphorylation defined as the ratio of pSMAD2 signal to tSMAD2 signal. mTβRII-mFc dose-dependently inhibited elevated SMAD2 phosphorylation levels caused by bleomycin challenge, with statistically significant effects at both 3 mg/kg and 10 mg/kg doses of TβRII-mFc (FIG. 6A).

The extent of lung fibrosis was assessed using expression analysis of pro-fibrotic genes (SerpinE1, FN1 and Colla1) and immunohistochemistry (IHC) for aSMA and Collagen-1. Gene expression for SerpinE1 (PAI-1) (FIG. 6B), Colla1 (collagen-1) (FIG. 6C) and FN1 (fibronectin) (FIG. 6D) in lung tissue homogenate was determined by qPCR performed on a CFX Connect™ Real-time PCR detection system (Bio-Rad, CA). Phosphorylation of SMAD2 leads to downstream up-regulation of PAI-1, which contributes to fibrosis by inhibiting degradation of matrix proteins like collagen. Collagen-1 and fibronectin are major components of fibrotic lesions. Bleomycin administration increased expression of SerpinE1, Colla1 and FN1, effects that were all significantly reduced by mTβRII-mFc treatment at 10 mg/kg (FIGS. 6B-6D).

Matrix Metalloproteinase-12 (MMP12) can also be a down-stream target of TGF-β signaling, and increases in its expression have been linked to pro-inflammatory responses and lung diseases such as emphysema. At the doses tested, mTβRII-mFc showed no effect for increasing MMP12 levels in either saline or bleomycin-infused mice (FIG. 6E).

TGF-β induces lung fibroblasts to transition to aSMA-expressing myofibroblasts that deposit aberrant levels of collagen to form fibrotic plaques. To measure the extent of lung fibrosis, aSMA and collagen-1A levels in lung tissue collected at day 34, day 35, or day 36 were measured by IHC with quantitative image analysis using HALO software (Indica Lab Albuquerque, NM). Bleomycin-infusion resulted in significant elevations in the percent of lung tissue area staining positive for aSMA and collagen-1 (FIGS. 6F and 6G, respectively, comparing “Vehicle” (Saline) to “Vehicle” (bleomycin)). mTβRII-mFc dose-dependently reduced positive area of staining for both aSMA and collagen-1A compared to “Vehicle” (bleomycin), with significant effects at doses of 3 mg/kg and 10 mg/kg mTβRII-mFc (FIGS. 6F and 6G, respectively).

Together, these data demonstrate that mTβRII-mFc treatment suppresses lung fibrosis in an animal model. Moreover, these data indicate that other mTβRII-mFc fusion proteins may be useful in the treatment or prevention of lung fibrosis. A representative TβRII antagonist of the present disclosure was capable of decreasing levels of pro-fibrotic biomarkers and suppressing lung fibrosis in a fibrosis animal model. Accordingly, Applicants envision that the TβRII antagonists of the present disclosure may demonstrate similar anti-fibrotic effects in a patient with HPS, particularly those with one or more complications associated with HPS such as pulmonary fibrosis and/or ILD.

### Example 4. Common Bile Duct Ligation (CBDL) Mouse Model for Intrapulmonary Vasodilation

The methods and compositions of the present disclosure may be implemented in an animal model for intrapulmonary vasodilation and hepatopulmonary syndrome. To generate a model for intrapulmonary vasodilation and hepatopulmonary syndrome, male Sprague-Dawley rats will undergo common bile duct ligation (CBDL) as described in Fallon et. al., Am J Physiol. 1997 April; 272 (4 Pt 1): G779-84. In this procedure, the bile duct is mechanically obstructed using surgical knots. Laparotomy is performed on anesthetized rats to access the bile duct, followed by isolation and double ligation of the bile duct. Sham animals will undergo mobilization of the common bile duct without ligation. Cholestatic hepatic injury is expected to progress with the onset of increased portal pressure within two weeks following the CBDL procedure. Biliary cirrhosis and lung abnormalities are expected to develop by five weeks after CBDL. Measurements of plasma aspartate aminotransferase (AAT), alkaline phosphatase, total bilirubin, and hepatic histology will be performed on the CBDL and sham rats to assess the presence, absence, and/or development of hepatic injury during the weeks following surgery. A vascular catheter will be placed into the CBDL and sham rats within 24 to 48 hours prior to hemodynamic analysis.

In order to conduct hemodynamic analysis, including determining the presence and degree of intrapulmonary shunting, anesthetized animals will undergo placement of indwelling femoral arterial and venous catheters. Arterial blood gases will be drawn during the following day via extension tubing connected to the femoral arterial line. The catheters may be irrigated with heparinized saline and connected to a physiological pressure transducer for heart rate, mean aortic and pulmonary artery pressure measurements. Cardiac output may be measured using the dye-dilution method. The dye dilution method for measuring cardiac output is based on rapidly injecting a known quantity of a dye at one site into the circulatory system, and withdrawing blood at a distal site for determination of a concentration curve of the dye. Total systemic resistance and total pulmonary resistance may be calculated by dividing mean aortic and pulmonary artery pressures by cardiac index, where cardiac index is cardiac output per 100 g body weight.

Hemodynamic parameters may also be assessed using microspheres. Labeled cross-linked polystyrene-divinylbenzene microspheres (with a size and count range of approximately 5.5-10 μm and 2.5×10⁶ microspheres, respectively) in sterile water will be injected through the femoral vein catheter. To determine the amount of microspheres passing through the pulmonary microcirculation, the number of microspheres per mL of blood (as determined by a reference blood sample) is multiplied by the estimated blood volume. The blood volume of each animal will be calculated using the formula: blood volume (mL)=0.06× bodyweight (g)+0.77. The degree of intrapulmonary shunting will then be calculated as an intrapulmonary shunt fraction, where intrapulmonary shunt fraction (%)=(total microspheres passing through the pulmonary microcirculation/total beads injected into the venous circulation)×100. CBDL rats with increased intrapulmonary shunting will exhibit an increase in the number and size of microspheres that pass through the lungs as biliary cirrhosis and lung abnormalities develop over time (e.g., after five weeks following CBDL).

Finally, portal pressure will be assessed by direct puncture of the portal vein with a needle connected to a differential pressure transducer. 8-12 μm sections of the upper and lower lung fields from each lung will be obtained from each animal. Each section will be postfixed in acid/ethanol and stained with hematoxylin or substantially similar tissue stain. The lung sections will be analyzed for the presence of inflammatory cells, architectural abnormalities, and lung edema. Lung sections will also be dried and assessed for lung edema.

After approximately five weeks, cirrhotic and sham rats will be assessed for physiological and histological changes. Within two weeks from CBDL, rats will develop progressive intrapulmonary vasodilation. Progressive intrapulmonary vasodilation will correlate temporally with increasing portal pressure, progressive hepatic injury, and deteriorating arterial gas-exchange. At this time, CBDL rats will have developed progressive cholestatic hepatic injury. Cirrhotic rats are expected to exhibit lower basal total pulmonary resistance and impaired pulmonary gas exchange. Due to the presence of severe hepatic fibrosis, cirrhotic rats are expected to have marked increases in plasma alkaline phosphatase and total bilirubin, and are also expected to show hepatosplenomegaly. Systemic and pulmonary vasodilation in the cirrhotic group will produce increases in cardiac outputs with a decreases in total systemic and pulmonary vascular resistances. Rats with intrapulmonary shunts will display increased AaPO₂ when breathing 100% O₂ in comparison to sham-operated control rats. The onset and degree of intrapulmonary vasodilation expected in the cirrhotic rat model correlates with the development and severity of gas-exchange abnormalities typically observed in humans.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A method for treating hepatopulmonary syndrome (HPS), comprising administering to a subject in need thereof a transforming growth factor β receptor II (TβRII) antagonist.

2. The method of claim 1, wherein the subject has one or more complications of the lungs associated with HPS.

3. The method of claim 2, wherein the one or more complications of the lungs is selected from the group consisting of interstitial lung disease (ILD), pulmonary fibrosis, idiopathic pulmonary fibrosis, alveolitis, recurrent aspiration, and pulmonary vasculopathy.

4-13. (canceled)

14. A method for treating chronic liver disease, comprising administering to a subject in need thereof a transforming growth factor β receptor II (TβRII) antagonist.

15. The method of claim 14, wherein the chronic liver disease is selected from the group consisting of: primary sclerosing cholangitis, portal hypertension, cryptogenic cirrhosis, postnecrotic cirrhosis, alcoholic cirrhosis, autoimmune cirrhosis, primary biliary cirrhosis, primary biliary cholangitis, chronic active hepatitis, alpha-1 antitrypsin deficiency, cystic fibrosis, nonalcoholic fatty liver disease, galactosemia, alagille syndrome, autoimmune hepatitis, Wilson's disease, sarcoidosis, hemochromatosis, biliary atresia, portal hypertension, tyrosinemia, Gaucher disease, schistosomiasis, nodular regenerative hyperplasia of the liver, hepatic allograft rejection, Langerhans cell histiocytosis, hepatic graft-versus host disease following hematopoietic stem cell transplantation, and short telomere syndrome.

16. The method of claim 15, wherein the chronic liver disease is primary sclerosing cholangitis.

17. The method of claim 15, wherein the chronic liver disease is portal hypertension.

18. (canceled)

19. A method for treating one or more complications of chronic liver disease, comprising administering to a subject in need thereof a transforming growth factor β receptor II (TβRII) antagonist, wherein the one or more complications of chronic liver disease is selected from the group consisting of spider nevi (spider angiomata, spider angiomas, or nevi araneus), hyperdynamic circulation, clubbing of the fingers or toes, hypertrophic osteoarthropathy, orthodeoxia, cyanosis weakness, fatigue, anorexia, gastrointestinal bleeding, inability to concentrate, menstrual irregularities, decreased libido, skin fragility, bruising, pruritus, ascites, anasarca, nail changes, splenomegaly, a large or small liver, caput medusae, gynecomastia, palmar erythema, testicular atrophy, and/or jaundice.

20-132. (canceled)

133. The method of claim 1, wherein the TβRII antagonist comprises a TβRII extracellular domain, wherein the TβRII extracellular domain comprises an amino acid sequence at least 80% identical to:
i) a sequence beginning at any of positions 23 to 35 of SEQ ID NO: 1 and ending at any of positions 153 to 159 of SEQ ID NO: 1 or;
ii) a sequence beginning at any of positions 23 to 60 of SEQ ID NO: 2 and ending at any of positions 178 to 184 of SEQ ID NO: 2.

134-141. (canceled)

142. The method of claim 133, wherein the TβRII extracellular domain comprises the amino acid sequence of SEQ ID NO: 18.

143. The method of claim 142, wherein the TβRII antagonist is a fusion protein further comprising a heterologous domain.

144-161. (canceled)

162. The method of claim 1, wherein the TβRII antagonist comprises an amino acid sequence at least 95% identical to SEQ ID NO: 48.

163-170. (canceled)

171. The method of claim 14, wherein the TβRII antagonist comprises an amino acid sequence at least 95% identical to SEQ ID NO: 48.

172. The method of claim 19, wherein the TβRII antagonist comprises an amino acid sequence at least 95% identical to SEQ ID NO: 48.


SEQUENCE LISTING (88 sequences)

SEQ ID NO: 1 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 3 - protein, 4 aa, synthetic construct
  REGION 1..4: note = Description of Artificial Sequence: Synthetic peptide
TGGG

SEQ ID NO: 4 - protein, 11 aa, synthetic construct
  REGION 1..11: note = Description of Artificial Sequence: Synthetic peptide
TGGGGSGGGGS

SEQ ID NO: 5 - protein, 16 aa, synthetic construct
  REGION 1..16: note = Description of Artificial Sequence: Synthetic peptide
TGGGGSGGGGSGGGGS

SEQ ID NO: 6 - protein, 21 aa, synthetic construct
  REGION 1..21: note = Description of Artificial Sequence: Synthetic peptide
TGGGGSGGGGSGGGGSGGGGS

SEQ ID NO: 7 - protein, 10 aa, synthetic construct
  REGION 1..10: note = Description of Artificial Sequence: Synthetic peptide
TGGGPKSCDK

SEQ ID NO: 8 - DNA, 1248 bp, synthetic construct
  misc_feature 1..1248: note = Description of Artificial Sequence: Synthetic polynucleotide
  source 1..1248: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 415 aa, synthetic construct
  REGION 1..415: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1284 bp, synthetic construct
  misc_feature 1..1284: note = Description of Artificial Sequence: Synthetic polynucleotide
  source 1..1284: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - protein, 427 aa, synthetic construct
  REGION 1..427: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1299 bp, synthetic construct
  misc_feature 1..1299: note = Description of Artificial Sequence: Synthetic polynucleotide
  source 1..1299: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - protein, 432 aa, synthetic construct
  REGION 1..432: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1269 bp, synthetic construct
  misc_feature 1..1269: note = Description of Artificial Sequence: Synthetic polynucleotide
  source 1..1269: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 422 aa, synthetic construct
  REGION 1..422: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1266 bp, synthetic construct
  misc_feature 1..1266: note = Description of Artificial Sequence: Synthetic polynucleotide
  source 1..1266: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggacccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 17 - protein, 421 aa, synthetic construct
  REGION 1..421: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 162 aa, synthetic construct
  REGION 1..162: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 19 - protein, 5 aa, synthetic construct
  REGION 1..5: note = Description of Artificial Sequence: Synthetic peptide
GGGGS

SEQ ID NO: 20 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 10 aa, synthetic construct
  REGION 1..10: note = Description of Artificial Sequence: Synthetic peptide
GGGGSGGGGS

SEQ ID NO: 22 - protein, 22 aa, unidentified
  REGION 1..22: note = Description of Unknown: Native leader sequence
MGRGLLRGLWPLHIVLWTRIAS

SEQ ID NO: 23 - protein, 22 aa, unidentified
  REGION 1..22: note = Description of Unknown: Tissue plasminogen activator sequence
MDAMKRGLCCVLLLCGAVFVSP

SEQ ID NO: 24 - protein, 21 aa, Apis cerana
MKFLVNVALVFMVVYISYIYA

SEQ ID NO: 25 - protein, 26 aa, synthetic construct
  REGION 1..26: note = Description of Artificial Sequence: Synthetic peptide
TGGGGSGGGGSGGGGSGGGGSGGGGS

SEQ ID NO: 26 - protein, 31 aa, synthetic construct
  REGION 1..31: note = Description of Artificial Sequence: Synthetic polypeptide
TGGGGSGGGGSGGGGSGGGGSGGGGSGGGGS

SEQ ID NO: 27 - protein, 137 aa, synthetic construct
  REGION 1..137: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 28 - protein, 131 aa, synthetic construct
  REGION 1..131: note = Description of Artificial Sequence: Synthetic polypeptide
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 29 - protein, 125 aa, synthetic construct
  REGION 1..125: note = Description of Artificial Sequence: Synthetic polypeptide
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 30 - protein, 131 aa, synthetic construct
  REGION 1..131: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 31 - protein, 125 aa, synthetic construct
  REGION 1..125: note = Description of Artificial Sequence: Synthetic polypeptide
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 32 - protein, 119 aa, synthetic construct
  REGION 1..119: note = Description of Artificial Sequence: Synthetic polypeptide
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 33 - protein, 156 aa, synthetic construct
  REGION 1..156: note = Description of Artificial Sequence: Synthetic polypeptide
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - protein, 156 aa, synthetic construct
  REGION 1..156: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 35 - protein, 150 aa, synthetic construct
  REGION 1..150: note = Description of Artificial Sequence: Synthetic polypeptide
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 36 - protein, 137 aa, synthetic construct
  REGION 1..137: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 37 - protein, 162 aa, synthetic construct
  REGION 1..162: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 38 - protein, 131 aa, synthetic construct
  REGION 1..131: note = Description of Artificial Sequence: Synthetic polypeptide
TIPPHVQKSVNNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 39 - protein, 156 aa, synthetic construct
  REGION 1..156: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 40 - protein, 36 aa, synthetic construct
  REGION 1..36: note = Description of Artificial Sequence: Synthetic polypeptide
TGGGGSGGGGSGGGGSGGGGSGGGGSGGGGSGGGGS

SEQ ID NO: 41 - protein, 36 aa, synthetic construct
  REGION 1..36: note = Description of Artificial Sequence: Synthetic polypeptide
GRCKIRHIGSNNRLQRSTCQNTGWESAHVMKTPGFR

SEQ ID NO: 42 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 233 aa, Homo sapiens
GGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 437 aa, synthetic construct
  REGION 1..437: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 442 aa, synthetic construct
  REGION 1..442: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1314 bp, synthetic construct
  misc_feature 1..1314: note = Description of Artificial Sequence: Synthetic polynucleotide
  source 1..1314: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 47 - DNA, 1329 bp, synthetic construct
  misc_feature 1..1329: note = Description of Artificial Sequence: Synthetic polynucleotide
  source 1..1329: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggaagtggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 48 - protein, 408 aa, synthetic construct
  REGION 1..408: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 162 aa, synthetic construct; identical to SEQ ID NO: 18
  REGION 1..162: note = Description of Artificial Sequence: Synthetic polypeptide

SEQ ID NO: 50 - protein, 410 aa, synthetic construct
  REGION 1..410: note = Description of Artificial Sequence: Synthetic polypeptide
GATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 408 aa, synthetic construct; identical to SEQ ID NO: 48
  REGION 1..408: note = Description of Artificial Sequence: Synthetic polypeptide

SEQ ID NO: 52 - protein, 409 aa, synthetic construct
  REGION 1..409: note = Description of Artificial Sequence: Synthetic polypeptide
ATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 407 aa, synthetic construct
  REGION 1..407: note = Description of Artificial Sequence: Synthetic polypeptide
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 406 aa, synthetic construct
  REGION 1..406: note = Description of Artificial Sequence: Synthetic polypeptide
PPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 405 aa, synthetic construct
  REGION 1..405: note = Description of Artificial Sequence: Synthetic polypeptide
PHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 404 aa, synthetic construct
  REGION 1..404: note = Description of Artificial Sequence: Synthetic polypeptide
HVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 10 aa, synthetic construct
  REGION 1..10: note = Description of Artificial Sequence: Synthetic peptide
  REGION 1..10: note = See specification as filed for detailed description of substitutions and preferred embodiments
GGGGSGGGGS

SEQ ID NO: 58 - skipped

SEQ ID NO: 59 - protein, 20 aa, synthetic construct
  REGION 1..20: note = Description of Artificial Sequence: Synthetic peptide
GGGGSGGGGSGGGGSGGGGS

SEQ ID NO: 60 - skipped

SEQ ID NO: 61 - protein, 6 aa, synthetic construct
  REGION 1..6: note = Description of Artificial Sequence: Synthetic 6xHis tag
HHHHHH

SEQ ID NO: 62 - protein, 17 aa, synthetic construct
  REGION 1..17: note = Description of Artificial Sequence: Synthetic peptide
NTSNPDTGGGTHTCPPC

SEQ ID NO: 63 - protein, 24 aa, synthetic construct
  REGION 1..24: note = Description of Artificial Sequence: Synthetic peptide
NTSNPDTGGGGSGGGGSTHTCPPC

SEQ ID NO: 64 - protein, 29 aa, synthetic construct
  REGION 1..29: note = Description of Artificial Sequence: Synthetic peptide
NTSNPDTGGGGSGGGGSGGGGSTHTCPPC

SEQ ID NO: 65 - protein, 34 aa, synthetic construct
  REGION 1..34: note = Description of Artificial Sequence: Synthetic polypeptide
NTSNPDTGGGGSGGGGSGGGGSGGGGSTHTCPPC

SEQ ID NO: 66 - protein, 23 aa, synthetic construct
  REGION 1..23: note = Description of Artificial Sequence: Synthetic peptide
NTSNPDTGGGPKSCDKTHTCPPC

SEQ ID NO: 67 - protein, 407 aa, synthetic construct
  REGION 1..407: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 426 aa, synthetic construct
  REGION 1..426: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 431 aa, synthetic construct
  REGION 1..431: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 421 aa, synthetic construct
  REGION 1..421: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 420 aa, synthetic construct
  REGION 1..420: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 224 aa, synthetic construct
  REGION 1..224: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 222 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 74 - protein, 232 aa, Homo sapiens
GGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 436 aa, synthetic construct
  REGION 1..436: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 441 aa, synthetic construct
  REGION 1..441: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 409 aa, synthetic construct
  REGION 1..409: note = Description of Artificial Sequence: Synthetic polypeptide
GATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 407 aa, synthetic construct; identical to SEQ ID NO: 67
  REGION 1..407: note = Description of Artificial Sequence: Synthetic polypeptide

SEQ ID NO: 79 - protein, 408 aa, synthetic construct
  REGION 1..408: note = Description of Artificial Sequence: Synthetic polypeptide
ATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 406 aa, synthetic construct
  REGION 1..406: note = Description of Artificial Sequence: Synthetic polypeptide
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 405 aa, synthetic construct
  REGION 1..405: note = Description of Artificial Sequence: Synthetic polypeptide
PPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 404 aa, synthetic construct
  REGION 1..404: note = Description of Artificial Sequence: Synthetic polypeptide
PHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 403 aa, synthetic construct
  REGION 1..403: note = Description of Artificial Sequence: Synthetic polypeptide
HVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 427 aa, synthetic construct
  REGION 1..427: note = Description of Artificial Sequence: Synthetic polypeptide
MDAMKRGLCCVLLLCGAVFVSPGAIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 85 - protein, 403 aa, synthetic construct
  REGION 1..403: note = Description of Artificial Sequence: Synthetic polypeptide
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 86 - protein, 402 aa, synthetic construct
  REGION 1..402: note = Description of Artificial Sequence: Synthetic polypeptide
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLG

SEQ ID NO: 87 - DNA, 1284 bp, synthetic construct
  misc_feature 1..1284: note = Description of Artificial Sequence: Synthetic polynucleotide
  source 1..1284: mol_type = other DNA
atggatgcaatgaagagagggctctgctgtgtgctgctgctgtgtggagcagtcttcgtttcgcccggcgccatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgacaccggtgggggtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 88 - protein, 414 aa, synthetic construct
  REGION 1..414: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG